(19) United States
(12) Patent Application Publication
(10) Pub. No.: US 2025/0074950 A1
(43) Pub. Date: Mar. 6, 2025

(54) METHODS FOR PRODUCTION OF THERAPEUTIC IMMUNE CELLS HAVING ENHANCED METABOLIC FITNESS AND COMPOSITIONS THEREOF

(71) Applicant: BOARD OF REGENTS, THE UNIVERSITY OF TEXAS SYSTEM, Austin, TX (US)

(72) Inventors: May DAHER, Houston, TX (US); Rafet BASAR, Houston, TX (US); Sunil ACHARYA, Houston, TX (US); Katy REZVANI, Houston, TX (US); Ana Karen NUNEZ CORTES, Houston, TX (US); Nadima UPRETY, Houston, TX (US); Emily ENSLEY, Houston, TX (US)

(73) Assignee: BOARD OF REGENTS, THE UNIVERSITY OF TEXAS SYSTEM, Austin, TX (US)

(21) Appl. No.: 18/700,483

(22) PCT Filed: Oct. 21, 2022
(86) PCT No.: PCT/US2022/078495

§ 371 Date: Apr. 11, 2024

Related U.S. Application Data
(60) Provisional application No. 63/270,423, filed on Oct. 21, 2021.

(51) Int. Cl. C07K 14/005; A61K 39/00; A61P 35/02; C07K 14/54; C07K 14/705; C07K 14/725; C07K 16/28; C12N 5/0783

(52) U.S. Cl. CPC C07K 14/005; A61K 40/15; A61K 40/31; A61K 40/4224; A61P 35/02; C07K 14/5443; C07K 14/7051; C07K 14/70517; C07K 14/70521; C07K 16/2875; C12N 5/0646; C07K 2317/53; C07K 2319/02; C07K 2319/03; C12N 2510/00; C12N 2710/10022; C12N 2710/24122; C12N 2770/24122

(57) ABSTRACT

Aspects of the present disclosure include methods and compositions related to therapeutic immune cells having enhanced metabolic fitness. In certain aspects, polynucleotides encoding one or more viral, bacterial, and/or fungal genes capable of manipulating cell metabolism and, optionally, one or more antigen-specific receptors, are disclosed. In some aspects, disclosed are methods for enhancing the metabolic fitness of an immune cell comprising introducing into the immune cell a polynucleotide encoding one or more viral, bacterial, and/or fungal genes capable of manipulating cell metabolism. Cells (e.g., NK cells, T cells) expressing polynucleotides encoding one or more viral, bacterial, and/or fungal genes capable of manipulating cell metabolism and, optionally, one or more antigen-specific receptors are described. Also described are therapeutic methods using polynucleotides of the disclosure.

### CROSS-REFERENCE TO RELATED APPLICATIONS

This application claims priority to U.S. Provisional Application Ser. No. 63/270,423, filed Oct. 21, 2021, which is incorporated by reference herein in its entirety.

### SEQUENCE LISTING

The instant application contains a Sequence Listing which has been submitted in XML format and is hereby incorporated by reference in its entirety. Said XML copy, created on Sep. 30, 2022, is named “MDACP1315WO-Sequence-Listing.xml” and is 195,595 bytes in size.

### BACKGROUND

### I. Technical Field

Aspects of the disclosure include at least the fields of cell biology, molecular biology, immunology, and medicine, including cancer medicine.

### II. Background

Recent discoveries in the field of immunometabolism provides us with the exciting opportunities to metabolically reprogram immune cells to enhance and optimize their therapeutic efficacy. Cellular metabolism relies on two important components to extract energy: glycolysis in the cytosol followed by mitochondrial oxidative phosphorylation (OXPHOS) under aerobic conditions. In the absence of oxygen, cells rely on glycolysis rather than mitochondrial metabolism for their energy supply. In immune cells, glycolysis has been asserted as a prerequisite for effector functions, such as increased production of granzyme B and IFN-γ,¹⁻⁴ whereas OXPHOS has been shown to enhance memory formation and persistence.⁵⁻⁶ Hence both glycolysis and OXPHOS are essential for the optimal antitumor activity of immune cells. Developing strategies to make these processes more efficient in adoptively transferred immune cells will enable them to compete with the high metabolic demands of tumor cells and function better in the adverse, often hypoxic and nutrient deplete, tumor microenvironment.

There exists a need for methods and compositions for enhancing the metabolic fitness of therapeutic immune cells for cancer treatment.

### SUMMARY

Aspects of the disclosure encompass methods and compositions related to polynucleotides that encode one or more viral, bacterial, and/or fungal genes capable of manipulating cell metabolism, and optionally, one or more antigen-specific receptors, including as chimeric antigen receptors (CARs), immune cell engagers (e.g., bispecific or multispecific engagers), and the like. In certain aspects, disclosed are polynucleotides that encode one or more viral, bacterial, and/or fungal genes capable of increasing glycolysis, oxidative phosphorylation, fatty acid synthesis, glutaminolysis, or a combination thereof in a cell, including an immune cell. In certain aspects, expression by a cell of the one or more viral, bacterial, and/or fungal genes capable of manipulating cell metabolism and/or the one or more antigen-specific receptors encoded by the polynucleotides enhances the metabolic fitness of the cell, for example, by increasing the cell's metabolism, and/or enhances one or more anti-tumor activities of the cell. In certain aspects, disclosed are polynucleotides encoding engineered polypeptides such as CARs and TCRs comprising an antigen-binding region. In specific aspects, the polypeptides of the disclosure that target one or more antigens are expressed by and comprised on the surface of cells of any kind, including immune cells.

Aspects of the present disclosure include polynucleotides, polypeptides, vectors, expression constructs, viral genes, bacterial genes, fungal genes, viral proteins, bacterial proteins, fungal proteins, engineered receptors, chimeric antigen receptors, pharmaceutical compositions, methods for generating and expressing polynucleotides, methods for expressing viral protein(s), methods for expressing bacterial protein(s), methods for expressing fungal protein(s), methods for generating and expressing an antigen-specific receptor, methods for generating and expressing a CAR, methods for generating and expressing a TCR, methods for generating a cell expressing viral protein(s), methods for generating a cell expressing bacterial protein(s), methods for generating a cell expressing fungal protein(s), methods for generating a cell expressing viral protein(s), bacterial protein(s), and/or fungal proteins(s) and antigen-specific receptor(s), methods for generating a CAR T cell, methods for generating a CAR NK cell that also expresses viral protein(s), bacterial protein(s), and/or fungal proteins(s), methods for generating a CAR T cell that also expresses viral protein(s), bacterial protein(s), and/or fungal proteins(s), methods for generating a CAR NK cell that also expresses viral protein(s), bacterial protein(s), and/or fungal proteins(s), methods for treating a subject for cancer, and methods for enhancing the metabolic fitness of cells (e.g., immune cells). Polypeptides of the disclosure can include at least 1, 2, 3, or more of: viral gene(s) encoding viral proteins, bacterial gene(s) encoding bacterial proteins, fungal gene(s) encoding fungal proteins, antigen binding regions, a CD70-binding region, a variable heavy chain region, a variable light chain region, a transmembrane domain, an intracellular domain, a costimulatory domain, a hinge region, a signal peptide, a polypeptide linker, and an immune cell binding region. Any one of more of the preceding components may be excluded from polypeptides of the disclosure in certain aspects.

In particular aspects, disclosed are polypeptides (e.g., viral, bacterial, and/or fungal genes, antibodies, chimeric antigen receptors, immune cell engagers) comprising a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with any of SEQ ID NOs: 41-94. In some aspects, disclosed are polypeptides comprising any one or more of SEQ ID NOs: 41-94.

Also presented herein are vectors comprising a polynucleotide of the disclosure. Vectors contemplated herein include viral vectors (e.g., adenoviral vectors, adeno-associated viral vectors, lentiviral vectors, and retroviral vectors) and non-viral vectors (e.g., plasmids).

Aspects of the disclosure include immune cells of any kind comprising any polynucleotide and/or polypeptide encompassed herein. In specific aspects, the immune cell is a NK cell, T cell, gamma delta (γδ) T cell, alpha beta (αβ) T cell, invariant NKT (INKT) cell, B cell, macrophage, MSC, or dendritic cell. In cases wherein the immune cell is an NK cell, the NK cell may be derived from cord blood (including pooled cord blood units), peripheral blood, induced pluripotent stem cells, bone marrow, and/or from a cell line. In specific aspects, the NK cell line is NK-92 cell line or another NK cell line derived from a tumor or from a healthy NK cell or a progenitor cell. In cases where the immune cell is a T cell, the T cell may be derived from cord blood (including pooled cord blood units), peripheral blood, induced pluripotent stem cells, bone marrow, and/or from a cell line.

In specific aspects, the immune cell is an NK cell, such as one derived from cord blood, such as from a cord blood mononuclear cell. The NK cell may be a CD56⁺ NK cell, in specific cases. The NK cells may express one or more exogenously provided cytokines, such as IL-15, IL-2, IL-12, IL-18, IL-21, IL-23, IL-7, or a combination thereof. Particular aspects include populations of immune cells of any kind of the disclosure, and the cells may be present in a suitable medium or a suitable carrier of any kind.

Methods of treating or preventing cancer of any kind are encompassed herein, including by administering cells expressing particular viral proteins, and/or other proteins derived from other microorganisms (e.g., bacteria, fungi), and/or antigen-specific receptors at a therapeutically effective amount to decrease tumor burden or increase survival of a subject having the cancer, ameliorate or prevent the cancer, or reduce the risk of the cancer, reduce the severity of the cancer, prevent metastasis or risk thereof, or delay the onset of the cancer. In some aspects, disclosed is a method of killing cancer cells in an individual, which may or may not be positive for one or more antigens disclosed herein, comprising administering to the individual an effective amount of cells harboring any polynucleotide and/or polypeptide of the disclosure (e.g., one or more viral, bacterial, and/or fungal genes capable of manipulating cell metabolism and/or the one or more antigen-specific receptors).

Methods of enhancing the metabolic fitness of cells of any kind are encompassed herein, including by introducing into the cells polynucleotide(s) encoding particular viral proteins, and/or other proteins derived from other microorganisms (e.g., bacteria, fungi), and/or antigen-specific receptors to increase metabolism of the cells.

In specific aspects, the cells are NK cells, T cells, gamma delta T cells, alpha beta T cells, invariant NKT (INKT) cells, B cells, macrophages, mesenchymal stromal cells (MSCs), or dendritic cells. NK cells may be derived from cord blood, peripheral blood, induced pluripotent stem cells, hematopoietic stem cells, bone marrow, or from a cell line. NK cells may be derived from cord blood mononuclear cells. In some cases, the cancer cells are from hematopoietic cancers or solid tumors. The cells may be allogeneic or autologous with respect to the individual, who may or may not be a human. The cells may be administered to the individual by injection, intravenously, intraarterially, intraperitoneally, intrapleurally, intratracheally, intratumorally, intramuscularly, endoscopically, intralesionally, intracranially, percutaneously, subcutaneously, regionally, by perfusion, in a tumor microenvironment, or a combination thereof.

In particular aspects of the methods, the cells may be administered to the individual once or more than once. The duration of time between administrations of the cells to the individual may be 1-24 hours, 1-7 days, 1-4 weeks, 1-12 months, or 1 or more years. The methods may further comprise the step of providing to the individual an effective amount of an additional therapy, such as surgery, radiation, gene therapy, immunotherapy, and/or hormone therapy. The additional therapy may comprise one or more antibodies or antibody-based agents, in some cases. In some aspects to the methods, they may further comprise the step of identifying antigen-positive cancer cells in the individual.

Disclosed herein, in some aspects, is one or more polynucleotides encoding one or more viral, bacterial, and/or fungal genes capable of manipulating cell metabolism and one or more antigen-specific receptors. In some aspects, the one or more viral, bacterial, and/or fungal genes are capable of increasing glycolysis, oxidative phosphorylation, fatty acid synthesis, glutaminolysis, or a combination thereof in a cell.

In some aspects of the one or more polynucleotides, the one or more viral genes comprise an adenovirus, vaccinia virus, hepatitis C virus (HCV), hepatitis B virus (HBV), Epstein-Barr virus (EBV), and/or Dengue virus (DENV) gene. In specific aspects, the adenovirus gene comprises E4ORF-1. In specific aspects, the vaccinia virus gene comprises C16. In specific aspects, the DENV gene comprises NS3. In specific aspects, the HCV gene comprises NS5A. In specific aspects, the HBV gene comprises ORFx. In specific aspects, the EBV gene comprises LMP1.

In some aspects of the one or more polynucleotides, the one or more viral, bacterial, and/or fungal genes and one or more antigen-specific receptors are encoded by the same polynucleotide. In other aspects, the one or more viral, bacterial, and/or fungal genes and one or more antigen-specific receptors are encoded by different polynucleotides.

In some aspects the polynucleotides are introduced alone or as part of engineered receptor constructs via stable viral vectors, in other aspects the polynucleotides can be introduced by electroporation for transient expression of mRNA that would be translated to protein inside the cells, and in other aspects the polynucleotides can be introduced using knock-in approaches using gene editing technologies including but not limited to CRISPR, TALENS, Zinc fingers, retrons among others. The knock-in approaches can introduce the polynucleotides in specific favorable genomic locations such as under the promoter of hypoxia-inducible factor-1 α (HIF-1 α), or other promoters that are activated in the tumor microenvironment.

In some aspects of the one or more polynucleotides, the one or more antigen-specific receptors each comprise: (a) one or more antigen binding regions; (b) a transmembrane domain; and (c) one or more intracellular domains.

In some aspects, the antigen binding region comprises a linker.

In some aspects, the transmembrane domain is a transmembrane domain from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, DAP10, or DAP12. In some aspects, the transmembrane domain is a CD28 transmembrane domain. In some aspects, the intracellular domain is an intracellular domain from CD3 zeta, CD27, CD28, 4-1BB, DAP12, NKG2D, OX-40 (CD134), DAP10, CD40L, 2B4, DNAM, CS1, CD48, NKp30, NKp44, NKp46, NKp80, or any ITAM-containing signaling domain.

In some aspects, the intracellular domain is a CD28 intracellular domain. In some aspects, the intracellular domain is a CD3 zeta intracellular domain. In some aspects, the one or more antigen-specific receptors comprise two or more intracellular domains. In some aspects, the two or more intracellular domains comprise a CD3 zeta intracellular domain and an additional intracellular domain selected from a CD28, DAP10, DAP12, 4-1BB, NKG2D, ICOS, and 2B4 intracellular domain. In specific aspects, the two or more intracellular domains comprise a CD3 zeta intracellular domain and a CD28 intracellular domain.

In some aspects, the one or more antigen-specific receptors further comprise a hinge between the antigen binding domain and the transmembrane domain. In some aspects, the hinge is an IgG hinge, a CD28 hinge, or a CD8a hinge. In some aspects, the hinge is IgG1 hinge, IgG2 hinge, IgG3 hinge, or IgG4 hinge. In specific aspects, the hinge is an IgG1 hinge. In some aspects, the hinge is a CD28 hinge.

In some aspects of the one or more polynucleotides, the one or more polynucleotides further encode a signal peptide. In some aspects, the signal peptide is a signal peptide from CD8, CD27, granulocyte-macrophage colony-stimulating factor receptor (GMSCF-R), Ig heavy chain, a killer cell immunoglobulin-like receptor (KIR), CD3, or CD4. In specific aspects, the signal peptide is a CD8 signal peptide.

In some aspects of the one or more polynucleotides, the one or more polynucleotides further encode an additional polypeptide. In some aspects, the additional polypeptide is a therapeutic protein or a protein that enhances cell activity, expansion, and/or persistence. In some aspects, the additional polypeptide is a suicide gene, a cytokine, or a human or viral protein that enhances proliferation, expansion and/or metabolic fitness. In specific aspects, the additional polypeptide is a cytokine. In some aspects, the cytokine is IL-15, IL-2, IL-12, IL-18, IL-21, IL-23, or IL-7. In specific aspects, the cytokine is IL-15. In specific aspects, the cytokine is IL-21. In specific aspects, the cytokine is IL-12.

In specific aspects of the one or more polynucleotides, the one or more antigen-specific engineered receptors comprise a chimeric antigen receptor (CAR). In specific aspects of the polynucleotide, the one or more antigen-specific engineered receptors comprise a T-cell receptor (TCR).

In some aspects of the one or more polynucleotides, the one or more antigen-specific engineered receptors bind one or more antigens comprising 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRVIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors. In some aspects, the one or more antigen-specific engineered receptors bind one or more antigens comprising CD70, CD5, CD19, CD22, BCMA, CS1, CD123, CD38, CLL-1, CD97, and/or HLA-G. In specific aspects, the one or more antigen-specific engineered receptors bind CD70.

Also disclosed herein, in some aspects, is a vector comprising a polynucleotide encoding one or more viral, bacterial, and/or fungal genes capable of manipulating cell metabolism and one or more antigen-specific receptors. In specific aspects, the vector is a viral vector. In some aspects, the viral vector is an adenoviral vector, adeno-associated viral vector, lentiviral vector, or retroviral vector. In specific aspects, the vector is a non-viral vector. In some aspects, the non-viral vector is a plasmid.

Also disclosed herein, in some aspects, is an immune cell comprising a polynucleotide encoding one or more viral, bacterial, and/or fungal genes capable of manipulating cell metabolism and one or more antigen-specific receptors or a vector comprising a polynucleotide encoding one or more viral, bacterial, and/or fungal genes capable of manipulating cell metabolism and one or more antigen-specific receptors. In some aspects, the immune cell is a natural killer (NK) cell, T cell, gamma delta T cell, alpha beta T cell, invariant NKT (INKT) cell, B cell, macrophage, mesenchymal stromal cell, or dendritic cell. In specific aspects, the immune cell is an NK cell. In some aspects, the NK cell is derived from cord blood, peripheral blood, induced pluripotent stem cells, hematopoietic stem cells, bone marrow, or from a cell line. In some aspects, the NK cell is derived from a cell line, wherein the NK cell line is NK-92. In some aspects, the NK cell is derived from a cord blood mononuclear cell. In some aspects, the NK cell is a CD56⁺ NK cell. In some aspects, the NK cell expresses a recombinant cytokine. In some aspects, the cytokine is IL-15, IL-2, IL-12, IL-18, IL-21, IL-7, or IL-23. In specific aspects, the cytokine is IL-15. In specific aspects, the cytokine is IL-21. In specific aspects, the cytokine is IL-12.

In some aspects, expression by the immune cell of the one or more viral, bacterial, and/or fungal genes capable of manipulating cell metabolism and/or the one or more antigen-specific receptors encoded by the polynucleotide enhances the metabolic fitness of the immune cell and/or enhances one or more anti-tumor activities of the immune cell. In some aspects, the metabolism of the immune cell is increased compared to an immune cell into which the polynucleotide encoding one or more viral, bacterial, and/or fungal genes capable of manipulating cell metabolism has not been introduced. In some aspects, glycolysis, oxidative phosphorylation, fatty acid synthesis, glutaminolysis, or a combination thereof is increased by the immune cell. In some aspects, glycolysis is increased by the immune cell.

Also disclosed herein, in some aspects, is a population of immune cells comprising immune cells comprising a polynucleotide encoding one or more viral, bacterial, and/or fungal genes capable of manipulating cell metabolism and one or more antigen-specific receptors or a vector comprising a polynucleotide encoding one or more viral, bacterial, and/or fungal genes capable of manipulating cell metabolism and one or more antigen-specific receptors.

Disclosed herein, in some aspects, is an immune cell comprising a polynucleotide encoding one or more viral, bacterial, and/or fungal genes capable of manipulating cell metabolism. In some aspects, the immune cell is a natural killer (NK) cell, T cell, gamma delta T cell, alpha beta T cell, invariant NKT (INKT) cell, B cell, macrophage, mesenchymal stromal cell, or dendritic cell. In specific aspects, the immune cell is an NK cell. In some aspects, the NK cell is derived from cord blood, peripheral blood, induced pluripotent stem cells, hematopoietic stem cells, bone marrow, or from a cell line. In some aspects, the NK cell is derived from a cell line, wherein the NK cell line is NK-92. In some aspects, the NK cell is derived from a cord blood mononuclear cell. In specific aspects, the NK cell is a CD56˜ NK cell. In some aspects, the NK cell expresses a recombinant cytokine. In some aspects, the cytokine is IL-15, IL-2, IL-12, IL-18, IL-21, IL-7, or IL-23. In specific aspects, the cytokine is IL-15. In specific aspects, the cytokine is IL-21. In specific aspects, the cytokine is IL-12.

In some aspects of the immune cell, the one or more viral genes comprise an adenovirus, vaccinia virus, HCV, HBV, and/or DENV gene. In specific aspects, the adenovirus gene comprises E4ORF-1. In specific aspects, the vaccinia virus gene comprises C16. In specific aspects, the DENV gene comprises NS3. In specific aspects, the HCV gene comprises NS5A. In specific aspects, the HBV gene comprises ORFx. In specific aspects, the EBV gene comprises LMP1.

In some aspects of the immune cell, the polynucleotide encoding one or more viral, bacterial, and/or fungal genes capable of manipulating cell metabolism is comprised in a vector. In specific aspects, the vector is a viral vector. In some aspects, the viral vector is an adenoviral vector, adeno-associated viral vector, lentiviral vector, or retroviral vector. In specific aspects, the vector is a non-viral vector. In some aspects, the non-viral vector is a plasmid.

In some aspects of the immune cell, expression by the immune cell of the one or more viral, bacterial, and/or fungal genes capable of manipulating cell metabolism encoded by the polynucleotide enhances the metabolic fitness of the immune cell. In some aspects, the metabolism of the immune cell is increased compared to an immune cell into which the polynucleotide encoding one or more viral, bacterial, and/or fungal genes capable of manipulating cell metabolism has not been introduced. In some aspects, glycolysis, oxidative phosphorylation, fatty acid synthesis, glutaminolysis, or a combination thereof is increased by the immune cell. In some aspects, glycolysis is increased by the immune cell.

Also disclosed herein, in some aspects, is a population of immune cells comprising a polynucleotide encoding one or more viral, bacterial, and/or fungal genes capable of manipulating cell metabolism.

Disclosed herein, in some aspects, is a pharmaceutical composition comprising: (a) an immune cell or population thereof comprising a polynucleotide encoding one or more viral, bacterial, and/or fungal genes capable of manipulating cell metabolism and one or more antigen-specific receptors or a vector comprising a polynucleotide encoding one or more viral, bacterial, and/or fungal genes capable of manipulating cell metabolism and one or more antigen-specific receptors; or an immune cell or population thereof comprising a polynucleotide encoding one or more viral, bacterial, and/or fungal genes capable of manipulating cell metabolism; and (b) a pharmaceutically acceptable excipient. In some aspects, the pharmaceutical composition further comprises an additional therapeutic. In some aspects, the additional therapeutic is a chemotherapeutic.

Disclosed herein, in some aspects, is a method for treating a subject for cancer, the method comprising administering to the subject a therapeutically effective amount of (i) (a) an immune cell or population thereof comprising a polynucleotide encoding one or more viral, bacterial, and/or fungal genes capable of manipulating cell metabolism and one or more antigen-specific receptors or a vector comprising a polynucleotide encoding one or more viral, bacterial, and/or fungal genes capable of manipulating cell metabolism and one or more antigen-specific receptors; (ii) an immune cell or population thereof comprising a polynucleotide encoding one or more viral, bacterial, and/or fungal genes capable of manipulating cell metabolism; or (iii) a pharmaceutical composition comprising: (a) an immune cell or population thereof comprising a polynucleotide encoding one or more viral, bacterial, and/or fungal genes capable of manipulating cell metabolism and one or more antigen-specific receptors or a vector comprising a polynucleotide encoding one or more viral, bacterial, and/or fungal genes capable of manipulating cell metabolism and one or more antigen-specific receptors; or an immune cell or population thereof comprising a polynucleotide encoding one or more viral, bacterial, and/or fungal genes capable of manipulating cell metabolism; and (b) a pharmaceutically acceptable excipient.

In some aspects, administration of a therapeutically effective amount of (i), (ii), or (iii) decreases tumor burden or increases survival of the subject. In some aspects, the subject has lymphoma, leukemia, glioblastoma, melanoma, non-small cell lung cancer, renal cell carcinoma, pancreatic cancer, ovarian cancer, or breast cancer.

In some aspects, the method further comprises administering to the subject an additional therapy. In some aspects, the additional therapy is radiotherapy, chemotherapy, or immunotherapy.

Disclosed herein, in some aspects, is a method for enhancing the metabolic fitness of an immune cell, the method comprising introducing into the immune cell a polynucleotide encoding one or more viral, bacterial, and/or fungal genes capable of manipulating cell metabolism, wherein the metabolism of the immune cell is increased compared to an immune cell into which the polynucleotide encoding one or more viral, bacterial, and/or fungal genes capable of manipulating cell metabolism has not been introduced. In some aspects, glycolysis, oxidative phosphorylation, fatty acid synthesis, glutaminolysis, or a combination thereof is increased by the immune cell. In some aspects, glycolysis is increased by the immune cell.

In some aspects of the method, the one or more viral genes comprise an adenovirus, vaccinia virus, HCV, HBV, and/or DENV gene. In specific aspects, the adenovirus gene comprises E4ORF-1. In specific aspects, the vaccinia virus gene comprises C16. In specific aspects, the DENV gene comprises NS3. In specific aspects, the HCV gene comprises NS5A. In specific aspects, the HBV gene comprises ORFx. In specific aspects, the EBV gene comprises LMP1.

In some aspects, the method further comprises introducing into the immune cell a polynucleotide encoding one or more antigen-specific engineered receptors. In specific aspects, the one or more viral, bacterial, and/or fungal genes and one or more antigen-specific receptors are encoded by the same polynucleotide. In specific aspects, the one or more viral, bacterial, and/or fungal genes and one or more antigen-specific receptors are encoded by different polynucleotides.

In some aspects of the method, the one or more antigen-specific receptors each comprise: (a) one or more antigen binding regions; (b) a transmembrane domain; and (c) one or more intracellular domains.

In some aspects, the antigen binding region comprises a linker.

In some aspects, the transmembrane domain is a transmembrane domain from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, DAP10, or DAP12. In some aspects, the transmembrane domain is a CD28 transmembrane domain. In some aspects, the intracellular domain is an intracellular domain from CD3 zeta, CD27, CD28, 4-1BB, DAP12, NKG2D, OX-40 (CD134), DAP10, CD40L, 2B4, DNAM, CS1, CD48, NKp30, NKp44, NKp46, or NKp80.

In some aspects, the intracellular domain is a CD28 intracellular domain. In some aspects, the intracellular domain is a CD3 zeta intracellular domain. In some aspects, the one or more antigen-specific receptors comprise two or more intracellular domains. In some aspects, the two or more intracellular domains comprise a CD3 zeta intracellular domain and an additional intracellular domain selected from a CD28, DAP10, DAP12, 4-1BB, NKG2D, ICOS, and 2B4 intracellular domain. In specific aspects, the two or more intracellular domains comprise a CD3 zeta intracellular domain and a CD28 intracellular domain.

In some aspects, the one or more antigen-specific receptors further comprise a hinge between the antigen binding domain and the transmembrane domain. In some aspects, the hinge is an IgG hinge, a CD28 hinge, or a CD8a hinge. In some aspects, the hinge is IgG1 hinge, IgG2 hinge, IgG3 hinge, or IgG4 hinge. In specific aspects, the hinge is an IgG1 hinge. In some aspects, the hinge is a CD28 hinge.

In some aspects of the method, the polynucleotide further encodes a signal peptide. In some aspects, the signal peptide is a signal peptide from CD8, CD27, granulocyte-macrophage colony-stimulating factor receptor (GMSCF-R), Ig heavy chain, a killer cell immunoglobulin-like receptor (KIR), CD3, or CD4. In specific aspects, the signal peptide is a CD8 signal peptide.

In some aspects of the method, the polynucleotide further encodes an additional polypeptide. In some aspects, the additional polypeptide is a therapeutic protein or a protein that enhances cell activity, expansion, and/or persistence. In some aspects, the additional polypeptide is a suicide gene, a cytokine, or a human or viral protein that enhances proliferation, expansion and/or metabolic fitness. In specific aspects, the additional polypeptide is a cytokine. In some aspects, the cytokine is IL-15, IL-2, IL-12, IL-18, IL-21, IL-23, or IL-7. In specific aspects, the cytokine is IL-15. In specific aspects, the cytokine is IL-21. In specific aspects, the cytokine is IL-12.

In specific aspects of the method, the one or more antigen-specific engineered receptors comprise a chimeric antigen receptor (CAR). In specific aspects of the polynucleotide, the one or more antigen-specific engineered receptors comprise a T-cell receptor (TCR).

In some aspects of the method, the one or more antigen-specific engineered receptors bind one or more antigens comprising 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRVIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors. In some aspects, the one or more antigen-specific engineered receptors bind one or more antigens comprising CD70, CD5, CD19, CD22, BCMA, CS1, CD123, CD38, CLL-1, CD97, and/or HLA-G. In specific aspects, the one or more antigen-specific engineered receptors bind CD70.

In some aspects of the method, the polynucleotide encoding one or more viral, bacterial, and/or fungal genes capable of manipulating cell metabolism is comprised in a vector. In specific aspects, the vector is a viral vector. In some aspects, the viral vector is an adenoviral vector, adeno-associated viral vector, lentiviral vector, or retroviral vector. In specific aspects, the vector is a non-viral vector. In some aspects, the non-viral vector is a plasmid.

In some aspects, the method further comprises administering a therapeutically effective amount of the immune cells having enhanced metabolic fitness or a pharmaceutical composition comprising the immune cells having enhanced metabolic fitness and a pharmaceutically acceptable excipient to a subject having cancer. In some aspects, the pharmaceutical composition further comprises an additional therapeutic. In specific aspects, the additional therapeutic is a chemotherapeutic. In some aspects, administration of a therapeutically effective amount of the immune cells having enhanced metabolic fitness or the pharmaceutical composition comprising the immune cells having enhanced metabolic fitness and a pharmaceutically acceptable excipient decreases tumor burden or increases survival of the subject. In some aspects, the subject has lymphoma, leukemia, glioblastoma, melanoma, non-small cell lung cancer, renal cell carcinoma, pancreatic cancer, ovarian cancer, or breast cancer.

In some aspects, the method further comprises administering to the subject an additional therapy. In some aspects, the additional therapy is radiotherapy, chemotherapy, or immunotherapy.

It is specifically contemplated that any limitation discussed with respect to one aspect of the disclosure may apply to any other aspect of the disclosure. Furthermore, any composition of the disclosure may be used in any method of the disclosure, and any method of the disclosure may be used to produce or to utilize any composition of the disclosure. Any aspect discussed with respect to one embodiment of the disclosure applies to other embodiments of the disclosure as well and vice versa. For example, any step in a method described herein can apply to any other method. Moreover, any method described herein may have an exclusion of any step or combination of steps. Aspects of an embodiment set forth in the Examples are also aspects that may be implemented in the context of aspects discussed elsewhere in a different Example or elsewhere in the application, such as in the Summary, Detailed Description, Claims, and Brief Description of the Drawings.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIGS. 1A-1B show CD70 expression on AML patient samples using Tsne plots.

FIG. 2 shows that E4ORF-1 increases the metabolic fitness of CAR-NK cells as measured by a seahorse glycostress assay showing increased extracellular acidification rate (ECAR) in E4ORF-1-modified cells compared to control non-transduced (NT) and CD70/IL-15 transduced NK cells.

FIG. 3 shows that E4ORF-1 increases the anti-tumor activity of CAR-NK cells as measured by bioluminescent imaging (BLI), which shows a difference in tumor burden (THP-1 AML cells transduced with firefly luciferase) between mice receiving control CD70 CAR-NK cells versus E4ORF-1-modified CD70 CAR-NK cells.

FIG. 4 shows that E4ORF-1 increases the anti-tumor activity of CAR-NK cells based on the increased survival of mice receiving E4ORF-1-modified CD70 CAR-NK cells compared to mice receiving control CD70 CAR-NK cells.

FIG. 5 shows a schematic diagram of enhanced glycolysis and glutamine-mediated oxidative phosphorylation upon E4ORF-1 expression due to upregulation of the Akt, mTORC1, c-myc pathways.

FIGS. 6A-6E show E4ORF-1 enhances the metabolic fitness of CAR27/IL-15 NK cells. FIG. 6A. Graph showing ECAR as a measure of glycolytic capacity of the various NK conditions. FIG. 6B. Graph showing OCR as a measure of oxidative phosphorylation. FIG. 6C. Bar graph showing the MFI of 2-NDBG uptake by E4ORF-modified CAR-NK cells compared to control CAR-NK cells. FIG. 6D. Western blot showing expression of various proteins associated with the glycolytic pathway in E4ORF-modified CAR27/IL-15 NK cells vs control CAR27/IL-15 NK cells. FIG. 6E. Western blot showing expression of proteins associated with glutamine metabolism in E4ORF-modified CAR27/IL-15 NK cells vs control CAR27/IL-15 NK cells.

FIG. 7 shows E4ORF-1 enhances the killing of renal cell carcinoma cells (UMRC3). Graph showing normalized cell index of UMRC3 cultured alone or after addition of various NK cell conditions at 1:1 effector to target (E:T) ratio. E4ORF-1-modified NK cells (non-engineered, engineered with IL-15 or with CAR27/IL-15 NK cells) cause decrease in normalized cell index a surrogate of higher cytotoxicity, compared to their respective controls lacking E4ORF-1.

FIG. 8 shows E4ORF-1 enhances the cytotoxicity of NK cells against renal cell carcinoma cells (UMRC3) after repeat tumor challenge. Graph showing normalized cell index of UMRC3 cultured alone or after addition of various NK cell conditions at 1:1 effector to target (E:T) ratio. E4ORF-1-expressing CAR-NK cells cause a decrease in the normalized tumor cell index, a surrogate for higher cytotoxicity, compared to their respective controls lacking E4ORF-1, and the advantage persists after a second tumor rechallenge. Black arrows indicate time points at which fresh tumor cells were added to the culture.

FIGS. 9A-9B show E4ORF-1 enhances the cytotoxicity of NK cells against pancreatic tumor cells (Panc1) under reduced glucose and reduced glutamine conditions. FIG. 9A. Graph showing normalized cell index of Panc1 cultured alone or after addition of various NK cell conditions at 2:1 effector to target (E:T) ratio under reduced glucose concentration (50%). E4ORF-1-expressing NK cells (non-engineered NK cells or NK cells engineered to express IL-15 or CAR27/IL-15) caused a decrease in normalized tumor cell index (a surrogate for higher cytotoxicity), compared to their respective controls lacking E4ORF-1. FIG. 9B. Graph showing normalized cell index of Panc1 cultured alone or in the presence of different NK cell conditions at 2:1 effector to target (E:T) ratio under reduced glutamine concentration (50%). E4ORF-1-modified NK cells (non-engineered or engineered with IL-15 or with CAR27/IL-15) cause a decrease in the normalized tumor cell index, compared to their respective controls lacking E4ORF-1.

### DETAILED DESCRIPTION

The present disclosure is based, at least in part, on the development of polynucleotides encoding one or more viral, bacterial, and/or fungal genes capable of manipulating cell metabolism and/or one or more antigen-specific receptors, including scFvs, portions thereof, and various polypeptides (e.g., antibodies, CARs, engagers) comprising such scFvs or portions thereof. To enhance the metabolic fitness of immune cells, e.g., NK cells, the inventors have devised a strategy inspired by how viruses alter the metabolism of infected host cells. For example, viruses hijack host cell metabolism to support the bioenergetic and biosynthetic demands of viral replication and provide the molecular building blocks needed to produce a large number of viral progenies.⁸ Each virus can use a unique mechanism to manipulate the host cell metabolism. For example, adenovirus relies on the protein E4ORF-1 for activation of c-MYC⁹ to enhance glucose and glutamine uptake in infected cells. This can lead to increased glycolysis and oxidative phosphorylation (OXPHOS) and nucleotide biosynthesis in the cell, enabling optimal adenovirus replication. Since glycolysis and OXPHOS are important for NK cell cytotoxicity against tumors, in some aspects, introducing E4ORF-1 in a CAR construct enhances the metabolic fitness and anti-leukemic activity of NK cells against cancer.

Accordingly, provided herein, in certain aspects, are methods and compositions concerning viral, bacterial, and/or fungal genes and corresponding viral, bacterial, or fungal proteins, optionally expressed in combination with engineered antigen-specific polypeptides, for therapy to target cancers by enhancing the metabolic fitness of immune cells, e.g., NK cells. Certain aspects of the present disclosure are directed to polynucleotides encoding viral protein polypeptides, antigen-targeted polypeptides (e.g., chimeric antigen receptors or T cell receptors) and therapeutic methods of use. Additionally, described are methods for cancer treatment comprising use of polypeptides encoding viral protein polypeptides and/or antigen-targeted polypeptides (e.g., chimeric antigen receptors or T cell receptors) of the disclosure and cells comprising such polynucleotides or polypeptides, as well as methods for enhancing the metabolic fitness of immune cells comprising such polynucleotides or polypeptides.

### I. Examples of Definitions

In keeping with long-standing patent law convention, the words “a” and “an” when used in the present specification in concert with the word comprising, including the claims, may mean “one” but is also consistent with the meaning of “one or more,” “at least one,” and “one or more than one.” Some aspects of the disclosure may consist of or consist essentially of one or more elements, method steps, and/or methods of the disclosure. It is contemplated that any method or composition described herein can be implemented with respect to any other method or composition described herein and that different aspects may be combined.

Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

Reference throughout this specification to “one aspect,” “an aspect,” “a particular aspect,” “a related aspect,” “a certain aspect,” “an additional aspect,” or “a further aspect” or combinations thereof means that a particular feature, structure, or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect.

Throughout this application, the term “about” is used according to its plain and ordinary meaning in the area of cell and molecular biology to indicate that a value includes the standard deviation of error for the measurement or quantitation device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, such as that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides that are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “sample,” as used herein, generally refers to a biological sample. The sample may be taken from tissue or cells from an individual. In some examples, the sample may comprise, or be derived from, a tissue biopsy, blood (e.g., whole blood), blood plasma, extracellular fluid, dried blood spots, cultured cells, discarded tissue. The sample may have been isolated from the source prior to collection. Non-limiting examples include blood, cerebral spinal fluid, pleural fluid, amniotic fluid, lymph fluid, saliva, urine, stool, tears, sweat, or mucosal excretions, and other bodily fluids isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some cases, the primary source is homogenized prior to further processing. The sample may be filtered or centrifuged to remove buffy coat, lipids, or particulate matter. The sample may also be purified or enriched for nucleic acids or may be treated with RNases. The sample may contain tissues or cells that are intact, fragmented, or partially degraded.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may be undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

### II. Viral Activation of Cellular Metabolism

One or more viral genes capable of manipulating cell metabolism may be encoded by a polynucleotide and expressed by a cell, e.g., an immune cell, as described herein. In some aspects, the one or more viral genes can increase the metabolism, e.g., glycolysis, oxidative phosphorylation, fatty acid synthesis, glutaminolysis, or a combination thereof, in a cell, such that expression by a cell, e.g., an immune cell, of the one or more viral genes capable of manipulating cell metabolism encoded by the polynucleotide enhances the metabolic fitness of the cell and/or enhances one or more anti-tumor activities of the cell.

To ensure optimal environments for their replication and spread, viruses have evolved to alter many host cell pathways. Viruses are obligate intracellular parasites, and their reproduction entirely relies on the host cell machinery for the synthesis of viral components such as nucleic acids, proteins, and membranes. Most viruses consist of single-stranded RNA or double-stranded DNA genome which is surrounded by either capsid proteins (non-enveloped viruses) or both capsid proteins and a lipid/protein membrane (enveloped viruses). After host cell attachment, viruses are internalized by clathrin-mediated endocytosis or micropinocytosis and subsequently escape from the endosomal vacuole into the cytosol. Here, the viral genome is released and transported to cellular compartments, where viral replication occurs: DNA viruses and some RNA viruses enter the nucleus, whereas most RNA viruses remain in the cytosol. After synthesis of the viral genome and proteins which assemble to new virus particles (virions), a complex release/egress process from the host cell is initiated: enveloped viruses egress by budding or exocytosis whereas most non-enveloped viruses are released by host cell lysis.

Virus formation depends on the metabolic capacity of the host cell to provide the necessary low molecular metabolites, i.e., nucleotides, amino acids and fatty acids (FAs)/lipids and energy in form of ATP. Most viruses manipulate the host cell's metabolism to optimize the biosynthetic needs of the virus through proviral metabolic changes. In particular, many viruses induce aerobic glycolysis also known as the Warburg effect. Many viruses also induce fatty acid synthesis as well as glutaminolysis. These modifications of carbon source utilization by infected cells can increase available energy for virus replication and virion production, provide specific cellular substrates for virus particles and create viral replication niches while increasing infected cell survival.

Viruses pursue different strategies to meet these metabolic requirements, but most viruses interact at some point during their replication cycle with the PI3K/Akt/mTOR pathway through binding of viral factors to the p85 adaptor or the p110 catalytic subunit of PI3K to inhibit host cell death and/or to modulate cellular metabolism. This signaling pathway is critically involved in the regulation of cell growth, (anti-) apoptosis, translation, but also the basic carbon metabolism. Several other signal pathways and regulatory factors converge with the PI3K/Akt/mTOR pathway at various points thereby positively or negatively affecting these processes. Viral components may modulate directly or indirectly this pathway at different steps in a virus-specific manner.

Another frequent target for viral factors affecting the host cell metabolism is AMPK. Activated AMPK stimulates energy-producing processes but inhibits energy-consuming anabolic processes, especially protein synthesis, by antagonizing mTOR kinase. AMPK and mTOR are therefore crucial regulators for cellular metabolism, energy homeostasis and growth.

Viruses change the central carbon metabolism of the infected host cells, sometimes by the permanent activation of cellular (proto)-oncogenes (e.g., Myc), the inactivation of tumor suppressors (e.g., p53) or by the introduction of virus-specific oncogenes as in case of certain tumor DNA viruses (e.g., large and small T antigens of simian virus 40 (SV40)).

Oxygen tension may also significantly affect the replication of several DNA and RNA viruses by modulating the rate of the host energy metabolism. This occurs often through stabilization of HIF-1a and manipulation of the HIF-1 pathway which, as further outlined below, also represents a frequent target for specific viral products.

Autophagy is a mechanism of host immune defense against viral infections by delivering viral antigens to the endosomal/lysosomal compartments for major histocompatibility complex (MHC)-mediated presentation or through direct elimination of the viruses by xenophagic degradation. But some viruses may actively subvert autophagy for their benefit by a variety of mechanisms, among others by providing additional nutrients for the host cell metabolism thereby supporting viral replication.

These metabolic alterations, which may be caused by the interaction of virus-specific factors with these host cell targets, lead to: (a) the induction of core catabolic pathways, i.e., glycolysis, PPP, TCA, β-oxidation of FAs (FAO), as well as anabolic pathways, i.e., enhanced biosynthesis of nucleotides, FAs/lipids and amino acids; and (b) induction of virus-specific biosynthetic processes (e.g., synthesis of virus-specific FAs and lipids) and modifications of viral components (e.g., virus-specific protein glycosylation or modifications of cellular FAs). These and other host cell metabolic changes in response to viral infection are described in, e.g., Eisenreich W. et al. (2019). Frontiers in Cellular and Infection Microbiology 9:42, incorporated by reference herein in its entirety.

Together, the virus-mediated metabolic reprogramming force the host cells to provide increased amounts of nucleotides necessary for viral nucleic acid replication, of amino acids necessary for virion assembly and of FA/lipids required for membrane formation necessary for the viral replication machinery and eventually for membrane envelopes. The increased generation of ATP is necessary for nucleic acid replication and virion packaging. In addition, virus-specific modifications of proteins (e.g., by glycosylation), nucleic acids, and FAs may be required for the generation of infectious virus particles.

As disclosed herein, in some cases, one or more viral genes capable of manipulating cell metabolism are present on the same polynucleotide or vector molecule as an engineered antigen-specific receptor, although in other cases they are on separate polynucleotides or vector molecules. In particular aspects, one or more viral genes are co-expressed from the same polynucleotide or vector as the engineered antigen-specific receptor. One or more viral gene products may be produced as a separate polypeptide from an antigen-specific receptor.

As one example, adenovirus E4ORF-1 is utilized as the viral gene capable of manipulating cell metabolism. E4ORF-1 may be employed because, for example, E4ORF-1 can activate glycolysis and nucleotide synthesis for DNA replication or glutaminolysis to generate amino acids and hexosamine pathway intermediates. Immune cells, e.g., NK cells, expressing E4ORF-1 may be utilized and are capable of manipulating cell metabolism, which is useful for enhancing the metabolic fitness of the immune cells such that the immune cells are better able to compete with the high metabolic demands of tumor cells and to function better in the adverse, often hypoxic and nutrient depleted, tumor microenvironment.

As another example, vaccinia virus C16 is utilized as the viral gene capable of manipulating cell metabolism. C16 may be employed because, for example, stabilizes HIF-1 through binding to the prolylhydroxylase domain-containing protein (PHD)2, a cellular oxygen sensor, which may activate glutaminolysis. Immune cells, e.g., NK cells, expressing C16 may be utilized and are capable of manipulating cell metabolism, which is useful for enhancing the metabolic fitness of the immune cells such that the immune cells are better able to compete with the high metabolic demands of tumor cells and to function better in the adverse, often hypoxic and nutrient depleted, tumor microenvironment.

As another example, Dengue virus nonstructural protein 3 (NS3) is utilized as the viral gene capable of manipulating cell metabolism. NS3 may be employed because, for example, stimulates fatty acid synthase activity to increase overall fatty acid synthesis in host cells. Immune cells, e.g., NK cells, expressing NS3 may be utilized and are capable of manipulating cell metabolism, which is useful for enhancing the metabolic fitness of the immune cells such that the immune cells are better able to compete with the high metabolic demands of tumor cells and to function better in the adverse, often hypoxic and nutrient depleted, tumor microenvironment.

In specific aspects, immune cells, e.g., NK cells, express one or more exogenously provided viral genes capable of manipulating cell metabolism. The viral gene(s) capable of manipulating cell metabolism may be exogenously provided to the immune cells, e.g., NK cells, because it is expressed from an expression vector within the cell. In cases wherein the viral gene(s) capable of manipulating cell metabolism are provided on an expression construct to the cell, the viral gene(s) may be encoded from the same vector as an antigen-specific receptor and/or a suicide gene. The viral gene(s) may be expressed as a separate polypeptide molecule from an antigen-specific receptor and/or a suicide gene. In some aspects, the present disclosure concerns co-utilization of CAR and/or TCR vectors with viral gene(s), particularly in NK cells.

### A. Glycolysis

In specific aspects, the one or more exogenously provided viral genes capable of manipulating cell metabolism expressed by immune cells, e.g., NK cells, are capable of increasing glycolysis.

Primary mammalian cells under standard growth conditions predominantly utilize glucose for oxidative phosphorylation in the mitochondria. Glucose is metabolized to pyruvate through multiple steps. Pyruvate is then translocated to the mitochondria, where it enters the TCA cycle and ultimately drives the electron transport chain, in a process that requires oxygen. In anaerobic conditions, glucose is primarily utilized for glycolysis where it is metabolized to pyruvate and then is converted to lactate and pumped out of the cell. In most cancer cells glucose is primarily utilized for the production of lactic acid even in the presence of abundant oxygen, a process often referred to as aerobic glycolysis or the Warburg effect. While oxidative phosphorylation provides significantly more ATP per glucose, glycolysis is a much faster process providing ATP rapidly. However, utilizing glycolysis as the main metabolic pathway for glucose requires increased uptake of extracellular glucose to match the increased metabolic rate.

If glucose uptake and utilization is increased, ATP can be produced more rapidly through aerobic glycolysis. Therefore, viruses may have evolved to induce glycolysis for a rapid source of ATP for replication. It is also possible that glycolysis increases biomass for a growing cell that viruses can use for replication or for the maintenance of latently infected cells. Increased glucose uptake may also feed other metabolic pathways during viral infection, including fatty acid synthesis.

The mechanisms for achieving this goal include: (a) activation of central signaling cascades modulating cellular metabolism (especially of PI3K/Akt/mTORC1, HIF-1a, and AMPK) by specific viral factors, (b) inhibition or degradation of the tumor suppressor p53 by the interaction of specific viral proteins with p53 directly (thereby inhibiting the transcriptional activity of p53) or with the proteasomal degradation machinery (thereby enhancing degradation of p53), (c) direct interaction of viral factors with specific metabolic enzymes, and (d) interaction of viral factors with specific metabolic regulators, like the carbohydrate-responsive element-binding protein (ChREBP) and/or the sterol regulatory element-binding protein (SREBP).

Adenovirus (ADV), a non-enveloped double stranded DNA virus, induces glycolysis, leading to increased glucose consumption and lactic acid production with a concurrent decrease in oxygen consumption. Adenovirus infection increases in glycolysis are mediated by early adenovirus gene product E4ORF1 (also known as OR “E4orf1” OR “E4 open reading frame 1” OR “early region 4 open reading frame 1”) binding to cellular MYC to direct transcription of specific glycolytic enzymes, including hexokinase-2 (HK2) and phosphofructokinase (PFK). Expression of E4ORF1 is sufficient to induce glycolysis. E4ORF1 induces and co-immunoprecipitates with MYC, and its interaction with MYC facilitates the induction of glycolysis. Adenovirus infection leads to increased carbon flux into nucleotides, and when radiolabeled glucose is added to infected cell media, adenovirus DNA is labeled. Carbon flux into nucleotides and adenovirus DNA replication is blunted when cells are infected with the adenovirus mutant where E4ORF1 could not activate MYC, and an adenovirus containing the D68A point mutation in E4ORF1 that prevents binding to MYC does not replicate as well. Therefore, in some aspects, adenovirus induces MYC to activate glycolysis and nucleotide synthesis for adenovirus DNA replication. E4ORF1 also targets PI3K. Another ADV-encoded protein involved in metabolic reprogramming includes E1A, which also targets MYC.

Human cytomegalovirus (HCMV) infection also induces the production of glycolytic intermediates. Flux analysis has demonstrated an increased flux of glucose carbon through glycolysis ultimately leading to an increase in lactate production. Subsequent studies examined the mechanism of HCMV induction of glucose uptake and glycolysis. Viral protein expression is required for HCMV induced glycolysis, and early genes are used for HCMV induction of glycolysis. HCMV also alters glucose transporter expression. Mechanistically, HCMV-encoded major immediate-early protein IE72 alters expression of glucose transporters in infected cells; it eliminates the ubiquitously expressed glucose transporter-1 (GLUT1) protein and increases mRNA and protein levels of GLUT4, which has three times higher affinity for glucose than GLUT1 and is a more efficient glucose transporter that can accelerate glucose uptake. GLUT4 upregulation is dependent on carbohydrate-response element binding protein (ChREBP), which is highly elevated at both the mRNA and protein levels during HCMV infection, and ChREBP knockdown reduces GLUT4 mRNA levels and subsequently glucose consumption and lactate production. ChREBP knockdown in host cells also decreases HCMV replication. Therefore, HCMV induces a shift in glucose transporter expression allowing increased glucose accumulation in infected cells. Other HCMV-encoded proteins involved in metabolic reprogramming include IE1 and IE2, which target Akt; pUL38, which targets TSC/AMPK; and pUL37x1, which targets mTORC1 and/or CaMKK/AMPK.

Human papillomavirus (HPV) is a double-stranded DNA virus and is the oncogenic virus found in the largest number of cancer cases. HPV infection produces a number of viral proteins that affect host cell metabolism. The HPV viral proteins E6 and E7 augment hypoxia-inducible factor 1-α (HIF-1a), which may result in an enhanced glycolytic phenotype in a hypoxic solid tumor microenvironment. E6 stabilizes HIF-1a under hypoxic conditions by inhibiting von Hippel-Lindau E3 ubiquitin ligase association with and ubiquitination of HIF-1a. In cells treated with hypoxia mimetic deferoxamine mesylate, E7 is able to enhance HIF-1a activation of target genes. HPV type 16 E7 interacts directly with pyruvate kinase M2 (PKM2) and promotes its dimeric state. This decreases PKM2's affinity for phosphoenolpyruvate (PEP) in the final step of glycolysis and may be a means of diverting glycolytic intermediates for anabolic purposes while compensating for the decreased energy production with upregulated glutamine metabolism. Additionally, the HPV viral protein E2 has been shown to interact directly with the mitochondrial membrane and induce release of ROS as well as to upregulate HIF-1α. HPV-encoded E6, E7, and E2 proteins involved in metabolic reprogramming also target Akt/TORC1, SGLT1, and PI3K/Akt.

Hepatitis B virus (HBV) is a double-strand DNA virus associated with the development of hepatocellular carcinoma (HCC). HBV infection has broad effects on host cell metabolism, impacting lipid, glucose, amino acid, nucleic acid, vitamin, and bile acid metabolism. HBV core protein (HBc) has been shown to upregulate multiple metabolic pathways, including glycolysis and amino acid metabolism. The HBV pre-S2 mutant protein upregulates GLUT1 expression and plasma membrane localization. HBV X protein (HBx), which is encoded by ORFx, upregulates glucose-6-phosphate dehydrogenase (G6PD) and expression of multiple genes involved in gluconeogenesis.

Hepatitis C virus (HCV), a positive strand RNA virus associated with the development of HCC, also induces glycolysis. Hepatitis C virus infection decreases host cell oxidative phosphorylation and increases dependence on extracellular glucose. In addition to an increased requirement for glucose, there is an increase in lactate production in HCV-infected cells. HCV infection perturbs glucose metabolism, resulting in increased insulin resistance and gluconeogenesis, which manifests as decreased insulin resistance and increased IRS1/2 expression. Transgenic mice with expression of HCV core protein in the liver demonstrate evidence of increased insulin resistance. At the cellular level, HCV core protein has been observed to increase IRS1 phosphorylation and impair insulin activation of Akt. Core also decreases IRS1 and IRS2 levels and inhibits 6-phosphofructo-2-kinase activation. The HCV nonstructural protein NS5A has been found to increase hepatic gluconeogenesis through induction of ROS, leading to increased phosphoenolpyruvate carboxykinase (PEPCK) and glucose 6-phosphatase (G6Pase) expression and decreased glucokinase expression. The HCV NS5A protein also interacts with HK2 to enhance its activity and is sufficient to induce increased glucose uptake and lactic acid production. The HCV-regulated microRNA 130a enhances the activity of pyruvate kinase, another key enzyme in glycolysis.

Epstein-Barr virus (EBV) is the causative agent of infectious mononucleosis and also causes a number of malignancies including Burkitt's lymphoma and nasopharyngeal carcinoma (NPC). EBV infected NPC cell lines have high levels of glycolysis, an effect recapitulated by the expression of a known EBV oncogene expressed during many forms of latency, latent membrane protein 1 (LMP-1). Studies suggest that LMP1 promotes glycolysis via FGF2 and FGR1 activation and that this mechanism is also important for the infected cells' transformation characteristics, including proliferation, migration, and invasiveness. LMP 1 also enhances glycolysis by upregulating HK2, a change that correlates with increased cell viability and proliferation. Increased HK2 expression was also noted in some cases of EBV-associated NPC and was negatively correlated with survival. LMP1 enhances expression, stability, and plasma localization of GLUT1, contributing to increased glycolysis. Studies also suggest that LMP1 may upregulate glycolysis by repressing HOX genes. LMP1 also promotes glycolysis by upregulating pyruvate dehydrogenase kinase 1 (PDK1) and PKM2 via upregulation of HIF-1a. LMP1 promotes HIF-1a stabilization by enhancing the degradation of prolyl HIF-hydroxylases PHD1 and PHD3. Additionally, the EBV viral proteins EBNA3 and EBNA5 bind to PHD2 and PHD1, respectively, representing another mechanism by which EBV infection stabilizes HIF-1α to promote glycolysis. EBV infection also produces the miRNA EBV-miR-Bart1-5P that has been shown to promote a glycolytic phenotype.

Latent viral infections can also induce glycolysis. Kaposi's sarcoma-associated herpesvirus (KSHV), also known as human herpesvirus 8, is an oncogenic γ-herpesvirus known to cause Kaposi's sarcoma. Upon infection of endothelial cells, KSHV establishes a predominantly latent infection. A metabolomics study of endothelial cells latently infected with KSHV found that glycolytic metabolites are induced during latency. KSHV encoded microRNAs are sufficient to induce aerobic glycolysis. KSHV encodes over 17 distinct microRNA species from 12 loci. The microRNAs are encoded in the major latent locus and are expressed during latent infection. Ten of the 12 KSHV miRNA loci are intergenic. When these 10 intergenic viral microRNAs were overexpressed, there is an increase in lactic acid production and a decrease in oxygen utilization. The microRNA cluster also induces hypoxia induced factor 1 and upregulates the expression of glucose transporter 1. Viral-encoded microRNAs are important for inducing the alterations in glucose metabolism, by repressing the expression of the metabolic regulator genes EGLN2 (encoding Egl nine homolog 2) and HSPA9 (encoding Stress-70 protein, mitochondrial), which then results in increased glycolysis and GLUT1 expression. Another KSHV-encoded protein involved in metabolic reprogramming includes LANA, which targets p53 and/or HIF-1a.

Merkel cell polyomavirus (MCPyV), a relatively recently discovered oncogenic polyomavirus, which is associated with Merkel cell carcinoma (MCC), utilizes the MCPyV small tumor antigen (ST) is able to promote a glycolytic phenotype by upregulating multiple glycolytic genes, including SLC16A1 (MCT1) and SLC2A1 (GLUT1).

SV40-encoded T-Ag protein is also involved in metabolic reprogramming and targets p53, AMPK, and/or mTOR.

HIV-encoded Vpr and Env proteins are also involved in metabolic reprogramming and targets HIF-1 and/or mTOR.

### B. Fatty Acid Synthesis

In specific aspects, the one or more exogenously provided viral genes capable of manipulating cell metabolism expressed by immune cells, e.g., NK cells, are capable of increasing fatty acid synthesis.

Fatty acid synthesis supports the creation of lipid material in the cell and is important for increased membrane production as well as other cellular needs. The core of fatty acid synthesis is the production of palmitate from acetyl-CoA and malonyl-CoA in a reaction that requires NADPH and is catalyzed by fatty acid synthase (FAS). In mammalian cells, the carbon substrates for fatty acid synthesis are generally derived from citrate, an intermediate of the TCA cycle. Once synthesized, palmitate can be further metabolized into a number of long chain fatty acids that can then be used in lipid production for membrane biosynthesis and lipid droplet formation. Lipid droplets are storage organelles for lipids, triacylglycerides and sterol esters and are also beneficial as energy storage for cells. Lipid droplet formation can be indicative of increased fatty acid synthesis and prepares the cell for rapid membrane generation and maintains an energy cache. Fatty acids can also be broken down by beta-oxidation to produce energy.

EBV infection alters lipid metabolism in part through EBV-encoded RNAs (EBERs), which leads to upregulation of FAS and low-density lipoprotein receptor (LDLR). During lytic reactivation, expression of one of the EBV immediate-early proteins, BRLF1, also results in FAS upregulation. The reactivation of EBV lytic replication is blocked by FAS inhibitors, apparently in a BRLF dependent fashion at early times after the induction.

RNA viruses that replicate in the cytoplasm alter lipids in the cytoplasm to create a beneficial environment for replication. For example, HCV uses low-density lipoprotein receptor as a co-factor for entry, HCV replication occurs on lipid raft-like domains, often referred to as membranous webs, and HCV assembly appears to occur on lipid droplets. HCV induces activation of the SREBPs and also induces FAS to increase fatty acid synthesis. Many of the HCV-induced alterations in lipid metabolism stem from the HCV core protein. HCV core protein-expressing transgenic mice develop hepatic steatosis at grades correlative to the HCV core protein level, and subsequent liver lesions with histologic similarity to HCC. HCV core protein within the cell accumulates in a globular pattern around the lipid droplets by means of interaction with DGAT1, and DGAT1−/− mice do not develop steatosis induced by HCV core protein. Studies have suggested that HCV core protein can alter lipid metabolism through inhibition of microsomal triglyceride transfer protein (MTP), activation of the Srebp-1c promoter (HCV nonstructural protein 2 has also been suggested to perform this function), and increasing proteolytic cleavage of sterol regulatory element binding proteins to their mature forms (HCV nonstructural protein S4B has also been suggested to play a role in this) among others. Additionally, transcriptomics studies suggest that the HCV microRNA miR-146a-5p upregulates transcription of genes involved in fatty acid metabolism.

Dengue virus (DENV) also rearranges specific membrane structures for replication and requires fatty acid synthesis for replication. A directed siRNA screen showed that FAS and acetyl-CoA carboxylase (ACC) were required for efficient dengue virus replication. Dengue virus does not appear to increase the expression level of FAS but rather causes a relocalization of FAS to the novel membrane structures induced by the virus. The dengue virus nonstructural protein, nonstructural protein 3 (NS3) drives relocalization of FAS by recruiting FAS to sites of DENV particle replication and stimulates FAS activity and this relocalization appears to involve Rab18 binding to NS3. Dengue virus infection increases overall fatty acid synthesis in host cells as determined by increased uptake of radiolabeled acetate with the highest amount of label found in the subcellular fractions that contained dengue virus RNA. In some aspects, the increase in fatty acid synthesis leads to the increased lipid droplet formation found in dengue virus infected cells. Other DENV-encoded proteins involved in metabolic reprogramming include NS4A, which promotes autophagy and lipid metabolism, and NS1, which targets GAPDH.

HBV transgenic mice also have higher transcription of lipid biosynthesis genes. Similarly, transgenic mice with the HBV pre-S2 mutant antigen exhibited increased lipid droplet accumulation and upregulation of several lipogenic enzymes. HBV X protein (HBx), encoded by ORFx, has been shown to activate lipid synthesis and uptake and inhibit ApoB secretion.

KSHV latent infection induces lipid droplet formation and alters lipid metabolism by upregulating lipid biosynthesis as well as peroxisome biosynthesis and associated proteins involved in very long chain fatty acid metabolism. KSHV viral miRNAs also inhibit cholesterol synthesis, possibly suppressing cellular innate immune functions

### C. Glutaminolysis

In specific aspects, the one or more exogenously provided viral genes capable of manipulating cell metabolism expressed by immune cells, e.g., NK cells, are capable of increasing glutaminolysis.

While glutamine is a non-essential amino acid, extracellular glutamine is often imported for multiple cellular metabolic pathways. Glutamine can be utilized for glutathione production, ammonia production, and purine synthesis through nitrogen donation among other uses. Importantly, glutamine can be utilized in glutaminolysis. In glutaminolysis, glutamine is converted to glutamate and then to alpha-ketoglutarate. Alpha-ketoglutarate can enter the mitochondria where it can be utilized as an intermediate of the TCA cycle. Cancer cells often become glutamine addicted. In many cancer cells, glucose carbon is shunted away from the TCA cycle both into lactic acid production as well as fatty acid synthesis. Glutamine is then required as an anaplerotic substrate to replenish the TCA cycle. A number of viruses have also been shown to require glutamine for replication. Viruses appear to induce glutaminolysis when glucose carbon is shunted away from the TCA cycle.

Vaccinia virus is one of the few viruses that does not require glycolysis for replication in cultured cells. A metabolomics study of cells infected with vaccinia showed that there is no increase in glycolytic metabolites but there is an increase in intracellular glutamine and glutamate. Removal of glutamine, but not glucose, from the media led to a significant drop in virus production. In the absence of glutamine, late genes were expressed at low levels though the maturation of processed late genes occurred. Electron microscopy studies showed that in the absence of glutamine, immature and mature virus particles are produced but at drastically reduced levels, with only small virus factories in the cytoplasm. Virus factory levels and the production of infectious virus can be restored by supplementation with alpha-ketoglutarate as well as other TCA cycle intermediates. Therefore, glutamine is utilized as an anaplerotic substrate for the TCA cycle. Vaccinia virus deleted for the C16 protein, a protein that stabilizes HIF-1 through binding to the prolylhydroxylase domain-containing protein (PHD) 2, a cellular oxygen sensor, has lower levels of glutamine metabolites compared to wild type infection indicating that, in some aspects, the induction of glutaminolysis may, at least in part, be due to this viral protein.

In addition to altering cellular glucose metabolism, adenovirus infection also results in increased glutamine consumption and activity of glutaminase (GLS). Glutamine tracing studies show that glutamine undergoes reductive carboxylation during adenovirus infection, potentially as a source of citrate. Additionally, glutamine is used to generate amino acids and hexosamine pathway intermediates. These changes in glutamine metabolism are all dependent on E4ORF1 binding to cellular MYC.

### III. Bacterial Activation of Cellular Metabolism

One or more bacterial genes capable of manipulating cell metabolism may be encoded by a polynucleotide and expressed by a cell, e.g., an immune cell, as described herein. In some aspects, the one or more bacterial genes are capable of increasing the metabolism, e.g., glycolysis, oxidative phosphorylation, fatty acid synthesis, glutaminolysis, or a combination thereof, in a cell, such that expression by a cell, e.g., an immune cell, of the one or more bacterial genes capable of manipulating cell metabolism encoded by the polynucleotide enhances the metabolic fitness of the cell and/or enhances one or more anti-tumor activities of the cell. In specific aspects, the one or more exogenously provided bacterial genes capable of manipulating cell metabolism expressed by immune cells, e.g., NK cells, are capable of increasing glycolysis. In specific aspects, the one or more exogenously provided bacterial genes capable of manipulating cell metabolism expressed by immune cells, e.g., NK cells, are capable of increasing fatty acid synthesis. In specific aspects, the one or more exogenously provided bacterial genes capable of manipulating cell metabolism expressed by immune cells, e.g., NK cells, are capable of increasing glutaminolysis.

To ensure optimal environments for their replication and spread, bacteria have evolved to alter many host cell pathways. Many bacteria can manipulate the host cell's metabolism in order to optimize the biosynthetic needs of the bacteria through probacterial metabolic changes. Bacteria can use as a major energy source various host-derived energy-rich carbon compounds that are not as essential for the host cell as glucose. These include mainly C3-metabolites like pyruvate or glycerol, Ser, and Cys which can be converted to pyruvate. Pyruvate is then further oxidized to acetyl-CoA, which feeds the tricarboxylic acid cycle (TCA) yielding important intermediates and ATP by oxidative phosphorylation (OXPHOS) or substrate phosphorylation (via acetyl-phosphate to acetate). It may also enter the gluconeogenesis pathway. Alternatively, FAs or cholesterol (CL) can be used as energy-rich components.

De novo biosynthesis performed by bacteria within host cells can be limited to those compounds that cannot be provided by the host cells. This includes, e.g., cell wall components. For the implementation of these biosynthetic pathways, bacteria can use limited amounts of host cell-derived glucose, glucose-6-phosphate, or other carbohydrates that can be converted to glucose-6-phosphate. Thus, the intracellular replication of bacteria utilizes low molecular metabolites from the host cell. Most other low molecular metabolites, including most amino acids, nucleotides, FAs, and vitamins, can be imported from the host cell. Exceptions are the three non-essential amino acids Ala, Asp, and Glu, which can be de novo synthesized by bacteria. This bacterial metabolic strategy also allows the expression of the virulence factors that are essential for intracellular replication. Their expression is often under catabolite repression, i.e., blocked when glucose is the major carbon source.

Many studies on bacterial replication have used as host cells different established cell lines, including MO- and MP-like cell lines (e.g., J774A.1, P388.D1, RAW264.7, THP-1, U-937) as well as epithelial and fibroblast cell lines (e.g., Caco-2, HeLa, Hep-2, HEK293, MDCK, NIH3T3, and others). These studies show that these host cells allow highly efficient intracellular replication of most bacteria. Most of these cell lines perform already in the un-infected state a highly activated metabolism, in most cases caused by the permanent activation of oncogenes (e.g., Myc in J774 MPs) or the inactivation of tumor suppressors (e.g., p53 in Caco-2, HeLa, U-937, THP-1). This host cell metabolism is characterized in general by enhanced glucose uptake, aerobic glycolysis, increased PPP activity, eventually enhanced glutaminolysis, and increased anabolic activities, which resembles the metabolic program widely seen in proliferating cancer cells known as the Warburg effect (or aerobic glycolysis).

In particular, bacteria can influence the activity of central metabolic regulators of their host cells. Bacterial factors activate components of the PI3K/Akt/mTOR cascade and Myc or alter the concentration and/or activity of p53 and HIF-1. Most of these interactions lead to enhanced glucose uptake, increased aerobic glycolysis, and enhanced PPP activity as well as to activation of anabolic pathways in the infected host cells. Activation of Myc by some bacteria also enhances Gln uptake and glutaminolysis. In some aspects, bacterial infection may cause a switch from to induced glucose uptake, aerobic glycolysis combined with lactate production, enhanced PPP and decreased TCA activities. In some aspects, bacterial infection may cause enhanced FAO, OXPHOS, and increased intracellular levels of unconsumed glucose. These and other host cell metabolic changes in response to bacterial infection are described in, e.g., Eisenreich W. et al. (2019). Frontiers in Cellular and Infection Microbiology 9:42; and Escoll P. & Buchrieser C. (March 2018). The FEBS Journal 285:2146-2160, each incorporated by reference herein in its entirety.

In some aspects, these host metabolic changes meet the metabolic requirements of many bacteria for efficient intracellular replication and proliferation and no further metabolic reprogramming in these host cells is necessary to satisfy the nutritional needs of bacteria for efficient intracellular growth. Exceptions include Chlamydia pneumoniae infection of Hep-2 cells, which can lead to additional stabilization of HIF-1a resulting in further enhanced glucose uptake during the early phase of infection which favors bacterial proliferation.

As disclosed herein, in some cases, one or more bacterial genes capable of manipulating cell metabolism are present on the same polynucleotide or vector molecule as an engineered antigen-specific receptor, although in other cases they are on separate polynucleotides or vector molecules. In particular aspects, one or more bacterial genes are co-expressed from the same polynucleotide or vector as the engineered antigen-specific receptor. One or more bacterial gene products may be produced as a separate polypeptide from an antigen-specific receptor.

In specific aspects, immune cells, e.g., NK cells, express one or more exogenously provided bacterial genes capable of manipulating cell metabolism. The bacterial gene(s) capable of manipulating cell metabolism may be exogenously provided to the immune cells, e.g., NK cells, because it is expressed from an expression vector within the cell. In cases wherein the bacterial gene(s) capable of manipulating cell metabolism are provided on an expression construct to the cell, the bacterial gene(s) may be encoded from the same vector as an antigen-specific receptor and/or a suicide gene. The bacterial gene(s) may be expressed as a separate polypeptide molecule from an antigen-specific receptor and/or a suicide gene. In some aspects, the present disclosure concerns co-utilization of CAR and/or TCR vectors with bacterial gene(s), particularly in NK cells.

### IV. Fungal Activation of Cellular Metabolism

One or more fungal genes capable of manipulating cell metabolism may be encoded by a polynucleotide and expressed by a cell, e.g., an immune cell, as described herein. In some aspects, the one or more fungal genes are capable of increasing the metabolism, e.g., glycolysis, oxidative phosphorylation, fatty acid synthesis, glutaminolysis, or a combination thereof, in a cell, such that expression by a cell, e.g., an immune cell, of the one or more fungal genes capable of manipulating cell metabolism encoded by the polynucleotide enhances the metabolic fitness of the cell and/or enhances one or more anti-tumor activities of the cell. In specific aspects, the one or more exogenously provided fungal genes capable of manipulating cell metabolism expressed by immune cells, e.g., NK cells, are capable of increasing glycolysis. In specific aspects, the one or more exogenously provided fungal genes capable of manipulating cell metabolism expressed by immune cells, e.g., NK cells, are capable of increasing fatty acid synthesis. In specific aspects, the one or more exogenously provided fungal genes capable of manipulating cell metabolism expressed by immune cells, e.g., NK cells, are capable of increasing glutaminolysis.

Recognition of fungal pathogens triggers a host-protective metabolic shift but provides opportunities for fungal immune evasion. Pathogen recognition receptors on host cells can recognize pathogen associated molecular patterns on fungal cell walls, prompting a shift to increased glycolysis and the production of lactate. Glycolytic metabolism enables immune cells to deploy antimicrobial effectors. Predominantly, a Warburg effect is triggered, whereby not only glycolysis is enhanced, but mitochondrial oxidative phosphorylation is repressed. For C. albicans this is triggered by the recognition of fungal cell wall β-glucan by the host receptor Dectin-1. In A. fumigatus, internalized conidia shed melanin, which signals to macrophages to onset Warburg metabolism. Melanin is able to sequester calcium within the phagosome, which in turn triggers glycolysis-promoting signals through the direct recruitment of the intracellular sensor mTOR, the master regulator of glucose metabolism. This then mediates the increased expression of the transcription factor HIF-1α and activation of the downstream glycolytic genes. By hijacking some of these metabolic responses, fungi are able to attempt immune evasion. These and other host cell metabolic changes in response to fungal infection are described in, e.g., Pellon A. et al. (2022). Pathogens 11:184; and Weerasinghe H. & Traven A. (2022). Current Opinion in Microbiology 58:32-40, each incorporated by reference herein in its entirety.

As disclosed herein, in some cases, one or more fungal genes capable of manipulating cell metabolism are present on the same polynucleotide or vector molecule as an engineered antigen-specific receptor, although in other cases they are on separate polynucleotides or vector molecules. In particular aspects, one or more fungal genes are co-expressed from the same polynucleotide or vector as the engineered antigen-specific receptor. One or more fungal gene products may be produced as a separate polypeptide from an antigen-specific receptor.

In specific aspects, immune cells, e.g., NK cells, express one or more exogenously provided fungal genes capable of manipulating cell metabolism. The fungal gene(s) capable of manipulating cell metabolism may be exogenously provided to the immune cells, e.g., NK cells, because it is expressed from an expression vector within the cell. In cases wherein the fungal gene(s) capable of manipulating cell metabolism are provided on an expression construct to the cell, the fungal gene(s) may be encoded from the same vector as an antigen-specific receptor and/or a suicide gene. The fungal gene(s) may be expressed as a separate polypeptide molecule from an antigen-specific receptor and/or a suicide gene. In some aspects, the present disclosure concerns co-utilization of CAR and/or TCR vectors with fungal gene(s), particularly in NK cells.

### V. Polypeptides

As used herein, a “protein” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleotide substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleotides, or any range derivable therein, of SEQ ID NOs: 1-97.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs: 41-94.

In some aspects, the protein or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs: 41-94.

In some aspects, the polypeptide or protein may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs: 41-94 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs: 41-94.

In some aspects there is a polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOs: 41-94 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOs: 1-97.

The nucleotide as well as the protein, polypeptide, and peptide sequences for various genes have been previously disclosed and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Variant Polypeptides

The following is a discussion of changing the amino acid subunits of a protein to create an equivalent, or even improved, variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted, or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### B. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the present disclosure, those that are within ±1 are included, and in other aspects of the present disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some aspects of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### C. Sequences

The amino acid sequence of certain polypeptides, including viral genes, antibodies, chimeric antigen receptors, chimeric polypeptides, immune cell engagers, and portions, regions, and domains thereof, are provided in Table 1.

TABLE 1

| | SEQ | |
|---|---|---|
| | ID | |
| NAME | NO | SEQUENCE |
| E4ORF-1 | 41 | MAESLYAFIDSPGGIAPVQEGASNRYIFFCPESFHIPPHGVI |
| | | LLHLRVSVLVPTGYQGRFMALNDYHARGILTQSDVIFAG |
| | | RRHDLSVLLFNHTDRFLYVREGHPVGTLLLERVIFPSVRI |
| | | ATLV |
| C16 | 42 | MISLSFLIHNPLKKWKLKPSISINGYRSTFTMAFPCAQFRP |
| | | CHCHATKDSLNTVADVRHCLTEYILWVSHRWTHRESAG |
| | | SLYRLLISFRTDATELFGGELKDSLPWDNIDNCVEIIKCFI |
| | | RNDSMKTAEELRAIIGLCTQSAIVSGRVENDKYIDILLML |
| | | RKILNENDYLTLLDHIRTAKY |
| NS3 | 43 | SGVLWDTPSPPEVERAVLDNGIYRILQRGLLGRSQVGVG |
| | | VFQEGVFHTMWHVTRGAVLMYQGKRLEPSWASVKKDL |
| | | ISYGGGWRFQGSWNTGEEVQVIAVEPGKNPKNVQTTPG |
| | | TFKTPEGEIGAIALDFKPGTSGSPIVNREGKIVGLYGNGV |
| | | VTTSGTYVSAIAQAKVSQEGPLPEIEDEVFRK |
| CO | 44 | MALPVTALLLPLALLLHAARPQAVVTQEPSLTVSPGGTV |
| CAR.CD70 | | TLTCGLKSGSVTSDNFPTWYQQTPGQAPRLLIYNTNTRH |
| 42D12 VLVH | | SGVPDRFSGSILGNKAALTITGAQADDEAEYFCALFISNP |
| | | SVEFGGGTQLTVLGGSTSGSGKPGSGEGSTKGEVQLVES |
| | | GGGLVQPGGSLRLSCAASGFTFSVYYMNWVRQAPGKGL |
| | | EWVSDINNEGGTTYYADSVKGRFTISRDNSKNSLYLQM |
| | | NSLRAEDTAVYYCARDAGYSNHVPIFDSWGQGTLVTVS |
| | | S |
| CAR.CD70 | 45 | MGMALPVTALLLPLALLLHAARPEVQLVESGGGLVQPG |
| 42D12 VHVL | | GSLRLSCAASGFTFSVYYMNWVRQAPGKGLEWVSDINN |
| | | EGGTTYYADSVKGRFTISRDNSKNSLYLQMNSLRAEDTA |
| | | VYYCARDAGYSNHVPIFDSWGQGTLVTVSSGSTSGSGKP |
| | | GSGEGSTKGQAVVTQEPSLTVSPGGTVTLTCGLKSGSVT |
| | | SDNFPTWYQQTPGQAPRLLIYNTNTRHSGVPDRFSGSILG |
| | | NKAALTITGAQADDEAEYFCALFISNPSVEFGGGTQLTVL |
| | | G |
| CAR.CD70 | 46 | MGMALPVTALLLPLALLLHAARPEVOLVESGGGLVQPG |
| 42D12 VLVH | | GSLRLSCAASGFTFSVYYMNWVRQAPGKGLEWVSDINN |
| | | EGGTTYYADSVKGRFTISRDNSKNSLYLQMNSLRAEDTA |
| | | VYYCARDAGYSNHVPIFDSWGQGTLVTVSSGSTSGSGKP |
| | | GSGEGSTKGQAVVTQEPSLTVSPGGTVTLTCGLKSGSVT |
| | | SDNFPTWYQQTPGQAPRLLIYNTNTRHSGVPDRFSGSILG |
| | | NKAALTITGAQADDEAEYFCALFISNPSVEFGGGTQLTVL |
| | | G |
| CD28 | 47 | FWVLVVVGGVLACYSLLVTVAFIIFWV |
| transmembrane | | |
| domain | | |
| CD8 | 48 | TTTPAPRPPTPAPTIASQPLSLRPEACRPAAGGAVHTRGL |
| transmembrane | | DFACDIYIWAPLAGTCGVLLLSLVIT |
| domain | | |
| 4-1BB | 49 | IISFFLALTSTALLFLLFFLTLRFSVV |
| transmembrane | | |
| domain | | |
| DAP10 | 50 | LLAGLVAADAVASLLIVGAVF |
| transmembrane | | |
| domain | | |
| DAP12 | 51 | GVLAGIVMGDLVLTVLIALAV |
| transmembrane | | |
| domain | | |
| NKG2D | 52 | AVMIIFRIGMAVAIFCCFFFP |
| transmembrane | | |
| domain | | |
| CD3zeta | 53 | TRKKFSRSADAPAYQQGQNQLYNELNLGRREEYDVLDK |
| intracellular | | RRGRDPEMGGKPRRKNPQEGLYNELQKDKMAEAYSEIG |
| domain-1 | | MKGERRRGKGHDGLYQGLSTATKDTYDALHMQALPPR |
| | | G |
| CD3zeta | 54 | KRVKFSRSADAPAYQQGQNQLYNELNLGRREEYDVLDK |
| intracellular | | RRGRDPEMGGKPRRKNPQEGLYNELQKDKMAEAYSEIG |
| domain-2 | | MKGERRRGKGHDGLYQGLSTATKDTYDALHMQALPPR |
| | | G |
| CD3zeta | 55 | RVKFSRSADAPAYQQGQNQLYNELNLGRREEYDVLDKR |
| intracellular | | RGRDPEMGGKPRRKNPQEGLYNELQKDKMAEAYSEIG |
| domain 3 | | MKGERRRGKGHDGLYQGLSTATKDTYDALHMQALPPR |
| | | G |
| 4-1BB | 56 | KRGRKKLLYIFKQPFMRPVQTTQEEDGCSCRFPEEEEGG |
| intracellular | | CEL |
| domain | | |
| DAP10 | 57 | LCARPRRSPAQEDGKVYINMPGRG |
| intracellular | | |
| domain | | |
| DAP12 | 58 | YFLGRLVPRGRGAAEAATRKQRITETESPYQELQGQRSD |
| intracellular | | VYSDLNTQRPYYK |
| domain | | |
| NKG2D | 59 | SANERCKSKVVPCRQKQWRTSFDSKKLDLNYNHFESME |
| intracellular | | WSHRSRRGRIWGM |
| domain | | |
| IgG Hinge | 60 | TVTVSSQDPAEPKSPDKTHTCPPCPAPELLGGPSVFLFPPK |
| | | PKDTLMISRTPEVTCVVVDVSHEDPEVKFNWYVDGVEV |
| | | HNAKTKPREEQYNSTYRVVSVLTVLHQDWLNGKEYKC |
| | | KVSNKALPAPIEKTISKAKGQPREPQVYTLPPSRDELTKN |
| | | QVSLTCLVKGFYPSDIAVEWESNGQPENNYKTTPPVLDS |
| | | DGSFFLYSKLTVDKSRWQQGNVFSCSVMHEAL |
| CD28 Hinge | 61 | IEVMYPPPYLDNEKSNGTIIHVKGKHLCPSPLFPGPSKP |
| IL-15 | 62 | ISKPHLRSISIQCYLCLLLNSHFLTEAGIHVFILGCFSAGLP |
| | | KTEANWVNVISDLKKIEDLIQSMHIDATLYTESDVHPSCK |
| | | VTAMKCFLLELQVISLESGDASIHDTVENLIILANNSLSSN |
| | | GNVTESGCKECEELEEKNIKEFLQSFVHIVQMFINTS |
| E2A | 63 | QCTNYALLKLAGDVESNPGP |
| T2A | 64 | EGRGSLLTCGDVEENPGP |
| P2A | 65 | ATNFSLLKQAGDVEENPGP |
| F2A | 66 | VKQTLNFDLLKLAGDVESNPGP |
| CD27 signal | 67 | MARPHPWWLCVLGTLVGLS |
| peptide | | |
| GMCSF- | 68 | MLLLVTSLLLCELPHPAFLLIP |
| Receptor | | |
| CD27 | 69 | ATPAPKSCPERHYWAQGKLCCQMCEPGTFLVKDCDQHR |
| extracellular | | KAAQCDPCIPGVSFSPDHHTRPHCESCRHCNSGLLVRNC |
| domain | | TITANAECACRNGWQCRDKECTECDPLPNPSLTARSSQA |
| | | LSPHPQPTHLPYVSEMLEARTAGHMQTLADFRQLPARTL |
| | | STHWPPQRSLCSSDFIR |
| CD27 | 70 | ILVIFSGMFLVFTLAGALFLH |
| transmembrane | | |
| domain | | |
| CD27tr28tdmCD3zIL15 | 71 | MARPHPWWLCVLGTLVGLSATPAPKSCPERHYWAQGK |
| | | LCCQMCEPGTFLVKDCDQHRKAAQCDPCIPGVSFSPDHH |
| | | TRPHCESCRHCNSGLLVRNCTITANAECACRNGWQCRD |
| | | KECTECDPLPNPSLTARSSQALSPHPQPTHLPYVSEMLEA |
| | | RTAGHMQTLADFRQLPARTLSTHWPPQRSLCSSDFIRFW |
| | | VLVVVGGVLACYSLLVTVAFIIFWV |
| CD27Tr28tmd41BBicd3zIL15 | 72 | MTRVINSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVKRGRKKLLYIFKQPFMRP |
| | | VQTTQEEDGCSCRFPEEEEGGCELKRVKFSRSADAPAYQ |
| | | QGQNQLYNELNLGRREEYDVLDKRRGRDPEMGGKPRR |
| | | KNPQEGLYNELQKDKMAEAYSEIGMKGERRRGKGHDG |
| | | LYQGLSTATKDTYDALHMQALPPRGPQCTNYALLKLAG |
| | | DVESNPGPMRISKPHLRSISIQCYLCLLLNSHFLTEAGIHV |
| | | FILGCFSAGLPKTEANWVNVISDLKKIEDLIQSMHIDATL |
| | | YTESDVHPSCKVTAMKCFLLELQVISLESGDASIHDTVEN |
| | | LIILANNSLSSNGNVTESGCKECEELEEKNIKEFLQSFVHI |
| | | VQMFINTS |
| GSPco27Tr28tmd41BBicCD3zIL15 | 73 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVKRGRKKLLYIFKQPFMRP |
| | | VQTTQEEDGCSCRFPEEEEGGCELKRVKFSRSADAPAYQ |
| | | QGQNQLYNELNLGRREEYDVLDKRRGRDPEMGGKPRR |
| | | KNPQEGLYNELQKDKMAEAYSEIGMKGERRRGKGHDG |
| | | LYQGLSTATKDTYDALHMQALPPRGPQCTNYALLKLAG |
| | | DVESNPGPMRISKPHLRSISIQCYLCLLLNSHFLTEAGIHV |
| | | FILGCFSAGLPKTEANWVNVISDLKKIEDLIQSMHIDATL |
| | | YTESDVHPSCKVTAMKCFLLELQVISLESGDASIHDTVEN |
| | | LIILANNSLSSNGNVTESGCKECEELEEKNIKEFLQSFVHI |
| | | VQMFINTS |
| CD27Tr28tmdDAP10icd3z15 | 74 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVLCARPRRSPAQEDGKVYI |
| | | NMPGRGKRVKFSRSADAPAYQQGQNQLYNELNLGRREE |
| | | YDVLDKRRGRDPEMGGKPRRKNPQEGLYNELQKDKMA |
| | | EAYSEIGMKGERRRGKGHDGLYQGLSTATKDTYDALH |
| | | MQALPPRGPQCTNYALLKLAGDVESNPGPMRISKPHLRS |
| | | ISIQCYLCLLLNSHFLTEAGIHVFILGCFSAGLPKTEANWV |
| | | NVISDLKKIEDLIQSMHIDATLYTESDVHPSCKVTAMKCF |
| | | LLELQVISLESGDASIHDTVENLIILANNSLSSNGNVTESG |
| | | CKECEELEEKNIKEFLQSFVHIVQMFINTS |
| GSPco27Tr28tmdDAP10IL15 | 75 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVLCARPRRSPAQEDGKVYI |
| | | NMPGRGKRVKFSRSADAPAYQQGQNQLYNELNLGRREE |
| | | YDVLDKRRGRDPEMGGKPRRKNPQEGLYNELQKDKMA |
| | | EAYSEIGMKGERRRGKGHDGLYQGLSTATKDTYDALH |
| | | MQALPPRGPQCTNYALLKLAGDVESNPGPMRISKPHLRS |
| | | ISIQCYLCLLLNSHFLTEAGIHVFILGCFSAGLPKTEANWV |
| | | NVISDLKKIEDLIQSMHIDATLYTESDVHPSCKVTAMKCF |
| | | LLELQVISLESGDASIHDTVENLIILANNSLSSNGNVTESG |
| | | CKECEELEEKNIKEFLQSFVHIVQMFINTS |
| CD27Tr28tmdDAP12icd3z15 | 76 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVYFLGRLVPRGRGAAEAAT |
| | | RKQRITETESPYQELQGQRSDVYSDLNTQRPYYKKRVKF |
| | | SRSADAPAYQQGQNQLYNELNLGRREEYDVLDKRRGRD |
| | | PEMGGKPRRKNPQEGLYNELQKDKMAEAYSEIGMKGER |
| | | RRGKGHDGLYQGLSTATKDTYDALHMQALPPRGPQCTN |
| | | YALLKLAGDVESNPGPMRISKPHLRSISIQCYLCLLLNSH |
| | | FLTEAGIHVFILGCFSAGLPKTEANWVNVISDLKKIEDLIQ |
| | | SMHIDATLYTESDVHPSCKVTAMKCFLLELQVISLESGD |
| | | ASIHDTVENLIILANNSLSSNGNVTESGCKECEELEEKNIK |
| | | EFLQSFVHIVQMFINTS |
| GSPco27Tr28tmddap12icd15 | 77 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVYFLGRLVPRGRGAAEAAT |
| | | RKQRITETESPYQELQGQRSDVYSDLNTQRPYYKKRVKF |
| | | SRSADAPAYQQGQNQLYNELNLGRREEYDVLDKRRGRD |
| | | PEMGGKPRRKNPQEGLYNELQKDKMAEAYSEIGMKGER |
| | | RRGKGHDGLYQGLSTATKDTYDALHMQALPPRGPQCTN |
| | | YALLKLAGDVESNPGPMRISKPHLRSISIQCYLCLLLNSH |
| | | FLTEAGIHVFILGCFSAGLPKTEANWVNVISDLKKIEDLIQ |
| | | SMHIDATLYTESDVHPSCKVTAMKCFLLELQVISLESGD |
| | | ASIHDTVENLIILANNSLSSNGNVTESGCKECEELEEKNIK |
| | | EFLQSFVHIVQMFINTS |
| CD27Tr28tmdNKG2Dic3z15 | 78 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVSANERCKSKVVPCRQKQ |
| | | WRTSFDSKKLDLNYNHFESMEWSHRSRRGRIWGMKRV |
| | | KFSRSADAPAYQQGQNQLYNELNLGRREEYDVLDKRRG |
| | | RDPEMGGKPRRKNPQEGLYNELQKDKMAEAYSEIGMK |
| | | GERRRGKGHDGLYQGLSTATKDTYDALHMQALPPRGPQ |
| | | CTNYALLKLAGDVESNPGPMRISKPHLRSISIQCYLCLLL |
| | | NSHFLTEAGIHVFILGCFSAGLPKTEANWVNVISDLKKIE |
| | | DLIQSMHIDATLYTESDVHPSCKVTAMKCFLLELQVISLE |
| | | SGDASIHDTVENLIILANNSLSSNGNVTESGCKECEELEE |
| | | KNIKEFLQSFVHIVQMFINTS |
| GSPco27Tr28tmdNKG2Dicd3z15 | 79 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVSANERCKSKVVPCRQKQ |
| | | WRTSFDSKKLDLNYNHFESMEWSHRSRRGRIWGMKRV |
| | | KFSRSADAPAYQQGQNQLYNELNLGRREEYDVLDKRRG |
| | | RDPEMGGKPRRKNPQEGLYNELQKDKMAEAYSEIGMK |
| | | GERRRGKGHDGLYQGLSTATKDTYDALHMQALPPRGPQ |
| | | CTNYALLKLAGDVESNPGPMRISKPHLRSISIQCYLCLLL |
| | | NSHFLTEAGIHVFILGCFSAGLPKTEANWVNVISDLKKIE |
| | | DLIQSMHIDATLYTESDVHPSCKVTAMKCFLLELQVISLE |
| | | SGDASIHDTVENLIILANNSLSSNGNVTESGCKECEELEE |
| | | KNIKEFLQSFVHIVQMFINTS |
| CD27Tr41BBicd3z15 | 80 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHKRGRKKLLYIFKQPFMRPVQTTQEE |
| | | DGCSCRFPEEEEGGCELKRVKFSRSADAPAYQQGQNQL |
| | | YNELNLGRREEYDVLDKRRGRDPEMGGKPRRKNPQEGL |
| | | YNELQKDKMAEAYSEIGMKGERRRGKGHDGLYQGLST |
| | | ATKDTYDALHMQALPPRGPQCTNYALLKLAGDVESNPG |
| | | PMRISKPHLRSISIQCYLCLLLNSHFLTEAGIHVFILGCFSA |
| | | GLPKTEANWVNVISDLKKIEDLIQSMHIDATLYTESDVHP |
| | | SCKVTAMKCFLLELQVISLESGDASIHDTVENLIILANNSL |
| | | SSNGNVTESGCKECEELEEKNIKEFLQSFVHIVQMFINTS |
| GSPco27Tr41BBicd3z15 | 81 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHKRGRKKLLYIFKQPFMRPVQTTQEE |
| | | DGCSCRFPEEEEGGCELKRVKFSRSADAPAYQQGQNQL |
| | | YNELNLGRREEYDVLDKRRGRDPEMGGKPRRKNPQEGL |
| | | YNELQKDKMAEAYSEIGMKGERRRGKGHDGLYQGLST |
| | | ATKDTYDALHMQALPPRGPQCTNYALLKLAGDVESNPG |
| | | PMRISKPHLRSISIQCYLCLLLNSHFLTEAGIHVFILGCFSA |
| | | GLPKTEANWVNVISDLKKIEDLIQSMHIDATLYTESDVHP |
| | | SCKVTAMKCFLLELQVISLESGDASIHDTVENLIILANNSL |
| | | SSNGNVTESGCKECEELEEKNIKEFLQSFVHIVQMFINTS |
| CD27TrCD3ZIL15 | 82 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHKRVKFSRSADAPAYQQGQNQLYNE |
| | | LNLGRREEYDVLDKRRGRDPEMGGKPRRKNPQEGLYNE |
| | | LQKDKMAEAYSEIGMKGERRRGKGHDGLYQGLSTATK |
| | | DTYDALHMQALPPRGPQCTNYALLKLAGDVESNPGPMR |
| | | ISKPHLRSISIQCYLCLLLNSHFLTEAGIHVFILGCFSAGLP |
| | | KTEANWVNVISDLKKIEDLIQSMHIDATLYTESDVHPSCK |
| | | VTAMKCFLLELQVISLESGDASIHDTVENLIILANNSLSSN |
| | | GNVTESGCKECEELEEKNIKEFLQSFVHIVQMFINTS |
| GSPco27Tr3z15 | 83 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHKRVKFSRSADAPAYQQGQNQLYNE |
| | | LNLGRREEYDVLDKRRGRDPEMGGKPRRKNPQEGLYNE |
| | | LQKDKMAEAYSEIGMKGERRRGKGHDGLYQGLSTATK |
| | | DTYDALHMQALPPRGPQCTNYALLKLAGDVESNPGPMR |
| | | ISKPHLRSISIQCYLCLLLNSHFLTEAGIHVFILGCFSAGLP |
| | | KTEANWVNVISDLKKIEDLIQSMHIDATLYTESDVHPSCK |
| | | VTAMKCFLLELQVISLESGDASIHDTVENLIILANNSLSSN |
| | | GNVTESGCKECEELEEKNIKEFLQSFVHIVQMFINTS |
| CD27TrCD28icd3z15 | 84 | MTRVINSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHRSKRSRLLHSDYMNMTPRRPGPTRK |
| | | HYQPYAPPRDFAAYRSRVKFSRSADAPAYQQGQNQLYN |
| | | ELNLGRREEYDVLDKRRGRDPEMGGKPRRKNPQEGLYN |
| | | ELQKDKMAEAYSEIGMKGERRRGKGHDGLYQGLSTAT |
| | | KDTYDALHMQALPPRGPQCTNYALLKLAGDVESNPGPM |
| | | RISKPHLRSISIQCYLCLLLNSHFLTEAGIHVFILGCFSAGL |
| | | PKTEANWVNVISDLKKIEDLIQSMHIDATLYTESDVHPSC |
| | | KVTAMKCFLLELQVISLESGDASIHDTVENLIILANNSLSS |
| | | NGNVTESGCKECEELEEKNIKEFLQSFVHIVQMFINTS |
| GSPco27Tr28CD3z15 | 85 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRIL VIFSG |
| | | MFLVFTLAGALFLHRSKRSRLLHSDYMNMTPRRPGPTRK |
| | | HYQPYAPPRDFAAYRSRVKFSRSADAPAYQQGQNQLYN |
| | | ELNLGRREEYDVLDKRRGRDPEMGGKPRRKNPQEGLYN |
| | | ELQKDKMAEAYSEIGMKGERRRGKGHDGLYQGLSTAT |
| | | KDTYDALHMQALPPRGPQCTNYALLKLAGDVESNPGPM |
| | | RISKPHLRSISIQCYLCLLLNSHFLTEAGIHVFILGCFSAGL |
| | | PKTEANWVNVISDLKKIEDLIQSMHIDATLYTESDVHPSC |
| | | KVTAMKCFLLELQVISLESGDASIHDTVENLIILANNSLSS |
| | | NGNVTESGCKECEELEEKNIKEFLQSFVHIVQMFINTS |
| CD27TrCD28tmdicd3z15 | 86 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVRSKRSRLLHSDYMNMTPR |
| | | RPGPTRKHYQPYAPPRDFAAYRSRVKFSRSADAPAYQQ |
| | | GQNQLYNELNLGRREEYDVLDKRRGRDPEMGGKPRRK |
| | | NPQEGLYNELQKDKMAEAYSEIGMKGERRRGKGHDGL |
| | | YQGLSTATKDTYDALHMQALPPRGPQCTNYALLKLAGD |
| | | VESNPGPMRISKPHLRSISIQCYLCLLLNSHFLTEAGIHVFI |
| | | LGCFSAGLPKTEANWVNVISDLKKIEDLIQSMHIDATLYT |
| | | ESDVHPSCKVTAMKCFLLELQVISLESGDASIHDTVENLII |
| | | LANNSLSSNGNVTESGCKECEELEEKNIKEFLQSFVHIVQ |
| | | MFINTS |
| GSPco27Tr28tmdicCD3z15 | 87 | MTRVINSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRFWVLVV |
| | | VGGVLACYSLLVTVAFIIFWVRSKRSRLLHSDYMNMTPR |
| | | RPGPTRKHYQPYAPPRDFAAYRSRVKFSRSADAPAYQQ |
| | | GQNQLYNELNLGRREEYDVLDKRRGRDPEMGGKPRRK |
| | | NPQEGLYNELQKDKMAEAYSEIGMKGERRRGKGHDGL |
| | | YQGLSTATKDTYDALHMQALPPRGPQCTNYALLKLAGD |
| | | VESNPGPMRISKPHLRSISIQCYLCLLLNSHFLTEAGIHVFI |
| | | LGCFSAGLPKTEANWVNVISDLKKIEDLIQSMHIDATLYT |
| | | ESDVHPSCKVTAMKCFLLELQVISLESGDASIHDTVENLII |
| | | LANNSLSSNGNVTESGCKECEELEEKNIKEFLQSFVHIVQ |
| | | MFINTS |
| CD27TrDAP10icd3z15 | 88 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHLCARPRRSPAQEDGKVYINMPGRG |
| | | KRVKFSRSADAPAYQQGQNQLYNELNLGRREEYDVLDK |
| | | RRGRDPEMGGKPRRKNPQEGLYNELQKDKMAEAYSEIG |
| | | MKGERRRGKGHDGLYQGLSTATKDTYDALHMQALPPR |
| | | GPQCTNYALLKLAGDVESNPGPMRISKPHLRSISIQCYLC |
| | | LLLNSHFLTEAGIHVFILGCFSAGLPKTEANWVNVISDLK |
| | | KIEDLIQSMHIDATLYTESDVHPSCKVTAMKCFLLELQVI |
| | | SLESGDASIHDTVENLIILANNSLSSNGNVTESGCKECEEL |
| | | EEKNIKEFLQSFVHIVQMFINTS |
| GSPco27FLdap10icd3z15 | 89 | MTRVINSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRIL VIFSG |
| | | MFLVFTLAGALFLHLCARPRRSPAQEDGKVYINMPGRG |
| | | KRVKFSRSADAPAYQQGQNQLYNELNLGRREEYDVLDK |
| | | RRGRDPEMGGKPRRKNPQEGLYNELQKDKMAEAYSEIG |
| | | MKGERRRGKGHDGLYQGLSTATKDTYDALHMQALPPR |
| | | GPQCTNYALLKLAGDVESNPGPMRISKPHLRSISIQCYLC |
| | | LLLNSHFLTEAGIHVFILGCFSAGLPKTEANWVNVISDLK |
| | | KIEDLIQSMHIDATLYTESDVHPSCKVTAMKCFLLELQVI |
| | | SLESGDASIHDTVENLIILANNSLSSNGNVTESGCKECEEL |
| | | EEKNIKEFLQSFVHIVQMFINTS |
| CD27TrDAP12icd3z15 | 90 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHYFLGRLVPRGRGAAEAATRKQRITE |
| | | TESPYQELQGQRSDVYSDLNTQRPYYKKRVKFSRSADAP |
| | | AYQQGQNQLYNELNLGRREEYDVLDKRRGRDPEMGGK |
| | | PRRKNPQEGLYNELQKDKMAEAYSEIGMKGERRRGKGH |
| | | DGLYQGLSTATKDTYDALHMQALPPRGPQCTNYALLKL |
| | | AGDVESNPGPMRISKPHLRSISIQCYLCLLLNSHELTEAGI |
| | | HVFILGCFSAGLPKTEANWVNVISDLKKIEDLIQSMHIDA |
| | | TLYTESDVHPSCKVTAMKCFLLELQVISLESGDASIHDTV |
| | | ENLIILANNSLSSNGNVTESGCKECEELEEKNIKEFLQSFV |
| | | HIVQMFINTS |
| GSPco27FLdap12icd3z15 | 91 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHYFLGRLVPRGRGAAEAATRKQRITE |
| | | TESPYQELQGQRSDVYSDLNTQRPYYKKRVKFSRSADAP |
| | | AYQQGQNQLYNELNLGRREEYDVLDKRRGRDPEMGGK |
| | | PRRKNPQEGLYNELQKDKMAEAYSEIGMKGERRRGKGH |
| | | DGLYQGLSTATKDTYDALHMQALPPRGPQCTNYALLKL |
| | | AGDVESNPGPMRISKPHLRSISIQCYLCLLLNSHFLTEAGI |
| | | HVFILGCFSAGLPKTEANWVNVISDLKKIEDLIQSMHIDA |
| | | TLYTESDVHPSCKVTAMKCFLLELQVISLESGDASIHDTV |
| | | ENLIILANNSLSSNGNVTESGCKECEELEEKNIKEFLQSFV |
| | | HIVQMFINTS |
| CD27TrNKG2Dic3z15 | 92 | MTRVINSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMAR |
| | | PHPWWLCVLGTLVGLSATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHSANERCKSKVVPCRQKQWRTSFDS |
| | | KKLDLNYNHFESMEWSHRSRRGRIWGMKRVKFSRSAD |
| | | APAYQQGQNQLYNELNLGRREEYDVLDKRRGRDPEMG |
| | | GKPRRKNPQEGLYNELQKDKMAEAYSEIGMKGERRRGK |
| | | GHDGLYQGLSTATKDTYDALHMQALPPRGPQCTNYALL |
| | | KLAGDVESNPGPMRISKPHLRSISIQCYLCLLLNSHFLTEA |
| | | GIHVFILGCFSAGLPKTEANWVNVISDLKKIEDLIQSMHID |
| | | ATLYTESDVHPSCKVTAMKCFLLELQVISLESGDASIHDT |
| | | VENLIILANNSLSSNGNVTESGCKECEELEEKNIKEFLQSF |
| | | VHIVQMFINTS |
| GSPco27TrNKG2Dicd3z15 | 93 | MTRVTNSPSLQAHLQALYLVQHEVWRPLAAAYQEQLD |
| | | RPVVPHPYRVGDTVWVRRHQTKNLEPRWKGPYTVLLTT |
| | | PTALKVDGIAAWIHAAHVKAADPGGGPSSRLPCSRMLLL |
| | | VTSLLLCELPHPAFLLIPATPAPKSCPERHYWAQGKLCCQ |
| | | MCEPGTFLVKDCDQHRKAAQCDPCIPGVSFSPDHHTRPH |
| | | CESCRHCNSGLLVRNCTITANAECACRNGWQCRDKECT |
| | | ECDPLPNPSLTARSSQALSPHPQPTHLPYVSEMLEARTAG |
| | | HMQTLADFRQLPARTLSTHWPPQRSLCSSDFIRILVIFSG |
| | | MFLVFTLAGALFLHSANERCKSKVVPCRQKQWRTSFDS |
| | | KKLDLNYNHFESMEWSHRSRRGRIWGMKRVKFSRSAD |
| | | APAYQQGQNQLYNELNLGRREEYDVLDKRRGRDPEMG |
| | | GKPRRKNPQEGLYNELQKDKMAEAYSEIGMKGERRRGK |
| | | GHDGLYQGLSTATKDTYDALHMQALPPRGPQCTNYALL |
| | | KLAGDVESNPGPMRISKPHLRSISIQCYLCLLLNSHFLTEA |
| | | GIHVFILGCFSAGLPKTEANWVNVISDLKKIEDLIQSMHID |
| | | ATLYTESDVHPSCKVTAMKCFLLELQVISLESGDASIHDT |
| | | VENLIILANNSLSSNGNVTESGCKECEELEEKNIKEFLQSF |
| | | VHIVQMFINTS |
| CD8 Hinge | 94 | KPTTTPAPRPPTPAPTIASQPLSLRPEACRPAAGGAVHTR |
| | | GLDFACDIYIWAPLAGTCGVLLLSLVITLYCNHRN |

In some aspects, the disclosed polypeptides comprise an antigen binding domain that does not employ an antibody or antibody fragment. In some aspects, a polypeptide of the disclosure comprises a sequence having at least 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NO:69.

### D. CD70

The present disclosure encompasses CD70 CARs. CD70, also known as CD70 antigen, CD27 ligand, and Tumor necrosis factor ligand superfamily member 7, is encoded by the CD70 gene (also known as TNFSF7). A CD70 mRNA sequence is provided by RefSeq accession number NM_001252, which is incorporated by reference herein in its entirety. A CD70 protein sequence is provided by RefSeq accession number NP_001243, which is incorporated by reference herein in its entirety.

### E. CD27

The present disclosure encompasses anti-CD70 CARs that comprise an antigen binding domain that does not employ an antibody or antibody fragment. In particular aspects, instead of the anti-CD70 CARs utilizing an antibody or antibody fragment as an antigen binding domain, the CAR instead utilizes part or all of CD27 in the CAR, including using the extracellular domain of CD27 as the antigen binding domain of the CAR, in some cases. CD27, also known as CD27 molecule, CD27L receptor, and Tumor necrosis factor receptor superfamily member 7, is encoded by the CD27 gene (also known as TNFRSF7). As a matter of reference, the Homo sapiens CD27 molecule (CD27) on chromosome 12 is provided at National Center for Biotechnology Information (NCBI) GenBank® Accession No. NG_031995.1, which is incorporated by reference herein in its entirety. One example of a full wild-type CD27 protein sequence is at NCBI GenBank® Accession No. P26842 (and is also identical to the amino acid sequence in Accession No. NG_031995.1), which is incorporated by reference herein in its entirety.

### VI. Nucleic Acids

In certain aspects, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides encoding a chimeric polypeptide, polynucleotides encoding a chimeric antigen receptor, polynucleotides encoding an immune cell engager, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Mutation

Changes can be introduced by mutation into a nucleic acid, thereby leading to changes in the amino acid sequence of a polypeptide (e.g., an antibody or antibody derivative, a chimeric polypeptide, etc.) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, e.g., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### B. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another aspect, the nucleic acid molecules may be used as probes or PCR primers for specific sequences. For instance, a nucleic acid molecule probe may be used in diagnostic methods, or a nucleic acid molecule PCR primer may be used to amplify regions of DNA that could be used, inter alia, to isolate nucleic acid sequences for use in producing variable domains of antibodies. See, e.g., Gaily Kivi et al., BMC Biotechnol. 16:2 (2016). In some aspects, the nucleic acid molecules are oligonucleotides. In some aspects, the oligonucleotides are from highly variable regions of the heavy and light chains of the antibody of interest. In some aspects, the oligonucleotides encode all or part of one or more CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### C. Sequences

The nucleic acid sequences encoding certain polypeptides, including viral genes, antibodies, chimeric antigen receptors, chimeric polypeptides, immune cell engagers, and portions, regions, and domains thereof, are provided in Table 2.

TABLE 2

| | SEQ | |
|---|---|---|
| | ID | |
| NAME | NO | SEQUENCES |
| E4ORF-1 | 1 | ATGGCTGAATCTCTGTATGCTTTCATAGATAGCCCTGGAGGG |
| (DNA) | | ATCGCTCCCGTCCAGGAAGGGGCTAGCAATAGATATATCTTC |
| | | TTTTGCCCCGAATCTTTCCACATTCCTCCGCATGGGGTGATA |
| | | TTGCTTCACCTCAGAGTGAGCGTGCTGGTTCCTACTGGATAT |
| | | CAGGGCAGATTTATGGCCTTGAATGACTACCATGCCAGGGGC |
| | | ATACTAACCCAGTCCGATGTGATATTTGCCGGGAGAAGACAT |
| | | GATCTCTCTGTGCTGCTCTTTAACCACACGGACCGATTTTTG |
| | | TATGTCCGCGAGGGCCACCCAGTGGGAACCCTGCTGCTGGAG |
| | | AGAGTGATTTTTCCTTCAGTGAGAATAGCCACCCTGGTTTAG |
| C16 (DNA) | 2 | ATGATATCAC TCTCATTCCT CATCCACAAC CCATTAAAAA |
| | | AATGGAAATT AAAGCCCTCT ATTAGCATAA ACGGCTACAG |
| | | GTCTACCTTT ACAATGGCCT TTCCTTGTGC CCAGTTCAGA |
| | | CCCTGTCATT GCCACGCTAC TAAGGACTCC CTGAATACCG |
| | | TGGCCGACGT CAGACATTGT CTGACTGAAT ACATCCTGTG |
| | | GGTTTCTCAT AGATGGACCC ATAGAGAAAG CGCAGGGTCT |
| | | CTCTACAGGC TTCTCATCTC TTTCAGAACT GATGCAACGG |
| | | AGCTCTTTGG TGGTGAGTTG AAGGATTCAC TTCCGTGGGA |
| | | CAATATCGAC AATTGCGTGG AGATCATTAA ATGTTTCATC |
| | | AGAAATGACT CCATGAAAAC CGCCGAAGAA CTTCGTGCAA |
| | | TCATTGGACT TTGTACTCAA TCAGCTATCG TCTCTGGAAG |
| | | AGTCTTCAAC GATAAGTATA TCGACATACT ACTTATGCTG |
| | | CGAAAGATTC TGAACGAGAA CGACTATCTC ACCCTCTTGG |
| | | ATCATATCCG CACTGCTAAA TACTAA |
| NS3 (DNA) | 3 | TCAGGAGTGC TATGGGACAC ACCCAGCCCT CCAGAAGTGG |
| | | AAAGAGCAGT CCTTGACAAT GGCATCTATA GAATTCTCCA |
| | | AAGAGGATTG TTGGGCAGGT CTCAAGTAGG AGTAGGAGTT |
| | | TTTCAAGAAG GTGTGTTCCA CACAATGTGG CACGTCACCA |
| | | GGGGAGCTGT TCTCATGTAC CAAGGGAAGA GATTGGAACC |
| | | AAGTTGGGCT AGTGTCAAGA AAGACTTGAT CTCATATGGA |
| | | GGAGGTTGGA GATTTCAAGG ATCCTGGAAC ACGGGAGAAG |
| | | AGGTGCAGGT GATTGCTGTT GAACCGGGGA AGAACCCCAA |
| | | AAATGTACAG ACAACGCCAG GCACCTTCAA GACCCCTGAA |
| | | GGTGAAATTG GAGCTATAGC TCTAGACTTC AAACCCGGCA |
| | | CATCTGGATC TCCTATCGTG AACAGAGAGG GAAAAATAGT |
| | | GGGTCTTTAT GGAAATGGAG TGGTGACAAC AAGTGGAACC |
| | | TATGTCAGTG CCATAGCTCA AGCTAAAGTA TCACAAGAAG |
| | | GGCCCCTACC AGAGATTGAG GACGAGGTGT TTAGGAAA |
| CO CAR.CD70 | 4 | ATGGCCCTGCCTGTGACAGCTCTGCTCCTCCCTCTGGCCC |
| 42D12 VLVH | | TGCTGCTCCATGCCGCCAGACCCCAGGCAGTTGTGACCCA |
| (DNA) | | GGAGCCTTCCCTGACAGTGTCTCCAGGAGGGACGGTCACG |
| | | CTCACCTGCGGCCTCAAATCTGGGTCTGTCACTTCCGATA |
| | | ACTTCCCCACTTGGTACCAGCAGACACCAGGCCAGGCTCC |
| | | CCGATTGCTTATCTACAACACAAACACCCGTCACTCTGGC |
| | | GTCCCCGACCGCTTCTCCGGATCCATCCTGGGCAACAAAG |
| | | CCGCCCTCACCATCACGGGGGCCCAGGCCGACGACGAGGC |
| | | CGAATATTTCTGTGCTCTGTTCATAAGTAATCCTAGTGTT |
| | | GAGTTCGGCGGAGGGACCCAACTGACCGTCCTAGGTGGCA |
| | | GCACCAGCGGCTCCGGCAAGCCTGGCTCTGGCGAGGGCAG |
| | | CACAAAGGGAGAGGTGCAGCTCGTGGAGTCTGGGGGAGGC |
| | | TTGGTGCAGCCTGGGGGGTCTCTGAGACTCTCCTGTGCAG |
| | | CCTCTGGATTCACCTTCAGTGTCTACTACATGAACTGGGT |
| | | CCGCCAGGCTCCAGGGAAGGGGCTCGAGTGGGTCTCAGAT |
| | | ATTAATAATGAAGGTGGTACTACATACTATGCAGACTCCG |
| | | TGAAGGGCCGATTCACCATCTCCAGAGACAACTCTAAGAA |
| | | CAGCCTGTATCTGCAAATGAACAGCCTGCGCGCCGAGGAC |
| | | ACGGCCGTGTACTACTGCGCGAGAGATGCCGGATATAGCA |
| | | ACCATGTACCCATCTTTGATTCTTGGGGCCAGGGGACCCT |
| | | GGTCACTGTCTCCTCA |
| CAR.CD70 | 5 | ATGGCCCTGCCTGTGACAGCTCTGCTCCTCCCTCTGGCCC |
| 42D12 VHVL | | TGCTGCTCCATGCCGCCAGACCCGAGGTGCAGCTCGTGGA |
| (DNA) | | GTCTGGGGGAGGCTTGGTGCAGCCTGGGGGGTCTCTGAGA |
| | | CTCTCCTGTGCAGCCTCTGGATTCACCTTCAGTGTCTACT |
| | | ACATGAACTGGGTCCGCCAGGCTCCAGGGAAGGGGCTCGA |
| | | GTGGGTCTCAGATATTAATAATGAAGGTGGTACTACATAC |
| | | TATGCAGACTCCGTGAAGGGCCGATTCACCATCTCCAGAG |
| | | ACAACTCTAAGAACAGCCTGTATCTGCAAATGAACAGCCT |
| | | GCGCGCCGAGGACACGGCCGTGTACTACTGCGCGAGAGAT |
| | | GCCGGATATAGCAACCATGTACCCATCTTTGATTCTTGGG |
| | | GCCAGGGGACCCTGGTCACTGTCTCCTCAGGCAGCACCAG |
| | | CGGCTCCGGCAAGCCTGGCTCTGGCGAGGGCAGCACAAAG |
| | | GGACAGGCAGTGGTGACCCAGGAGCCTTCCCTGACAGTGT |
| | | CTCCAGGAGGGACGGTCACGCTCACCTGCGGCCTCAAATC |
| | | TGGGTCTGTCACTTCCGATAACTTCCCCACTTGGTACCAG |
| | | CAGACACCAGGCCAGGCTCCCCGATTGCTTATCTACAACA |
| | | CAAACACCCGTCACTCTGGCGTCCCCGACCGCTTCTCCGG |
| | | ATCCATCCTGGGCAACAAAGCCGCCCTCACCATCACGGGG |
| | | GCCCAGGCCGACGACGAGGCCGAATATTTCTGTGCTCTGT |
| | | TCATAAGTAATCCTAGTGTTGAGTTCGGCGGAGGGACCCA |
| | | ACTGACCGTCCTAGGT |
| CAR.CD70 | 6 | ATGGCCCTGCCTGTGACAGCTCTGCTCCTCCCTCTGGCCC |
| 42D12 VLVH | | TGCTGCTCCATGCCGCCAGACCCGAGGTGCAGCTCGTGGA |
| (DNA) | | GTCTGGGGGAGGCTTGGTGCAGCCTGGGGGGTCTCTGAGA |
| | | CTCTCCTGTGCAGCCTCTGGATTCACCTTCAGTGTCTACT |
| | | ACATGAACTGGGTCCGCCAGGCTCCAGGGAAGGGGCTTGA |
| | | GTGGGTCTCAGATATTAATAATGAAGGTGGTACTACATAC |
| | | TATGCAGACTCCGTGAAGGGCCGATTCACCATCTCCAGAG |
| | | ACAACTCTAAGAACAGCCTGTATCTGCAAATGAACAGCCT |
| | | GCGCGCCGAGGACACGGCCGTGTACTACTGCGCGAGAGAT |
| | | GCCGGATATAGCAACCATGTACCCATCTTTGATTCTTGGG |
| | | GCCAGGGGACCCTGGTCACTGTCTCCTCAGGCAGCACCAG |
| | | CGGCTCCGGCAAGCCTGGCTCTGGCGAGGGCAGCACAAAG |
| | | GGACAGGCAGTGGTGACCCAGGAGCCTTCCCTGACAGTGT |
| | | CTCCAGGAGGGACGGTCACGCTCACCTGCGGCCTCAAATC |
| | | TGGGTCTGTCACTTCCGATAACTTCCCCACTTGGTACCAG |
| | | CAGACACCAGGCCAGGCTCCCCGATTGCTTATCTACAACA |
| | | CAAACACCCGTCACTCTGGCGTCCCCGACCGCTTCTCCGG |
| | | ATCCATCCTGGGCAACAAAGCCGCCCTCACCATCACGGGG |
| | | GCCCAGGCCGACGACGAGGCCGAATATTTCTGTGCTCTGT |
| | | TCATAAGTAATCCTAGTGTTGAGTTCGGCGGAGGGACCCA |
| | | ACTGACCGTCCTAGGT |
| CD28 | 7 | TTTTGGGTGCTGGTGGTGGTTGGTGGAGTCCTGGCTTGCT |
| transmembrane | | ATAGCTTGCTAGTAACAGTGGCCTTTATTATTTTCTGGGT |
| domain (DNA) | | G |
| CD3zeta | 8 | CGCGTGAAGTTCAGCAGGAGCGCAGACGCCCCCGCGTACC |
| intracellular | | AGCAGGGCCAGAACCAGCTCTATAACGAGCTCAATCTAGG |
| domain 3 | | ACGAAGAGAGGAGTACGATGTTTTGGACAAAAGACGTGGC |
| (DNA) | | CGGGACCCTGAGATGGGGGGAAAGCCGAGAAGGAAGAACC |
| | | CTCAGGAAGGCCTGTACAATGAACTGCAGAAAGATAAGAT |
| | | GGCGGAGGCCTACAGTGAGATTGGGATGAAAGGCGAGCGC |
| | | CGGAGGGGCAAGGGGCACGATGGCCTTTACCAGGGTCTCA |
| | | GTACAGCCACCAAGGACACCTACGACGCCCTTCACATGCA |
| | | GGCCCTGCCCCCTCGCGGAC |
| 4-1BB | 9 | AAACGGGGCAGAAAGAAACTCCTGTATATATTCAAACAAC |
| cytoplasmic | | CATTTATGAGACCAGTACAAACTACTCAAGAGGAAGATGG |
| domain (DNA) | | CTGTAGCTGCCGATTTCCAGAAGAAGAAGAAGGAGGATGT |
| | | GAACTG |
| DAP10 | 10 | CTTTGCGCACGCCCACGCCGCAGCCCCGCCCAAGAAGATG |
| intracytoplasmic | | GCAAAGTCTACATCAACATGCCAGGCAGGGGC |
| domain | | |
| (DNA) | | |
| NKG2D | 11 | AGCGCGAACGAACGCTGCAAAAGCAAAGTGGTGCCGTGCC |
| intraprotoplasmic | | GCCAGAAACAGTGGCGCACCAGCTTTGATAGCAAAAAACT |
| domain | | GGATCTGAACTATAACCATTTTGAAAGCATGGAATGGAGC |
| (DNA) | | CATCGCAGCCGCCGCGGCCGCATTTGGGGCATG |
| DAP12 (DNA) | 12 | TACTTCCTGGGCCGGCTGGTCCCTCGGGGGCGAGGGGCTG |
| | | CGGAGGCAGCGACCCGGAAACAGCGTATCACTGAGACCGA |
| | | GTCGCCTTATCAGGAGCTCCAGGGTCAGAGGTCGGATGTC |
| | | TACAGCGACCTCAACACACAGAGGCCGTATTACAAA |
| CD27 signal | 13 | ATGGCACGGCCACATCCCTGGTGGCTGTGCGTTCTGGGGA |
| peptide (DNA) | | CCCTGGTGGGGCTCTCA |
| GMCSF- | 14 | ATGCTTCTCCTGGTGACAAGCCTTCTGCTCTGTGAGTTAC |
| Receptor | | CACACCCAGCATTCCTCCTGATCCCA |
| (DNA) | | |
| CD27 | 15 | GCTACTCCAGCCCCCAAGAGCTGCCCAGAGAGGCACTACT |
| extracellular | | GGGCTCAGGGAAAGCTGTGCTGCCAGATGTGTGAGCCAGG |
| domain (DNA) | | AACATTCCTCGTGAAGGACTGTGACCAGCATAGAAAGGCT |
| | | GCTCAGTGTGATCCTTGCATACCGGGGGTCTCCTTCTCTC |
| | | CTGACCACCACACCCGGCCCCACTGTGAGAGCTGTCGGCA |
| | | CTGTAACTCTGGTCTTCTCGTTCGCAACTGCACCATCACT |
| | | GCCAATGCTGAGTGTGCCTGTCGCAATGGCTGGCAGTGCA |
| | | GGGACAAGGAGTGCACCGAGTGTGATCCTCTTCCAAACCC |
| | | TTCGCTGACCGCTCGGTCGTCTCAGGCCCTGAGCCCACAC |
| | | CCTCAGCCCACCCACTTACCTTATGTCAGTGAGATGCTGG |
| | | AGGCCAGGACAGCTGGGCACATGCAGACTCTGGCTGACTT |
| | | CAGGCAGCTGCCTGCCCGGACTCTCTCTACCCACTGGCCA |
| | | CCCCAAAGATCCCTGTGCAGCTCCGATTTTATTCGC |
| Codon- | 16 | GCTACACCGGCTCCGAAGTCCTGCCCGGAGCGGCATTATT |
| optimized | | GGGCACAGGGCAAGTTGTGTTGTCAAATGTGTGAGCCGGG |
| CD27 | | AACCTTTCTCGTGAAGGATTGCGATCAGCATCGGAAGGCC |
| extracellular | | GCGCAGTGCGACCCATGTATACCAGGGGTCTCATTTTCCC |
| domain (DNA) | | CAGATCACCATACGAGGCCGCACTGTGAGTCTTGCAGGCA |
| | | TTGTAATTCCGGCTTGTTGGTCCGCAACTGTACTATTACT |
| | | GCGAATGCAGAGTGTGCTTGTAGAAACGGATGGCAGTGCA |
| | | GGGACAAAGAATGTACGGAGTGTGATCCACTGCCTAACCC |
| | | CAGTCTTACAGCAAGATCTTCACAGGCCCTCAGCCCGCAT |
| | | CCTCAACCAACACATCTTCCTTACGTGTCAGAAATGTTGG |
| | | AGGCGCGAACCGCAGGCCATATGCAGACCCTGGCGGACTT |
| | | TCGGCAGCTGCCAGCACGCACACTTAGTACACACTGGCCA |
| | | CCACAACGCAGCTTGTGCTCTTCCGATTTCATCCGC |
| CD27 | 17 | ATCCTTGTGATCTTCTCTGGAATGTTCCTTGTTTTCACCC |
| transmembrane | | TGGCCGGGGCCCTGTTCCTCCAT |
| domain (DNA) | | |
| CD27tr28tdmC | 18 | ATGGCACGGCCACATCCCTGGTGGCTGTGCGTTCTGGGGA |
| D3zIL 15 | | CCCTGGTGGGGCTCTCAGCTACTCCAGCCCCCAAGAGCTG |
| (DNA) | | CCCAGAGAGGCACTACTGGGCTCAGGGAAAGCTGTGCTGC |
| | | CAGATGTGTGAGCCAGGAACATTCCTCGTGAAGGACTGTG |
| | | ACCAGCATAGAAAGGCTGCTCAGTGTGATCCTTGCATACC |
| | | GGGGGTCTCCTTCTCTCCTGACCACCACACCCGGCCCCAC |
| | | TGTGAGAGCTGTCGGCACTGTAACTCTGGTCTTCTCGTTC |
| | | GCAACTGCACCATCACTGCCAATGCTGAGTGTGCCTGTCG |
| | | CAATGGCTGGCAGTGCAGGGACAAGGAGTGCACCGAGTGT |
| | | GATCCTCTTCCAAACCCTTCGCTGACCGCTCGGTCGTCTC |
| | | AGGCCCTGAGCCCACACCCTCAGCCCACCCACTTACCTTA |
| | | TGTCAGTGAGATGCTGGAGGCCAGGACAGCTGGGCACATG |
| | | CAGACTCTGGCTGACTTCAGGCAGCTGCCTGCCCGGACTC |
| | | TCTCTACCCACTGGCCACCCCAAAGATCCCTGTGCAGCTC |
| | | CGATTTTATTCGCTTTTGGGTGCTGGTGGTGGTTGGTGGA |
| | | GTCCTGGCTTGCTATAGCTTGCTAGTAACAGTGGCCTTTA |
| | | TTATTTTCTGGGTG |
| CD27Tr28tmd | 19 | ATGACAAGAGTTACTAACAGCCCCTCTCTCCAAGCTCACT |
| 41BBicd3zIL1 | | TACAGGCTCTCTACTTAGTCCAGCACGAAGTCTGGAGACC |
| 5 (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCTTTTGGGTGCTGGTGGT |
| | | GGTTGGTGGAGTCCTGGCTTGCTATAGCTTGCTAGTAACA |
| | | GTGGCCTTTATTATTTTCTGGGTGAAACGGGGCAGAAAGA |
| | | AACTCCTGTATATATTCAAACAACCATTTATGAGACCAGT |
| | | ACAAACTACTCAAGAGGAAGATGGCTGTAGCTGCCGATTT |
| | | CCAGAAGAAGAAGAAGGAGGATGTGAACTGAAACGCGTGA |
| | | AGTTCAGCAGGAGCGCAGACGCCCCCGCGTACCAGCAGGG |
| | | CCAGAACCAGCTCTATAACGAGCTCAATCTAGGACGAAGA |
| | | GAGGAGTACGATGTTTTGGACAAAAGACGTGGCCGGGACC |
| | | CTGAGATGGGGGGAAAGCCGAGAAGGAAGAACCCTCAGGA |
| | | AGGCCTGTACAATGAACTGCAGAAAGATAAGATGGCGGAG |
| | | GCCTACAGTGAGATTGGGATGAAAGGCGAGCGCCGGAGGG |
| | | GCAAGGGGCACGATGGCCTTTACCAGGGTCTCAGTACAGC |
| | | CACCAAGGACACCTACGACGCCCTTCACATGCAGGCCCTG |
| | | CCCCCTCGCGGACCGCAGTGTACTAATTATGCTCTCTTGA |
| | | AATTGGCTGGAGATGTTGAGAGCAATCCCGGGCCCATGCG |
| | | CATTAGCAAGCCCCACCTGCGGAGCATCAGCATCCAGTGC |
| | | TACCTGTGCCTGCTGCTGAACAGCCACTTCCTGACCGAGG |
| | | CCGGCATCCACGTGTTCATCCTGGGCTGCTTCAGCGCCGG |
| | | ACTGCCCAAGACCGAGGCCAACTGGGTGAACGTGATCAGC |
| | | GACCTGAAGAAGATCGAGGACCTGATCCAGAGCATGCACA |
| | | TCGACGCCACCCTGTACACCGAGAGCGACGTGCACCCCAG |
| | | CTGCAAGGTGACCGCCATGAAGTGCTTTCTGCTGGAACTG |
| | | CAGGTGATCAGCCTGGAAAGCGGCGACGCCAGCATCCACG |
| | | ACACCGTGGAGAACCTGATCATCCTGGCCAACAACAGCCT |
| | | GAGCAGCAACGGCAACGTGACCGAGAGCGGCTGCAAAGAG |
| | | TGCGAGGAACTGGAAGAGAAGAACATCAAAGAGTTTCTGC |
| | | AGAGCTTCGTGCACATCGTGCAGATGTTCATCAACACCAG |
| | | CTGA |
| GSPco27Tr28t | 20 | ATGACAAGAGTTACTAACAGCCCCTCTCTCCAAGCTCACT |
| md41BBicCD3 | | TACAGGCTCTCTACTTAGTCCAGCACGAAGTCTGGAGACC |
| zIL15 (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCTTTTGGGT |
| | | GCTGGTGGTGGTTGGTGGAGTCCTGGCTTGCTATAGCTTG |
| | | CTAGTAACAGTGGCCTTTATTATTTTCTGGGTGAAACGGG |
| | | GCAGAAAGAAACTCCTGTATATATTCAAACAACCATTTAT |
| | | GAGACCAGTACAAACTACTCAAGAGGAAGATGGCTGTAGC |
| | | TGCCGATTTCCAGAAGAAGAAGAAGGAGGATGTGAACTGA |
| | | AACGCGTGAAGTTCAGCAGGAGCGCAGACGCCCCCGCGTA |
| | | CCAGCAGGGCCAGAACCAGCTCTATAACGAGCTCAATCTA |
| | | GGACGAAGAGAGGAGTACGATGTTTTGGACAAAAGACGTG |
| | | GCCGGGACCCTGAGATGGGGGGAAAGCCGAGAAGGAAGAA |
| | | CCCTCAGGAAGGCCTGTACAATGAACTGCAGAAAGATAAG |
| | | ATGGCGGAGGCCTACAGTGAGATTGGGATGAAAGGCGAGC |
| | | GCCGGAGGGGCAAGGGGCACGATGGCCTTTACCAGGGTCT |
| | | CAGTACAGCCACCAAGGACACCTACGACGCCCTTCACATG |
| | | CAGGCCCTGCCCCCTCGCGGACCGCAGTGTACTAATTATG |
| | | CTCTCTTGAAATTGGCTGGAGATGTTGAGAGCAATCCCGG |
| | | GCCCATGCGCATTAGCAAGCCCCACCTGCGGAGCATCAGC |
| | | ATCCAGTGCTACCTGTGCCTGCTGCTGAACAGCCACTTCC |
| | | TGACCGAGGCCGGCATCCACGTGTTCATCCTGGGCTGCTT |
| | | CAGCGCCGGACTGCCCAAGACCGAGGCCAACTGGGTGAAC |
| | | GTGATCAGCGACCTGAAGAAGATCGAGGACCTGATCCAGA |
| | | GCATGCACATCGACGCCACCCTGTACACCGAGAGCGACGT |
| | | GCACCCCAGCTGCAAGGTGACCGCCATGAAGTGCTTTCTG |
| | | CTGGAACTGCAGGTGATCAGCCTGGAAAGCGGCGACGCCA |
| | | GCATCCACGACACCGTGGAGAACCTGATCATCCTGGCCAA |
| | | CAACAGCCTGAGCAGCAACGGCAACGTGACCGAGAGCGGC |
| | | TGCAAAGAGTGCGAGGAACTGGAAGAGAAGAACATCAAAG |
| | | AGTTTCTGCAGAGCTTCGTGCACATCGTGCAGATGTTCAT |
| | | CAACACCAGCTGA |
| CD27Tr28tmd | 21 | ATGACAAGAGTTACTAACAGCCCCTCTCTCCAAGCTCACT |
| DAP10icd3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCTTTTGGGTGCTGGTGGT |
| | | GGTTGGTGGAGTCCTGGCTTGCTATAGCTTGCTAGTAACA |
| | | GTGGCCTTTATTATTTTCTGGGTGCTTTGCGCACGCCCAC |
| | | GCCGCAGCCCCGCCCAAGAAGATGGCAAAGTCTACATCAA |
| | | CATGCCAGGCAGGGGCAAACGCGTGAAGTTCAGCAGGAGC |
| | | GCAGACGCCCCCGCGTACCAGCAGGGCCAGAACCAGCTCT |
| | | ATAACGAGCTCAATCTAGGACGAAGAGAGGAGTACGATGT |
| | | TTTGGACAAAAGACGTGGCCGGGACCCTGAGATGGGGGGA |
| | | AAGCCGAGAAGGAAGAACCCTCAGGAAGGCCTGTACAATG |
| | | AACTGCAGAAAGATAAGATGGCGGAGGCCTACAGTGAGAT |
| | | TGGGATGAAAGGCGAGCGCCGGAGGGGCAAGGGGCACGAT |
| | | GGCCTTTACCAGGGTCTCAGTACAGCCACCAAGGACACCT |
| | | ACGACGCCCTTCACATGCAGGCCCTGCCCCCTCGCGGACC |
| | | GCAGTGTACTAATTATGCTCTCTTGAAATTGGCTGGAGAT |
| | | GTTGAGAGCAATCCCGGGCCCATGCGCATTAGCAAGCCCC |
| | | ACCTGCGGAGCATCAGCATCCAGTGCTACCTGTGCCTGCT |
| | | GCTGAACAGCCACTTCCTGACCGAGGCCGGCATCCACGTG |
| | | TTCATCCTGGGCTGCTTCAGCGCCGGACTGCCCAAGACCG |
| | | AGGCCAACTGGGTGAACGTGATCAGCGACCTGAAGAAGAT |
| | | CGAGGACCTGATCCAGAGCATGCACATCGACGCCACCCTG |
| | | TACACCGAGAGCGACGTGCACCCCAGCTGCAAGGTGACCG |
| | | CCATGAAGTGCTTTCTGCTGGAACTGCAGGTGATCAGCCT |
| | | GGAAAGCGGCGACGCCAGCATCCACGACACCGTGGAGAAC |
| | | CTGATCATCCTGGCCAACAACAGCCTGAGCAGCAACGGCA |
| | | ACGTGACCGAGAGCGGCTGCAAAGAGTGCGAGGAACTGGA |
| | | AGAGAAGAACATCAAAGAGTTTCTGCAGAGCTTCGTGCAC |
| | | ATCGTGCAGATGTTCATCAACACCAGCTGA |
| GSPco27Tr28t | 22 | ATGACAAGAGTTACTAACAGCCCCTCTCTCCAAGCTCACT |
| mdDAP10IL15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCTTTTGGGT |
| | | GCTGGTGGTGGTTGGTGGAGTCCTGGCTTGCTATAGCTTG |
| | | CTAGTAACAGTGGCCTTTATTATTTTCTGGGTGCTTTGCG |
| | | CACGCCCACGCCGCAGCCCCGCCCAAGAAGATGGCAAAGT |
| | | CTACATCAACATGCCAGGCAGGGGCAAACGCGTGAAGTTC |
| | | AGCAGGAGCGCAGACGCCCCCGCGTACCAGCAGGGCCAGA |
| | | ACCAGCTCTATAACGAGCTCAATCTAGGACGAAGAGAGGA |
| | | GTACGATGTTTTGGACAAAAGACGTGGCCGGGACCCTGAG |
| | | ATGGGGGGAAAGCCGAGAAGGAAGAACCCTCAGGAAGGCC |
| | | TGTACAATGAACTGCAGAAAGATAAGATGGCGGAGGCCTA |
| | | CAGTGAGATTGGGATGAAAGGCGAGCGCCGGAGGGGCAAG |
| | | GGGCACGATGGCCTTTACCAGGGTCTCAGTACAGCCACCA |
| | | AGGACACCTACGACGCCCTTCACATGCAGGCCCTGCCCCC |
| | | TCGCGGACCGCAGTGTACTAATTATGCTCTCTTGAAATTG |
| | | GCTGGAGATGTTGAGAGCAATCCCGGGCCCATGCGCATTA |
| | | GCAAGCCCCACCTGCGGAGCATCAGCATCCAGTGCTACCT |
| | | GTGCCTGCTGCTGAACAGCCACTTCCTGACCGAGGCCGGC |
| | | ATCCACGTGTTCATCCTGGGCTGCTTCAGCGCCGGACTGC |
| | | CCAAGACCGAGGCCAACTGGGTGAACGTGATCAGCGACCT |
| | | GAAGAAGATCGAGGACCTGATCCAGAGCATGCACATCGAC |
| | | GCCACCCTGTACACCGAGAGCGACGTGCACCCCAGCTGCA |
| | | AGGTGACCGCCATGAAGTGCTTTCTGCTGGAACTGCAGGT |
| | | GATCAGCCTGGAAAGCGGCGACGCCAGCATCCACGACACC |
| | | GTGGAGAACCTGATCATCCTGGCCAACAACAGCCTGAGCA |
| | | GCAACGGCAACGTGACCGAGAGCGGCTGCAAAGAGTGCGA |
| | | GGAACTGGAAGAGAAGAACATCAAAGAGTTTCTGCAGAGC |
| | | TTCGTGCACATCGTGCAGATGTTCATCAACACCAGCTGA |
| CD27Tr28tmd | 23 | ATGACAAGAGTTACTAACAGCCCCTCTCTCCAAGCTCACT |
| DAP12icd3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCTTTTGGGTGCTGGTGGT |
| | | GGTTGGTGGAGTCCTGGCTTGCTATAGCTTGCTAGTAACA |
| | | GTGGCCTTTATTATTTTCTGGGTGTACTTCCTGGGCCGGC |
| | | TGGTCCCTCGGGGGCGAGGGGCTGCGGAGGCAGCGACCCG |
| | | GAAACAGCGTATCACTGAGACCGAGTCGCCTTATCAGGAG |
| | | CTCCAGGGTCAGAGGTCGGATGTCTACAGCGACCTCAACA |
| | | CACAGAGGCCGTATTACAAAAAACGCGTGAAGTTCAGCAG |
| | | GAGCGCAGACGCCCCCGCGTACCAGCAGGGCCAGAACCAG |
| | | CTCTATAACGAGCTCAATCTAGGACGAAGAGAGGAGTACG |
| | | ATGTTTTGGACAAAAGACGTGGCCGGGACCCTGAGATGGG |
| | | GGGAAAGCCGAGAAGGAAGAACCCTCAGGAAGGCCTGTAC |
| | | AATGAACTGCAGAAAGATAAGATGGCGGAGGCCTACAGTG |
| | | AGATTGGGATGAAAGGCGAGCGCCGGAGGGGCAAGGGGCA |
| | | CGATGGCCTTTACCAGGGTCTCAGTACAGCCACCAAGGAC |
| | | ACCTACGACGCCCTTCACATGCAGGCCCTGCCCCCTCGCG |
| | | GACCGCAGTGTACTAATTATGCTCTCTTGAAATTGGCTGG |
| | | AGATGTTGAGAGCAATCCCGGGCCCATGCGCATTAGCAAG |
| | | CCCCACCTGCGGAGCATCAGCATCCAGTGCTACCTGTGCC |
| | | TGCTGCTGAACAGCCACTTCCTGACCGAGGCCGGCATCCA |
| | | CGTGTTCATCCTGGGCTGCTTCAGCGCCGGACTGCCCAAG |
| | | ACCGAGGCCAACTGGGTGAACGTGATCAGCGACCTGAAGA |
| | | AGATCGAGGACCTGATCCAGAGCATGCACATCGACGCCAC |
| | | CCTGTACACCGAGAGCGACGTGCACCCCAGCTGCAAGGTG |
| | | ACCGCCATGAAGTGCTTTCTGCTGGAACTGCAGGTGATCA |
| | | GCCTGGAAAGCGGCGACGCCAGCATCCACGACACCGTGGA |
| | | GAACCTGATCATCCTGGCCAACAACAGCCTGAGCAGCAAC |
| | | GGCAACGTGACCGAGAGCGGCTGCAAAGAGTGCGAGGAAC |
| | | TGGAAGAGAAGAACATCAAAGAGTTTCTGCAGAGCTTCGT |
| | | GCACATCGTGCAGATGTTCATCAACACCAGCTGA |
| GSPco27Tr28t | 24 | ATGACAAGAGTTACTAACAGCCCCTCTCTCCAAGCTCACT |
| mddap12icd15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCTTTTGGGT |
| | | GCTGGTGGTGGTTGGTGGAGTCCTGGCTTGCTATAGCTTG |
| | | CTAGTAACAGTGGCCTTTATTATTTTCTGGGTGTACTTCC |
| | | TGGGCCGGCTGGTCCCTCGGGGGCGAGGGGCTGCGGAGGC |
| | | AGCGACCCGGAAACAGCGTATCACTGAGACCGAGTCGCCT |
| | | TATCAGGAGCTCCAGGGTCAGAGGTCGGATGTCTACAGCG |
| | | ACCTCAACACACAGAGGCCGTATTACAAAAAACGCGTGAA |
| | | GTTCAGCAGGAGCGCAGACGCCCCCGCGTACCAGCAGGGC |
| | | CAGAACCAGCTCTATAACGAGCTCAATCTAGGACGAAGAG |
| | | AGGAGTACGATGTTTTGGACAAAAGACGTGGCCGGGACCC |
| | | TGAGATGGGGGGAAAGCCGAGAAGGAAGAACCCTCAGGAA |
| | | GGCCTGTACAATGAACTGCAGAAAGATAAGATGGCGGAGG |
| | | CCTACAGTGAGATTGGGATGAAAGGCGAGCGCCGGAGGGG |
| | | CAAGGGGCACGATGGCCTTTACCAGGGTCTCAGTACAGCC |
| | | ACCAAGGACACCTACGACGCCCTTCACATGCAGGCCCTGC |
| | | CCCCTCGCGGACCGCAGTGTACTAATTATGCTCTCTTGAA |
| | | ATTGGCTGGAGATGTTGAGAGCAATCCCGGGCCCATGCGC |
| | | ATTAGCAAGCCCCACCTGCGGAGCATCAGCATCCAGTGCT |
| | | ACCTGTGCCTGCTGCTGAACAGCCACTTCCTGACCGAGGC |
| | | CGGCATCCACGTGTTCATCCTGGGCTGCTTCAGCGCCGGA |
| | | CTGCCCAAGACCGAGGCCAACTGGGTGAACGTGATCAGCG |
| | | ACCTGAAGAAGATCGAGGACCTGATCCAGAGCATGCACAT |
| | | CGACGCCACCCTGTACACCGAGAGCGACGTGCACCCCAGC |
| | | TGCAAGGTGACCGCCATGAAGTGCTTTCTGCTGGAACTGC |
| | | AGGTGATCAGCCTGGAAAGCGGCGACGCCAGCATCCACGA |
| | | CACCGTGGAGAACCTGATCATCCTGGCCAACAACAGCCTG |
| | | AGCAGCAACGGCAACGTGACCGAGAGCGGCTGCAAAGAGT |
| | | GCGAGGAACTGGAAGAGAAGAACATCAAAGAGTTTCTGCA |
| | | GAGCTTCGTGCACATCGTGCAGATGTTCATCAACACCAGC |
| | | TGA |
| CD27Tr28tmd | 25 | ATGACAAGAGTTACTAACAGCCCCTCTCTCCAAGCTCACT |
| NKG2Dic3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCTTTTGGGTGCTGGTGGT |
| | | GGTTGGTGGAGTCCTGGCTTGCTATAGCTTGCTAGTAACA |
| | | GTGGCCTTTATTATTTTCTGGGTGAGCGCGAACGAACGCT |
| | | GCAAAAGCAAAGTGGTGCCGTGCCGCCAGAAACAGTGGCG |
| | | CACCAGCTTTGATAGCAAAAAACTGGATCTGAACTATAAC |
| | | CATTTTGAAAGCATGGAATGGAGCCATCGCAGCCGCCGCG |
| | | GCCGCATTTGGGGCATGAAACGCGTGAAGTTCAGCAGGAG |
| | | CGCAGACGCCCCCGCGTACCAGCAGGGCCAGAACCAGCTC |
| | | TATAACGAGCTCAATCTAGGACGAAGAGAGGAGTACGATG |
| | | TTTTGGACAAAAGACGTGGCCGGGACCCTGAGATGGGGGG |
| | | AAAGCCGAGAAGGAAGAACCCTCAGGAAGGCCTGTACAAT |
| | | GAACTGCAGAAAGATAAGATGGCGGAGGCCTACAGTGAGA |
| | | TTGGGATGAAAGGCGAGCGCCGGAGGGGCAAGGGGCACGA |
| | | TGGCCTTTACCAGGGTCTCAGTACAGCCACCAAGGACACC |
| | | TACGACGCCCTTCACATGCAGGCCCTGCCCCCTCGCGGAC |
| | | CGCAGTGTACTAATTATGCTCTCTTGAAATTGGCTGGAGA |
| | | TGTTGAGAGCAATCCCGGGCCCATGCGCATTAGCAAGCCC |
| | | CACCTGCGGAGCATCAGCATCCAGTGCTACCTGTGCCTGC |
| | | TGCTGAACAGCCACTTCCTGACCGAGGCCGGCATCCACGT |
| | | GTTCATCCTGGGCTGCTTCAGCGCCGGACTGCCCAAGACC |
| | | GAGGCCAACTGGGTGAACGTGATCAGCGACCTGAAGAAGA |
| | | TCGAGGACCTGATCCAGAGCATGCACATCGACGCCACCCT |
| | | GTACACCGAGAGCGACGTGCACCCCAGCTGCAAGGTGACC |
| | | GCCATGAAGTGCTTTCTGCTGGAACTGCAGGTGATCAGCC |
| | | TGGAAAGCGGCGACGCCAGCATCCACGACACCGTGGAGAA |
| | | CCTGATCATCCTGGCCAACAACAGCCTGAGCAGCAACGGC |
| | | AACGTGACCGAGAGCGGCTGCAAAGAGTGCGAGGAACTGG |
| | | AAGAGAAGAACATCAAAGAGTTTCTGCAGAGCTTCGTGCA |
| | | CATCGTGCAGATGTTCATCAACACCAGCTGA |
| GSPco27Tr28t | 26 | ATGACAAGAGTTACTAACAGCCCCTCTCTCCAAGCTCACT |
| mdNKG2Dicd | | TACAGGCTCTCTACTTAGTCCAGCACGAAGTCTGGAGACC |
| 3z15 (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCTTTTGGGT |
| | | GCTGGTGGTGGTTGGTGGAGTCCTGGCTTGCTATAGCTTG |
| | | CTAGTAACAGTGGCCTTTATTATTTTCTGGGTGAGCGCGA |
| | | ACGAACGCTGCAAAAGCAAAGTGGTGCCGTGCCGCCAGAA |
| | | ACAGTGGCGCACCAGCTTTGATAGCAAAAAACTGGATCTG |
| | | AACTATAACCATTTTGAAAGCATGGAATGGAGCCATCGCA |
| | | GCCGCCGCGGCCGCATTTGGGGCATGAAACGCGTGAAGTT |
| | | CAGCAGGAGCGCAGACGCCCCCGCGTACCAGCAGGGCCAG |
| | | AACCAGCTCTATAACGAGCTCAATCTAGGACGAAGAGAGG |
| | | AGTACGATGTTTTGGACAAAAGACGTGGCCGGGACCCTGA |
| | | GATGGGGGGAAAGCCGAGAAGGAAGAACCCTCAGGAAGGC |
| | | CTGTACAATGAACTGCAGAAAGATAAGATGGCGGAGGCCT |
| | | ACAGTGAGATTGGGATGAAAGGCGAGCGCCGGAGGGGCAA |
| | | GGGGCACGATGGCCTTTACCAGGGTCTCAGTACAGCCACC |
| | | AAGGACACCTACGACGCCCTTCACATGCAGGCCCTGCCCC |
| | | CTCGCGGACCGCAGTGTACTAATTATGCTCTCTTGAAATT |
| | | GGCTGGAGATGTTGAGAGCAATCCCGGGCCCATGCGCATT |
| | | AGCAAGCCCCACCTGCGGAGCATCAGCATCCAGTGCTACC |
| | | TGTGCCTGCTGCTGAACAGCCACTTCCTGACCGAGGCCGG |
| | | CATCCACGTGTTCATCCTGGGCTGCTTCAGCGCCGGACTG |
| | | CCCAAGACCGAGGCCAACTGGGTGAACGTGATCAGCGACC |
| | | TGAAGAAGATCGAGGACCTGATCCAGAGCATGCACATCGA |
| | | CGCCACCCTGTACACCGAGAGCGACGTGCACCCCAGCTGC |
| | | AAGGTGACCGCCATGAAGTGCTTTCTGCTGGAACTGCAGG |
| | | TGATCAGCCTGGAAAGCGGCGACGCCAGCATCCACGACAC |
| | | CGTGGAGAACCTGATCATCCTGGCCAACAACAGCCTGAGC |
| | | AGCAACGGCAACGTGACCGAGAGCGGCTGCAAAGAGTGCG |
| | | AGGAACTGGAAGAGAAGAACATCAAAGAGTTTCTGCAGAG |
| | | CTTCGTGCACATCGTGCAGATGTTCATCAACACCAGCTGA |
| CD27Tr41BBi | 27 | ATGACAAGAGTTACTAACAGCCCCTCTCTCCAAGCTCACT |
| cd3z15 (DNA) | | TACAGGCTCTCTACTTAGTCCAGCACGAAGTCTGGAGACC |
| | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCATCCTTGTGATCTTCTC |
| | | TGGAATGTTCCTTGTTTTCACCCTGGCCGGGGCCCTGTTC |
| | | CTCCATAAACGGGGCAGAAAGAAACTCCTGTATATATTCA |
| | | AACAACCATTTATGAGACCAGTACAAACTACTCAAGAGGA |
| | | AGATGGCTGTAGCTGCCGATTTCCAGAAGAAGAAGAAGGA |
| | | GGATGTGAACTGAAACGCGTGAAGTTCAGCAGGAGCGCAG |
| | | ACGCCCCCGCGTACCAGCAGGGCCAGAACCAGCTCTATAA |
| | | CGAGCTCAATCTAGGACGAAGAGAGGAGTACGATGTTTTG |
| | | GACAAAAGACGTGGCCGGGACCCTGAGATGGGGGGAAAGC |
| | | CGAGAAGGAAGAACCCTCAGGAAGGCCTGTACAATGAACT |
| | | GCAGAAAGATAAGATGGCGGAGGCCTACAGTGAGATTGGG |
| | | ATGAAAGGCGAGCGCCGGAGGGGCAAGGGGCACGATGGCC |
| | | TTTACCAGGGTCTCAGTACAGCCACCAAGGACACCTACGA |
| | | CGCCCTTCACATGCAGGCCCTGCCCCCTCGCGGACCGCAG |
| | | TGTACTAATTATGCTCTCTTGAAATTGGCTGGAGATGTTG |
| | | AGAGCAATCCCGGGCCCATGCGCATTAGCAAGCCCCACCT |
| | | GCGGAGCATCAGCATCCAGTGCTACCTGTGCCTGCTGCTG |
| | | AACAGCCACTTCCTGACCGAGGCCGGCATCCACGTGTTCA |
| | | TCCTGGGCTGCTTCAGCGCCGGACTGCCCAAGACCGAGGC |
| | | CAACTGGGTGAACGTGATCAGCGACCTGAAGAAGATCGAG |
| | | GACCTGATCCAGAGCATGCACATCGACGCCACCCTGTACA |
| | | CCGAGAGCGACGTGCACCCCAGCTGCAAGGTGACCGCCAT |
| | | GAAGTGCTTTCTGCTGGAACTGCAGGTGATCAGCCTGGAA |
| | | AGCGGCGACGCCAGCATCCACGACACCGTGGAGAACCTGA |
| | | TCATCCTGGCCAACAACAGCCTGAGCAGCAACGGCAACGT |
| | | GACCGAGAGCGGCTGCAAAGAGTGCGAGGAACTGGAAGAG |
| | | AAGAACATCAAAGAGTTTCTGCAGAGCTTCGTGCACATCG |
| | | TGCAGATGTTCATCAACACCAGCTGA |
| GSPco27Tr41 | 28 | ATGACAAGAGTTACTAACAGCCCCTCTCTCCAAGCTCACT |
| BBicd3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCATACTGGT |
| | | CATCTTTTCTGGAATGTTCCTTGTGTTCACCCTGGCAGGA |
| | | GCCCTGTTCCTTCACAAACGGGGCAGAAAGAAACTCCTGT |
| | | ATATATTCAAACAACCATTTATGAGACCAGTACAAACTAC |
| | | TCAAGAGGAAGATGGCTGTAGCTGCCGATTTCCAGAAGAA |
| | | GAAGAAGGAGGATGTGAACTGAAACGCGTGAAGTTCAGCA |
| | | GGAGCGCAGACGCCCCCGCGTACCAGCAGGGCCAGAACCA |
| | | GCTCTATAACGAGCTCAATCTAGGACGAAGAGAGGAGTAC |
| | | GATGTTTTGGACAAAAGACGTGGCCGGGACCCTGAGATGG |
| | | GGGGAAAGCCGAGAAGGAAGAACCCTCAGGAAGGCCTGTA |
| | | CAATGAACTGCAGAAAGATAAGATGGCGGAGGCCTACAGT |
| | | GAGATTGGGATGAAAGGCGAGCGCCGGAGGGGCAAGGGGC |
| | | ACGATGGCCTTTACCAGGGTCTCAGTACAGCCACCAAGGA |
| | | CACCTACGACGCCCTTCACATGCAGGCCCTGCCCCCTCGC |
| | | GGACCGCAGTGTACTAATTATGCTCTCTTGAAATTGGCTG |
| | | GAGATGTTGAGAGCAATCCCGGGCCCATGCGCATTAGCAA |
| | | GCCCCACCTGCGGAGCATCAGCATCCAGTGCTACCTGTGC |
| | | CTGCTGCTGAACAGCCACTTCCTGACCGAGGCCGGCATCC |
| | | ACGTGTTCATCCTGGGCTGCTTCAGCGCCGGACTGCCCAA |
| | | GACCGAGGCCAACTGGGTGAACGTGATCAGCGACCTGAAG |
| | | AAGATCGAGGACCTGATCCAGAGCATGCACATCGACGCCA |
| | | CCCTGTACACCGAGAGCGACGTGCACCCCAGCTGCAAGGT |
| | | GACCGCCATGAAGTGCTTTCTGCTGGAACTGCAGGTGATC |
| | | AGCCTGGAAAGCGGCGACGCCAGCATCCACGACACCGTGG |
| | | AGAACCTGATCATCCTGGCCAACAACAGCCTGAGCAGCAA |
| | | CGGCAACGTGACCGAGAGCGGCTGCAAAGAGTGCGAGGAA |
| | | CTGGAAGAGAAGAACATCAAAGAGTTTCTGCAGAGCTTCG |
| | | TGCACATCGTGCAGATGTTCATCAACACCAGCTGA |
| CD27TrCD3ZI | 29 | ATGACAAGAGTTACTAACAGCCCCTCTCTCCAAGCTCACT |
| L15 (DNA) | | TACAGGCTCTCTACTTAGTCCAGCACGAAGTCTGGAGACC |
| | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCATCCTTGTGATCTTCTC |
| | | TGGAATGTTCCTTGTTTTCACCCTGGCCGGGGCCCTGTTC |
| | | CTCCATAAACGCGTGAAGTTCAGCAGGAGCGCAGACGCCC |
| | | CCGCGTACCAGCAGGGCCAGAACCAGCTCTATAACGAGCT |
| | | CAATCTAGGACGAAGAGAGGAGTACGATGTTTTGGACAAA |
| | | AGACGTGGCCGGGACCCTGAGATGGGGGGAAAGCCGAGAA |
| | | GGAAGAACCCTCAGGAAGGCCTGTACAATGAACTGCAGAA |
| | | AGATAAGATGGCGGAGGCCTACAGTGAGATTGGGATGAAA |
| | | GGCGAGCGCCGGAGGGGCAAGGGGCACGATGGCCTTTACC |
| | | AGGGTCTCAGTACAGCCACCAAGGACACCTACGACGCCCT |
| | | TCACATGCAGGCCCTGCCCCCTCGCGGACCGCAGTGTACT |
| | | AATTATGCTCTCTTGAAATTGGCTGGAGATGTTGAGAGCA |
| | | ATCCCGGGCCCATGCGCATTAGCAAGCCCCACCTGCGGAG |
| | | CATCAGCATCCAGTGCTACCTGTGCCTGCTGCTGAACAGC |
| | | CACTTCCTGACCGAGGCCGGCATCCACGTGTTCATCCTGG |
| | | GCTGCTTCAGCGCCGGACTGCCCAAGACCGAGGCCAACTG |
| | | GGTGAACGTGATCAGCGACCTGAAGAAGATCGAGGACCTG |
| | | ATCCAGAGCATGCACATCGACGCCACCCTGTACACCGAGA |
| | | GCGACGTGCACCCCAGCTGCAAGGTGACCGCCATGAAGTG |
| | | CTTTCTGCTGGAACTGCAGGTGATCAGCCTGGAAAGCGGC |
| | | GACGCCAGCATCCACGACACCGTGGAGAACCTGATCATCC |
| | | TGGCCAACAACAGCCTGAGCAGCAACGGCAACGTGACCGA |
| | | GAGCGGCTGCAAAGAGTGCGAGGAACTGGAAGAGAAGAAC |
| | | ATCAAAGAGTTTCTGCAGAGCTTCGTGCACATCGTGCAGA |
| | | TGTTCATCAACACCAGCTGA |
| GSPco27Tr3z15 | 30 | ATGACAAGAGTTACTAACAGCCCCTCTCTCCAAGCTCACT |
| (DNA) | | TACAGGCTCTCTACTTAGTCCAGCACGAAGTCTGGAGACC |
| | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCATACTGGT |
| | | CATCTTTTCTGGAATGTTCCTTGTGTTCACCCTGGCAGGA |
| | | GCCCTGTTCCTTCACCGCGTGAAGTTCAGCAGGAGCGCAG |
| | | ACGCCCCCGCGTACCAGCAGGGCCAGAACCAGCTCTATAA |
| | | CGAGCTCAATCTAGGACGAAGAGAGGAGTACGATGTTTTG |
| | | GACAAAAGACGTGGCCGGGACCCTGAGATGGGGGGAAAGC |
| | | CGAGAAGGAAGAACCCTCAGGAAGGCCTGTACAATGAACT |
| | | GCAGAAAGATAAGATGGCGGAGGCCTACAGTGAGATTGGG |
| | | ATGAAAGGCGAGCGCCGGAGGGGCAAGGGGCACGATGGCC |
| | | TTTACCAGGGTCTCAGTACAGCCACCAAGGACACCTACGA |
| | | CGCCCTTCACATGCAGGCCCTGCCCCCTCGCGGACCGCAG |
| | | TGTACTAATTATGCTCTCTTGAAATTGGCTGGAGATGTTG |
| | | AGAGCAATCCCGGGCCCATGCGCATTAGCAAGCCCCACCT |
| | | GCGGAGCATCAGCATCCAGTGCTACCTGTGCCTGCTGCTG |
| | | AACAGCCACTTCCTGACCGAGGCCGGCATCCACGTGTTCA |
| | | TCCTGGGCTGCTTCAGCGCCGGACTGCCCAAGACCGAGGC |
| | | CAACTGGGTGAACGTGATCAGCGACCTGAAGAAGATCGAG |
| | | GACCTGATCCAGAGCATGCACATCGACGCCACCCTGTACA |
| | | CCGAGAGCGACGTGCACCCCAGCTGCAAGGTGACCGCCAT |
| | | GAAGTGCTTTCTGCTGGAACTGCAGGTGATCAGCCTGGAA |
| | | AGCGGCGACGCCAGCATCCACGACACCGTGGAGAACCTGA |
| | | TCATCCTGGCCAACAACAGCCTGAGCAGCAACGGCAACGT |
| | | GACCGAGAGCGGCTGCAAAGAGTGCGAGGAACTGGAAGAG |
| | | AAGAACATCAAAGAGTTTCTGCAGAGCTTCGTGCACATCG |
| | | TGCAGATGTTCATCAACACCAGCTGA |
| CD27TrCD28i | 31 | ATGACAAGAGTTACTAACAGCCCCTCTCTCCAAGCTCACT |
| cd3z15 (DNA) | | TACAGGCTCTCTACTTAGTCCAGCACGAAGTCTGGAGACC |
| | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCATCCTTGTGATCTTCTC |
| | | TGGAATGTTCCTTGTTTTCACCCTGGCCGGGGCCCTGTTC |
| | | CTCCATAGGAGTAAGAGGAGCAGGCTCCTGCACAGTGACT |
| | | ACATGAACATGACTCCCCGCCGCCCCGGGCCCACCCGCAA |
| | | GCATTACCAGCCCTATGCCCCACCACGCGACTTCGCAGCC |
| | | TATCGCTCACGCGTGAAGTTCAGCAGGAGCGCAGACGCCC |
| | | CCGCGTACCAGCAGGGCCAGAACCAGCTCTATAACGAGCT |
| | | CAATCTAGGACGAAGAGAGGAGTACGATGTTTTGGACAAA |
| | | AGACGTGGCCGGGACCCTGAGATGGGGGGAAAGCCGAGAA |
| | | GGAAGAACCCTCAGGAAGGCCTGTACAATGAACTGCAGAA |
| | | AGATAAGATGGCGGAGGCCTACAGTGAGATTGGGATGAAA |
| | | GGCGAGCGCCGGAGGGGCAAGGGGCACGATGGCCTTTACC |
| | | AGGGTCTCAGTACAGCCACCAAGGACACCTACGACGCCCT |
| | | TCACATGCAGGCCCTGCCCCCTCGCGGACCGCAGTGTACT |
| | | AATTATGCTCTCTTGAAATTGGCTGGAGATGTTGAGAGCA |
| | | ATCCCGGGCCCATGCGCATTAGCAAGCCCCACCTGCGGAG |
| | | CATCAGCATCCAGTGCTACCTGTGCCTGCTGCTGAACAGC |
| | | CACTTCCTGACCGAGGCCGGCATCCACGTGTTCATCCTGG |
| | | GCTGCTTCAGCGCCGGACTGCCCAAGACCGAGGCCAACTG |
| | | GGTGAACGTGATCAGCGACCTGAAGAAGATCGAGGACCTG |
| | | ATCCAGAGCATGCACATCGACGCCACCCTGTACACCGAGA |
| | | GCGACGTGCACCCCAGCTGCAAGGTGACCGCCATGAAGTG |
| | | CTTTCTGCTGGAACTGCAGGTGATCAGCCTGGAAAGCGGC |
| | | GACGCCAGCATCCACGACACCGTGGAGAACCTGATCATCC |
| | | TGGCCAACAACAGCCTGAGCAGCAACGGCAACGTGACCGA |
| | | GAGCGGCTGCAAAGAGTGCGAGGAACTGGAAGAGAAGAAC |
| | | ATCAAAGAGTTTCTGCAGAGCTTCGTGCACATCGTGCAGA |
| | | TGTTCATCAACACCAGCTGA |
| GSPco27Tr28 | 32 | ATGACAAGAGTTACTAACAGCCCCTCTCTCCAAGCTCACT |
| CD3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCATACTGGT |
| | | CATCTTTTCTGGAATGTTCCTTGTGTTCACCCTGGCAGGA |
| | | GCCCTGTTCCTTCACAGGAGTAAGAGGAGCAGGCTCCTGC |
| | | ACAGTGACTACATGAACATGACTCCCCGCCGCCCCGGGCC |
| | | CACCCGCAAGCATTACCAGCCCTATGCCCCACCACGCGAC |
| | | TTCGCAGCCTATCGCTCACGCGTGAAGTTCAGCAGGAGCG |
| | | CAGACGCCCCCGCGTACCAGCAGGGCCAGAACCAGCTCTA |
| | | TAACGAGCTCAATCTAGGACGAAGAGAGGAGTACGATGTT |
| | | TTGGACAAAAGACGTGGCCGGGACCCTGAGATGGGGGGAA |
| | | AGCCGAGAAGGAAGAACCCTCAGGAAGGCCTGTACAATGA |
| | | ACTGCAGAAAGATAAGATGGCGGAGGCCTACAGTGAGATT |
| | | GGGATGAAAGGCGAGCGCCGGAGGGGCAAGGGGCACGATG |
| | | GCCTTTACCAGGGTCTCAGTACAGCCACCAAGGACACCTA |
| | | CGACGCCCTTCACATGCAGGCCCTGCCCCCTCGCGGACCG |
| | | CAGTGTACTAATTATGCTCTCTTGAAATTGGCTGGAGATG |
| | | TTGAGAGCAATCCCGGGCCCATGCGCATTAGCAAGCCCCA |
| | | CCTGCGGAGCATCAGCATCCAGTGCTACCTGTGCCTGCTG |
| | | CTGAACAGCCACTTCCTGACCGAGGCCGGCATCCACGTGT |
| | | TCATCCTGGGCTGCTTCAGCGCCGGACTGCCCAAGACCGA |
| | | GGCCAACTGGGTGAACGTGATCAGCGACCTGAAGAAGATC |
| | | GAGGACCTGATCCAGAGCATGCACATCGACGCCACCCTGT |
| | | ACACCGAGAGCGACGTGCACCCCAGCTGCAAGGTGACCGC |
| | | CATGAAGTGCTTTCTGCTGGAACTGCAGGTGATCAGCCTG |
| | | GAAAGCGGCGACGCCAGCATCCACGACACCGTGGAGAACC |
| | | TGATCATCCTGGCCAACAACAGCCTGAGCAGCAACGGCAA |
| | | CGTGACCGAGAGCGGCTGCAAAGAGTGCGAGGAACTGGAA |
| | | GAGAAGAACATCAAAGAGTTTCTGCAGAGCTTCGTGCACA |
| | | TCGTGCAGATGTTCATCAACACCAGCTGA |
| CD27TrCD28t | 33 | ATGACAAGAGTTACTAACAGCCCCTCTCTCCAAGCTCACT |
| mdicd3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCTTTTGGGTGCTGGTGGT |
| | | GGTTGGTGGAGTCCTGGCTTGCTATAGCTTGCTAGTAACA |
| | | GTGGCCTTTATTATTTTCTGGGTGAGGAGTAAGAGGAGCA |
| | | GGCTCCTGCACAGTGACTACATGAACATGACTCCCCGCCG |
| | | CCCCGGGCCCACCCGCAAGCATTACCAGCCCTATGCCCCA |
| | | CCACGCGACTTCGCAGCCTATCGCTCACGCGTGAAGTTCA |
| | | GCAGGAGCGCAGACGCCCCCGCGTACCAGCAGGGCCAGAA |
| | | CCAGCTCTATAACGAGCTCAATCTAGGACGAAGAGAGGAG |
| | | TACGATGTTTTGGACAAAAGACGTGGCCGGGACCCTGAGA |
| | | TGGGGGGAAAGCCGAGAAGGAAGAACCCTCAGGAAGGCCT |
| | | GTACAATGAACTGCAGAAAGATAAGATGGCGGAGGCCTAC |
| | | AGTGAGATTGGGATGAAAGGCGAGCGCCGGAGGGGCAAGG |
| | | GGCACGATGGCCTTTACCAGGGTCTCAGTACAGCCACCAA |
| | | GGACACCTACGACGCCCTTCACATGCAGGCCCTGCCCCCT |
| | | CGCGGACCGCAGTGTACTAATTATGCTCTCTTGAAATTGG |
| | | CTGGAGATGTTGAGAGCAATCCCGGGCCCATGCGCATTAG |
| | | CAAGCCCCACCTGCGGAGCATCAGCATCCAGTGCTACCTG |
| | | TGCCTGCTGCTGAACAGCCACTTCCTGACCGAGGCCGGCA |
| | | TCCACGTGTTCATCCTGGGCTGCTTCAGCGCCGGACTGCC |
| | | CAAGACCGAGGCCAACTGGGTGAACGTGATCAGCGACCTG |
| | | AAGAAGATCGAGGACCTGATCCAGAGCATGCACATCGACG |
| | | CCACCCTGTACACCGAGAGCGACGTGCACCCCAGCTGCAA |
| | | GGTGACCGCCATGAAGTGCTTTCTGCTGGAACTGCAGGTG |
| | | ATCAGCCTGGAAAGCGGCGACGCCAGCATCCACGACACCG |
| | | TGGAGAACCTGATCATCCTGGCCAACAACAGCCTGAGCAG |
| | | CAACGGCAACGTGACCGAGAGCGGCTGCAAAGAGTGCGAG |
| | | GAACTGGAAGAGAAGAACATCAAAGAGTTTCTGCAGAGCT |
| | | TCGTGCACATCGTGCAGATGTTCATCAACACCAGCTGA |
| GSPco27Tr28t | 34 | ATGACAAGAGTTACTAACAGCCCCTCTCTCCAAGCTCACT |
| mdicCD3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCTTTTGGGT |
| | | GCTGGTGGTGGTTGGTGGAGTCCTGGCTTGCTATAGCTTG |
| | | CTAGTAACAGTGGCCTTTATTATTTTCTGGGTGAGGAGTA |
| | | AGAGGAGCAGGCTCCTGCACAGTGACTACATGAACATGAC |
| | | TCCCCGCCGCCCCGGGCCCACCCGCAAGCATTACCAGCCC |
| | | TATGCCCCACCACGCGACTTCGCAGCCTATCGCTCACGCG |
| | | TGAAGTTCAGCAGGAGCGCAGACGCCCCCGCGTACCAGCA |
| | | GGGCCAGAACCAGCTCTATAACGAGCTCAATCTAGGACGA |
| | | AGAGAGGAGTACGATGTTTTGGACAAAAGACGTGGCCGGG |
| | | ACCCTGAGATGGGGGGAAAGCCGAGAAGGAAGAACCCTCA |
| | | GGAAGGCCTGTACAATGAACTGCAGAAAGATAAGATGGCG |
| | | GAGGCCTACAGTGAGATTGGGATGAAAGGCGAGCGCCGGA |
| | | GGGGCAAGGGGCACGATGGCCTTTACCAGGGTCTCAGTAC |
| | | AGCCACCAAGGACACCTACGACGCCCTTCACATGCAGGCC |
| | | CTGCCCCCTCGCGGACCGCAGTGTACTAATTATGCTCTCT |
| | | TGAAATTGGCTGGAGATGTTGAGAGCAATCCCGGGCCCAT |
| | | GCGCATTAGCAAGCCCCACCTGCGGAGCATCAGCATCCAG |
| | | TGCTACCTGTGCCTGCTGCTGAACAGCCACTTCCTGACCG |
| | | AGGCCGGCATCCACGTGTTCATCCTGGGCTGCTTCAGCGC |
| | | CGGACTGCCCAAGACCGAGGCCAACTGGGTGAACGTGATC |
| | | AGCGACCTGAAGAAGATCGAGGACCTGATCCAGAGCATGC |
| | | ACATCGACGCCACCCTGTACACCGAGAGCGACGTGCACCC |
| | | CAGCTGCAAGGTGACCGCCATGAAGTGCTTTCTGCTGGAA |
| | | CTGCAGGTGATCAGCCTGGAAAGCGGCGACGCCAGCATCC |
| | | ACGACACCGTGGAGAACCTGATCATCCTGGCCAACAACAG |
| | | CCTGAGCAGCAACGGCAACGTGACCGAGAGCGGCTGCAAA |
| | | GAGTGCGAGGAACTGGAAGAGAAGAACATCAAAGAGTTTC |
| | | TGCAGAGCTTCGTGCACATCGTGCAGATGTTCATCAACAC |
| | | CAGCTGA |
| CD27TrDAP10 | 35 | ATGACAAGAGTTACTAACAGCCCCTCTCTCCAAGCTCACT |
| icd3z15 (DNA) | | TACAGGCTCTCTACTTAGTCCAGCACGAAGTCTGGAGACC |
| | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCATACTGGTCATCTTTTC |
| | | TGGAATGTTCCTTGTGTTCACCCTGGCAGGAGCCCTGTTC |
| | | CTTCACCTTTGCGCACGCCCACGCCGCAGCCCCGCCCAAG |
| | | AAGATGGCAAAGTCTACATCAACATGCCAGGCAGGGGCAA |
| | | ACGCGTGAAGTTCAGCAGGAGCGCAGACGCCCCCGCGTAC |
| | | CAGCAGGGCCAGAACCAGCTCTATAACGAGCTCAATCTAG |
| | | GACGAAGAGAGGAGTACGATGTTTTGGACAAAAGACGTGG |
| | | CCGGGACCCTGAGATGGGGGGAAAGCCGAGAAGGAAGAAC |
| | | CCTCAGGAAGGCCTGTACAATGAACTGCAGAAAGATAAGA |
| | | TGGCGGAGGCCTACAGTGAGATTGGGATGAAAGGCGAGCG |
| | | CCGGAGGGGCAAGGGGCACGATGGCCTTTACCAGGGTCTC |
| | | AGTACAGCCACCAAGGACACCTACGACGCCCTTCACATGC |
| | | AGGCCCTGCCCCCTCGCGGACCGCAGTGTACTAATTATGC |
| | | TCTCTTGAAATTGGCTGGAGATGTTGAGAGCAATCCCGGG |
| | | CCCATGCGCATTAGCAAGCCCCACCTGCGGAGCATCAGCA |
| | | TCCAGTGCTACCTGTGCCTGCTGCTGAACAGCCACTTCCT |
| | | GACCGAGGCCGGCATCCACGTGTTCATCCTGGGCTGCTTC |
| | | AGCGCCGGACTGCCCAAGACCGAGGCCAACTGGGTGAACG |
| | | TGATCAGCGACCTGAAGAAGATCGAGGACCTGATCCAGAG |
| | | CATGCACATCGACGCCACCCTGTACACCGAGAGCGACGTG |
| | | CACCCCAGCTGCAAGGTGACCGCCATGAAGTGCTTTCTGC |
| | | TGGAACTGCAGGTGATCAGCCTGGAAAGCGGCGACGCCAG |
| | | CATCCACGACACCGTGGAGAACCTGATCATCCTGGCCAAC |
| | | AACAGCCTGAGCAGCAACGGCAACGTGACCGAGAGCGGCT |
| | | GCAAAGAGTGCGAGGAACTGGAAGAGAAGAACATCAAAGA |
| | | GTTTCTGCAGAGCTTCGTGCACATCGTGCAGATGTTCATC |
| | | AACACCAGCTGA |
| GSPco27FLda | 36 | ATGACAAGAGTTACTAACAGCCCCTCTCTCCAAGCTCACT |
| p10icd3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCATACTGGT |
| | | CATCTTTTCTGGAATGTTCCTTGTGTTCACCCTGGCAGGA |
| | | GCCCTGTTCCTTCACCTTTGCGCACGCCCACGCCGCAGCC |
| | | CCGCCCAAGAAGATGGCAAAGTCTACATCAACATGCCAGG |
| | | CAGGGGCAAACGCGTGAAGTTCAGCAGGAGCGCAGACGCC |
| | | CCCGCGTACCAGCAGGGCCAGAACCAGCTCTATAACGAGC |
| | | TCAATCTAGGACGAAGAGAGGAGTACGATGTTTTGGACAA |
| | | AAGACGTGGCCGGGACCCTGAGATGGGGGGAAAGCCGAGA |
| | | AGGAAGAACCCTCAGGAAGGCCTGTACAATGAACTGCAGA |
| | | AAGATAAGATGGCGGAGGCCTACAGTGAGATTGGGATGAA |
| | | AGGCGAGCGCCGGAGGGGCAAGGGGCACGATGGCCTTTAC |
| | | CAGGGTCTCAGTACAGCCACCAAGGACACCTACGACGCCC |
| | | TTCACATGCAGGCCCTGCCCCCTCGCGGACCGCAGTGTAC |
| | | TAATTATGCTCTCTTGAAATTGGCTGGAGATGTTGAGAGC |
| | | AATCCCGGGCCCATGCGCATTAGCAAGCCCCACCTGCGGA |
| | | GCATCAGCATCCAGTGCTACCTGTGCCTGCTGCTGAACAG |
| | | CCACTTCCTGACCGAGGCCGGCATCCACGTGTTCATCCTG |
| | | GGCTGCTTCAGCGCCGGACTGCCCAAGACCGAGGCCAACT |
| | | GGGTGAACGTGATCAGCGACCTGAAGAAGATCGAGGACCT |
| | | GATCCAGAGCATGCACATCGACGCCACCCTGTACACCGAG |
| | | AGCGACGTGCACCCCAGCTGCAAGGTGACCGCCATGAAGT |
| | | GCTTTCTGCTGGAACTGCAGGTGATCAGCCTGGAAAGCGG |
| | | CGACGCCAGCATCCACGACACCGTGGAGAACCTGATCATC |
| | | CTGGCCAACAACAGCCTGAGCAGCAACGGCAACGTGACCG |
| | | AGAGCGGCTGCAAAGAGTGCGAGGAACTGGAAGAGAAGAA |
| | | CATCAAAGAGTTTCTGCAGAGCTTCGTGCACATCGTGCAG |
| | | ATGTTCATCAACACCAGCTGA |
| CD27TrDAP12 | 37 | ATGACAAGAGTTACTAACAGCCCCTCTCTCCAAGCTCACT |
| icd3z15 (DNA) | | TACAGGCTCTCTACTTAGTCCAGCACGAAGTCTGGAGACC |
| | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCATCCTTGTGATCTTCTC |
| | | TGGAATGTTCCTTGTTTTCACCCTGGCCGGGGCCCTGTTC |
| | | CTCCATTACTTCCTGGGCCGGCTGGTCCCTCGGGGGCGAG |
| | | GGGCTGCGGAGGCAGCGACCCGGAAACAGCGTATCACTGA |
| | | GACCGAGTCGCCTTATCAGGAGCTCCAGGGTCAGAGGTCG |
| | | GATGTCTACAGCGACCTCAACACACAGAGGCCGTATTACA |
| | | AAAAACGCGTGAAGTTCAGCAGGAGCGCAGACGCCCCCGC |
| | | GTACCAGCAGGGCCAGAACCAGCTCTATAACGAGCTCAAT |
| | | CTAGGACGAAGAGAGGAGTACGATGTTTTGGACAAAAGAC |
| | | GTGGCCGGGACCCTGAGATGGGGGGAAAGCCGAGAAGGAA |
| | | GAACCCTCAGGAAGGCCTGTACAATGAACTGCAGAAAGAT |
| | | AAGATGGCGGAGGCCTACAGTGAGATTGGGATGAAAGGCG |
| | | AGCGCCGGAGGGGCAAGGGGCACGATGGCCTTTACCAGGG |
| | | TCTCAGTACAGCCACCAAGGACACCTACGACGCCCTTCAC |
| | | ATGCAGGCCCTGCCCCCTCGCGGACCGCAGTGTACTAATT |
| | | ATGCTCTCTTGAAATTGGCTGGAGATGTTGAGAGCAATCC |
| | | CGGGCCCATGCGCATTAGCAAGCCCCACCTGCGGAGCATC |
| | | AGCATCCAGTGCTACCTGTGCCTGCTGCTGAACAGCCACT |
| | | TCCTGACCGAGGCCGGCATCCACGTGTTCATCCTGGGCTG |
| | | CTTCAGCGCCGGACTGCCCAAGACCGAGGCCAACTGGGTG |
| | | AACGTGATCAGCGACCTGAAGAAGATCGAGGACCTGATCC |
| | | AGAGCATGCACATCGACGCCACCCTGTACACCGAGAGCGA |
| | | CGTGCACCCCAGCTGCAAGGTGACCGCCATGAAGTGCTTT |
| | | CTGCTGGAACTGCAGGTGATCAGCCTGGAAAGCGGCGACG |
| | | CCAGCATCCACGACACCGTGGAGAACCTGATCATCCTGGC |
| | | CAACAACAGCCTGAGCAGCAACGGCAACGTGACCGAGAGC |
| | | GGCTGCAAAGAGTGCGAGGAACTGGAAGAGAAGAACATCA |
| | | AAGAGTTTCTGCAGAGCTTCGTGCACATCGTGCAGATGTT |
| | | CATCAACACCAGCTGA |
| GSPco27FLda | 38 | ATGACAAGAGTTACTAACAGCCCCTCTCTCCAAGCTCACT |
| p12icd3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCATACTGGT |
| | | CATCTTTTCTGGAATGTTCCTTGTGTTCACCCTGGCAGGA |
| | | GCCCTGTTCCTTCACTACTTCCTGGGCCGGCTGGTCCCTC |
| | | GGGGGCGAGGGGCTGCGGAGGCAGCGACCCGGAAACAGCG |
| | | TATCACTGAGACCGAGTCGCCTTATCAGGAGCTCCAGGGT |
| | | CAGAGGTCGGATGTCTACAGCGACCTCAACACACAGAGGC |
| | | CGTATTACAAAAAACGCGTGAAGTTCAGCAGGAGCGCAGA |
| | | CGCCCCCGCGTACCAGCAGGGCCAGAACCAGCTCTATAAC |
| | | GAGCTCAATCTAGGACGAAGAGAGGAGTACGATGTTTTGG |
| | | ACAAAAGACGTGGCCGGGACCCTGAGATGGGGGGAAAGCC |
| | | GAGAAGGAAGAACCCTCAGGAAGGCCTGTACAATGAACTG |
| | | CAGAAAGATAAGATGGCGGAGGCCTACAGTGAGATTGGGA |
| | | TGAAAGGCGAGCGCCGGAGGGGCAAGGGGCACGATGGCCT |
| | | TTACCAGGGTCTCAGTACAGCCACCAAGGACACCTACGAC |
| | | GCCCTTCACATGCAGGCCCTGCCCCCTCGCGGACCGCAGT |
| | | GTACTAATTATGCTCTCTTGAAATTGGCTGGAGATGTTGA |
| | | GAGCAATCCCGGGCCCATGCGCATTAGCAAGCCCCACCTG |
| | | CGGAGCATCAGCATCCAGTGCTACCTGTGCCTGCTGCTGA |
| | | ACAGCCACTTCCTGACCGAGGCCGGCATCCACGTGTTCAT |
| | | CCTGGGCTGCTTCAGCGCCGGACTGCCCAAGACCGAGGCC |
| | | AACTGGGTGAACGTGATCAGCGACCTGAAGAAGATCGAGG |
| | | ACCTGATCCAGAGCATGCACATCGACGCCACCCTGTACAC |
| | | CGAGAGCGACGTGCACCCCAGCTGCAAGGTGACCGCCATG |
| | | AAGTGCTTTCTGCTGGAACTGCAGGTGATCAGCCTGGAAA |
| | | GCGGCGACGCCAGCATCCACGACACCGTGGAGAACCTGAT |
| | | CATCCTGGCCAACAACAGCCTGAGCAGCAACGGCAACGTG |
| | | ACCGAGAGCGGCTGCAAAGAGTGCGAGGAACTGGAAGAGA |
| | | AGAACATCAAAGAGTTTCTGCAGAGCTTCGTGCACATCGT |
| | | GCAGATGTTCATCAACACCAGCTGA |
| CD27TrNKG2 | 39 | ATGACAAGAGTTACTAACAGCCCCTCTCTCCAAGCTCACT |
| Dic3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGGCACGGCCACATCCCTGGTGGCTGTGC |
| | | GTTCTGGGGACCCTGGTGGGGCTCTCAGCTACTCCAGCCC |
| | | CCAAGAGCTGCCCAGAGAGGCACTACTGGGCTCAGGGAAA |
| | | GCTGTGCTGCCAGATGTGTGAGCCAGGAACATTCCTCGTG |
| | | AAGGACTGTGACCAGCATAGAAAGGCTGCTCAGTGTGATC |
| | | CTTGCATACCGGGGGTCTCCTTCTCTCCTGACCACCACAC |
| | | CCGGCCCCACTGTGAGAGCTGTCGGCACTGTAACTCTGGT |
| | | CTTCTCGTTCGCAACTGCACCATCACTGCCAATGCTGAGT |
| | | GTGCCTGTCGCAATGGCTGGCAGTGCAGGGACAAGGAGTG |
| | | CACCGAGTGTGATCCTCTTCCAAACCCTTCGCTGACCGCT |
| | | CGGTCGTCTCAGGCCCTGAGCCCACACCCTCAGCCCACCC |
| | | ACTTACCTTATGTCAGTGAGATGCTGGAGGCCAGGACAGC |
| | | TGGGCACATGCAGACTCTGGCTGACTTCAGGCAGCTGCCT |
| | | GCCCGGACTCTCTCTACCCACTGGCCACCCCAAAGATCCC |
| | | TGTGCAGCTCCGATTTTATTCGCATCCTTGTGATCTTCTC |
| | | TGGAATGTTCCTTGTTTTCACCCTGGCCGGGGCCCTGTTC |
| | | CTCCATAGCGCGAACGAACGCTGCAAAAGCAAAGTGGTGC |
| | | CGTGCCGCCAGAAACAGTGGCGCACCAGCTTTGATAGCAA |
| | | AAAACTGGATCTGAACTATAACCATTTTGAAAGCATGGAA |
| | | TGGAGCCATCGCAGCCGCCGCGGCCGCATTTGGGGCATGA |
| | | AACGCGTGAAGTTCAGCAGGAGCGCAGACGCCCCCGCGTA |
| | | CCAGCAGGGCCAGAACCAGCTCTATAACGAGCTCAATCTA |
| | | GGACGAAGAGAGGAGTACGATGTTTTGGACAAAAGACGTG |
| | | GCCGGGACCCTGAGATGGGGGGAAAGCCGAGAAGGAAGAA |
| | | CCCTCAGGAAGGCCTGTACAATGAACTGCAGAAAGATAAG |
| | | ATGGCGGAGGCCTACAGTGAGATTGGGATGAAAGGCGAGC |
| | | GCCGGAGGGGCAAGGGGCACGATGGCCTTTACCAGGGTCT |
| | | CAGTACAGCCACCAAGGACACCTACGACGCCCTTCACATG |
| | | CAGGCCCTGCCCCCTCGCGGACCGCAGTGTACTAATTATG |
| | | CTCTCTTGAAATTGGCTGGAGATGTTGAGAGCAATCCCGG |
| | | GCCCATGCGCATTAGCAAGCCCCACCTGCGGAGCATCAGC |
| | | ATCCAGTGCTACCTGTGCCTGCTGCTGAACAGCCACTTCC |
| | | TGACCGAGGCCGGCATCCACGTGTTCATCCTGGGCTGCTT |
| | | CAGCGCCGGACTGCCCAAGACCGAGGCCAACTGGGTGAAC |
| | | GTGATCAGCGACCTGAAGAAGATCGAGGACCTGATCCAGA |
| | | GCATGCACATCGACGCCACCCTGTACACCGAGAGCGACGT |
| | | GCACCCCAGCTGCAAGGTGACCGCCATGAAGTGCTTTCTG |
| | | CTGGAACTGCAGGTGATCAGCCTGGAAAGCGGCGACGCCA |
| | | GCATCCACGACACCGTGGAGAACCTGATCATCCTGGCCAA |
| | | CAACAGCCTGAGCAGCAACGGCAACGTGACCGAGAGCGGC |
| | | TGCAAAGAGTGCGAGGAACTGGAAGAGAAGAACATCAAAG |
| | | AGTTTCTGCAGAGCTTCGTGCACATCGTGCAGATGTTCAT |
| | | CAACACCAGCTGA |
| GSPco27TrNK | 40 | ATGACAAGAGTTACTAACAGCCCCTCTCTCCAAGCTCACT |
| G2Dicd3z15 | | TACAGGCTCTCTACTTAGTCCAGCACGAAGTCTGGAGACC |
| (DNA) | | TCTGGCGGCAGCCTACCAAGAACAACTGGACCGACCGGTG |
| | | GTACCTCACCCTTACCGAGTCGGCGACACAGTGTGGGTCC |
| | | GCCGACACCAGACTAAGAACCTAGAACCTCGCTGGAAAGG |
| | | ACCTTACACAGTCCTGCTGACCACCCCCACCGCCCTCAAA |
| | | GTAGACGGCATCGCAGCTTGGATACACGCCGCCCACGTGA |
| | | AGGCTGCCGACCCCGGGGGTGGACCATCCTCTAGACTGCC |
| | | ATGCTCGAGGATGCTTCTCCTGGTGACAAGCCTTCTGCTC |
| | | TGTGAGTTACCACACCCAGCATTCCTCCTGATCCCAGCTA |
| | | CACCGGCTCCGAAGTCCTGCCCGGAGCGGCATTATTGGGC |
| | | ACAGGGCAAGTTGTGTTGTCAAATGTGTGAGCCGGGAACC |
| | | TTTCTCGTGAAGGATTGCGATCAGCATCGGAAGGCCGCGC |
| | | AGTGCGACCCATGTATACCAGGGGTCTCATTTTCCCCAGA |
| | | TCACCATACGAGGCCGCACTGTGAGTCTTGCAGGCATTGT |
| | | AATTCCGGCTTGTTGGTCCGCAACTGTACTATTACTGCGA |
| | | ATGCAGAGTGTGCTTGTAGAAACGGATGGCAGTGCAGGGA |
| | | CAAAGAATGTACGGAGTGTGATCCACTGCCTAACCCCAGT |
| | | CTTACAGCAAGATCTTCACAGGCCCTCAGCCCGCATCCTC |
| | | AACCAACACATCTTCCTTACGTGTCAGAAATGTTGGAGGC |
| | | GCGAACCGCAGGCCATATGCAGACCCTGGCGGACTTTCGG |
| | | CAGCTGCCAGCACGCACACTTAGTACACACTGGCCACCAC |
| | | AACGCAGCTTGTGCTCTTCCGATTTCATCCGCATACTGGT |
| | | CATCTTTTCTGGAATGTTCCTTGTGTTCACCCTGGCAGGA |
| | | GCCCTGTTCCTTCACAGCGCGAACGAACGCTGCAAAAGCA |
| | | AAGTGGTGCCGTGCCGCCAGAAACAGTGGCGCACCAGCTT |
| | | TGATAGCAAAAAACTGGATCTGAACTATAACCATTTTGAA |
| | | AGCATGGAATGGAGCCATCGCAGCCGCCGCGGCCGCATTT |
| | | GGGGCATGAAACGCGTGAAGTTCAGCAGGAGCGCAGACGC |
| | | CCCCGCGTACCAGCAGGGCCAGAACCAGCTCTATAACGAG |
| | | CTCAATCTAGGACGAAGAGAGGAGTACGATGTTTTGGACA |
| | | AAAGACGTGGCCGGGACCCTGAGATGGGGGGAAAGCCGAG |
| | | AAGGAAGAACCCTCAGGAAGGCCTGTACAATGAACTGCAG |
| | | AAAGATAAGATGGCGGAGGCCTACAGTGAGATTGGGATGA |
| | | AAGGCGAGCGCCGGAGGGGCAAGGGGCACGATGGCCTTTA |
| | | CCAGGGTCTCAGTACAGCCACCAAGGACACCTACGACGCC |
| | | CTTCACATGCAGGCCCTGCCCCCTCGCGGACCGCAGTGTA |
| | | CTAATTATGCTCTCTTGAAATTGGCTGGAGATGTTGAGAG |
| | | CAATCCCGGGCCCATGCGCATTAGCAAGCCCCACCTGCGG |
| | | AGCATCAGCATCCAGTGCTACCTGTGCCTGCTGCTGAACA |
| | | GCCACTTCCTGACCGAGGCCGGCATCCACGTGTTCATCCT |
| | | GGGCTGCTTCAGCGCCGGACTGCCCAAGACCGAGGCCAAC |
| | | TGGGTGAACGTGATCAGCGACCTGAAGAAGATCGAGGACC |
| | | TGATCCAGAGCATGCACATCGACGCCACCCTGTACACCGA |
| | | GAGCGACGTGCACCCCAGCTGCAAGGTGACCGCCATGAAG |
| | | TGCTTTCTGCTGGAACTGCAGGTGATCAGCCTGGAAAGCG |
| | | GCGACGCCAGCATCCACGACACCGTGGAGAACCTGATCAT |
| | | CCTGGCCAACAACAGCCTGAGCAGCAACGGCAACGTGACC |
| | | GAGAGCGGCTGCAAAGAGTGCGAGGAACTGGAAGAGAAGA |
| | | ACATCAAAGAGTTTCTGCAGAGCTTCGTGCACATCGTGCA |
| | | GATGTTCATCAACACCAGCTGA |
| CO CAR.CD70 | 95 | ATGGCCCTGCCTGTGACAGCTCTGCTCCTCCCTCTGGCCC |
| 42D12. | | TGCTGCTCCATGCCGCCAGACCCCAGGCAGTIGTGACCCA |
| VLVH.IgG1.C | | GGAGCCTTCCCTGACAGTGTCTCCAGGAGGGACGGTCACG |
| D28.CD3z-2A- | | CTCACCTGCGGCCTCAAATCTGGGTCTGTCACTTCCGATA |
| IL15 | | ACTTCCCCACTTGGTACCAGCAGACACCAGGCCAGGCTCC |
| | | CCGATTGCTTATCTACAACACAAACACCCGTCACTCTGGC |
| | | GTCCCCGACCGCTTCTCCGGATCCATCCTGGGCAACAAAG |
| | | CCGCCCTCACCATCACGGGGGCCCAGGCCGACGACGAGGC |
| | | CGAATATTTCTGTGCTCTGTTCATAAGTAATCCTAGTGTT |
| | | GAGTTCGGCGGAGGGACCCAACTGACCGTCCTAGGTGGCA |
| | | GCACCAGCGGCTCCGGCAAGCCTGGCTCTGGCGAGGGCAG |
| | | CACAAAGGGAGAGGTGCAGCTCGTGGAGTCTGGGGGAGGC |
| | | TTGGTGCAGCCTGGGGGGTCTCTGAGACTCTCCTGTGCAG |
| | | CCTCTGGATTCACCTTCAGTGTCTACTACATGAACTGGGT |
| | | CCGCCAGGCTCCAGGGAAGGGGCTCGAGTGGGTCTCAGAT |
| | | ATTAATAATGAAGGTGGTACTACATACTATGCAGACTCCG |
| | | TGAAGGGCCGATTCACCATCTCCAGAGACAACTCTAAGAA |
| | | CAGCCTGTATCTGCAAATGAACAGCCTGCGCGCCGAGGAC |
| | | ACGGCCGTGTACTACTGCGCGAGAGATGCCGGATATAGCA |
| | | ACCATGTACCCATCTTTGATTCTTGGGGCCAGGGGACCCT |
| | | GGTCACTGTCTCCTCACGTACGGTCACTGTCTCTTCACAG |
| | | GATCCCGCCGAGCCCAAATCTCCTGACAAAACTCACACAT |
| | | GCCCACCGTGCCCAGCACCTGAACTCCTGGGGGGACCGTC |
| | | AGTCTTCCTCTTCCCCCCAAAACCCAAGGACACCCTCATG |
| | | ATCTCCCGGACCCCTGAGGTCACATGCGTGGTGGTGGACG |
| | | TGAGCCACGAAGACCCTGAGGTCAAGTTCAACTGGTACGT |
| | | GGACGGCGTGGAGGTGCATAATGCCAAGACAAAGCCGCGG |
| | | GAGGAGCAGTACAACAGCACGTACCGTGTGGTCAGCGTCC |
| | | TCACCGTCCTGCACCAGGACTGGCTGAATGGCAAGGAGTA |
| | | CAAGTGCAAGGTCTCCAACAAAGCCCTCCCAGCCCCCATC |
| | | GAGAAAACCATCTCCAAAGCCAAAGGGCAGCCCCGAGAAC |
| | | CACAGGTGTACACCCTGCCCCCATCCCGGGATGAGCTGAC |
| | | CAAGAACCAGGTCAGCCTGACCTGCCTGGTCAAAGGCTTC |
| | | TATCCCAGCGACATCGCCGTGGAGTGGGAGAGCAATGGGC |
| | | AACCGGAGAACAACTACAAGACCACGCCTCCCGTGCTGGA |
| | | CTCCGACGGCTCCTTCTTCCTCTACAGCAAGCTCACCGTG |
| | | GACAAGAGCAGGTGGCAGCAGGGGAACGTCTTCTCATGCT |
| | | CCGTGATGCATGAGGCTCTGCACAACCACTACACGCAGAA |
| | | GAGCCTCTCCCTGTCTCCGGGTAAAAAAGATCCCAAATTT |
| | | TGGGTGCTGGTGGTGGTTGGTGGAGTCCTGGCTTGCTATA |
| | | GCTTGCTAGTAACAGTGGCCTTTATTATTTTCTGGGTGAG |
| | | GAGTAAGAGGAGCAGGCTCCTGCACAGTGACTACATGAAC |
| | | ATGACTCCCCGCCGCCCCGGGCCCACCCGCAAGCATTACC |
| | | AGCCCTATGCCCCACCACGCGACTTCGCAGCCTATCGCTC |
| | | ACGCGTGAAGTTCAGCAGGAGCGCAGACGCCCCCGCGTAC |
| | | CAGCAGGGCCAGAACCAGCTCTATAACGAGCTCAATCTAG |
| | | GACGAAGAGAGGAGTACGATGTTTTGGACAAAAGACGTGG |
| | | CCGGGACCCTGAGATGGGGGGAAAGCCGAGAAGGAAGAAC |
| | | CCTCAGGAAGGCCTGTACAATGAACTGCAGAAAGATAAGA |
| | | TGGCGGAGGCCTACAGTGAGATTGGGATGAAAGGCGAGCG |
| | | CCGGAGGGGCAAGGGGCACGATGGCCTTTACCAGGGTCTC |
| | | AGTACAGCCACCAAGGACACCTACGACGCCCTTCACATGC |
| | | AGGCCCTGCCCCCTCGCGGACCGCAGTGTACTAATTATGC |
| | | TCTCTTGAAATTGGCTGGAGATGTTGAGAGCAATCCCGGG |
| | | CCCATGCGCATTAGCAAGCCCCACCTGCGGAGCATCAGCA |
| | | TCCAGTGCTACCTGTGCCTGCTGCTGAACAGCCACTTCCT |
| | | GACCGAGGCCGGCATCCACGTGTTCATCCTGGGCTGCTTC |
| | | AGCGCCGGACTGCCCAAGACCGAGGCCAACTGGGTGAACG |
| | | TGATCAGCGACCTGAAGAAGATCGAGGACCTGATCCAGAG |
| | | CATGCACATCGACGCCACCCTGTACACCGAGAGCGACGTG |
| | | CACCCCAGCTGCAAGGTGACCGCCATGAAGTGCTTTCTGC |
| | | TGGAACTGCAGGTGATCAGCCTGGAAAGCGGCGACGCCAG |
| | | CATCCACGACACCGTGGAGAACCTGATCATCCTGGCCAAC |
| | | AACAGCCTGAGCAGCAACGGCAACGTGACCGAGAGCGGCT |
| | | GCAAAGAGTGCGAGGAACTGGAAGAGAAGAACATCAAAGA |
| | | GTTTCTGCAGAGCTTCGTGCACATCGTGCAGATGTTCATC |
| | | AACACCAGCTGA |
| CO CAR.CD70 | 96 | ATGGCCCTGCCTGTGACAGCTCTGCTCCTCCCTCTGGCCC |
| 42D12 | | TGCTGCTCCATGCCGCCAGACCCGAGGTGCAGCTCGTGGA |
| VHVL.IgG1.C | | GTCTGGGGGAGGCTTGGTGCAGCCTGGGGGGTCTCTGAGA |
| D28.CD3z-2A- | | CTCTCCTGTGCAGCCTCTGGATTCACCTTCAGTGTCTACT |
| IL15 | | ACATGAACTGGGTCCGCCAGGCTCCAGGGAAGGGGCTCGA |
| | | GTGGGTCTCAGATATTAATAATGAAGGTGGTACTACATAC |
| | | TATGCAGACTCCGTGAAGGGCCGATTCACCATCTCCAGAG |
| | | ACAACTCTAAGAACAGCCTGTATCTGCAAATGAACAGCCT |
| | | GCGCGCCGAGGACACGGCCGTGTACTACTGCGCGAGAGAT |
| | | GCCGGATATAGCAACCATGTACCCATCTTTGATTCTTGGG |
| | | GCCAGGGGACCCTGGTCACTGTCTCCTCAGGCAGCACCAG |
| | | CGGCTCCGGCAAGCCTGGCTCTGGCGAGGGCAGCACAAAG |
| | | GGACAGGCAGTGGTGACCCAGGAGCCTTCCCTGACAGTGT |
| | | CTCCAGGAGGGACGGTCACGCTCACCTGCGGCCTCAAATC |
| | | TGGGTCTGTCACTTCCGATAACTTCCCCACTTGGTACCAG |
| | | CAGACACCAGGCCAGGCTCCCCGATTGCTTATCTACAACA |
| | | CAAACACCCGTCACTCTGGCGTCCCCGACCGCTTCTCCGG |
| | | ATCCATCCTGGGCAACAAAGCCGCCCTCACCATCACGGGG |
| | | GCCCAGGCCGACGACGAGGCCGAATATTTCTGTGCTCTGT |
| | | TCATAAGTAATCCTAGTGTTGAGTTCGGCGGAGGGACCCA |
| | | ACTGACCGTCCTAGGTCGTACGGTCACTGTCTCTTCACAG |
| | | GATCCCGCCGAGCCCAAATCTCCTGACAAAACTCACACAT |
| | | GCCCACCGTGCCCAGCACCTGAACTCCTGGGGGGACCGTC |
| | | AGTCTTCCTCTTCCCCCCAAAACCCAAGGACACCCTCATG |
| | | ATCTCCCGGACCCCTGAGGTCACATGCGTGGTGGTGGACG |
| | | TGAGCCACGAAGACCCTGAGGTCAAGTTCAACTGGTACGT |
| | | GGACGGCGTGGAGGTGCATAATGCCAAGACAAAGCCGCGG |
| | | GAGGAGCAGTACAACAGCACGTACCGTGTGGTCAGCGTCC |
| | | TCACCGTCCTGCACCAGGACTGGCTGAATGGCAAGGAGTA |
| | | CAAGTGCAAGGTCTCCAACAAAGCCCTCCCAGCCCCCATC |
| | | GAGAAAACCATCTCCAAAGCCAAAGGGCAGCCCCGAGAAC |
| | | CACAGGTGTACACCCTGCCCCCATCCCGGGATGAGCTGAC |
| | | CAAGAACCAGGTCAGCCTGACCTGCCTGGTCAAAGGCTTC |
| | | TATCCCAGCGACATCGCCGTGGAGTGGGAGAGCAATGGGC |
| | | AACCGGAGAACAACTACAAGACCACGCCTCCCGTGCTGGA |
| | | CTCCGACGGCTCCTTCTTCCTCTACAGCAAGCTCACCGTG |
| | | GACAAGAGCAGGTGGCAGCAGGGGAACGTCTTCTCATGCT |
| | | CCGTGATGCATGAGGCTCTGCACAACCACTACACGCAGAA |
| | | GAGCCTCTCCCTGTCTCCGGGTAAAAAAGATCCCAAATTT |
| | | TGGGTGCTGGTGGTGGTTGGTGGAGTCCTGGCTTGCTATA |
| | | GCTTGCTAGTAACAGTGGCCTTTATTATTTTCTGGGTGAG |
| | | GAGTAAGAGGAGCAGGCTCCTGCACAGTGACTACATGAAC |
| | | ATGACTCCCCGCCGCCCCGGGCCCACCCGCAAGCATTACC |
| | | AGCCCTATGCCCCACCACGCGACTTCGCAGCCTATCGCTC |
| | | ACGCGTGAAGTTCAGCAGGAGCGCAGACGCCCCCGCGTAC |
| | | CAGCAGGGCCAGAACCAGCTCTATAACGAGCTCAATCTAG |
| | | GACGAAGAGAGGAGTACGATGTTTTGGACAAAAGACGTGG |
| | | CCGGGACCCTGAGATGGGGGGAAAGCCGAGAAGGAAGAAC |
| | | CCTCAGGAAGGCCTGTACAATGAACTGCAGAAAGATAAGA |
| | | TGGCGGAGGCCTACAGTGAGATTGGGATGAAAGGCGAGCG |
| | | CCGGAGGGGCAAGGGGCACGATGGCCTTTACCAGGGTCTC |
| | | AGTACAGCCACCAAGGACACCTACGACGCCCTTCACATGC |
| | | AGGCCCTGCCCCCTCGCGGACCGCAGTGTACTAATTATGC |
| | | TCTCTTGAAATTGGCTGGAGATGTTGAGAGCAATCCCGGG |
| | | CCCATGCGCATTAGCAAGCCCCACCTGCGGAGCATCAGCA |
| | | TCCAGTGCTACCTGTGCCTGCTGCTGAACAGCCACTTCCT |
| | | GACCGAGGCCGGCATCCACGTGTTCATCCTGGGCTGCTTC |
| | | AGCGCCGGACTGCCCAAGACCGAGGCCAACTGGGTGAACG |
| | | TGATCAGCGACCTGAAGAAGATCGAGGACCTGATCCAGAG |
| | | CATGCACATCGACGCCACCCTGTACACCGAGAGCGACGTG |
| | | CACCCCAGCTGCAAGGTGACCGCCATGAAGTGCTTTCTGC |
| | | TGGAACTGCAGGTGATCAGCCTGGAAAGCGGCGACGCCAG |
| | | CATCCACGACACCGTGGAGAACCTGATCATCCTGGCCAAC |
| | | AACAGCCTGAGCAGCAACGGCAACGTGACCGAGAGCGGCT |
| | | GCAAAGAGTGCGAGGAACTGGAAGAGAAGAACATCAAAGA |
| | | GTTTCTGCAGAGCTTCGTGCACATCGTGCAGATGTTCATC |
| | | AACACCAGCTGA |
| CAR.CD70 | 97 | ATGGCCCTGCCTGTGACAGCTCTGCTCCTCCCTCTGGCCC |
| 42D12 | | TGCTGCTCCATGCCGCCAGACCCCAGGCAGTIGTGACCCA |
| VLVH.IgG1.C | | GGAGCCTTCCCTGACAGTGTCTCCAGGAGGGACGGTCACG |
| D28.CD3z-2A- | | CTCACCTGCGGCCTCAAATCTGGGTCTGTCACTTCCGATA |
| IL15 | | ACTTCCCCACTTGGTACCAGCAGACACCAGGCCAGGCTCC |
| | | CCGATTGCTTATCTACAACACAAACACCCGTCACTCTGGC |
| | | GTCCCCGACCGCTTCTCCGGATCCATCCTGGGCAACAAAG |
| | | CCGCCCTCACCATCACGGGGGCCCAGGCCGACGACGAGGC |
| | | CGAATATTTCTGTGCTCTGTTCATAAGTAATCCTAGTGTT |
| | | GAGTTCGGCGGAGGGACCCAACTGACCGTCCTAGGTGGCA |
| | | GCACCAGCGGCTCCGGCAAGCCTGGCTCTGGCGAGGGCAG |
| | | CACAAAGGGAGAGGTGCAGCTCGTGGAGTCTGGGGGAGGC |
| | | TTGGTGCAGCCTGGGGGGTCTCTGAGACTCTCCTGTGCAG |
| | | CCTCTGGATTCACCTTCAGTGTCTACTACATGAACTGGGT |
| | | CCGCCAGGCTCCAGGGAAGGGGCTtGAGTGGGTCTCAGAT |
| | | ATTAATAATGAAGGTGGTACTACATACTATGCAGACTCCG |
| | | TGAAGGGCCGATTCACCATCTCCAGAGACAACTCTAAGAA |
| | | CAGCCTGTATCTGCAAATGAACAGCCTGCGCGCCGAGGAC |
| | | ACGGCCGTGTACTACTGCGCGAGAGATGCCGGATATAGCA |
| | | ACCATGTACCCATCTTTGATTCTTGGGGCCAGGGGACCCT |
| | | GGTCACTGTCTCCTCACGTACGGTCACTGTCTCTTCACAG |
| | | GATCCCGCCGAGCCCAAATCTCCTGACAAAACTCACACAT |
| | | GCCCACCGTGCCCAGCACCTGAACTCCTGGGGGGACCGTC |
| | | AGTCTTCCTCTTCCCCCCAAAACCCAAGGACACCCTCATG |
| | | ATCTCCCGGACCCCTGAGGTCACATGCGTGGTGGTGGACG |
| | | TGAGCCACGAAGACCCTGAGGTCAAGTTCAACTGGTACGT |
| | | GGACGGCGTGGAGGTGCATAATGCCAAGACAAAGCCGCGG |
| | | GAGGAGCAGTACAACAGCACGTACCGTGTGGTCAGCGTCC |
| | | TCACCGTCCTGCACCAGGACTGGCTGAATGGCAAGGAGTA |
| | | CAAGTGCAAGGTCTCCAACAAAGCCCTCCCAGCCCCCATC |
| | | GAGAAAACCATCTCCAAAGCCAAAGGGCAGCCCCGAGAAC |
| | | CACAGGTGTACACCCTGCCCCCATCCCGGGATGAGCTGAC |
| | | CAAGAACCAGGTCAGCCTGACCTGCCTGGTCAAAGGCTTC |
| | | TATCCCAGCGACATCGCCGTGGAGTGGGAGAGCAATGGGC |
| | | AACCGGAGAACAACTACAAGACCACGCCTCCCGTGCTGGA |
| | | CTCCGACGGCTCCTTCTTCCTCTACAGCAAGCTCACCGTG |
| | | GACAAGAGCAGGTGGCAGCAGGGGAACGTCTTCTCATGCT |
| | | CCGTGATGCATGAGGCTCTGCACAACCACTACACGCAGAA |
| | | GAGCCTCTCCCTGTCTCCGGGTAAAAAAGATCCCAAATTT |
| | | TGGGTGCTGGTGGTGGTTGGTGGAGTCCTGGCTTGCTATA |
| | | GCTTGCTAGTAACAGTGGCCTTTATTATTTTCTGGGTGAG |
| | | GAGTAAGAGGAGCAGGCTCCTGCACAGTGACTACATGAAC |
| | | ATGACTCCCCGCCGCCCCGGGCCCACCCGCAAGCATTACC |
| | | AGCCCTATGCCCCACCACGCGACTTCGCAGCCTATCGCTC |
| | | ACGCGTGAAGTTCAGCAGGAGCGCAGACGCCCCCGCGTAC |
| | | CAGCAGGGCCAGAACCAGCTCTATAACGAGCTCAATCTAG |
| | | GACGAAGAGAGGAGTACGATGTTTTGGACAAAAGACGTGG |
| | | CCGGGACCCTGAGATGGGGGGAAAGCCGAGAAGGAAGAAC |
| | | CCTCAGGAAGGCCTGTACAATGAACTGCAGAAAGATAAGA |
| | | TGGCGGAGGCCTACAGTGAGATTGGGATGAAAGGCGAGCG |
| | | CCGGAGGGGCAAGGGGCACGATGGCCTTTACCAGGGTCTC |
| | | AGTACAGCCACCAAGGACACCTACGACGCCCTTCACATGC |
| | | AGGCCCTGCCCCCTCGCGGACCGCAGTGTACTAATTATGC |
| | | TCTCTTGAAATTGGCTGGAGATGTTGAGAGCAATCCCGGG |
| | | CCCATGCGCATTAGCAAGCCCCACCTGCGGAGCATCAGCA |
| | | TCCAGTGCTACCTGTGCCTGCTGCTGAACAGCCACTTCCT |
| | | GACCGAGGCCGGCATCCACGTGTTCATCCTGGGCTGCTTC |
| | | AGCGCCGGACTGCCCAAGACCGAGGCCAACTGGGTGAACG |
| | | TGATCAGCGACCTGAAGAAGATCGAGGACCTGATCCAGAG |
| | | CATGCACATCGACGCCACCCTGTACACCGAGAGCGACGTG |
| | | CACCCCAGCTGCAAGGTGACCGCCATGAAGTGCTTTCTGC |
| | | TGGAACTGCAGGTGATCAGCCTGGAAAGCGGCGACGCCAG |
| | | CATCCACGACACCGTGGAGAACCTGATCATCCTGGCCAAC |
| | | AACAGCCTGAGCAGCAACGGCAACGTGACCGAGAGCGGCT |
| | | GCAAAGAGTGCGAGGAACTGGAAGAGAAGAACATCAAAGA |
| | | GTTTCTGCAGAGCTTCGTGCACATCGTGCAGATGTTCATC |
| | | AACACCAGCTGA |

### VII. Genetically Engineered Receptors

Immune cells of the present disclosure can be genetically engineered to express one or more antigen-binding receptors that target one or more antigens, e.g., CD70, such as engineered CARs or, alternatively, engineered TCRs. For example, the immune cells may be immune cells that are modified to express a CAR and/or TCR having antigenic specificity for CD70. Other CARs and/or TCRs may be expressed by the same cells as the CD70 antigen receptor-expressing cells, and they may be directed to different antigens. In some aspects, the immune cells are engineered to express the CD70-specific CAR or CD70-specific TCR by knock-in of the CAR or TCR using, for example, CRISPR/Cas technology.

Suitable methods of modification of cells are known in the art. See, for instance, Sambrook and Ausubel, supra. For example, the cells may be transduced to express a CAR or TCR having antigenic specificity for a cancer antigen using transduction techniques described in Heemskerk et al., 2008 and Johnson et al., 2009.

In some aspects, the cells comprise one or more nucleic acids introduced via genetic engineering that encode one or more antigen-targeting receptors (at least one of which may be directed against CD70), and genetically engineered products of such nucleic acids. In some aspects, the nucleic acids are heterologous, i.e., normally not present in a cell or sample obtained from the cell, such as one obtained from another organism or cell, which for example, is not ordinarily found in the cell being engineered and/or an organism from which such cell is derived. In some aspects, the nucleic acids are not naturally occurring, such as a nucleic acid not found in nature (e.g., chimeric).

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

### A. Chimeric Antigen Receptors

In particular aspects, an antigen-specific CAR is utilized that comprises at least: a) one or more intracellular signaling domains, b) a transmembrane domain, and c) an extracellular domain comprising at least one antigen binding region. In some aspects the antigen binding region is an antibody or functional fragment thereof. In other cases, the antigen binding region of the CAR is not an antibody or functional fragment thereof (such as a ligand, e.g., CD27 for CD70). In some aspects wherein the CAR is CD70-specific, the antigen binding region of the CAR does not comprise an extracellular domain, or antigen binding portion thereof, from CD27. In some aspects, the antigen-specific CAR binds only a single antigen, whereas in other cases the CAR as a single polypeptide is bispecific by comprising two or more antigen binding domains, one of which that binds a first antigen and the other of which binds another, non-identical antigen. In some aspects, the CD70-specific CAR binds only CD70, whereas in other cases the CAR as a single polypeptide is bispecific by comprising two or more antigen binding domains, one of which that binds CD70 and the other of which binds another, non-identical antigen.

In some aspects, the engineered antigen receptors include CARs, including activating or stimulatory CARs, or costimulatory CARs (see WO2014/055668). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric CAR construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

Certain aspects of the present disclosure concern the use of nucleic acids, including nucleic acids encoding an antigen-specific, e.g., a CD70-specific, CAR polypeptide, including in some cases a CAR that has been humanized to reduce immunogenicity (hCAR), comprising at least one intracellular signaling domain, a transmembrane domain, and an extracellular domain comprising one or more signaling motifs. In certain aspects, the antigen-specific, e.g., a CD70-specific, CAR may recognize an epitope comprising the shared space between one or more antigens. In certain aspects, the binding region can comprise complementary determining regions of a monoclonal antibody, variable regions of a monoclonal antibody, and/or antigen binding fragments thereof. In another aspect, that specificity is derived from a peptide (e.g., cytokine) that binds to a receptor.

It is contemplated that the human antigen-specific, e.g., a CD70-specific, CAR nucleic acids may be used to enhance cellular immunotherapy for human patients. In a specific aspect, the disclosure includes a full-length antigen-specific, e.g., a CD70-specific, CAR cDNA or coding region. The antigen binding regions or domain can comprise a fragment of the V_{H} and V_{L} chains of a single-chain variable fragment (scFv) derived from a particular human monoclonal antibody. The fragment can also be any number of different antigen binding domains of a human antigen-specific antibody. In a more specific aspect, the fragment is an antigen-specific, e.g., a CD70-specific, scFv encoded by a sequence that is optimized for human codon usage for expression in human cells.

The arrangement could be multimeric, such as a diabody or multimers. The multimers may be formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the C_{H}2 or C_{H}3 domain from human immunoglobulin. One could also use the hinge, C_{H}2 and C_{H}3 region of a human immunoglobulin that has been modified to improve dimerization. One could also use just the hinge portion of an immunoglobulin. One could also use portions of CD8alpha or CD28.

In some aspects, antigen-specific CAR is constructed with specificity for an antigen expressed on a diseased cell type. Thus, the CAR typically includes in its extracellular portion one or more antigen-binding molecules, such as one or more antigen-binding fragments, domains, antibody variable domains, and/or antibody molecules of any kind. In some aspects, CD70-specific CAR is constructed with specificity for CD70, such as CD70 being expressed on a diseased cell type. Thus, the CAR typically includes in its extracellular portion one or more CD70-binding molecules, such as one or more antigen-binding fragments, domains, antibody variable domains, and/or antibody molecules of any kind.

In some aspects, the antigen-specific CAR includes an antigen-binding portion or portions of an antibody molecule, such as a single-chain antibody fragment (scFv) derived from the variable heavy (V_{H}) and variable light (V_{L}) chains of a monoclonal antibody (mAb). In some aspects, the CD70-specific CAR includes an antigen-binding portion or portions of an antibody molecule, such as a single-chain antibody fragment (scFv) derived from the variable heavy (V_{H}) and variable light (V_{L}) chains of a monoclonal antibody (mAb). In specific aspects, the antibody or functional fragment thereof is or is derived from 41D12, 2H5. The antibody may also be one that is generated de novo against CD70, and the scFv sequence may be obtained, or derived, from such de novo antibodies.

In certain aspects, the CAR comprises an antigen binding domain, e.g., an extracellular domain, that is or comprises a receptor for the antigen targeted by the CAR. In certain aspects, the anti-CD70 CAR comprises an extracellular domain that is or comprises a receptor for CD70. In specific aspects, the anti-CD70 CAR comprises an extracellular domain from CD27, or fragments or mimetics thereof. In certain aspects, the anti-CD70 CAR does not comprise an extracellular domain from CD27.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof, as it is found that introns stabilize the mRNA. Also, it may be further advantageous to use endogenous or exogenous non-coding regions to stabilize the mRNA.

In some aspects, the antigen-specific binding, or recognition, component is linked to one or more transmembrane and intracellular signaling domains. In some aspects, the CAR includes a transmembrane domain fused to the extracellular domain of the CAR. In one aspect, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex. The transmembrane domain in some aspects is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, DAP12, DAP10, NKG2D, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, a KIR such as KIR2DL4, GITR/CD357, and so forth. Alternatively, the transmembrane domain in some aspects is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In some aspects, the antigen-specific, e.g., CD70-specific, CAR nucleic acid comprises a sequence encoding other costimulatory receptors, such as a transmembrane domain and one or more intracellular signaling domains. In addition to a primary T cell activation signal, such as may be initiated by CD3ζ and/or FcεRIγ, an additional stimulatory signal for immune effector cell proliferation and effector function following engagement of the chimeric receptor with the target antigen may be utilized. For example, part or all of a human costimulatory receptor for enhanced activation of cells may be utilized that could help improve in vivo persistence and improve the therapeutic success of the adoptive immunotherapy. Examples include costimulatory domains from molecules such as DAP12, DAP10, NKG2D, CD2, CD28, CD27, 4-1BB, (CD137), OX40, ICOS, (CD278), CD30, HVEM, CD40, LFA-1 (CD11a/CD18), ICAM-1, and/or a portion of a CD70 cytoplasmic domain capable of inducing an activating signal, although in specific alternative aspects any one of these listed may be excluded from use in the CAR.

In certain aspects, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-5, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

### B. Examples of Specific CAR Aspects

In particular aspects, specific antigen-targeting, e.g., CD70-targeting, CAR molecules are encompassed herein. In some cases, the antigen, e.g., CD70, binding domain of the CAR is a scFv, and any scFv that binds to the antigen, e.g., CD70, may be utilized herein. In cases wherein an scFv is utilized in the extracellular domain of the CAR, the variable heavy chain and the variable light chain for the scFv may be in any order in N-terminal to C-terminal direction. For example, the variable heavy chain may be on the N-terminal side of the variable light chain, or vice versa. The variable heavy chain and the variable light chain may be separated by a linker. The scFv and/or ligand that binds the antigen in the CAR may or may not be codon optimized.

In particular aspects, the antigen binding domain that targets CD70 is a natural receptor of CD70, such as the receptor CD27. In specific cases, part or all of CD27 is employed in the CAR molecule. In aspects of the disclosure, the antigen binding domain present in the anti-CD70 CAR molecule comprises part or all of the extracellular domain of CD27, and in specific cases the CAR molecule does or does not utilize the transmembrane domain of CD27.

In particular aspects, a vector encodes an antigen-specific, e.g., a CD70-specific, CAR and also encodes one or more other molecules. For example, a vector may encode an antigen-specific, e.g., a CD70-specific, CAR and also may encode another protein of interest, such as another engineered antigen receptor, a suicide gene, and/or a particular cytokine.

On the same molecule, the antigen-specific, e.g., a CD70-specific, CAR may comprise one or more antigen-specific extracellular domains, a specific hinge, a specific transmembrane domain, one or more specific costimulatory domains, and one or more specific activation signals. When more than one antigen-specific extracellular domain is utilized, such as for targeting two different antigens (one of which may be CD70), there may be a linker between the two antigen-specific extracellular domains.

In particular aspects of specific CAR molecules, a CAR may utilize DAP10, DAP12, 4-1BB, NKG2D, or other costimulatory domains (which may be referred to herein as an intracytoplasmic domain). In some cases, CD3zeta is utilized without any costimulatory domains. In particular aspects of specific CAR molecules, a CAR may utilize any suitable transmembrane domain, such as from DAP12, DAP10, 4-1BB, 2B4, OX40, CD27, NKG2D, CD8, or CD28.

In particular aspects, there is an expression construct comprising a sequence that encodes a particular CD70-specific engineered receptor. In particular aspects, any CD70-targeting CAR may comprise one of SEQ ID NOs: 44-46 or 69.

Examples of specific sequence aspects are provided below.

### 1. Antigen-Specific Extracellular Domains

Examples of specific sequence aspects are provided below.

In particular aspects, a vector encodes a CD70-specific CAR. For example, a vector may encode a CD70-specific CAR that may or may not be codon optimized (CO), and in specific cases the anti-CD70 scFv is the 42D12 scFv that may have the variable light chain upstream or downstream of the variable heavy chain.

Example CD70-binding region amino acid sequences, e.g., anti-CD70 scFvs, are as follows:

CO CAR.CD70 42D12 VLVH:
(SEQ ID NO: 44)
MALPVTALLLPLALLLHAARPQAVVTQEPSLTVSPGGTVTLTCGLKSGSV
TSDNFPTWYQQTPGQAPRLLIYNTNTRHSGVPDRFSGSILGNKAALTITG
AQADDEAEYFCALFISNPSVEFGGGTQLTVLGGSTSGSGKPGSGEGSTKG
EVQLVESGGGLVQPGGSLRLSCAASGFTFSVYYMNWVRQAPGKGLEWVSD
INNEGGTTYYADSVKGRFTISRDNSKNSLYLQMNSLRAEDTAVYYCARDA
GYSNHVPIFDSWGQGTLVTVSS

Any polypeptide encompassed by the present disclosure may comprise SEQ ID NO: 44 or a sequence that is at least 70, 75, 80, 85, 90, 95, 96, 97, 98, 99, or more % identical to SEQ ID NO:44.

CAR.CD70 42D12 VHVL:
(SEQ ID NO: 45)
MGMALPVTALLLPLALLLHAARPEVQLVESGGGLVQPGGSLRLSCAASGF
TFSVYYMNWVRQAPGKGLEWVSDINNEGGTTYYADSVKGRFTISRDNSKN
SLYLQMNSLRAEDTAVYYCARDAGYSNHVPIFDSWGQGTLVTVSSGSTSG
SGKPGSGEGSTKGQAVVTQEPSLTVSPGGTVTLTCGLKSGSVTSDNFPTW
YQQTPGQAPRLLIYNTNTRHSGVPDRFSGSILGNKAALTITGAQADDEAE
YFCALFISNPSVEFGGGTQLTVLG

Any polypeptide encompassed by the present disclosure may comprise SEQ ID NO: 45 or a sequence that is at least 70, 75, 80, 85, 90, 95, 96, 97, 98, 99, or more % identical to SEQ ID NO:45.

CAR.CD70 42D12 VLVH:
(SEQ ID NO: 46)
MGMALPVTALLLPLALLLHAARPEVQLVESGGGLVQPGGSLRLSCAASGF
TFSVYYMNWVRQAPGKGLEWVSDINNEGGTTYYADSVKGRFTISRDNSKN
SLYLQMNSLRAEDTAVYYCARDAGYSNHVPIFDSWGQGTLVTVSSGSTSG
SGKPGSGEGSTKGQAVVTQEPSLTVSPGGTVTLTCGLKSGSVTSDNFPTW
YQQTPGQAPRLLIYNTNTRHSGVPDRFSGSILGNKAALTITGAQADDEAE
YFCALFISNPSVEFGGGTQLTVLG

Any polypeptide encompassed by the present disclosure may comprise SEQ ID NO: 46 or a sequence that is at least 70, 75, 80, 85, 90, 95, 96, 97, 98, 99, or more % identical to SEQ ID NO:46.

In specific examples, a CD70-binding region that is utilized in a CAR molecule of the disclosure comprises, consists of, or consists essentially of amino acids 1-50, 1-51, 1-52, 1-53, 1-54, 1-55, 1-56, 1-57, 1-58, 1-59, 1-60, 1-61, 1-62, 1-63, 1-64, 1-65, 1-66, 1-67, 1-68, 1-69, 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, 1-100, 1-101, 1-102, 1-103, 1-104, 1-105, 1-106, 1-107, 1-108, 1-109, 1-110, 1-111, 1-112, 1-113, 1-114, 1-115, 1-116, 1-117, 1-118, 1-119, 1-120, 1-121, 1-122, 1-123, 1-124, 1-125, 1-126, 1-127, 1-128, 1-129, 1-130, 1-131, 1-132, 1-133, 1-134, 1-135, 1-136, 1-137, 1-138, 1-139, 1-140, 1-141, 1-142, 1-143, 1-144, 1-145, 1-146, 1-147, 1-148, 1-149, 1-150, 1-151, 1-152, 1-153, 1-154, 1-155, 1-156, 1-157, 1-158, 1-159, 1-160, 1-161, 1-162, 1-163, 1-164, 1-165, 1-166, 1-167, 1-168, 1-169, 1-170, 1-171, 1-172, 1-173, 1-174, 1-175, 1-176, 1-177, 1-178, 1-179, 1-180, 1-181, 1-182, 1-183, 1-184, 1-185, 1-186, 1-187, 1-188, 1-189, 1-190, 1-191, 1-192, 1-193, 1-194, 1-195, 1-196, 1-197, 1-198, 1-199, 1-200, 1-201, 1-202, 1-203, 1-204, 1-205, 1-206, 1-207, 1-208, 1-209, 1-210, 1-211, 1-212, 1-213, 1-214, 1-215, 1-216, 1-217, 1-218, 1-219, 1-220, or all of SEQ ID NOs: 44-46; in specific aspects, such amino acids in these ranges are contiguous. In some aspects, a region of SEQ ID NOs: 44-46 is utilized that has truncation at the N-terminus, such as 1, 2, 3, 4, 5, 6, 7, 8, 9, 10, 11, 12, 13, 14, 15, 16, 17, 18, 19, 20, or more amino acids from the N-terminus. In certain cases, there is truncation at that N-terminus of 1, 2, 3, 4, 5, 6, 7, 8, 9, 10, 11, 12, 13, 14, 15, 16, 17, 18, 19, 20, or more amino acids and there is truncation at the C-terminus.

In some aspects, instead of utilizing an antibody or antibody fragment for CD70-binding, a CD70-binding region comprises part or all of CD27, including using the extracellular domain of CD27 as the CD70-binding region, in some cases. A CD27 CD70-binding region of the disclosure may comprise SEQ ID NO:69.

### 2. Transmembrane Domains

Any suitable transmembrane domain may be utilized in an antigen-specific, e.g., a CD70-specific, CAR of the disclosure. Examples include at least transmembrane domains from DAP10, DAP12, CD28, NKG2D, CD3 epsilon, CD4, CD5, CD8, CD9, CD16, CD22, CD28, CD33, CD37, CD45, CD64, CD80, CD86, CD134, CD137, or CD154, from a T-cell receptor a or b chain, from a CD3 zeta chain, from ICOS, functional derivatives thereof, and combinations thereof. In specific cases, a transmembrane domain from DAP10, DAP12, CD28, CD8, or NKG2D is utilized. In some aspects, a transmembrane domain from CD70. Examples of particular transmembrane domain sequences may be used, as follows:

CD28 transmembrane domain amino acid sequence:

| (SEQ ID NO: 47) | |
|---|---|
| | FWVLVVVGGVLACYSLLVTVAFIIFWV |

CD27 transmembrane domain amino acid sequence:

| (SEQ ID NO: 70) | |
|---|---|
| | ILVIFSGMFLVFTLAGALFLH |

CD8 transmembrane domain amino acid sequence:

(SEQ ID NO: 48)
TTTPAPRPPTPAPTIASQPLSLRPEACRPAAGGAVHTRGLDFACDIYIWA
PLAGTCGVLLLSLVIT

4-1BB transmembrane domain amino acid sequence:

(SEQ ID NO: 49)
IISFFLALTSTALLFLLFFLTLRFSVV

DAP10 transmembrane domain amino acid sequence:

(SEQ ID NO: 50)
LLAGLVAADAVASLLIVGAVF

DAP12 transmembrane domain amino acid sequence:

(SEQ ID NO: 51)
GVLAGIVMGDLVLTVLIALAV

NKG2D transmembrane domain amino acid sequence:

(SEQ ID NO: 52)
AVMIIFRIGMAVAIFCCFFFP

Any polypeptide encompassed by the present disclosure may comprise one of SEQ ID NOs: 47-52 or 70, or a sequence that is at least 70, 75, 80, 85, 90, 95, 96, 97, 98, 99, or more % identical to one of SEQ ID NOs: 47-52 or 70.

### 3. Intracellular Domains

One or more intracellular domains (which may also be referred to herein as signal activation domains or costimulatory domains, in appropriate cases) may or may not be utilized in an antigen-specific, e.g., a CD70-specific, CAR of the disclosure. The one or more intracellular domains can be any ITAM-containing domain. Specific examples include intracellular domains from CD3 zeta, 4-1BB, NKG2D, OX-40, CD27, DAP10, DAP12, B7-1/CD80, CD28, 2B4, 4-1BBL, B7-2/CD86, CTLA-4, B7-H1/PD-L1, ICOS, B7-H2, PD-1, B7-H3, PD-L2, B7-H4, PDCD6, BTLA, or a combination thereof.

Examples of particular intracellular domains which may be used in a CAR of the disclosure are as follows:

An example CD3zeta intracellular domain amino acid sequence:

(SEQ ID NO: 53)
TRKKFSRSADAPAYQQGQNQLYNELNLGRREEYDVLDKRRGRDPEMGG
KPRRKNPQEGLYNELQKDKMAEAYSEIGMKGERRRGKGHDGLYQGLST
ATKDTYDALHMQALPPRG

An example CD3zeta intracellular domain amino acid sequence:

(SEQ ID NO: 54)
KRVKFSRSADAPAYQQGQNQLYNELNLGRREEYDVLDKRRGRDPEMGG
KPRRKNPQEGLYNELQKDKMAEAYSEIGMKGERRRGKGHDGLYQGLST
ATKDTYDALHMQALPPRG

An example CD3zeta intracellular domain amino acid sequence:

(SEQ ID NO: 55)
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPRG

4-1BB intracellular domain amino acid sequence:

(SEQ ID NO: 56)
KRGRKKLLYIFKQPFMRPVQTTQEEDGCSCRFPEEEEGGCEL

DAP10 intracellular domain amino acid sequence:

(SEQ ID NO: 57)
LCARPRRSPAQEDGKVYINMPGRG

DAP12 intracellular domain amino acid sequence:

(SEQ ID NO: 58)
YFLGRLVPRGRGAAEAATRKQRITETESPYQELQGQRSDVYSDLNTQRP
YYK

NKG2D intracellular domain amino acid sequence:

(SEQ ID NO: 59)
SANERCKSKVVPCRQKQWRTSFDSKKLDLNYNHFESMEWSHRSRRGRIW
GM

Any polypeptide encompassed by the present disclosure may comprise SEQ ID NOs: 53-59, or a sequence that is at least 70, 75, 80, 85, 90, 95, 96, 97, 98, 99, or more % identical to one of SEQ ID NOs: 53-59.

### 4. Hinge

In some aspects of the CARs, there is a hinge region between the one or more extracellular antigen binding domains and the transmembrane domain. In specific aspects, the hinge is of a particular length, such as 10-20, 10-15, 11-20, 11-15, 12-20, 12-15, or 15-20 amino acids in length, for example. The hinge may be any suitable hinge and includes a hinge from IgG, CD8, or CD28, in some cases. In specific aspects, the hinge is a small flexible polypeptide that connects C_{H}2-C_{H}3 and CHI domains of IgG Fc. For example, one may utilize C_{H}2-C_{H}3 hinge (part or all) from various IgG subclasses (IgG1-4, either modified or not). However, in some cases the entire C_{H}2-C_{H}3 hinge is not utilized but instead a portion of the hinge is used (such as C_{H}3 by itself or part of C_{H}3 by itself). In particular aspects, the C_{H}2-C_{H}3 hinge derived from IgG1 is utilized, and in some cases the entire C_{H}2-C_{H}3 hinge is used (all 229 amino acids), only the C_{H}3 hinge (119 amino acids) is used, or a short hinge (12 amino acids) is used.

In specific cases, one can modify the identity or length of the spacer and/or hinge to optimize efficiency of the CAR. See, e.g., Hudecek et al. (2014) and Jonnalagadda et al. (2015) In specific aspects, the CD70 CAR utilizes IgG4 hinge+C_{H}3 or utilizes CD8a stalk, for example.

Thus, in specific aspects the IgG hinge region that is utilized is typically IgG1 or IgG4, and in some cases the CAR comprises the C_{H}2-C_{H}3 domain of IgG Fc. The use of the IgG Fc domain can provide flexibility to the CAR, has low immunogenicity, facilitates detection of CAR expression using anti-Fc reagents, and allows removal of one or more C_{H}2 or C_{H}3 modules to accommodate different spacer lengths. However, in one aspect mutations in certain spacers to avoid FcγR binding may improve CAR+ T cell engraftment and antitumor efficacy to avoid binding of soluble and cell surface Fc gamma receptors, for example, yet maintain the activity to mediate antigen-specific lysis. For example, one can employ IgG4-Fc spacers that have either been modified in the C_{H}2 region. For example, the C_{H}2 region may be mutated, including point mutations and/or deletions. Specific modifications have been demonstrated at two sites (L235E; N297Q) within the C_{H}2 region and/or incorporate a C_{H}2 deletion (Jonnalagadda et al, 2015). In specific aspects, one may employ the IgG4 hinge-C_{H}2-C_{H}3 domain (229 aa in length) or only the hinge domain (12 aa in length) (Hudececk et al., 2015).

In specific aspects, the hinge is from IgG, CD28, CD-8 alpha, 4-1BB, 0X40, CD3-zeta, T cell receptor a or b chain, a CD3 zeta chain, CD28, CD3e, CD45, CD4, CD5, CD8, CD9, CD16, CD22, CD33, CD37, CD64, CD80, CD86, CD134, CD137, ICOS, or CD154.

Examples of specific sequences of hinges that may be utilized include at least the following:

IgG Hinge amino acid sequence:

(SEQ ID NO: 60)
TVTVSSQDPAEPKSPDKTHTCPPCPAPELLGGPSVFLFPPKPKDTLMIS
RTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSD
GSFFLYSKLTVDKSRWQQGNVFSCSVMHEAL

CD28 Hinge amino acid sequence:

(SEQ ID NO: 61)
IEVMYPPPYLDNEKSNGTIIHVKGKHLCPSPLFPGPSKP

CD8 hinge amino acid sequence

(SEQ ID NO: 94)
KPTTTPAPRPPTPAPTIASQPLSLRPEACRPAAGGAVHTRGLDFACDIY
IWAPLAGTCGVLLLSLVITLYCNHRN

Any polypeptide encompassed by the present disclosure may comprise SEQ ID NO: 60, 61, or 94 or a sequence that is at least 70, 75, 80, 85, 90, 95, 96, 97, 98, 99, or more % identical to SEQ ID NO:60, 61, or 94.

### 5. Signal Peptides

In particular aspects, signal peptides are employed for the CAR, and examples include the CD27 or GMCSF-R signal peptides, or both may be used.

In some cases, the CD27 signal peptide is utilized (MARPHPWWLCVLGTLVGLS; SEQ ID NO:67) in the CAR, or a sequence that is at least 70, 75, 80, 85, 90, 95, 96, 97, 98, 99, or more % identical to SEQ ID NO:67. In some cases, the GMCSF-R signal peptide (MLLLVTSLLLCELPHPAFLLIP; SEQ ID NO:68) is used in the CAR, or a sequence that is at least 70, 75, 80, 85, 90, 95, 96, 97, 98, 99, or more % identical to SEQ ID NO:68. In some cases, the signal peptide is from CD8. In some cases, the signal peptide is from IgH.

### 6. Other Proteins

In some aspects, one or more other proteins are utilized with an antigen-specific, e.g., a CD70-specific, CAR of the disclosure. The one or more other proteins may be utilized for any reason, including to facilitate efficacy of the CAR itself and/or of any kind of cells expressing the CAR. In some cases, the other protein facilitates treatment of an individual receiving cells expressing the CAR as therapy, whether or not the other protein(s) directly or indirectly impact activity of the CAR or the cells. In some cases, the other protein is one or more antibodies or one or more bispecific or multispecific immune cell engagers. In some cases, the other protein is a suicide gene, one or more cytokines, or both. In specific aspects, the one or more other proteins are produced from one or more vectors and ultimately are produced as separate polypeptides. In specific aspects, the one or more other proteins are produced from the same vector and ultimately are produced as separate polypeptides. For example, the antigen-specific, e.g., a CD70-specific, CAR and the other protein(s) may be separated by a 2A sequence or by an IRES.

In specific aspects, a cytokine such as IL-15 is utilized in conjunction with the anti-CD70 CAR.

One example of an IL-15 sequence is as follows:

IL-15 amino acid sequence:

(SEQ ID NO: 62)
ISKPHLRSISIQCYLCLLLNSHFLTEAGIHVFILGCFSAGLPKTEANWV
NVISDLKKIEDLIQSMHIDATLYTESDVHPSCKVTAMKCFLLELQVISL
ESGDASIHDTVENLIILANNSLSSNGNVTESGCKECEELEEKNIKEFLQ
SFVHIVQMFINTS

In other aspects, the cytokine IL-21 is utilized in conjunction with the anti-CD70 CAR. In other aspects, the cytokine IL-12 is utilized in conjunction with the anti-CD70 CAR.

In cases where the CAR and another protein in the same vector are intended to be produced into two different polypeptides, a specific 2A sequence may be utilized.

An E2A amino acid sequence may be utilized as follows:

(SEQ ID NO: 63)
QCTNYALLKLAGDVESNPGP

Other 2A examples may be utilized and are as follows:

(SEQ ID NO: 64)
T2A: EGRGSLLTCGDVEENPGP
(SEQ ID NO: 65)
P2A: ATNFSLLKQAGDVEENPGP
(SEQ ID NO: 66)
F2A: VKQTLNFDLLKLAGDVESNPGP

The disclosure also encompasses specific CAR molecules, including for expression in any type of immune effector cells (e.g., T cells, NK cells, NKT cells, etc.).

In some aspects, an antigen-specific, e.g., a CD70-specific, CAR comprising an antigen binding, e.g., a CD70-binding, domain, an IgG1 hinge, a CD28 intracellular domain, and a CD35 intracellular domain is utilized. In a vector, the CAR may be expressed with IL-15, such as may be separated from the CAR by a 2A sequence.

Examples of specific vector molecules including an antigen-specific, e.g., a CD70-specific, CAR and IL15 encompass at least the following:
CO CAR.CD70 42D12. VLVH.IgG1.CD28.CD3z-2A-IL15
CO CAR.CD70 42D12 VHVL.IgG1.CD28.CD3z-2A-IL15
CAR.CD70 42D12 VLVH.IgG1.CD28.CD3z-2A-IL15
CAR.CD70 42D12 VHVL.IgG1.CD28.CD3z-2A-IL15

The full DNA sequence for the vector comprising CO CAR.CD70 42D12. VLVH.IgG1.CD28.CD3z-2A-IL15 is as follows:

(SEQ ID NO: 95)
ATGGCCCTGCCTGTGACAGCTCTGCTCCTCCCTCTGGCCCTGCTGCTCC
ATGCCGCCAGACCCCAGGCAGTIGTGACCCAGGAGCCTTCCCTGACAGTGTCTCC
AGGAGGGACGGTCACGCTCACCTGCGGCCTCAAATCTGGGTCTGTCACTTCCGAT
AACTTCCCCACTTGGTACCAGCAGACACCAGGCCAGGCTCCCCGATTGCTTATCT
ACAACACAAACACCCGTCACTCTGGCGTCCCCGACCGCTTCTCCGGATCCATCCT
GGGCAACAAAGCCGCCCTCACCATCACGGGGGCCCAGGCCGACGACGAGGCCGA
ATATTTCTGTGCTCTGTTCATAAGTAATCCTAGTGTTGAGTTCGGCGGAGGGACC
CAACTGACCGTCCTAGGTGGCAGCACCAGCGGCTCCGGCAAGCCTGGCTCTGGC
GAGGGCAGCACAAAGGGAGAGGTGCAGCTCGTGGAGTCTGGGGGAGGCTTGGT
GCAGCCTGGGGGGTCTCTGAGACTCTCCTGTGCAGCCTCTGGATTCACCTTCAGT
GTCTACTACATGAACTGGGTCCGCCAGGCTCCAGGGAAGGGGCTCGAGTGGGTC
TCAGATATTAATAATGAAGGTGGTACTACATACTATGCAGACTCCGTGAAGGGCC
GATTCACCATCTCCAGAGACAACTCTAAGAACAGCCTGTATCTGCAAATGAACA
GCCTGCGCGCCGAGGACACGGCCGTGTACTACTGCGCGAGAGATGCCGGATATA
GCAACCATGTACCCATCTTTGATTCTTGGGGCCAGGGGACCCTGGTCACTGTCTC
CTCACGTACGGTCACTGTCTCTTCACAGGATCCCGCCGAGCCCAAATCTCCTGAC
AAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTG
AGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGG
AGCAGTACAACAGCACGTACCGTGTGGTCAGCGTCCTCACCGTCCTGCACCAGG
ACTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACAGG
TGTACACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAGAGCA
ATGGGCAACCGGAGAACAACTACAAGACCACGCCTCCCGTGCTGGACTCCGACG
GCTCCTTCTTCCTCTACAGCAAGCTCACCGTGGACAAGAGCAGGTGGCAGCAGG
GGAACGTCTTCTCATGCTCCGTGATGCATGAGGCTCTGCACAACCACTACACGCA
GAAGAGCCTCTCCCTGTCTCCGGGTAAAAAAGATCCCAAATTTTGGGTGCTGGTG
GTGGTTGGTGGAGTCCTGGCTTGCTATAGCTTGCTAGTAACAGTGGCCTTTATTAT
TTTCTGGGTGAGGAGTAAGAGGAGCAGGCTCCTGCACAGTGACTACATGAACAT
GACTCCCCGCCGCCCCGGGCCCACCCGCAAGCATTACCAGCCCTATGCCCCACCA
CGCGACTTCGCAGCCTATCGCTCACGCGTGAAGTTCAGCAGGAGCGCAGACGCC
CCCGCGTACCAGCAGGGCCAGAACCAGCTCTATAACGAGCTCAATCTAGGACGA
AGAGAGGAGTACGATGTTTTGGACAAAAGACGTGGCCGGGACCCTGAGATGGGG
GGAAAGCCGAGAAGGAAGAACCCTCAGGAAGGCCTGTACAATGAACTGCAGAA
AGATAAGATGGCGGAGGCCTACAGTGAGATTGGGATGAAAGGCGAGCGCCGGA
GGGGCAAGGGGCACGATGGCCTTTACCAGGGTCTCAGTACAGCCACCAAGGACA
CCTACGACGCCCTTCACATGCAGGCCCTGCCCCCTCGCGGACCGCAGTGTACTAA
TTATGCTCTCTTGAAATTGGCTGGAGATGTTGAGAGCAATCCCGGGCCCATGCGC
ATTAGCAAGCCCCACCTGCGGAGCATCAGCATCCAGTGCTACCTGTGCCTGCTGC
TGAACAGCCACTTCCTGACCGAGGCCGGCATCCACGTGTTCATCCTGGGCTGCTT
CAGCGCCGGACTGCCCAAGACCGAGGCCAACTGGGTGAACGTGATCAGCGACCT
GAAGAAGATCGAGGACCTGATCCAGAGCATGCACATCGACGCCACCCTGTACAC
CGAGAGCGACGTGCACCCCAGCTGCAAGGTGACCGCCATGAAGTGCTTTCTGCT
GGAACTGCAGGTGATCAGCCTGGAAAGCGGCGACGCCAGCATCCACGACACCGT
GGAGAACCTGATCATCCTGGCCAACAACAGCCTGAGCAGCAACGGCAACGTGAC
CGAGAGCGGCTGCAAAGAGTGCGAGGAACTGGAAGAGAAGAACATCAAAGAGT
TTCTGCAGAGCTTCGTGCACATCGTGCAGATGTTCATCAACACCAGCTGA

In some aspects, a codon optimized CO CAR.CD70 42D12 VHVL.IgG1.CD28.CD3z-2A-IL 15 vector is employed. A full DNA sequence for the following construct CO CAR.CD70 42D12 VHVL.IgG1.CD28.CD3z-2A-IL15 is as follows:

(SEQ ID NO: 96)
ATGGCCCTGCCTGTGACAGCTCTGCTCCTCCCTCTGGCCCTGCTGCTCC
ATGCCGCCAGACCCGAGGTGCAGCTCGTGGAGTCTGGGGGAGGCTTGGTGCAGC
CTGGGGGGTCTCTGAGACTCTCCTGTGCAGCCTCTGGATTCACCTTCAGTGTCTAC
TACATGAACTGGGTCCGCCAGGCTCCAGGGAAGGGGCTCGAGTGGGTCTCAGAT
ATTAATAATGAAGGTGGTACTACATACTATGCAGACTCCGTGAAGGGCCGATTCA
CCATCTCCAGAGACAACTCTAAGAACAGCCTGTATCTGCAAATGAACAGCCTGC
GCGCCGAGGACACGGCCGTGTACTACTGCGCGAGAGATGCCGGATATAGCAACC
ATGTACCCATCTTTGATTCTTGGGGCCAGGGGACCCTGGTCACTGTCTCCTCAGG
CAGCACCAGCGGCTCCGGCAAGCCTGGCTCTGGCGAGGGCAGCACAAAGGGACA
GGCAGTGGTGACCCAGGAGCCTTCCCTGACAGTGTCTCCAGGAGGGACGGTCAC
GCTCACCTGCGGCCTCAAATCTGGGTCTGTCACTTCCGATAACTTCCCCACTTGGT
ACCAGCAGACACCAGGCCAGGCTCCCCGATTGCTTATCTACAACACAAACACCC
GTCACTCTGGCGTCCCCGACCGCTTCTCCGGATCCATCCTGGGCAACAAAGCCGC
CCTCACCATCACGGGGGCCCAGGCCGACGACGAGGCCGAATATTTCTGTGCTCTG
TTCATAAGTAATCCTAGTGTTGAGTTCGGCGGAGGGACCCAACTGACCGTCCTAG
GTCGTACGGTCACTGTCTCTTCACAGGATCCCGCCGAGCCCAAATCTCCTGACAA
AACTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAGG
TCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGT
ACACCCTGCCCCCATCCCGGGATGAGCTGACCAAGAACCAGGTCAGCCTGACCT
GCCTGGTCAAAGGCTTCTATCCCAGCGACATCGCCGTGGAGTGGGAGAGCAATG
GGCAACCGGAGAACAACTACAAGACCACGCCTCCCGTGCTGGACTCCGACGGCT
CCTTCTTCCTCTACAGCAAGCTCACCGTGGACAAGAGCAGGTGGCAGCAGGGGA
ACGTCTTCTCATGCTCCGTGATGCATGAGGCTCTGCACAACCACTACACGCAGAA
GAGCCTCTCCCTGTCTCCGGGTAAAAAAGATCCCAAATTTTGGGTGCTGGTGGTG
GTTGGTGGAGTCCTGGCTTGCTATAGCTTGCTAGTAACAGTGGCCTTTATTATTTT
CTGGGTGAGGAGTAAGAGGAGCAGGCTCCTGCACAGTGACTACATGAACATGAC
TCCCCGCCGCCCCGGGCCCACCCGCAAGCATTACCAGCCCTATGCCCCACCACGC
GACTTCGCAGCCTATCGCTCACGCGTGAAGTTCAGCAGGAGCGCAGACGCCCCC
GCGTACCAGCAGGGCCAGAACCAGCTCTATAACGAGCTCAATCTAGGACGAAGA
GAGGAGTACGATGTTTTGGACAAAAGACGTGGCCGGGACCCTGAGATGGGGGGA
AAGCCGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGAGGG
GCAAGGGGCACGATGGCCTTTACCAGGGTCTCAGTACAGCCACCAAGGACACCT
ACGACGCCCTTCACATGCAGGCCCTGCCCCCTCGCGGACCGCAGTGTACTAATTA
TGCTCTCTTGAAATTGGCTGGAGATGTTGAGAGCAATCCCGGGCCCATGCGCATT
AGCAAGCCCCACCTGCGGAGCATCAGCATCCAGTGCTACCTGTGCCTGCTGCTGA
ACAGCCACTTCCTGACCGAGGCCGGCATCCACGTGTTCATCCTGGGCTGCTTCAG
CGCCGGACTGCCCAAGACCGAGGCCAACTGGGTGAACGTGATCAGCGACCTGAA
GAAGATCGAGGACCTGATCCAGAGCATGCACATCGACGCCACCCTGTACACCGA
GAGCGACGTGCACCCCAGCTGCAAGGTGACCGCCATGAAGTGCTTTCTGCTGGA
ACTGCAGGTGATCAGCCTGGAAAGCGGCGACGCCAGCATCCACGACACCGTGGA
GAACCTGATCATCCTGGCCAACAACAGCCTGAGCAGCAACGGCAACGTGACCGA
GAGCGGCTGCAAAGAGTGCGAGGAACTGGAAGAGAAGAACATCAAAGAGTTTC
TGCAGAGCTTCGTGCACATCGTGCAGATGTTCATCAACACCAGCTGA

Non-codon-optimized CARs may also be employed, such as a CAR.CD70 42D12 VLVH.IgG1.CD28.CD3z-2A-IL15 Vector, and a sequence is provided below:

(SEQ ID NO: 97)
ATGGCCCTGCCTGTGACAGCTCTGCTCCTCCCTCTGGCCCTGCTGCTCC
ATGCCGCCAGACCCCAGGCAGTIGTGACCCAGGAGCCTTCCCTGACAGTGTCTCC
AGGAGGGACGGTCACGCTCACCTGCGGCCTCAAATCTGGGTCTGTCACTTCCGAT
AACTTCCCCACTTGGTACCAGCAGACACCAGGCCAGGCTCCCCGATTGCTTATCT
ACAACACAAACACCCGTCACTCTGGCGTCCCCGACCGCTTCTCCGGATCCATCCT
GGGCAACAAAGCCGCCCTCACCATCACGGGGGCCCAGGCCGACGACGAGGCCGA
ATATTTCTGTGCTCTGTTCATAAGTAATCCTAGTGTTGAGTTCGGCGGAGGGACC
CAACTGACCGTCCTAGGTGGCAGCACCAGCGGCTCCGGCAAGCCTGGCTCTGGC
GAGGGCAGCACAAAGGGAGAGGTGCAGCTCGTGGAGTCTGGGGGAGGCTTGGT
GCAGCCTGGGGGGTCTCTGAGACTCTCCTGTGCAGCCTCTGGATTCACCTTCAGT
GTCTACTACATGAACTGGGTCCGCCAGGCTCCAGGGAAGGGGCTtGAGTGGGTCT
CAGATATTAATAATGAAGGTGGTACTACATACTATGCAGACTCCGTGAAGGGCC
GATTCACCATCTCCAGAGACAACTCTAAGAACAGCCTGTATCTGCAAATGAACA
GCCTGCGCGCCGAGGACACGGCCGTGTACTACTGCGCGAGAGATGCCGGATATA
GCAACCATGTACCCATCTTTGATTCTTGGGGCCAGGGGACCCTGGTCACTGTCTC
CTCACGTACGGTCACTGTCTCTTCACAGGATCCCGCCGAGCCCAAATCTCCTGAC
AAAACTCACACATGCCCACCGTGCCCAGCACCTGAACTCCTGGGGGGACCGTCA
GTCTTCCTCTTCCCCCCAAAACCCAAGGACACCCTCATGATCTCCCGGACCCCTG
AGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGG
AGCAGTACAACAGCACGTACCGTGTGGTCAGCGTCCTCACCGTCCTGCACCAGG
ACTGGCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCACAGG
TGTACACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAGAGCA
ATGGGCAACCGGAGAACAACTACAAGACCACGCCTCCCGTGCTGGACTCCGACG
GCTCCTTCTTCCTCTACAGCAAGCTCACCGTGGACAAGAGCAGGTGGCAGCAGG
GGAACGTCTTCTCATGCTCCGTGATGCATGAGGCTCTGCACAACCACTACACGCA
GAAGAGCCTCTCCCTGTCTCCGGGTAAAAAAGATCCCAAATTTTGGGTGCTGGTG
GTGGTTGGTGGAGTCCTGGCTTGCTATAGCTTGCTAGTAACAGTGGCCTTTATTAT
TTTCTGGGTGAGGAGTAAGAGGAGCAGGCTCCTGCACAGTGACTACATGAACAT
GACTCCCCGCCGCCCCGGGCCCACCCGCAAGCATTACCAGCCCTATGCCCCACCA
CGCGACTTCGCAGCCTATCGCTCACGCGTGAAGTTCAGCAGGAGCGCAGACGCC
CCCGCGTACCAGCAGGGCCAGAACCAGCTCTATAACGAGCTCAATCTAGGACGA
AGAGAGGAGTACGATGTTTTGGACAAAAGACGTGGCCGGGACCCTGAGATGGGG
GGAAAGCCGAGAAGGAAGAACCCTCAGGAAGGCCTGTACAATGAACTGCAGAA
AGATAAGATGGCGGAGGCCTACAGTGAGATTGGGATGAAAGGCGAGCGCCGGA
GGGGCAAGGGGCACGATGGCCTTTACCAGGGTCTCAGTACAGCCACCAAGGACA
CCTACGACGCCCTTCACATGCAGGCCCTGCCCCCTCGCGGACCGCAGTGTACTAA
TTATGCTCTCTTGAAATTGGCTGGAGATGTTGAGAGCAATCCCGGGCCCATGCGC
ATTAGCAAGCCCCACCTGCGGAGCATCAGCATCCAGTGCTACCTGTGCCTGCTGC
TGAACAGCCACTTCCTGACCGAGGCCGGCATCCACGTGTTCATCCTGGGCTGCTT
CAGCGCCGGACTGCCCAAGACCGAGGCCAACTGGGTGAACGTGATCAGCGACCT
GAAGAAGATCGAGGACCTGATCCAGAGCATGCACATCGACGCCACCCTGTACAC
CGAGAGCGACGTGCACCCCAGCTGCAAGGTGACCGCCATGAAGTGCTTTCTGCT
GGAACTGCAGGTGATCAGCCTGGAAAGCGGCGACGCCAGCATCCACGACACCGT
GGAGAACCTGATCATCCTGGCCAACAACAGCCTGAGCAGCAACGGCAACGTGAC
CGAGAGCGGCTGCAAAGAGTGCGAGGAACTGGAAGAGAAGAACATCAAAGAGT
TTCTGCAGAGCTTCGTGCACATCGTGCAGATGTTCATCAACACCAGCTGA.

Additional examples of specific molecules including an antigen-specific, e.g., a CD70-specific, CAR and IL15 include those disclosed in, e.g., U.S. Provisional Patent Application Nos. 63/216,753 and 63/236,475, both incorporated by reference herein in their entirety.

In some aspects, an antigen-specific, e.g., a CD70-specific, CAR comprising an antigen binding, e.g., a CD70-binding, domain, is utilized. In some aspects a CD70-specific CAR comprising a CD27-derived CD70-binding domain is utilized.

In specific examples, such a CAR may have the following nucleotide sequence:

CD27tr28tdmCD3zIL15:
(SEQ ID NO: 18)
ATGGCACGGCCACATCCCTGGTGGCTGTGCGTTCTGGGGACCCTGGTG
GGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTG
AAGGACTGTGACCAGCATAGAAAGGCTGCTCAGTGTGATCCTTGCATACCG
GGGGTCTCCTTCTCTCCTGACCACCACACCCGGCCCCACTGTGAGAGCTGT
CGGCACTGTAACTCTGGTCTTCTCGTTCGCAACTGCACCATCACTGCCAAT
GCTGAGTGTGCCTGTCGCAATGGCTGGCAGTGCAGGGACAAGGAGTGCACC
GAGTGTGATCCTCTTCCAAACCCTTCGCTGACCGCTCGGTCGTCTCAGGCC
CTGAGCCCACACCCTCAGCCCACCCACTTACCTTATGTCAGTGAGATGCTG
GAGGCCAGGACAGCTGGGCACATGCAGACTCTGGCTGACTTCAGGCAGCTG
CCTGCCCGGACTCTCTCTACCCACTGGCCACCCCAAAGATCCCTGTGCAGC
TCCGATTTTATTCGCTTTTGGGTGCTGGTGGTGGTTGGTGGAGTCCTGGCT
TGCTATAGCTTGCTAGTAACAGTGGCCTTTATTATTTTCTGGGTG

A corresponding amino acid sequence for CD27tr28tdmCD3zIL15 is as follows:

(SEQ ID NO: 71)
MARPHPWWLCVLGTLVGLSATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRFWVLVVVGGVLACYSLLVTVAFIIFWV
CD27Tr28tmd41BBicd3zIL15:
(SEQ ID NO: 19)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCTTTTGGGTGCTGGTGGTGGT
TGGTGGAGTCCTGGCTTGCTATAGCTTGCTAGTAACAGTGGCCTTTATTATTTTCT
GGGTGAAACGGGGCAGAAAGAAACTCCTGTATATATTCAAACAACCATTTATGA
GACCAGTACAAACTACTCAAGAGGAAGATGGCTGTAGCTGCCGATTTCCAGAAG
AAGAAGAAGGAGGATGTGAACTGAAACGCGTGAAGTTCAGCAGGAGCGCAGAC
GCCCCCGCGTACCAGCAGGGCCAGAACCAGCTCTATAACGAGCTCAATCTAGGA
CGAAGAGAGGAGTACGATGTTTTGGACAAAAGACGTGGCCGGGACCCTGAGATG
GGGGGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGG
ACACCTACGACGCCCTTCACATGCAGGCCCTGCCCCCTCGCGGACCGCAGTGTAC
TAATTATGCTCTCTTGAAATTGGCTGGAGATGTTGAGAGCAATCCCGGGCCCATG
CGCATTAGCAAGCCCCACCTGCGGAGCATCAGCATCCAGTGCTACCTGTGCCTGC
TGCTGAACAGCCACTTCCTGACCGAGGCCGGCATCCACGTGTTCATCCTGGGCTG
CTTCAGCGCCGGACTGCCCAAGACCGAGGCCAACTGGGTGAACGTGATCAGCGA
CCTGAAGAAGATCGAGGACCTGATCCAGAGCATGCACATCGACGCCACCCTGTA
CACCGAGAGCGACGTGCACCCCAGCTGCAAGGTGACCGCCATGAAGTGCTTTCT
GCTGGAACTGCAGGTGATCAGCCTGGAAAGCGGCGACGCCAGCATCCACGACAC
CGTGGAGAACCTGATCATCCTGGCCAACAACAGCCTGAGCAGCAACGGCAACGT
GACCGAGAGCGGCTGCAAAGAGTGCGAGGAACTGGAAGAGAAGAACATCAAAG
AGTTTCTGCAGAGCTTCGTGCACATCGTGCAGATGTTCATCAACACCAGCTGA

A corresponding amino acid sequence for CD27Tr28tmd41BBicd3zIL15 is as

(SEQ ID NO: 72)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRFWVLVVVGGVLACYSLLVTVAFIIFWVKR
GRKKLLYIFKQPFMRPVQTTQEEDGCSCRFPEEEEGGCELKRVKFSRSADAPAYQQG
QNQLYNELNLGRREEYDVLDKRRGRDPEMGGKPRRKNPQEGLYNELQKDKMAEA
YSEIGMKGERRRGKGHDGLYQGLSTATKDTYDALHMQALPPRGPQCTNYALLKLA
GDVESNPGPMRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISLESGDASIHD
TVENLIILANNSLSSNGNVTESGCKECEELEEKNIKEFLQSFVHIVQMFINTS
GSPco27Tr28tmd41BBicCD3zIL15:
(SEQ ID NO: 20)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCTTTTGGGTGCTG
GTGGTGGTTGGTGGAGTCCTGGCTTGCTATAGCTTGCTAGTAACAGTGGCCTTTA
TTATTTTCTGGGTGAAACGGGGCAGAAAGAAACTCCTGTATATATTCAAACAACC
ATTTATGAGACCAGTACAAACTACTCAAGAGGAAGATGGCTGTAGCTGCCGATTT
CCAGAAGAAGAAGAAGGAGGATGTGAACTGAAACGCGTGAAGTTCAGCAGGAG
CGCAGACGCCCCCGCGTACCAGCAGGGCCAGAACCAGCTCTATAACGAGCTCAA
TCTAGGACGAAGAGAGGAGTACGATGTTTTGGACAAAAGACGTGGCCGGGACCC
TGAGATGGGGGGAAAGCCGAGAAGGAAGAACCCTCAGGAAGGCCTGTACAATG
AACTGCAGAAAGATAAGATGGCGGAGGCCTACAGTGAGATTGGGATGAAAGGC
GAGCGCCGGAGGGGCAAGGGGCACGATGGCCTTTACCAGGGTCTCAGTACAGCC
ACCAAGGACACCTACGACGCCCTTCACATGCAGGCCCTGCCCCCTCGCGGACCGC
AGTGTACTAATTATGCTCTCTTGAAATTGGCTGGAGATGTTGAGAGCAATCCCGG
GCCCATGCGCATTAGCAAGCCCCACCTGCGGAGCATCAGCATCCAGTGCTACCTG
TGCCTGCTGCTGAACAGCCACTTCCTGACCGAGGCCGGCATCCACGTGTTCATCC
TGGGCTGCTTCAGCGCCGGACTGCCCAAGACCGAGGCCAACTGGGTGAACGTGA
TCAGCGACCTGAAGAAGATCGAGGACCTGATCCAGAGCATGCACATCGACGCCA
CCCTGTACACCGAGAGCGACGTGCACCCCAGCTGCAAGGTGACCGCCATGAAGT
GCTTTCTGCTGGAACTGCAGGTGATCAGCCTGGAAAGCGGCGACGCCAGCATCC
ACGACACCGTGGAGAACCTGATCATCCTGGCCAACAACAGCCTGAGCAGCAACG
GCAACGTGACCGAGAGCGGCTGCAAAGAGTGCGAGGAACTGGAAGAGAAGAAC
ATCAAAGAGTTTCTGCAGAGCTTCGTGCACATCGTGCAGATGTTCATCAACACCA
GCTGA

A corresponding amino acid sequence for GSPco27Tr28tmd41BBicCD3zIL15 is as follows:

(SEQ ID NO: 73)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRFWVLVVVGGVLACYSLLVTVAFIIFWVK
RGRKKLLYIFKQPFMRPVQTTQEEDGCSCRFPEEEEGGCELKRVKFSRSADAPAYQQ
GQNQLYNELNLGRREEYDVLDKRRGRDPEMGGKPRRKNPQEGLYNELQKDKMAE
AYSEIGMKGERRRGKGHDGLYQGLSTATKDTYDALHMQALPPRGPQCTNYALLKL
AGDVESNPGPMRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVISLESGDASI
HDTVENLIILANNSLSSNGNVTESGCKECEELEEKNIKEFLQSFVHIVQMFINTS
CD27Tr28tmdDAP10icd3z15:
(SEQ ID NO: 21)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCTTTTGGGTGCTGGTGGTGGT
TGGTGGAGTCCTGGCTTGCTATAGCTTGCTAGTAACAGTGGCCTTTATTATTTTCT
GGGTGCTTTGCGCACGCCCACGCCGCAGCCCCGCCCAAGAAGATGGCAAAGTCT
ACATCAACATGCCAGGCAGGGGCAAACGCGTGAAGTTCAGCAGGAGCGCAGAC
GCCCCCGCGTACCAGCAGGGCCAGAACCAGCTCTATAACGAGCTCAATCTAGGA
CGAAGAGAGGAGTACGATGTTTTGGACAAAAGACGTGGCCGGGACCCTGAGATG
GGGGGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGG
ACACCTACGACGCCCTTCACATGCAGGCCCTGCCCCCTCGCGGACCGCAGTGTAC
TAATTATGCTCTCTTGAAATTGGCTGGAGATGTTGAGAGCAATCCCGGGCCCATG
CGCATTAGCAAGCCCCACCTGCGGAGCATCAGCATCCAGTGCTACCTGTGCCTGC
TGCTGAACAGCCACTTCCTGACCGAGGCCGGCATCCACGTGTTCATCCTGGGCTG
CTTCAGCGCCGGACTGCCCAAGACCGAGGCCAACTGGGTGAACGTGATCAGCGA
CCTGAAGAAGATCGAGGACCTGATCCAGAGCATGCACATCGACGCCACCCTGTA
CACCGAGAGCGACGTGCACCCCAGCTGCAAGGTGACCGCCATGAAGTGCTTTCT
GCTGGAACTGCAGGTGATCAGCCTGGAAAGCGGCGACGCCAGCATCCACGACAC
CGTGGAGAACCTGATCATCCTGGCCAACAACAGCCTGAGCAGCAACGGCAACGT
GACCGAGAGCGGCTGCAAAGAGTGCGAGGAACTGGAAGAGAAGAACATCAAAG
AGTTTCTGCAGAGCTTCGTGCACATCGTGCAGATGTTCATCAACACCAGCTGA

A corresponding amino acid sequence for CD27Tr28tmdDAP10icd3z15 is as

(SEQ ID NO: 74)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRFWVLVVVGGVLACYSLLVTVAFIIFWVLC
ARPRRSPAQEDGKVYINMPGRGKRVKFSRSADAPAYQQGQNQLYNELNLGRREEY
DVLDKRRGRDPEMGGKPRRKNPQEGLYNELQKDKMAEAYSEIGMKGERRRGKGH
DGLYQGLSTATKDTYDALHMQALPPRGPQCTNYALLKLAGDVESNPGPMRISKPHL
RSISIQCYLCLLLNSHFLTEAGIHVFILGCFSAGLPKTEANWVNVISDLKKIEDLIQSMH
IDATLYTESDVHPSCKVTAMKCFLLELQVISLESGDASIHDTVENLIILANNSLSSNGN
VTESGCKECEELEEKNIKEFLQSFVHIVQMFINTS
GSPco27Tr28tmdDAP10IL15:
(SEQ ID NO: 22)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCTTTTGGGTGCTG
GTGGTGGTTGGTGGAGTCCTGGCTTGCTATAGCTTGCTAGTAACAGTGGCCTTTA
TTATTTTCTGGGTGCTTTGCGCACGCCCACGCCGCAGCCCCGCCCAAGAAGATGG
CAAAGTCTACATCAACATGCCAGGCAGGGGCAAACGCGTGAAGTTCAGCAGGAG
CGCAGACGCCCCCGCGTACCAGCAGGGCCAGAACCAGCTCTATAACGAGCTCAA
TCTAGGACGAAGAGAGGAGTACGATGTTTTGGACAAAAGACGTGGCCGGGACCC
TGAGATGGGGGGAAAGCCGAGAAGGAAGAACCCTCAGGAAGGCCTGTACAATG
AACTGCAGAAAGATAAGATGGCGGAGGCCTACAGTGAGATTGGGATGAAAGGC
GAGCGCCGGAGGGGCAAGGGGCACGATGGCCTTTACCAGGGTCTCAGTACAGCC
ACCAAGGACACCTACGACGCCCTTCACATGCAGGCCCTGCCCCCTCGCGGACCGC
AGTGTACTAATTATGCTCTCTTGAAATTGGCTGGAGATGTTGAGAGCAATCCCGG
GCCCATGCGCATTAGCAAGCCCCACCTGCGGAGCATCAGCATCCAGTGCTACCTG
TGCCTGCTGCTGAACAGCCACTTCCTGACCGAGGCCGGCATCCACGTGTTCATCC
TGGGCTGCTTCAGCGCCGGACTGCCCAAGACCGAGGCCAACTGGGTGAACGTGA
TCAGCGACCTGAAGAAGATCGAGGACCTGATCCAGAGCATGCACATCGACGCCA
CCCTGTACACCGAGAGCGACGTGCACCCCAGCTGCAAGGTGACCGCCATGAAGT
GCTTTCTGCTGGAACTGCAGGTGATCAGCCTGGAAAGCGGCGACGCCAGCATCC
ACGACACCGTGGAGAACCTGATCATCCTGGCCAACAACAGCCTGAGCAGCAACG
GCAACGTGACCGAGAGCGGCTGCAAAGAGTGCGAGGAACTGGAAGAGAAGAAC
ATCAAAGAGTTTCTGCAGAGCTTCGTGCACATCGTGCAGATGTTCATCAACACCA
GCTGA

A corresponding amino acid sequence for GSPco27Tr28tmdDAP10IL15 is as

(SEQ ID NO: 75)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRFWVLVVVGGVLACYSLLVTVAFIIFWVL
CARPRRSPAQEDGKVYINMPGRGKRVKFSRSADAPAYQQGQNQLYNELNLGRREEY
DVLDKRRGRDPEMGGKPRRKNPQEGLYNELQKDKMAEAYSEIGMKGERRRGKGH
DGLYQGLSTATKDTYDALHMQALPPRGPQCTNYALLKLAGDVESNPGPMRISKPHL
RSISIQCYLCLLLNSHFLTEAGIHVFILGCFSAGLPKTEANWVNVISDLKKIEDLIQSMH
IDATLYTESDVHPSCKVTAMKCFLLELQVISLESGDASIHDTVENLIILANNSLSSNGN
VTESGCKECEELEEKNIKEFLQSFVHIVQMFINTS
CD27Tr28tmdDAP12icd3z15:
(SEQ ID NO: 23)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCTTTTGGGTGCTGGTGGTGGT
TGGTGGAGTCCTGGCTTGCTATAGCTTGCTAGTAACAGTGGCCTTTATTATTTTCT
GGGTGTACTTCCTGGGCCGGCTGGTCCCTCGGGGGCGAGGGGCTGCGGAGGCAG
CGACCCGGAAACAGCGTATCACTGAGACCGAGTCGCCTTATCAGGAGCTCCAGG
GTCAGAGGTCGGATGTCTACAGCGACCTCAACACACAGAGGCCGTATTACAAAA
AACGCGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTTTTGG
ACAAAAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCT
TTACCAGGGTCTCAGTACAGCCACCAAGGACACCTACGACGCCCTTCACATGCAG
GCCCTGCCCCCTCGCGGACCGCAGTGTACTAATTATGCTCTCTTGAAATTGGCTG
GAGATGTTGAGAGCAATCCCGGGCCCATGCGCATTAGCAAGCCCCACCTGCGGA
GCATCAGCATCCAGTGCTACCTGTGCCTGCTGCTGAACAGCCACTTCCTGACCGA
GGCCGGCATCCACGTGTTCATCCTGGGCTGCTTCAGCGCCGGACTGCCCAAGACC
GAGGCCAACTGGGTGAACGTGATCAGCGACCTGAAGAAGATCGAGGACCTGATC
CAGAGCATGCACATCGACGCCACCCTGTACACCGAGAGCGACGTGCACCCCAGC
TGCAAGGTGACCGCCATGAAGTGCTTTCTGCTGGAACTGCAGGTGATCAGCCTGG
AAAGCGGCGACGCCAGCATCCACGACACCGTGGAGAACCTGATCATCCTGGCCA
ACAACAGCCTGAGCAGCAACGGCAACGTGACCGAGAGCGGCTGCAAAGAGTGC
GAGGAACTGGAAGAGAAGAACATCAAAGAGTTTCTGCAGAGCTTCGTGCACATC
GTGCAGATGTTCATCAACACCAGCTGA

A corresponding amino acid sequence for CD27Tr28tmdDAP12icd3z15 is as

(SEQ ID NO: 76)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRFWVLVVVGGVLACYSLLVTVAFIIFWVYF
LGRLVPRGRGAAEAATRKQRITETESPYQELQGQRSDVYSDLNTQRPYYKKRVKFSR
SADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPRGPQC
TNYALLKLAGDVESNPGPMRISKPHLRSISIQCYLCLLLNSHFLTEAGIHVFILGCFSA
GLPKTEANWVNVISDLKKIEDLIQSMHIDATLYTESDVHPSCKVTAMKCFLLELQVIS
LESGDASIHDTVENLIILANNSLSSNGNVTESGCKECEELEEKNIKEFLQSFVHIVQMFI
NTS
GSPco27Tr28tmddap12icd15:
(SEQ ID NO: 24)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCTTTTGGGTGCTG
GTGGTGGTTGGTGGAGTCCTGGCTTGCTATAGCTTGCTAGTAACAGTGGCCTTTA
TTATTTTCTGGGTGTACTTCCTGGGCCGGCTGGTCCCTCGGGGGCGAGGGGCTGC
GGAGGCAGCGACCCGGAAACAGCGTATCACTGAGACCGAGTCGCCTTATCAGGA
GCTCCAGGGTCAGAGGTCGGATGTCTACAGCGACCTCAACACACAGAGGCCGTA
TTACAAAAAACGCGTGAAGTTCAGCAGGAGCGCAGACGCCCCCGCGTACCAGCA
GGGCCAGAACCAGCTCTATAACGAGCTCAATCTAGGACGAAGAGAGGAGTACGA
TGTTTTGGACAAAAGACGTGGCCGGGACCCTGAGATGGGGGGAAAGCCGAGAAG
GAAGAACCCTCAGGAAGGCCTGTACAATGAACTGCAGAAAGATAAGATGGCGG
AGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTC
ACATGCAGGCCCTGCCCCCTCGCGGACCGCAGTGTACTAATTATGCTCTCTTGAA
ATTGGCTGGAGATGTTGAGAGCAATCCCGGGCCCATGCGCATTAGCAAGCCCCA
CCTGCGGAGCATCAGCATCCAGTGCTACCTGTGCCTGCTGCTGAACAGCCACTTC
CTGACCGAGGCCGGCATCCACGTGTTCATCCTGGGCTGCTTCAGCGCCGGACTGC
CCAAGACCGAGGCCAACTGGGTGAACGTGATCAGCGACCTGAAGAAGATCGAGG
ACCTGATCCAGAGCATGCACATCGACGCCACCCTGTACACCGAGAGCGACGTGC
ACCCCAGCTGCAAGGTGACCGCCATGAAGTGCTTTCTGCTGGAACTGCAGGTGAT
CAGCCTGGAAAGCGGCGACGCCAGCATCCACGACACCGTGGAGAACCTGATCAT
CCTGGCCAACAACAGCCTGAGCAGCAACGGCAACGTGACCGAGAGCGGCTGCAA
AGAGTGCGAGGAACTGGAAGAGAAGAACATCAAAGAGTTTCTGCAGAGCTTCGT
GCACATCGTGCAGATGTTCATCAACACCAGCTGA

A corresponding amino acid sequence for GSPco27Tr28tmddap12icd15 is as

(SEQ ID NO: 77)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRFWVLVVVGGVLACYSLLVTVAFIIFWVY
FLGRLVPRGRGAAEAATRKQRITETESPYQELQGQRSDVYSDLNTQRPYYKKRVKFS
RSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRGPQ
CTNYALLKLAGDVESNPGPMRISKPHLRSISIQCYLCLLLNSHFLTEAGIHVFILGCFS
AGLPKTEANWVNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQM
FINTS
CD27Tr28tmdNKG2Dic3z15:
(SEQ ID NO: 25)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCTTTTGGGTGCTGGTGGTGGT
TGGTGGAGTCCTGGCTTGCTATAGCTTGCTAGTAACAGTGGCCTTTATTATTTTCT
GGGTGAGCGCGAACGAACGCTGCAAAAGCAAAGTGGTGCCGTGCCGCCAGAAA
CAGTGGCGCACCAGCTTTGATAGCAAAAAACTGGATCTGAACTATAACCATTTTG
AAAGCATGGAATGGAGCCATCGCAGCCGCCGCGGCCGCATTTGGGGCATGAAAC
GCGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACA
AAAGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTACAGT
GAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGC
CCTGCCCCCTCGCGGACCGCAGTGTACTAATTATGCTCTCTTGAAATTGGCTGGA
GATGTTGAGAGCAATCCCGGGCCCATGCGCATTAGCAAGCCCCACCTGCGGAGC
ATCAGCATCCAGTGCTACCTGTGCCTGCTGCTGAACAGCCACTTCCTGACCGAGG
CCGGCATCCACGTGTTCATCCTGGGCTGCTTCAGCGCCGGACTGCCCAAGACCGA
GGCCAACTGGGTGAACGTGATCAGCGACCTGAAGAAGATCGAGGACCTGATCCA
GAGCATGCACATCGACGCCACCCTGTACACCGAGAGCGACGTGCACCCCAGCTG
CAAGGTGACCGCCATGAAGTGCTTTCTGCTGGAACTGCAGGTGATCAGCCTGGA
AAGCGGCGACGCCAGCATCCACGACACCGTGGAGAACCTGATCATCCTGGCCAA
CAACAGCCTGAGCAGCAACGGCAACGTGACCGAGAGCGGCTGCAAAGAGTGCG
AGGAACTGGAAGAGAAGAACATCAAAGAGTTTCTGCAGAGCTTCGTGCACATCG
TGCAGATGTTCATCAACACCAGCTGA

A corresponding amino acid sequence for CD27Tr28tmdNKG2Dic3z15 is as

(SEQ ID NO: 78)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRFWVLVVVGGVLACYSLLVTVAFIIFWVSA
NERCKSKVVPCRQKQWRTSFDSKKLDLNYNHFESMEWSHRSRRGRIWGMKRVKFS
RSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRGPQ
CTNYALLKLAGDVESNPGPMRISKPHLRSISIQCYLCLLLNSHFLTEAGIHVFILGCFS
AGLPKTEANWVNVISDLKKIEDLIQSMHIDATLYTESDVHPSCKVTAMKCFLLELQVI
SLESGDASIHDTVENLIILANNSLSSNGNVTESGCKECEELEEKNIKEFLQSFVHIVQM
FINTS
GSPco27Tr28tmdNKG2Dicd3z15:
(SEQ ID NO: 26)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCTTTTGGGTGCTG
GTGGTGGTTGGTGGAGTCCTGGCTTGCTATAGCTTGCTAGTAACAGTGGCCTTTA
TTATTTTCTGGGTGAGCGCGAACGAACGCTGCAAAAGCAAAGTGGTGCCGTGCC
GCCAGAAACAGTGGCGCACCAGCTTTGATAGCAAAAAACTGGATCTGAACTATA
ACCATTTTGAAAGCATGGAATGGAGCCATCGCAGCCGCCGCGGCCGCATTTGGG
GCATGAAACGCGTGAAGTTCAGCAGGAGCGCAGACGCCCCCGCGTACCAGCAGG
GCCAGAACCAGCTCTATAACGAGCTCAATCTAGGACGAAGAGAGGAGTACGATG
TTTTGGACAAAAGACGTGGCCGGGACCCTGAGATGGGGGGAAAGCCGAGAAGG
AAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCA
CATGCAGGCCCTGCCCCCTCGCGGACCGCAGTGTACTAATTATGCTCTCTTGAAA
TTGGCTGGAGATGTTGAGAGCAATCCCGGGCCCATGCGCATTAGCAAGCCCCAC
CTGCGGAGCATCAGCATCCAGTGCTACCTGTGCCTGCTGCTGAACAGCCACTTCC
TGACCGAGGCCGGCATCCACGTGTTCATCCTGGGCTGCTTCAGCGCCGGACTGCC
CAAGACCGAGGCCAACTGGGTGAACGTGATCAGCGACCTGAAGAAGATCGAGG
ACCTGATCCAGAGCATGCACATCGACGCCACCCTGTACACCGAGAGCGACGTGC
ACCCCAGCTGCAAGGTGACCGCCATGAAGTGCTTTCTGCTGGAACTGCAGGTGAT
CAGCCTGGAAAGCGGCGACGCCAGCATCCACGACACCGTGGAGAACCTGATCAT
CCTGGCCAACAACAGCCTGAGCAGCAACGGCAACGTGACCGAGAGCGGCTGCAA
AGAGTGCGAGGAACTGGAAGAGAAGAACATCAAAGAGTTTCTGCAGAGCTTCGT
GCACATCGTGCAGATGTTCATCAACACCAGCTGA

A corresponding amino acid sequence for GSPco27Tr28tmdNKG2Dicd3z15 is as

(SEQ ID NO: 79)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRFWVLVVVGGVLACYSLLVTVAFIIFWVS
ANERCKSKVVPCRQKQWRTSFDSKKLDLNYNHFESMEWSHRSRRGRIWGMKRVKF
SRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPRGP
QCTNYALLKLAGDVESNPGPMRISKPHLRSISIQCYLCLLLNSHFLTEAGIHVFILGCF
SAGLPKTEANWVNVISDLKKIEDLIQSMHIDATLYTESDVHPSCKVTAMKCFLLELQ
VISLESGDASIHDTVENLIILANNSLSSNGNVTESGCKECEELEEKNIKEFLQSFVHIVQ
MFINTS
CD27Tr41BBicd3z15:
(SEQ ID NO: 27)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCATCCTTGTGATCTTCTCTGG
AATGTTCCTTGTTTTCACCCTGGCCGGGGCCCTGTTCCTCCATAAACGGGGCAGA
AAGAAACTCCTGTATATATTCAAACAACCATTTATGAGACCAGTACAAACTACTC
AAGAGGAAGATGGCTGTAGCTGCCGATTTCCAGAAGAAGAAGAAGGAGGATGT
GAACTGAAACGCGTGAAGTTCAGCAGGAGCGCAGACGCCCCCGCGTACCAGCAG
GGCCAGAACCAGCTCTATAACGAGCTCAATCTAGGACGAAGAGAGGAGTACGAT
GTTTTGGACAAAAGACGTGGCCGGGACCCTGAGATGGGGGGAAAGCCGAGAAG
GAAGAACCCTCAGGAAGGCCTGTACAATGAACTGCAGAAAGATAAGATGGCGG
AGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTC
ACATGCAGGCCCTGCCCCCTCGCGGACCGCAGTGTACTAATTATGCTCTCTTGAA
ATTGGCTGGAGATGTTGAGAGCAATCCCGGGCCCATGCGCATTAGCAAGCCCCA
CCTGCGGAGCATCAGCATCCAGTGCTACCTGTGCCTGCTGCTGAACAGCCACTTC
CTGACCGAGGCCGGCATCCACGTGTTCATCCTGGGCTGCTTCAGCGCCGGACTGC
CCAAGACCGAGGCCAACTGGGTGAACGTGATCAGCGACCTGAAGAAGATCGAGG
ACCTGATCCAGAGCATGCACATCGACGCCACCCTGTACACCGAGAGCGACGTGC
ACCCCAGCTGCAAGGTGACCGCCATGAAGTGCTTTCTGCTGGAACTGCAGGTGAT
CAGCCTGGAAAGCGGCGACGCCAGCATCCACGACACCGTGGAGAACCTGATCAT
CCTGGCCAACAACAGCCTGAGCAGCAACGGCAACGTGACCGAGAGCGGCTGCAA
AGAGTGCGAGGAACTGGAAGAGAAGAACATCAAAGAGTTTCTGCAGAGCTTCGT
GCACATCGTGCAGATGTTCATCAACACCAGCTGA

A corresponding amino acid sequence for CD27Tr41BBicd3z15 is as follows:

(SEQ ID NO: 80)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRILVIFSGMFLVFTLAGALFLHKRGRKKLLY
IFKQPFMRPVQTTQEEDGCSCRFPEEEEGGCELKRVKFSRSADAPAYQQGQNQLYNE
LNLGRREEYDVLDKRRGRDPEMGGKPRRKNPQEGLYNELQKDKMAEAYSEIGMKG
ERRRGKGHDGLYQGLSTATKDTYDALHMQALPPRGPQCTNYALLKLAGDVESNPG
PMRISKPHLRSISIQCYLCLLLNSHFLTEAGIHVFILGCFSAGLPKTEANWVNVISDLK
KIEDLIQSMHIDATLYTESDVHPSCKVTAMKCFLLELQVISLESGDASIHDTVENLIILA
NNSLSSNGNVTESGCKECEELEEKNIKEFLQSFVHIVQMFINTS
GSPco27Tr41BBicd3z15:
(SEQ ID NO: 28)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCATACTGGTCAT
CTTTTCTGGAATGTTCCTTGTGTTCACCCTGGCAGGAGCCCTGTTCCTTCACAAAC
GGGGCAGAAAGAAACTCCTGTATATATTCAAACAACCATTTATGAGACCAGTAC
AAACTACTCAAGAGGAAGATGGCTGTAGCTGCCGATTTCCAGAAGAAGAAGAAG
GAGGATGTGAACTGAAACGCGTGAAGTTCAGCAGGAGCGCAGACGCCCCCGCGT
ACCAGCAGGGCCAGAACCAGCTCTATAACGAGCTCAATCTAGGACGAAGAGAGG
AGTACGATGTTTTGGACAAAAGACGTGGCCGGGACCCTGAGATGGGGGGAAAGC
CGAGAAGGAAGAACCCTCAGGAAGGCCTGTACAATGAACTGCAGAAAGATAAG
ATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGA
CGCCCTTCACATGCAGGCCCTGCCCCCTCGCGGACCGCAGTGTACTAATTATGCT
CTCTTGAAATTGGCTGGAGATGTTGAGAGCAATCCCGGGCCCATGCGCATTAGCA
AGCCCCACCTGCGGAGCATCAGCATCCAGTGCTACCTGTGCCTGCTGCTGAACAG
CCACTTCCTGACCGAGGCCGGCATCCACGTGTTCATCCTGGGCTGCTTCAGCGCC
GGACTGCCCAAGACCGAGGCCAACTGGGTGAACGTGATCAGCGACCTGAAGAAG
ATCGAGGACCTGATCCAGAGCATGCACATCGACGCCACCCTGTACACCGAGAGC
GACGTGCACCCCAGCTGCAAGGTGACCGCCATGAAGTGCTTTCTGCTGGAACTGC
AGGTGATCAGCCTGGAAAGCGGCGACGCCAGCATCCACGACACCGTGGAGAACC
TGATCATCCTGGCCAACAACAGCCTGAGCAGCAACGGCAACGTGACCGAGAGCG
GCTGCAAAGAGTGCGAGGAACTGGAAGAGAAGAACATCAAAGAGTTTCTGCAG
AGCTTCGTGCACATCGTGCAGATGTTCATCAACACCAGCTGA

A corresponding amino acid sequence for GSPco27Tr41BBicd3z15 is as follows:

(SEQ ID NO: 81)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRILVIFSGMFLVFTLAGALFLHKRGRKKLL
YIFKQPFMRPVQTTQEEDGCSCRFPEEEEGGCELKRVKFSRSADAPAYQQGQNQLYN
ELNLGRREEYDVLDKRRGRDPEMGGKPRRKNPQEGLYNELQKDKMAEAYSEIGMK
GERRRGKGHDGLYQGLSTATKDTYDALHMQALPPRGPQCTNYALLKLAGDVESNP
GPMRISKPHLRSISIQCYLCLLLNSHFLTEAGIHVFILGCFSAGLPKTEANWVNVISDL
KKIEDLIQSMHIDATLYTESDVHPSCKVTAMKCFLLELQVISLESGDASIHDTVENLIIL
ANNSLSSNGNVTESGCKECEELEEKNIKEFLQSFVHIVQMFINTS
CD27TrCD3ZIL15:
(SEQ ID NO: 29)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCATCCTTGTGATCTTCTCTGG
AATGTTCCTTGTTTTCACCCTGGCCGGGGCCCTGTTCCTCCATAAACGCGTGAAGT
TCAGCAGGAGCGCAGACGCCCCCGCGTACCAGCAGGGCCAGAACCAGCTCTATA
ACGAGCTCAATCTAGGACGAAGAGAGGAGTACGATGTTTTGGACAAAAGACGTG
GCCGGGACCCTGAGATGGGGGGAAAGCCGAGAAGGAAGAACCCTCAGGAAGGC
CTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCCTC
GCGGACCGCAGTGTACTAATTATGCTCTCTTGAAATTGGCTGGAGATGTTGAGAG
CAATCCCGGGCCCATGCGCATTAGCAAGCCCCACCTGCGGAGCATCAGCATCCA
GTGCTACCTGTGCCTGCTGCTGAACAGCCACTTCCTGACCGAGGCCGGCATCCAC
GTGTTCATCCTGGGCTGCTTCAGCGCCGGACTGCCCAAGACCGAGGCCAACTGGG
TGAACGTGATCAGCGACCTGAAGAAGATCGAGGACCTGATCCAGAGCATGCACA
TCGACGCCACCCTGTACACCGAGAGCGACGTGCACCCCAGCTGCAAGGTGACCG
CCATGAAGTGCTTTCTGCTGGAACTGCAGGTGATCAGCCTGGAAAGCGGCGACG
CCAGCATCCACGACACCGTGGAGAACCTGATCATCCTGGCCAACAACAGCCTGA
GCAGCAACGGCAACGTGACCGAGAGCGGCTGCAAAGAGTGCGAGGAACTGGAA
GAGAAGAACATCAAAGAGTTTCTGCAGAGCTTCGTGCACATCGTGCAGATGTTC
ATCAACACCAGCTGA

A corresponding amino acid sequence for CD27TrCD3ZIL15 is as follows:

(SEQ ID NO: 82)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRILVIFSGMFLVFTLAGALFLHKRVKFSRSA
DAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPRGPQCTN
YALLKLAGDVESNPGPMRISKPHLRSISIQCYLCLLLNSHFLTEAGIHVFILGCFSAGLP
KTEANWVNVISDLKKIEDLIQSMHIDATLYTESDVHPSCKVTAMKCFLLELQVISLES
GDASIHDTVENLIILANNSLSSNGNVTESGCKECEELEEKNIKEFLQSFVHIVQMFINTS
GSPco27Tr3z15:
(SEQ ID NO: 30)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCATCCTTGTGATCTTCTCTGG
AATGTTCCTTGTTTTCACCCTGGCCGGGGCCCTGTTCCTCCATAAACGCGTGAAGT
TCAGCAGGAGCGCAGACGCCCCCGCGTACCAGCAGGGCCAGAACCAGCTCTATA
ACGAGCTCAATCTAGGACGAAGAGAGGAGTACGATGTTTTGGACAAAAGACGTG
GCCGGGACCCTGAGATGGGGGGAAAGCCGAGAAGGAAGAACCCTCAGGAAGGC
CTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCCTC
GCGGACCGCAGTGTACTAATTATGCTCTCTTGAAATTGGCTGGAGATGTTGAGAG
CAATCCCGGGCCCATGCGCATTAGCAAGCCCCACCTGCGGAGCATCAGCATCCA
GTGCTACCTGTGCCTGCTGCTGAACAGCCACTTCCTGACCGAGGCCGGCATCCAC
GTGTTCATCCTGGGCTGCTTCAGCGCCGGACTGCCCAAGACCGAGGCCAACTGGG
TGAACGTGATCAGCGACCTGAAGAAGATCGAGGACCTGATCCAGAGCATGCACA
TCGACGCCACCCTGTACACCGAGAGCGACGTGCACCCCAGCTGCAAGGTGACCG
CCATGAAGTGCTTTCTGCTGGAACTGCAGGTGATCAGCCTGGAAAGCGGCGACG
CCAGCATCCACGACACCGTGGAGAACCTGATCATCCTGGCCAACAACAGCCTGA
GCAGCAACGGCAACGTGACCGAGAGCGGCTGCAAAGAGTGCGAGGAACTGGAA
GAGAAGAACATCAAAGAGTTTCTGCAGAGCTTCGTGCACATCGTGCAGATGTTC
ATCAACACCAGCTGA

A corresponding amino acid sequence for GSPco27Tr3z15 is as follows:

(SEQ ID NO: 83)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRILVIFSGMFLVFTLAGALFLHKRVKFSRSA
DAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPRGPQCTN
YALLKLAGDVESNPGPMRISKPHLRSISIQCYLCLLLNSHFLTEAGIHVFILGCFSAGLP
KTEANWVNVISDLKKIEDLIQSMHIDATLYTESDVHPSCKVTAMKCFLLELQVISLES
GDASIHDTVENLIILANNSLSSNGNVTESGCKECEELEEKNIKEFLQSFVHIVQMFINTS
CD27TrCD28icd3z15:
(SEQ ID NO: 31)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCATCCTTGTGATCTTCTCTGG
AATGTTCCTTGTTTTCACCCTGGCCGGGGCCCTGTTCCTCCATAGGAGTAAGAGG
AGCAGGCTCCTGCACAGTGACTACATGAACATGACTCCCCGCCGCCCCGGGCCC
ACCCGCAAGCATTACCAGCCCTATGCCCCACCACGCGACTTCGCAGCCTATCGCT
CACGCGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTTTTGG
ACAAAAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCT
TTACCAGGGTCTCAGTACAGCCACCAAGGACACCTACGACGCCCTTCACATGCAG
GCCCTGCCCCCTCGCGGACCGCAGTGTACTAATTATGCTCTCTTGAAATTGGCTG
GAGATGTTGAGAGCAATCCCGGGCCCATGCGCATTAGCAAGCCCCACCTGCGGA
GCATCAGCATCCAGTGCTACCTGTGCCTGCTGCTGAACAGCCACTTCCTGACCGA
GGCCGGCATCCACGTGTTCATCCTGGGCTGCTTCAGCGCCGGACTGCCCAAGACC
GAGGCCAACTGGGTGAACGTGATCAGCGACCTGAAGAAGATCGAGGACCTGATC
CAGAGCATGCACATCGACGCCACCCTGTACACCGAGAGCGACGTGCACCCCAGC
TGCAAGGTGACCGCCATGAAGTGCTTTCTGCTGGAACTGCAGGTGATCAGCCTGG
AAAGCGGCGACGCCAGCATCCACGACACCGTGGAGAACCTGATCATCCTGGCCA
ACAACAGCCTGAGCAGCAACGGCAACGTGACCGAGAGCGGCTGCAAAGAGTGC
GAGGAACTGGAAGAGAAGAACATCAAAGAGTTTCTGCAGAGCTTCGTGCACATC
GTGCAGATGTTCATCAACACCAGCTGA

A corresponding amino acid sequence for CD27TrCD28icd3z15 is as follows:

(SEQ ID NO: 84)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRILVIFSGMFLVFTLAGALFLHRSKRSRLLH
SDYMNMTPRRPGPTRKHYQPYAPPRDFAAYRSRVKFSRSADAPAYQQGQNQLYNE
LNLGRREEYDVLDKRRGRDPEMGGKPRRKNPQEGLYNELQKDKMAEAYSEIGMKG
ERRRGKGHDGLYQGLSTATKDTYDALHMQALPPRGPQCTNYALLKLAGDVESNPG
PMRISKPHLRSISIQCYLCLLLNSHFLTEAGIHVFILGCFSAGLPKTEANWVNVISDLK
KIEDLIQSMHIDATLYTESDVHPSCKVTAMKCFLLELQVISLESGDASIHDTVENLIILA
NNSLSSNGNVTESGCKECEELEEKNIKEFLQSFVHIVQMFINTS
GSPco27Tr28CD3z15:
(SEQ ID NO: 32)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCATACTGGTCAT
CTTTTCTGGAATGTTCCTTGTGTTCACCCTGGCAGGAGCCCTGTTCCTTCACAGGA
GTAAGAGGAGCAGGCTCCTGCACAGTGACTACATGAACATGACTCCCCGCCGCC
CCGGGCCCACCCGCAAGCATTACCAGCCCTATGCCCCACCACGCGACTTCGCAGC
CTATCGCTCACGCGTGAAGTTCAGCAGGAGCGCAGACGCCCCCGCGTACCAGCA
GGGCCAGAACCAGCTCTATAACGAGCTCAATCTAGGACGAAGAGAGGAGTACGA
TGTTTTGGACAAAAGACGTGGCCGGGACCCTGAGATGGGGGGAAAGCCGAGAAG
GAAGAACCCTCAGGAAGGCCTGTACAATGAACTGCAGAAAGATAAGATGGCGG
AGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTC
ACATGCAGGCCCTGCCCCCTCGCGGACCGCAGTGTACTAATTATGCTCTCTTGAA
ATTGGCTGGAGATGTTGAGAGCAATCCCGGGCCCATGCGCATTAGCAAGCCCCA
CCTGCGGAGCATCAGCATCCAGTGCTACCTGTGCCTGCTGCTGAACAGCCACTTC
CTGACCGAGGCCGGCATCCACGTGTTCATCCTGGGCTGCTTCAGCGCCGGACTGC
CCAAGACCGAGGCCAACTGGGTGAACGTGATCAGCGACCTGAAGAAGATCGAGG
ACCTGATCCAGAGCATGCACATCGACGCCACCCTGTACACCGAGAGCGACGTGC
ACCCCAGCTGCAAGGTGACCGCCATGAAGTGCTTTCTGCTGGAACTGCAGGTGAT
CAGCCTGGAAAGCGGCGACGCCAGCATCCACGACACCGTGGAGAACCTGATCAT
CCTGGCCAACAACAGCCTGAGCAGCAACGGCAACGTGACCGAGAGCGGCTGCAA
AGAGTGCGAGGAACTGGAAGAGAAGAACATCAAAGAGTTTCTGCAGAGCTTCGT
GCACATCGTGCAGATGTTCATCAACACCAGCTGA

A corresponding amino acid sequence for GSPco27Tr28CD3z15 is as follows:

(SEQ ID NO: 85)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRILVIFSGMFLVFTLAGALFLHRSKRSRLL
HSDYMNMTPRRPGPTRKHYQPYAPPRDFAAYRSRVKFSRSADAPAYQQGQNQLYN
ELNLGRREEYDVLDKRRGRDPEMGGKPRRKNPQEGLYNELQKDKMAEAYSEIGMK
GERRRGKGHDGLYQGLSTATKDTYDALHMQALPPRGPQCTNYALLKLAGDVESNP
GPMRISKPHLRSISIQCYLCLLLNSHFLTEAGIHVFILGCFSAGLPKTEANWVNVISDL
KKIEDLIQSMHIDATLYTESDVHPSCKVTAMKCFLLELQVISLESGDASIHDTVENLIIL
ANNSLSSNGNVTESGCKECEELEEKNIKEFLQSFVHIVQMFINTS
CD27TrCD28tmdicd3z15:
(SEQ ID NO: 33)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCTTTTGGGTGCTGGTGGTGGT
TGGTGGAGTCCTGGCTTGCTATAGCTTGCTAGTAACAGTGGCCTTTATTATTTTCT
GGGTGAGGAGTAAGAGGAGCAGGCTCCTGCACAGTGACTACATGAACATGACTC
CCCGCCGCCCCGGGCCCACCCGCAAGCATTACCAGCCCTATGCCCCACCACGCGA
CTTCGCAGCCTATCGCTCACGCGTGAAGTTCAGCAGGAGCGCAGACGCCCCCGC
GTACCAGCAGGGCCAGAACCAGCTCTATAACGAGCTCAATCTAGGACGAAGAGA
GGAGTACGATGTTTTGGACAAAAGACGTGGCCGGGACCCTGAGATGGGGGGAAA
GCCGAGAAGGAAGAACCCTCAGGAAGGCCTGTACAATGAACTGCAGAAAGATA
AGATGGCGGAGGCCTACAGTGAGATTGGGATGAAAGGCGAGCGCCGGAGGGGC
AAGGGGCACGATGGCCTTTACCAGGGTCTCAGTACAGCCACCAAGGACACCTAC
GACGCCCTTCACATGCAGGCCCTGCCCCCTCGCGGACCGCAGTGTACTAATTATG
CTCTCTTGAAATTGGCTGGAGATGTTGAGAGCAATCCCGGGCCCATGCGCATTAG
CAAGCCCCACCTGCGGAGCATCAGCATCCAGTGCTACCTGTGCCTGCTGCTGAAC
AGCCACTTCCTGACCGAGGCCGGCATCCACGTGTTCATCCTGGGCTGCTTCAGCG
CCGGACTGCCCAAGACCGAGGCCAACTGGGTGAACGTGATCAGCGACCTGAAGA
AGATCGAGGACCTGATCCAGAGCATGCACATCGACGCCACCCTGTACACCGAGA
GCGACGTGCACCCCAGCTGCAAGGTGACCGCCATGAAGTGCTTTCTGCTGGAACT
GCAGGTGATCAGCCTGGAAAGCGGCGACGCCAGCATCCACGACACCGTGGAGAA
CCTGATCATCCTGGCCAACAACAGCCTGAGCAGCAACGGCAACGTGACCGAGAG
CGGCTGCAAAGAGTGCGAGGAACTGGAAGAGAAGAACATCAAAGAGTTTCTGCA
GAGCTTCGTGCACATCGTGCAGATGTTCATCAACACCAGCTGA

A corresponding amino acid sequence for CD27TrCD28tmdicd3z15 is as follows:

(SEQ ID NO: 86)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRFWVLVVVGGVLACYSLLVTVAFIIFWVRS
KRSRLLHSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKMAEAYS
EIGMKGERRRGKGHDGLYQGLSTATKDTYDALHMQALPPRGPQCTNYALLKLAGD
VESNPGPMRISKPHLRSISIQCYLCLLLNSHFLTEAGIHVFILGCFSAGLPKTEANWVN
VISDLKKIEDLIQSMHIDATLYTESDVHPSCKVTAMKCFLLELQVISLESGDASIHDTV
ENLIILANNSLSSNGNVTESGCKECEELEEKNIKEFLQSFVHIVQMFINTS
GSPco27Tr28tmdicCD3z15:
(SEQ ID NO: 34)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCTTTTGGGTGCTG
GTGGTGGTTGGTGGAGTCCTGGCTTGCTATAGCTTGCTAGTAACAGTGGCCTTTA
TTATTTTCTGGGTGAGGAGTAAGAGGAGCAGGCTCCTGCACAGTGACTACATGA
ACATGACTCCCCGCCGCCCCGGGCCCACCCGCAAGCATTACCAGCCCTATGCCCC
ACCACGCGACTTCGCAGCCTATCGCTCACGCGTGAAGTTCAGCAGGAGCGCAGA
CGCCCCCGCGTACCAGCAGGGCCAGAACCAGCTCTATAACGAGCTCAATCTAGG
ACGAAGAGAGGAGTACGATGTTTTGGACAAAAGACGTGGCCGGGACCCTGAGAT
GGGGGGAAAGCCGAGAAGGAAGAACCCTCAGGAAGGCCTGTACAATGAACTGC
AGAAAGATAAGATGGCGGAGGCCTACAGTGAGATTGGGATGAAAGGCGAGCGC
CGGAGGGGCAAGGGGCACGATGGCCTTTACCAGGGTCTCAGTACAGCCACCAAG
GACACCTACGACGCCCTTCACATGCAGGCCCTGCCCCCTCGCGGACCGCAGTGTA
CTAATTATGCTCTCTTGAAATTGGCTGGAGATGTTGAGAGCAATCCCGGGCCCAT
GCGCATTAGCAAGCCCCACCTGCGGAGCATCAGCATCCAGTGCTACCTGTGCCTG
CTGCTGAACAGCCACTTCCTGACCGAGGCCGGCATCCACGTGTTCATCCTGGGCT
GCTTCAGCGCCGGACTGCCCAAGACCGAGGCCAACTGGGTGAACGTGATCAGCG
ACCTGAAGAAGATCGAGGACCTGATCCAGAGCATGCACATCGACGCCACCCTGT
ACACCGAGAGCGACGTGCACCCCAGCTGCAAGGTGACCGCCATGAAGTGCTTTC
TGCTGGAACTGCAGGTGATCAGCCTGGAAAGCGGCGACGCCAGCATCCACGACA
CCGTGGAGAACCTGATCATCCTGGCCAACAACAGCCTGAGCAGCAACGGCAACG
TGACCGAGAGCGGCTGCAAAGAGTGCGAGGAACTGGAAGAGAAGAACATCAAA
GAGTTTCTGCAGAGCTTCGTGCACATCGTGCAGATGTTCATCAACACCAGCTGA

A corresponding amino acid sequence for GSPco27Tr28tmdicCD3z15 is as

(SEQ ID NO: 87)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRFWVLVVVGGVLACYSLLVTVAFIIFWVR
SKRSRLLHSDYMNMTPRRPGPTRKHYQPYAPPRDFAAYRSRVKFSRSADAPAYQQG
QNQLYNELNLGRREEYDVLDKRRGRDPEMGGKPRRKNPQEGLYNELQKDKMAEA
YSEIGMKGERRRGKGHDGLYQGLSTATKDTYDALHMQALPPRGPQCTNYALLKLA
GDVESNPGPMRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISLESGDASIHD
TVENLIILANNSLSSNGNVTESGCKECEELEEKNIKEFLQSFVHIVQMFINTS
CD27TrDAP10icd3z15:
(SEQ ID NO: 35)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCATACTGGTCATCTTTTCTGG
AATGTTCCTTGTGTTCACCCTGGCAGGAGCCCTGTTCCTTCACCTTTGCGCACGCC
CACGCCGCAGCCCCGCCCAAGAAGATGGCAAAGTCTACATCAACATGCCAGGCA
GGGGCAAACGCGTGAAGTTCAGCAGGAGCGCAGACGCCCCCGCGTACCAGCAGG
GCCAGAACCAGCTCTATAACGAGCTCAATCTAGGACGAAGAGAGGAGTACGATG
TTTTGGACAAAAGACGTGGCCGGGACCCTGAGATGGGGGGAAAGCCGAGAAGG
AAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCA
CATGCAGGCCCTGCCCCCTCGCGGACCGCAGTGTACTAATTATGCTCTCTTGAAA
TTGGCTGGAGATGTTGAGAGCAATCCCGGGCCCATGCGCATTAGCAAGCCCCAC
CTGCGGAGCATCAGCATCCAGTGCTACCTGTGCCTGCTGCTGAACAGCCACTTCC
TGACCGAGGCCGGCATCCACGTGTTCATCCTGGGCTGCTTCAGCGCCGGACTGCC
CAAGACCGAGGCCAACTGGGTGAACGTGATCAGCGACCTGAAGAAGATCGAGG
ACCTGATCCAGAGCATGCACATCGACGCCACCCTGTACACCGAGAGCGACGTGC
ACCCCAGCTGCAAGGTGACCGCCATGAAGTGCTTTCTGCTGGAACTGCAGGTGAT
CAGCCTGGAAAGCGGCGACGCCAGCATCCACGACACCGTGGAGAACCTGATCAT
CCTGGCCAACAACAGCCTGAGCAGCAACGGCAACGTGACCGAGAGCGGCTGCAA
AGAGTGCGAGGAACTGGAAGAGAAGAACATCAAAGAGTTTCTGCAGAGCTTCGT
GCACATCGTGCAGATGTTCATCAACACCAGCTGA

A corresponding amino acid sequence for CD27TrDAP10icd3z15 is as follows:

(SEQ ID NO: 88)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRILVIFSGMFLVFTLAGALFLHLCARPRRSP
AQEDGKVYINMPGRGKRVKFSRSADAPAYQQGQNQLYNELNLGRREEYDVLDKRR
GRDPEMGGKPRRKNPQEGLYNELQKDKMAEAYSEIGMKGERRRGKGHDGLYQGLS
TATKDTYDALHMQALPPRGPQCTNYALLKLAGDVESNPGPMRISKPHLRSISIQCYL
CLLLNSHFLTEAGIHVFILGCFSAGLPKTEANWVNVISDLKKIEDLIQSMHIDATLYTE
SDVHPSCKVTAMKCFLLELQVISLESGDASIHDTVENLIILANNSLSSNGNVTESGCKE
CEELEEKNIKEFLQSFVHIVQMFINTS
GSPco27FLdap10icd3z15:
(SEQ ID NO: 36)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCATACTGGTCAT
CTTTTCTGGAATGTTCCTTGTGTTCACCCTGGCAGGAGCCCTGTTCCTTCACCTTT
GCGCACGCCCACGCCGCAGCCCCGCCCAAGAAGATGGCAAAGTCTACATCAACA
TGCCAGGCAGGGGCAAACGCGTGAAGTTCAGCAGGAGCGCAGACGCCCCCGCGT
ACCAGCAGGGCCAGAACCAGCTCTATAACGAGCTCAATCTAGGACGAAGAGAGG
AGTACGATGTTTTGGACAAAAGACGTGGCCGGGACCCTGAGATGGGGGGAAAGC
CGAGAAGGAAGAACCCTCAGGAAGGCCTGTACAATGAACTGCAGAAAGATAAG
ATGGCGGAGGCCTACAGTGAGATTGGGATGAAAGGCGAGCGCCGGAGGGGCAA
GGGGCACGATGGCCTTTACCAGGGTCTCAGTACAGCCACCAAGGACACCTACGA
CGCCCTTCACATGCAGGCCCTGCCCCCTCGCGGACCGCAGTGTACTAATTATGCT
CTCTTGAAATTGGCTGGAGATGTTGAGAGCAATCCCGGGCCCATGCGCATTAGCA
AGCCCCACCTGCGGAGCATCAGCATCCAGTGCTACCTGTGCCTGCTGCTGAACAG
CCACTTCCTGACCGAGGCCGGCATCCACGTGTTCATCCTGGGCTGCTTCAGCGCC
GGACTGCCCAAGACCGAGGCCAACTGGGTGAACGTGATCAGCGACCTGAAGAAG
ATCGAGGACCTGATCCAGAGCATGCACATCGACGCCACCCTGTACACCGAGAGC
GACGTGCACCCCAGCTGCAAGGTGACCGCCATGAAGTGCTTTCTGCTGGAACTGC
AGGTGATCAGCCTGGAAAGCGGCGACGCCAGCATCCACGACACCGTGGAGAACC
TGATCATCCTGGCCAACAACAGCCTGAGCAGCAACGGCAACGTGACCGAGAGCG
GCTGCAAAGAGTGCGAGGAACTGGAAGAGAAGAACATCAAAGAGTTTCTGCAG
AGCTTCGTGCACATCGTGCAGATGTTCATCAACACCAGCTGA

A corresponding amino acid sequence for GSPco27FLdap10icd3z15 is as follows:

(SEQ ID NO: 89)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRILVIFSGMFLVFTLAGALFLHLCARPRRS
PAQEDGKVYINMPGRGKRVKFSRSADAPAYQQGQNQLYNELNLGRREEYDVLDKR
RGRDPEMGGKPRRKNPQEGLYNELQKDKMAEAYSEIGMKGERRRGKGHDGLYQG
LSTATKDTYDALHMQALPPRGPQCTNYALLKLAGDVESNPGPMRISKPHLRSISIQCY
LCLLLNSHFLTEAGIHVFILGCFSAGLPKTEANWVNVISDLKKIEDLIQSMHIDATLYT
ESDVHPSCKVTAMKCFLLELQVISLESGDASIHDTVENLIILANNSLSSNGNVTESGCK
ECEELEEKNIKEFLQSFVHIVQMFINTS
CD27TrDAP12icd3z15:
(SEQ ID NO: 37)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCATCCTTGTGATCTTCTCTGG
AATGTTCCTTGTTTTCACCCTGGCCGGGGCCCTGTTCCTCCATTACTTCCTGGGCC
GGCTGGTCCCTCGGGGGCGAGGGGCTGCGGAGGCAGCGACCCGGAAACAGCGTA
TCACTGAGACCGAGTCGCCTTATCAGGAGCTCCAGGGTCAGAGGTCGGATGTCTA
CAGCGACCTCAACACACAGAGGCCGTATTACAAAAAACGCGTGAAGTTCAGCAG
GAGCGCAGACGCCCCCGCGTACCAGCAGGGCCAGAACCAGCTCTATAACGAGCT
CAATCTAGGACGAAGAGAGGAGTACGATGTTTTGGACAAAAGACGTGGCCGGGA
CCCTGAGATGGGGGGAAAGCCGAGAAGGAAGAACCCTCAGGAAGGCCTGTACA
ATGAACTGCAGAAAGATAAGATGGCGGAGGCCTACAGTGAGATTGGGATGAAA
GGCGAGCGCCGGAGGGGCAAGGGGCACGATGGCCTTTACCAGGGTCTCAGTACA
GCCACCAAGGACACCTACGACGCCCTTCACATGCAGGCCCTGCCCCCTCGCGGAC
CGCAGTGTACTAATTATGCTCTCTTGAAATTGGCTGGAGATGTTGAGAGCAATCC
CGGGCCCATGCGCATTAGCAAGCCCCACCTGCGGAGCATCAGCATCCAGTGCTA
CCTGTGCCTGCTGCTGAACAGCCACTTCCTGACCGAGGCCGGCATCCACGTGTTC
ATCCTGGGCTGCTTCAGCGCCGGACTGCCCAAGACCGAGGCCAACTGGGTGAAC
GTGATCAGCGACCTGAAGAAGATCGAGGACCTGATCCAGAGCATGCACATCGAC
GCCACCCTGTACACCGAGAGCGACGTGCACCCCAGCTGCAAGGTGACCGCCATG
AAGTGCTTTCTGCTGGAACTGCAGGTGATCAGCCTGGAAAGCGGCGACGCCAGC
ATCCACGACACCGTGGAGAACCTGATCATCCTGGCCAACAACAGCCTGAGCAGC
AACGGCAACGTGACCGAGAGCGGCTGCAAAGAGTGCGAGGAACTGGAAGAGAA
GAACATCAAAGAGTTTCTGCAGAGCTTCGTGCACATCGTGCAGATGTTCATCAAC
ACCAGCTGA

A corresponding amino acid sequence for CD27TrDAP12icd3z15 is as follows:

(SEQ ID NO: 90)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRILVIFSGMFLVFTLAGALFLHYFLGRLVPR
GRGAAEAATRKQRITETESPYQELQGQRSDVYSDLNTQRPYYKKRVKFSRSADAPA
YQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPRGPQCTNYAL
LKLAGDVESNPGPMRISKPHLRSISIQCYLCLLLNSHFLTEAGIHVFILGCFSAGLPKTE
ANWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTS
GSPco27FLdap12icd3z15:
(SEQ ID NO: 38)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCATACTGGTCAT
CTTTTCTGGAATGTTCCTTGTGTTCACCCTGGCAGGAGCCCTGTTCCTTCACTACT
TCCTGGGCCGGCTGGTCCCTCGGGGGCGAGGGGCTGCGGAGGCAGCGACCCGGA
AACAGCGTATCACTGAGACCGAGTCGCCTTATCAGGAGCTCCAGGGTCAGAGGT
CGGATGTCTACAGCGACCTCAACACACAGAGGCCGTATTACAAAAAACGCGTGA
AGTTCAGCAGGAGCGCAGACGCCCCCGCGTACCAGCAGGGCCAGAACCAGCTCT
ATAACGAGCTCAATCTAGGACGAAGAGAGGAGTACGATGTTTTGGACAAAAGAC
GTGGCCGGGACCCTGAGATGGGGGGAAAGCCGAGAAGGAAGAACCCTCAGGAA
GGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGG
TCTCAGTACAGCCACCAAGGACACCTACGACGCCCTTCACATGCAGGCCCTGCCC
CCTCGCGGACCGCAGTGTACTAATTATGCTCTCTTGAAATTGGCTGGAGATGTTG
AGAGCAATCCCGGGCCCATGCGCATTAGCAAGCCCCACCTGCGGAGCATCAGCA
TCCAGTGCTACCTGTGCCTGCTGCTGAACAGCCACTTCCTGACCGAGGCCGGCAT
CCACGTGTTCATCCTGGGCTGCTTCAGCGCCGGACTGCCCAAGACCGAGGCCAAC
TGGGTGAACGTGATCAGCGACCTGAAGAAGATCGAGGACCTGATCCAGAGCATG
CACATCGACGCCACCCTGTACACCGAGAGCGACGTGCACCCCAGCTGCAAGGTG
ACCGCCATGAAGTGCTTTCTGCTGGAACTGCAGGTGATCAGCCTGGAAAGCGGC
GACGCCAGCATCCACGACACCGTGGAGAACCTGATCATCCTGGCCAACAACAGC
CTGAGCAGCAACGGCAACGTGACCGAGAGCGGCTGCAAAGAGTGCGAGGAACT
GGAAGAGAAGAACATCAAAGAGTTTCTGCAGAGCTTCGTGCACATCGTGCAGAT
GTTCATCAACACCAGCTGA

A corresponding amino acid sequence for GSPco27FLdap12icd3z15 is as follows:

(SEQ ID NO: 91)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRILVIFSGMFLVFTLAGALFLHYFLGRLVP
RGRGAAEAATRKQRITETESPYQELQGQRSDVYSDLNTQRPYYKKRVKFSRSADAP
AYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPRGPQCTNYA
LLKLAGDVESNPGPMRISKPHLRSISIQCYLCLLLNSHFLTEAGIHVFILGCFSAGLPKT
EANWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINTS
CD27TrNKG2Dic3z15:
(SEQ ID NO: 39)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGGCACGGCCACATCCCTGGTGGCTGTGCGTTCTGGGGACCCTG
GTGGGGCTCTCAGCTACTCCAGCCCCCAAGAGCTGCCCAGAGAGGCACTACTGG
GCTCAGGGAAAGCTGTGCTGCCAGATGTGTGAGCCAGGAACATTCCTCGTGAAG
GACTGTGACCAGCATAGAAAGGCTGCTCAGTGTGATCCTTGCATACCGGGGGTCT
CCTTCTCTCCTGACCACCACACCCGGCCCCACTGTGAGAGCTGTCGGCACTGTAA
CTCTGGTCTTCTCGTTCGCAACTGCACCATCACTGCCAATGCTGAGTGTGCCTGTC
GCAATGGCTGGCAGTGCAGGGACAAGGAGTGCACCGAGTGTGATCCTCTTCCAA
ACCCTTCGCTGACCGCTCGGTCGTCTCAGGCCCTGAGCCCACACCCTCAGCCCAC
CCACTTACCTTATGTCAGTGAGATGCTGGAGGCCAGGACAGCTGGGCACATGCA
GACTCTGGCTGACTTCAGGCAGCTGCCTGCCCGGACTCTCTCTACCCACTGGCCA
CCCCAAAGATCCCTGTGCAGCTCCGATTTTATTCGCATCCTTGTGATCTTCTCTGG
AATGTTCCTTGTTTTCACCCTGGCCGGGGCCCTGTTCCTCCATAGCGCGAACGAA
CGCTGCAAAAGCAAAGTGGTGCCGTGCCGCCAGAAACAGTGGCGCACCAGCTTT
GATAGCAAAAAACTGGATCTGAACTATAACCATTTTGAAAGCATGGAATGGAGC
CATCGCAGCCGCCGCGGCCGCATTTGGGGCATGAAACGCGTGAAGTTCAGCAGG
AGCGCAGACGCCCCCGCGTACCAGCAGGGCCAGAACCAGCTCTATAACGAGCTC
AATCTAGGACGAAGAGAGGAGTACGATGTTTTGGACAAAAGACGTGGCCGGGAC
CCTGAGATGGGGGGAAAGCCGAGAAGGAAGAACCCTCAGGAAGGCCTGTACAA
TGAACTGCAGAAAGATAAGATGGCGGAGGCCTACAGTGAGATTGGGATGAAAG
GCGAGCGCCGGAGGGGCAAGGGGCACGATGGCCTTTACCAGGGTCTCAGTACAG
CCACCAAGGACACCTACGACGCCCTTCACATGCAGGCCCTGCCCCCTCGCGGACC
GCAGTGTACTAATTATGCTCTCTTGAAATTGGCTGGAGATGTTGAGAGCAATCCC
GGGCCCATGCGCATTAGCAAGCCCCACCTGCGGAGCATCAGCATCCAGTGCTAC
CTGTGCCTGCTGCTGAACAGCCACTTCCTGACCGAGGCCGGCATCCACGTGTTCA
TCCTGGGCTGCTTCAGCGCCGGACTGCCCAAGACCGAGGCCAACTGGGTGAACG
TGATCAGCGACCTGAAGAAGATCGAGGACCTGATCCAGAGCATGCACATCGACG
CCACCCTGTACACCGAGAGCGACGTGCACCCCAGCTGCAAGGTGACCGCCATGA
AGTGCTTTCTGCTGGAACTGCAGGTGATCAGCCTGGAAAGCGGCGACGCCAGCA
TCCACGACACCGTGGAGAACCTGATCATCCTGGCCAACAACAGCCTGAGCAGCA
ACGGCAACGTGACCGAGAGCGGCTGCAAAGAGTGCGAGGAACTGGAAGAGAAG
AACATCAAAGAGTTTCTGCAGAGCTTCGTGCACATCGTGCAGATGTTCATCAACA
CCAGCTGA

A corresponding amino acid sequence for CD27TrNKG2Dic3z15 is as follows:

(SEQ ID NO: 92)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMARPHPWWLCVLGTLVGLSATPAPKSCPERHYWAQGKLCCQMCEPGTFL
VKDCDQHRKAAQCDPCIPGVSFSPDHHTRPHCESCRHCNSGLLVRNCTITANAECAC
RNGWQCRDKECTECDPLPNPSLTARSSQALSPHPQPTHLPYVSEMLEARTAGHMQT
LADFRQLPARTLSTHWPPQRSLCSSDFIRILVIFSGMFLVFTLAGALFLHSANERCKSK
VVPCRQKQWRTSFDSKKLDLNYNHFESMEWSHRSRRGRIWGMKRVKFSRSADAPA
YQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPRGPQCTNYAL
LKLAGDVESNPGPMRISKPHLRSISIQCYLCLLLNSHFLTEAGIHVFILGCFSAGLPKTE
ANWVNVISDLKKIEDLIQSMHIDATLYTESDVHPSCKVTAMKCFLLELQVISLESGDA
SIHDTVENLIILANNSLSSNGNVTESGCKECEELEEKNIKEFLQSFVHIVQMFINTS
GSPco27TrNKG2Dicd3z15:
(SEQ ID NO: 40)
ATGACAAGAGTTACTAACAGCCCCTCTCTCCAAGCTCACTTACAGGCT
CTCTACTTAGTCCAGCACGAAGTCTGGAGACCTCTGGCGGCAGCCTACCAAGAAC
AACTGGACCGACCGGTGGTACCTCACCCTTACCGAGTCGGCGACACAGTGTGGG
TCCGCCGACACCAGACTAAGAACCTAGAACCTCGCTGGAAAGGACCTTACACAG
TCCTGCTGACCACCCCCACCGCCCTCAAAGTAGACGGCATCGCAGCTTGGATACA
CGCCGCCCACGTGAAGGCTGCCGACCCCGGGGGTGGACCATCCTCTAGACTGCC
ATGCTCGAGGATGCTTCTCCTGGTGACAAGCCTTCTGCTCTGTGAGTTACCACAC
CCAGCATTCCTCCTGATCCCAGCTACACCGGCTCCGAAGTCCTGCCCGGAGCGGC
ATTATTGGGCACAGGGCAAGTTGTGTTGTCAAATGTGTGAGCCGGGAACCTTTCT
CGTGAAGGATTGCGATCAGCATCGGAAGGCCGCGCAGTGCGACCCATGTATACC
AGGGGTCTCATTTTCCCCAGATCACCATACGAGGCCGCACTGTGAGTCTTGCAGG
CATTGTAATTCCGGCTTGTTGGTCCGCAACTGTACTATTACTGCGAATGCAGAGT
GTGCTTGTAGAAACGGATGGCAGTGCAGGGACAAAGAATGTACGGAGTGTGATC
CACTGCCTAACCCCAGTCTTACAGCAAGATCTTCACAGGCCCTCAGCCCGCATCC
TCAACCAACACATCTTCCTTACGTGTCAGAAATGTTGGAGGCGCGAACCGCAGGC
CATATGCAGACCCTGGCGGACTTTCGGCAGCTGCCAGCACGCACACTTAGTACAC
ACTGGCCACCACAACGCAGCTTGTGCTCTTCCGATTTCATCCGCATACTGGTCAT
CTTTTCTGGAATGTTCCTTGTGTTCACCCTGGCAGGAGCCCTGTTCCTTCACAGCG
CGAACGAACGCTGCAAAAGCAAAGTGGTGCCGTGCCGCCAGAAACAGTGGCGCA
CCAGCTTTGATAGCAAAAAACTGGATCTGAACTATAACCATTTTGAAAGCATGGA
ATGGAGCCATCGCAGCCGCCGCGGCCGCATTTGGGGCATGAAACGCGTGAAGTT
CAGCAGGAGCGCAGACGCCCCCGCGTACCAGCAGGGCCAGAACCAGCTCTATAA
CGAGCTCAATCTAGGACGAAGAGAGGAGTACGATGTTTTGGACAAAAGACGTGG
CCGGGACCCTGAGATGGGGGGAAAGCCGAGAAGGAAGAACCCTCAGGAAGGCC
TGTACAATGAACTGCAGAAAGATAAGATGGCGGAGGCCTACAGTGAGATTGGGA
TGAAAGGCGAGCGCCGGAGGGGCAAGGGGCACGATGGCCTTTACCAGGGTCTCA
GTACAGCCACCAAGGACACCTACGACGCCCTTCACATGCAGGCCCTGCCCCCTCG
CGGACCGCAGTGTACTAATTATGCTCTCTTGAAATTGGCTGGAGATGTTGAGAGC
AATCCCGGGCCCATGCGCATTAGCAAGCCCCACCTGCGGAGCATCAGCATCCAG
TGCTACCTGTGCCTGCTGCTGAACAGCCACTTCCTGACCGAGGCCGGCATCCACG
TGTTCATCCTGGGCTGCTTCAGCGCCGGACTGCCCAAGACCGAGGCCAACTGGGT
GAACGTGATCAGCGACCTGAAGAAGATCGAGGACCTGATCCAGAGCATGCACAT
CGACGCCACCCTGTACACCGAGAGCGACGTGCACCCCAGCTGCAAGGTGACCGC
CATGAAGTGCTTTCTGCTGGAACTGCAGGTGATCAGCCTGGAAAGCGGCGACGC
CAGCATCCACGACACCGTGGAGAACCTGATCATCCTGGCCAACAACAGCCTGAG
CAGCAACGGCAACGTGACCGAGAGCGGCTGCAAAGAGTGCGAGGAACTGGAAG
AGAAGAACATCAAAGAGTTTCTGCAGAGCTTCGTGCACATCGTGCAGATGTTCAT
CAACACCAGCTGA

A corresponding amino acid sequence for GSPco27TrNKG2Dicd3z15 is as follows:

(SEQ ID NO: 93)
MTRVTNSPSLQAHLQALYLVQHEVWRPLAAAYQEQLDRPVVPHPYRVG
DTVWVRRHQTKNLEPRWKGPYTVLLTTPTALKVDGIAAWIHAAHVKAADPGGGPS
SRLPCSRMLLLVTSLLLCELPHPAFLLIPATPAPKSCPERHYWAQGKLCCQMCEPGTF
LVKDCDQHRKAAQCDPCIPGVSFSPDHHTRPHCESCRHCNSGLLVRNCTITANAECA
CRNGWQCRDKECTECDPLPNPSLTARSSQALSPHPQPTHLPYVSEMLEARTAGHMQ
TLADFRQLPARTLSTHWPPQRSLCSSDFIRILVIFSGMFLVFTLAGALFLHSANERCKS
KVVPCRQKQWRTSFDSKKLDLNYNHFESMEWSHRSRRGRIWGMKRVKFSRSADAP
AYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPRGPQCTNYA
LLKLAGDVESNPGPMRISKPHLRSISIQCYLCLLLNSHFLTEAGIHVFILGCFSAGLPKT
EANWVNVISDLKKIEDLIQSMHIDATLYTESDVHPSCKVTAMKCFLLELQVISLESGD
ASIHDTVENLIILANNSLSSNGNVTESGCKECEELEEKNIKEFLQSFVHIVQMFINTS

### C. T Cell Receptor (TCR)

In some aspects, an antigen-targeting, e.g., a CD70-targeting, genetically engineered antigen receptor includes recombinant TCRs and/or TCRs cloned from naturally occurring T cells, or one or more portions thereof. A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to an MHC receptor. In some aspects, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some aspects, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some aspects, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e., MHC-peptide complex. An “antigen-binding portion” or antigen-binding fragment” of a TCR, which can be used interchangeably, refers to a molecule that contains a portion of the structural domains of a TCR, but that binds the antigen (e.g., MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some aspects, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some aspects, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some aspects, the variable region of the β-chain can contain a further hypervariability (HV4) region.

In some aspects, the TCR chains contain a constant domain. For example, like immunoglobulins, the extracellular portion of TCR chains (e.g., a-chain, β-chain) can contain two immunoglobulin domains, a variable domain (e.g., Vₐ or Vₚ; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some aspects, a TCR may have an additional cysteine residue in each of the α and β chains such that the TCR contains two disulfide bonds in the constant domains.

In some aspects, the TCR chains can contain a transmembrane domain. In some aspects, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some aspects, the TCR may be a heterodimer of two chains α and β (or optionally γ and δ) or it may be a single chain TCR construct. In some aspects, the TCR is a heterodimer containing two separate chains (α and β chains or γ and δ chains) that are linked, such as by a disulfide bond or disulfide bonds. In some aspects, a TCR for a target antigen (e.g., a cancer antigen) is identified and introduced into the cells. In some aspects, nucleic acid encoding the TCR can be obtained from a variety of sources, such as by polymerase chain reaction (PCR) amplification of publicly available TCR DNA sequences. In some aspects, the TCR is obtained from a biological source, such as from cells such as from a T cell (e.g., cytotoxic T cell), T cell hybridomas or other publicly available source. In some aspects, the T cells can be obtained from in vivo isolated cells. In some aspects, a high-affinity T cell clone can be isolated from a patient, and the TCR isolated. In some aspects, the T cells can be a cultured T cell hybridoma or clone. In some aspects, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some aspects, phage display is used to isolate TCRs against a target antigen (see, e.g., Varela-Rohena et al., 2008 and Li, 2005). In some aspects, the TCR or antigen-binding portion thereof can be synthetically generated from knowledge of the sequence of the TCR.

### VIII. Cytokines

One or more cytokines may be utilized with one or more antigen-targeting, e.g., CD70-targeting, genetically engineered receptors, such as antigen-specific, e.g., CD70-specific, CARs. In some cases, one or more cytokines are present on the same vector molecule as the engineered receptor, although in other cases they are on separate vector molecules. In particular aspects, one or more cytokines are co-expressed from the same vector as the engineered receptor. One or more cytokines may be produced as a separate polypeptide from the antigen-specific, e.g., CD70-specific, receptor. As one example, Interleukin-15 (IL-15), is utilized. IL-15 may be employed because, for example, it is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death. In addition to IL-15, other cytokines are envisioned. These include, but are not limited to, cytokines, chemokines, and other molecules that contribute to the activation and proliferation of cells used for human application. As one example, the one or more cytokines are IL-15, IL-12, IL-2, IL-18, IL-21, IL-23, IL-7, or combination thereof. As another example, the cytokine is IL-21. As another example, the cytokine is IL-12. NK cells expressing IL-15 or other cytokines disclosed herein may be utilized and are capable of continued supportive cytokine signaling, which is useful for their survival post-infusion.

In specific aspects, NK cells express one or more exogenously provided cytokines. The cytokine may be exogenously provided to the NK cells because it is expressed from an expression vector within the cell and/or because it is provided in a culture medium of the cells. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide gene. The cytokine may be expressed as a separate polypeptide molecule from a suicide gene and as a separate polypeptide from an engineered receptor of the cell. In some aspects, the present disclosure concerns co-utilization of CAR and/or TCR vectors with IL-15, particularly in NK cells. In some aspects, the present disclosure concerns co-utilization of CAR and/or TCR vectors with IL-21, particularly in NK cells. In some aspects, the present disclosure concerns co-utilization of CAR and/or TCR vectors with IL-12, particularly in NK cells.

### IX. Suicide Genes

In particular aspects, a suicide gene is utilized in conjunction with cell therapy of any kind to control its use and allow for termination of the cell therapy at a desired event and/or time. The suicide gene is employed in transduced cells for the purpose of eliciting death for the transduced cells when needed. The antigen-targeting, e.g., CD70-targeting, cells of the present disclosure that have been modified to harbor a vector encompassed by the disclosure may comprise one or more suicide genes. In some aspects, the term “suicide gene” as used herein is defined as a gene which, upon administration of a prodrug or other agent, effects transition of a gene product to a compound which kills its host cell. In other aspects, a suicide gene encodes a gene product that is, when desired, targeted by an agent (such as an antibody) that targets the suicide gene product.

Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk:Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes also include CD20, CD52, EGFRv3, or inducible caspase 9. In one aspect, a truncated version of EGFR variant III (EGFRv3) may be used as a suicide antigen that can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

In particular aspects, vectors that encode the antigen-targeting, e.g., CD70-targeting, CAR, or any vector in a NK cell encompassed herein, include one or more suicide genes. The suicide gene may or may not be on the same vector as an antigen-targeting, e.g., CD70-targeting, CAR. In cases wherein the suicide gene is present on the same vector as the antigen-targeting, e.g., CD70-targeting, CAR, the suicide gene and the CAR may be separated by an IRES or 2A element, for example.

### X. Antigens

Aspects of the disclosure are directed to polypeptides (e.g., antibodies, CARs, engagers, etc.) that target one or more particular antigens. Among the antigens targeted by the antibodies and/or engineered polypeptides of the disclosure are those expressed in the context of a disease, condition, or cell type to be targeted. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some aspects, the antigen is selectively expressed or overexpressed on cells of the disease or condition, e.g., the tumor or pathogenic cells, as compared to normal or non-targeted cells or tissues. In other aspects, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include CD19, EBNA, CD123, HER2, CA-125, TRAIL/DR4, CD20, CD22, CD70, CD3δ, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGSS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1. Examples of sequences for antigens are known in the art, for example, in the GenBank® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRVIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### XI. Vectors

Polypeptides encoding one or more viral proteins and/or one or more antigen-specific receptors (e.g., CARs, chimeric polypeptides, immune cell engagers, etc.) may be delivered to recipient immune cells by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the immune cell is transduced with a vector encoding the polypeptide encoding one or more viral proteins and/or one or more antigen-specific receptors and also requires transduction of another gene or genes into the cell, such as a suicide gene and/or cytokine and/or an optional therapeutic gene product, the viral protein polypeptide(s), antigen-targeting polypeptide(s), suicide gene, cytokine, and optional therapeutic gene may or may not be comprised on or with the same vector. In some cases, the viral protein polypeptide(s), antigen-targeting polypeptide(s), suicide gene, cytokine, and optional therapeutic gene are expressed from the same vector molecule, such as the same viral vector molecule. In such cases, the expression of the viral protein polypeptide(s), antigen-targeting polypeptide(s), suicide gene, cytokine, and optional therapeutic gene may or may not be regulated by the same regulatory element(s). When the viral protein polypeptide(s), antigen-targeting polypeptide(s), suicide gene, cytokine, and optional therapeutic gene are on the same vector, they may or may not be expressed as separate polypeptides. In cases wherein they are expressed as separate polypeptides, they may be separated on the vector by a 2A element or IRES element (or both kinds may be used on the same vector once or more than once), for example.

### A. General Aspects

One of skill in the art would be well-equipped to construct a vector through standard recombinant techniques (see, for example, Sambrook et al., 2001 and Ausubel et al., 1996, both incorporated herein by reference) for the expression of the viral proteins and/or antigen receptors of the present disclosure.

### 1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

### 2. Promoter/Enhancers

The expression constructs provided herein comprise a promoter to drive expression of the viral protein and/or antigen receptor and other cistron gene products. A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible aspect. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain aspects, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### 3. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain aspects, the use of internal ribosome entry sites (IRES) elements is used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific aspects, in a single vector the multiple 2A sequences are non-identical, although in alternative aspects the same vector utilizes two or more of the same 2A sequences. Examples of 2A sequences are provided in US 2011/0065779 which is incorporated by reference herein in its entirety.

### 4. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

### 5. Selection and Screenable Markers

In some aspects, NK cells comprising a viral protein and/or an antigen-targeting receptor construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### B. Multicistronic Vectors

In particular aspects, the viral protein, antigen-targeting receptor, optional suicide gene, optional cytokine, and/or optional therapeutic gene are expressed from a multicistronic vector (the term “cistron” as used herein refers to a nucleic acid sequence from which a gene product may be produced). In specific aspects, the multicistronic vector encodes the viral protein, antigen-targeting receptor, the suicide gene, and at least one cytokine, and/or engineered receptor, such as a T-cell receptor and/or an additional antigen-targeting CAR. In some cases, the multicistronic vector encodes at least one viral protein and/or antigen-targeting CAR, at least one TNF-alpha mutant, and at least one cytokine. The cytokine may be of a particular type of cytokine, such as human or mouse or any species. In specific cases, the cytokine is IL 15, IL 12, IL2, IL18, and/or IL21.

In certain aspects, the present disclosure provides a flexible, modular system (the term “modular” as used herein refers to a cistron or component of a cistron that allows for interchangeability thereof, such as by removal and replacement of an entire cistron or of a component of a cistron, respectively, for example by using standard recombination techniques) utilizing a polycistronic vector having the ability to express multiple cistrons at substantially identical levels. The system may be used for cell engineering allowing for combinatorial expression (including overexpression) of multiple genes. In specific aspects, one or more of the genes expressed by the vector includes one, two, or more viral proteins and/or antigen receptors. The multiple genes may comprise, but are not limited to, viral proteins, CARs, TCRs, cytokines, chemokines, homing receptors, CRISPR/Cas9-mediated gene mutations, decoy receptors, cytokine receptors, chimeric cytokine receptors, and so forth. The vector may further comprise: (1) one or more reporters, for example fluorescent or enzymatic reporters, such as for cellular assays and animal imaging; (2) one or more cytokines or other signaling molecules; and/or (3) a suicide gene.

In specific cases, the vector may comprise at least 4 cistrons separated by cleavage sites of any kind, such as 2A cleavage sites. The vector may or may not be Moloney Murine Leukemia Virus (MoMLV or MMLV)-based including the 3′ and 5′ LTR with the psi packaging sequence in a pUC19 backbone. The vector may comprise 4 or more cistrons with three or more 2A cleavage sites and multiple ORFs for gene swapping. The system allows for combinatorial overexpression of multiple genes (7 or more) that are flanked by restriction site(s) for rapid integration through subcloning, and the system also includes at least three 2A self-cleavage sites, in some aspects. Thus, the system allows for expression of multiple viral proteins, CARs, TCRs, signaling molecules, cytokines, cytokine receptors, and/or homing receptors. This system may also be applied to other viral and non-viral vectors, including but not limited lentivirus, adenovirus AAV, as well as non-viral plasmids.

The modular nature of the system also enables efficient subcloning of a gene into each of the 4 cistrons in the polycistronic expression vector and the swapping of genes, such as for rapid testing. Restriction sites strategically located in the polycistronic expression vector allow for swapping of genes with efficiency.

Aspects of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has aspects wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

Aspects of the disclosure include polycistronic vectors comprising at least two, at least three, or at least four cistrons each flanked by one or more restriction enzyme sites, wherein at least one cistron encodes for at least one antigen receptor. In some cases, two, three, four, or more of the cistrons are translated into a single polypeptide and cleaved into separate polypeptides, whereas in other cases multiple of the cistrons are translated into a single polypeptide and cleaved into separate polypeptides. Adjacent cistrons on the vector may be separated by a self-cleavage site, such as a 2A self-cleavage site. In some cases each of the cistrons express separate polypeptides from the vector. On particular cases, adjacent cistrons on the vector are separated by an IRES element.

In certain aspects, the present disclosure provides a system for cell engineering allowing for combinatorial expression, including overexpression, of multiple cistrons that may include one, two, or more antigen receptors, for example. In particular aspects, the use of a polycistronic vector as described herein allows for the vector to produce equimolar levels of multiple gene products from the same mRNA. The multiple genes may comprise, but are not limited to, viral proteins, CARs, TCRs, cytokines, chemokines, homing receptors, CRISPR/Cas9-mediated gene mutations, decoy receptors, cytokine receptors, chimeric cytokine receptors, and so forth. The vector may further comprise one or more fluorescent or enzymatic reporters, such as for cellular assays and animal imaging. The vector may also comprise a suicide gene product for termination of cells harboring the vector when they are no longer needed or become deleterious to a host to which they have been provided.

In specific aspects, the vector is a viral vector (retroviral vector, lentiviral vector, adenoviral vector, or adeno-associated viral vector, for example) or a non-viral vector. The vector may comprise a Moloney Murine Leukemia Virus (MMLV) 5′ LTR, 3′ LTR, and/or psi packaging element. In specific cases, the psi packaging is incorporated between the 5′ LTR and the antigen receptor coding sequence. The vector may or may not comprise pUC19 sequence. In some aspects of the vector, at least one cistron encodes for a cytokine (IL-15, IL-7, IL-21, IL-23, IL-18, IL-12, or IL-2, for example), chemokine, cytokine receptor, and/or homing receptor.

When 2A cleavages sites are utilized in the vector, the 2A cleavage site may comprise a P2A, T2A, E2A and/or F2A site.

A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

Exemplary vectors may be circular and by convention, where position 1 (12 o'clock position at the top of the circle, with the rest of the sequence in clockwise direction) is set at the start of 5′ LTR.

In aspects wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (Thosea asigna virus 2A) were also identified. The mechanism of 2A-mediated “self-cleavage” was discovered to be ribosome skipping the formation of a glycyl-prolyl peptide bond at the C-terminus of the 2A.

In specific cases, the vector may be a γ-retroviral transfer vector. The retroviral transfer vector may comprise a backbone based on a plasmid, such as the pUC19 plasmid (large fragment (2.63 kb) in between HindIII and EcoRI restriction enzyme sites). The backbone may carry viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, brackets the genetic cargo of interest, such as CD70-targeting CARs and associated components. The psi packaging sequence, which is a target site for packaging by nucleocapsid, is also incorporated in cis, sandwiched between the 5′ LTR and the CAR coding sequence. Thus, the basic structure of an example of a transfer vector can be configured as such: pUC19 sequence-5′ LTR-psi packaging sequence-genetic cargo of interest-3′ LTR-pUC19 sequence. This system may also be applied to other viral and non-viral vectors, including but not limited lentivirus, adenovirus AAV, as well as non-viral plasmids.

### XII. Cells

The present disclosure encompasses cells, including immune cells and stem cells of any kind, that harbor at least one vector that encodes viral protein polypeptide(s) and/or antigen-targeting polypeptide(s) (e.g., an antibody and/or CAR) and that also may encode at least one cytokine and/or at least one suicide gene. In some cases, different vectors encode the viral protein polypeptide(s) and/or antigen-targeting polypeptide(s) vs. encodes the suicide gene and/or cytokine. The immune cells, including NK cells, may be derived from cord blood (including pooled cord blood from multiple sources), peripheral blood, induced pluripotent stem cells (iPSCs), hematopoietic stem cells (HSCs), bone marrow, or a mixture thereof. The NK cells may be derived from a cell line such as, but not limited to, NK-92 cells, for example. The NK cell may be a cord blood mononuclear cell, such as a CD56⁺ NK cell.

The present disclosure encompasses immune or other cells of any kind, including conventional T cells, gamma-delta T cells, NKT and invariant NK T cells, regulatory T cells, macrophages, B cells, dendritic cells, mesenchymal stromal cells (MSCs), or a mixture thereof.

In some cases, the cells have been expanded in the presence of an effective amount of universal antigen presenting cells (UAPCs), including in any suitable ratio. The cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example. In some cases, the NK cells were expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

Following genetic modification with the vector(s), the NK cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the viral protein polypeptide(s) and/or antigen-targeting polypeptide(s) (e.g., an antibody and/or CAR) is expanded ex vivo. The clone selected for expansion demonstrates the capacity to specifically recognize and lyse antigen-expressing target cells. The recombinant immune cells may be expanded by stimulation with IL-2, or other cytokines that bind the common gamma-chain (e.g., IL-7, IL-12, IL-15, IL-21, IL-23, and others). The recombinant immune cells may be expanded by stimulation with artificial antigen presenting cells. In a further aspect, the genetically modified cells may be cryopreserved.

Aspects of the disclosure encompass cells that express one or more viral proteins and/or one or more antigen-targeting CARs and one or more suicide genes as encompassed herein. The NK cell comprises a recombinant nucleic acid that encodes one or more viral proteins and/or one or more antigen-targeting CARs and one or more engineered nonsecretable, membrane bound TNF-alpha mutant polypeptides, in specific aspects. In specific aspects, in addition to expressing one or more viral proteins and/or one or more antigen-targeting CARs and TNF-alpha mutant polypeptides, the cell also comprises a nucleic acid that encodes one or more therapeutic gene products.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to express the viral protein polypeptide(s) and/or antigen-targeting polypeptide(s), optional suicide gene, optional cytokine(s), and optional therapeutic gene product(s) (using standard techniques for transduction and expansion for adoptive cell therapy, for example), they may be provided back to the individual from which they were originally sourced. In some cases, the cells are stored for later use for the individual or another individual.

The immune cells may be comprised in a population of cells, and that population may have a majority that are transduced with one or more viral protein polypeptide(s) and/or one or more antigen-targeting polypeptide(s) and/or one or more suicide genes and/or one or more cytokines. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of immune cells that are transduced with one or more viral protein polypeptide(s) and/or one or more antigen-targeting polypeptide(s) and/or one or more suicide genes and/or one or more cytokines. The one or more viral protein polypeptide(s) and/or one or more antigen-targeting polypeptide(s) and/or one or more suicide genes and/or one or more cytokines may be separate polypeptides.

The immune cells may be produced with the one or more viral protein polypeptide(s) and/or one or more antigen-targeting polypeptide(s) and/or one or more suicide genes and/or one or more cytokines for the intent of being modular with respect to a specific purpose. For example, cells may be generated, including for commercial distribution, expressing one or more viral protein polypeptide(s) and/or one or more antigen-targeting polypeptide(s) and/or one or more suicide genes and/or one or more cytokines (or distributed with a nucleic acid that encodes the mutant for subsequent transduction), and a user may modify them to express one or more other genes of interest (including therapeutic genes) dependent upon their intended purpose(s). For instance, an individual interested in treating CD70-positive cells, including CD70-positive cancer, may obtain or generate suicide gene-expressing cells (or heterologous cytokine-expressing cells) and modify them to express viral protein(s) and/or a receptor comprising a CD70-targeting polypeptide, or vice versa.

In particular aspects, NK cells are utilized, and the genome of the transduced NK cells expressing the one or more viral protein polypeptide(s) and/or one or more antigen-targeting polypeptide(s) and/or one or more suicide genes and/or one or more cytokines may be modified. The genome may be modified in any manner, but in specific aspects the genome is modified by CRISPR gene editing, for example. The genome of the cells may be modified to enhance effectiveness of the cells for any purpose.

### XIII. Gene Editing of Cells

In particular aspects, cells comprising at least one or more viral protein polypeptide(s) and/or one or more antigen-targeting polypeptide(s) are gene edited to modify expression of one or more endogenous genes in the cell. In specific cases, cells are modified to have reduced levels of expression of one or more endogenous genes, including inhibition of expression of one or more endogenous genes (that may be referred to as knocked out). Such cells may or may not be expanded.

In some aspects the polynucleotides are introduced alone or as part of engineered receptor constructs via stable viral vectors, in other aspects the polynucleotides can be introduced by electroporation for transient expression of mRNA that would be translated to protein inside the cells, and in other aspects the polynucleotides can be introduced using knock-in approaches using gene editing technologies including but not limited to CRISPR, TALENS, Zinc fingers, and/or retrons, among others. The knock-in approaches can introduce the polynucleotides in specific favorable genomic locations, such as under the promoter of hypoxia-inducible factor-1 α (HIF-1 α), or other promoters that are activated in the tumor microenvironment.

In particular cases, one or more endogenous genes of the cells are modified, such as disrupted in expression where the expression is reduced in part or in full. In specific cases, one or more genes are knocked down or knocked out using processes of the disclosure. In specific cases, multiple genes are knocked down or knocked out, and this may or may not occur in the same step in their production. The genes that are edited in the cells may be of any kind, but in specific aspects the genes are genes whose gene products inhibit activity and/or proliferation of the cells, including antigen-specific, e.g., CD70-specific, CAR NK cells, such as those derived from cord blood, as one example. In specific cases the genes that are edited in the antigen-specific, e.g., CD70-specific, CAR cells allow the antigen-specific, e.g., CD70-specific, CAR cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, and CD7. In specific aspects, the TGFBR2 gene is knocked out or knocked down in the antigen-specific, e.g., CD70-specific, CAR cells.

### A. DNA-Binding Nucleic Acids

In some aspects, the gene editing is carried out using one or more DNA-binding nucleic acids, such as alteration via an RNA-guided endonuclease (RGEN) or retron library recombineering.

### 1. CRISPR/Cas

In some aspects, gene editing can be carried out using clustered regularly interspaced short palindromic repeats (CRISPR) and CRISPR-associated (Cas) proteins; in some aspects, CpF1 is utilized instead of Cas9. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other aspects, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other aspects, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some aspects, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (Cas12a) homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). In some cases, Cpf1 (Cas12a) may be used as an endonuclease instead of Cas9. The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some aspects, a Cas9 nickase may be used in combination with guide sequence(s), e.g., two guide sequences, which target respectively sense and antisense strands of the DNA target. This combination allows both strands to be nicked and used to induce NHEJ or HDR.

In some aspects, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some aspects, the degree of complementarity between a guide sequence and its corresponding target sequence, when optimally aligned using a suitable alignment algorithm, is about or more than about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US20110059502, incorporated herein by reference.

### 2. Retrons

In some aspects, the gene editing is carried out using retrons and retron recombineering. A retron is a distinct DNA sequence found in the genome of many bacteria species that codes for reverse transcriptase and a unique single-stranded DNA/RNA hybrid called multicopy single-stranded DNA (msDNA). Retron msr RNA is the non-coding RNA produced by retron elements and is the immediate precursor to the synthesis of msDNA. Retron elements are about 2000 kb long. They contain a single operon controlling the synthesis of an RNA transcript carrying three loci, msr, msd, and ret, that are involved in msDNA synthesis. The DNA portion of msDNA is encoded by the msd gene, the RNA portion is encoded by the msr gene, while the product of the ret gene is a reverse transcriptase similar to the RTs produced by retroviruses and other types of retroelements. Like other reverse transcriptases, the retron RT contains seven regions of conserved amino acids, including a highly conserved tyr-ala-asp-asp (YADD) sequence associated with the catalytic core. The ret gene product is responsible for processing the msd/msr portion of the RNA transcript into msDNA.

The retron msr RNA folds into a characteristic secondary structure that contains a conserved guanosine residue at the end of a stem loop. Synthesis of DNA by the retron-encoded reverse transcriptase (RT) results in a DNA/RNA chimera which is composed of small single-stranded DNA linked to small single-stranded RNA. The RNA strand is joined to the 5′ end of the DNA chain via a 2′-5′ phosphodiester linkage that occurs from the 2′ position of the conserved internal guanosine residue.

Materials and methods for gene editing using retrons and retron recombineering are disclosed in, e.g., Schubert M. G. et al. (April 2021). PNAS 118 (18): e2018181118, incorporated by reference herein in its entirety.

### B. Nucleases

In some aspects, the gene editing is carried out using one or more nucleases, such as one or more transcription activator-like effector nucleases (TALENs) and/or zinc-finger nucleases (ZFNs).

### 1. TALENS

TALENs are DNA-binding restriction enzymes engineered to cut specific sequences of DNA and can be made by fusing a transcription activator-like (TAL) effector DNA-binding domain to a DNA cleavage domain (a nuclease). TAL effectors are proteins secreted by Xanthomonas bacteria. The DNA binding domain contains a repeated highly conserved sequence of about 33-34 amino acids with divergent 12th and 13th amino acids, referred to as the Repeat Variable Diresidue (RVD), that are highly variable and show a strong correlation with specific nucleotide recognition. In some aspects, specific DNA-binding domains are engineered by selecting a combination of repeat segments containing the appropriate RVDs, and slight changes in the RVD and the incorporation of “nonconventional” RVD sequences can improve targeting specificity. The non-specific DNA cleavage domain from the end of the FokI endonuclease and/or variants thereof can be used to construct hybrid nucleases. The FokI domain functions as a dimer, having two constructs with unique DNA binding domains for sites in the target genome with proper orientation and spacing. Both the number of amino acid residues between the TAL effector DNA binding domain and the FokI cleavage domain and the number of bases between the two individual TALEN binding sites can be varied, in some aspects, to achieve high levels of activity and/or specificity.

TALEN constructs may be generated using publicly available software programs (e.g., DNAWorks) to calculate oligonucleotides suitable for assembly in a two-step PCR oligonucleotide assembly followed by whole gene amplification. Additionally, or alternatively, a number of modular assembly schemes may be used, such as those described in Cermak T. et al. (July 2011). Nucleic Acids Research. 39 (12): e82; Zhang F. (February 2011) et al. Nature Biotechnology. 29 (2): 149-53; Morbitzer R. et al. (July 2011). Nucleic Acids Research. 39 (13): 5790-9; Li T. et al. (August 2011). Nucleic Acids Research. 39 (14): 6315-25; Geissler R. et al. (2011). PLOS ONE. 6 (5): e19509; and Weber E. et al. (2011). PLOS ONE. 6 (5): e19722, all of which are incorporated by reference herein in their entirety. Once TALEN constructs have been assembled, they may be inserted into a viral or non-viral vector; target cells are then transfected with the vector, and gene products are expressed and can enter the nucleus to access the genome. TALEN can be used to edit genomes by inducing double-strand breaks (DSB), which cells respond to with repair mechanisms (e.g., non-homologous end joining and/or homology directed repair). Additionally, or alternatively, TALEN constructs can be delivered to the cells as mRNAs.

### 2. ZFNs

ZFNs are restriction enzymes generated by fusing a zinc finger DNA-binding domain to a DNA-cleavage domain. In some aspects, zinc finger domains can be engineered to target specific desired DNA sequences to enable zinc-finger nucleases to target unique sequences in genomes. The DNA-binding domains can contain between three and six individual zinc finger repeats (e.g., 3, 4, 5, or 6 repeats) and can each recognize between 9 and 18 base pairs (e.g., 9, 10, 11, 12, 13, 14, 15, 16, 17, or 18 base pairs). Upon recognition of a 3 base pair DNA sequence, ZFNs can generate a 3-finger array that can recognize a 9 base pair target site. Additionally, or alternatively, ZFNs can utilize either 1-finger or 2-finger modules to generate zinc-finger arrays with six or more individual zinc fingers. ZFN DNA-binding domains may be selected using, e.g., phage display, yeast one-hybrid systems, bacterial one-hybrid and two-hybrid systems, and mammalian cells to select proteins that bind a given DNA target from a large pool of partially randomized zinc-finger arrays. In some aspects, a bacterial two-hybrid system is used and combines pre-selected pools of ZFNs selected to bind a given 3 base pair DNA sequence followed by a second round of selection to obtain 3-finger arrays capable of binding a desired 9 base pair sequence. See, e.g., Maeder M L, et al. (September 2008). Mol. Cell. 31 (2): 294-301, incorporated by reference herein in its entirety.

The non-specific DNA cleavage domain (e.g., from the type IIs restriction endonuclease FokI) can be used as the cleavage domain in ZFNs. This cleavage domain dimerizes to cleave DNA, and in some aspects, a pair of ZFNs is used to target non-palindromic DNA sites. Standard ZFNs fuse the cleavage domain to the C-terminus of each zinc finger domain. To let the two cleavage domains dimerize and cleave DNA, the two individual ZFNs bind opposite strands of DNA with their C-termini a certain distance apart. In some aspects, the 5′ edge of each binding site is separated by 5 to 7 base pairs for the linker sequences between the zinc finger domain and the cleavage domain. Several different protein engineering techniques have been employed to improve both the activity and specificity of the nuclease domain used in ZFNs. For example, in some aspects, a FokI variant with enhanced cleavage activity generated using directed evolution is employed. See, e.g., Guo J. et al. (2010). Journal of Molecular Biology. 400 (1): 96-107, incorporated by reference herein in its entirety. Additional or alternatively, structure-based design can be employed to improve the cleavage specificity of FokI by modifying the dimerization interface so that only the intended heterodimeric species are active.

In some aspects, zinc-finger nickases (ZFNickases) may be used. ZFNickases can be created by inactivating the catalytic activity of one ZFN monomer in the ZFN dimer required for double-strand cleavage. ZFNickases demonstrate strand-specific nicking activity in vitro and can provide for highly specific single-strand breaks in DNA, which undergo the same cellular mechanisms for DNA that ZFNs exploit but show a significantly reduced frequency of mutagenic NHEJ repairs at their target nicking site. This reduction can bias for homologous recombination (HR)-mediated gene modifications.

### XIV. Therapeutic Methods

The compositions of the disclosure may be used for in vivo, in vitro, or ex vivo administration. The route of administration of the composition may be, for example, intracutaneous, subcutaneous, intravenous, intrapleural, local, topical, and intraperitoneal administrations.

One example of a therapeutic method for treating a subject for cancer comprises administering to a subject a therapeutically effective amount of an immune cell or population thereof disclosed herein. In some aspects, a polynucleotide encoding one or more viral, bacterial, and/or fungal genes capable of manipulating cell metabolism and/or one or more antigen-specific receptors has been introduced into the immune cell(s), and administration of a therapeutically effective amount of the immune cell(s) decreases tumor burden or increases survival of the subject. In some cases, expression by the immune cell of the one or more viral, bacterial, and/or fungal genes capable of manipulating cell metabolism and/or the one or more antigen-specific receptors encoded by the polynucleotide enhances the metabolic fitness of the immune cell and/or enhances one or more anti-tumor activities of the immune cell(s) such that administration of the immune cell(s) decreases tumor burden or increases survival of the subject.

### A. Cancer Therapy

In some aspects, the disclosed methods comprise administering a cancer therapy to a patient. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be an immune checkpoint therapy. Any of these cancer therapies may also be excluded. Combinations of these therapies may also be administered. For example, in some cases, immune cells expressing an antigen-specific receptor of the disclosure can be administered with one or more antibodies or one or more bispecific or multispecific immune cell engagers.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is recurrent cancer. In some aspects, the cancer is Stage I cancer. In some aspects, the cancer is Stage II cancer. In some aspects, the cancer is Stage III cancer. In some aspects, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### 1. Immunotherapy

In some aspects, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

a. Checkpoint Inhibitors and Combination Treatment

Aspects of the disclosure may include administration of immune checkpoint inhibitors, which are further described below.

### (1) PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL 1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### (2) CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### (3) LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1 and has been reported to play a role in Treg suppressive function. LAG3 also helps maintain CD8″ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. Immuno Therapy Cancer, 2016; 4 (Supp. 1): P195 (REGN3767); Yu, X., et al., mAbs, 2019; 11:6 (LBL-007) can be used in the methods disclosed herein. These and other anti-LAG-3 antibodies useful in the claimed disclosure can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another aspect, the antibody has at least about 70, 75, 80, 85, 90, 95, 97, or 99% (or any derivable range therein) variable region amino acid sequence identity with the above-mentioned antibodies.

### (4) TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8⁺ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed disclosure can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another aspect, the antibody has at least about 70, 75, 80, 85, 90, 95, 97, or 99% (or any derivable range therein) variable region amino acid sequence identity with the above-mentioned antibodies.

b. Activation of Co-Stimulatory Molecules

In some aspects, the immunotherapy comprises an activator of a co-stimulatory molecule. In some aspects, the activator comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Activators include agonistic antibodies, polypeptides, compounds, and nucleic acids.

c. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken-down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

d. CAR-T Cell and CAR-NK Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell or NK cell. The receptors are called chimeric because they are fused of parts from different sources. CAR-T cell therapy refers to a treatment that uses such transformed T cells for cancer therapy. CAR-NK cell therapy refers to a treatment that uses such transformed NK cells for cancer therapy.

e. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 2. Chemotherapies

In some aspects, the cancer therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adrenocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, intrapleural, subcutaneous, intratumoral, or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000-fold less than the chemotherapeutic effective dose of the chemotherapeutic agent. For example, the chemotherapeutic agent may be administered in an amount that is about 20-fold less, about 500-fold less or even about 5000-fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### 3. Radiotherapy

In some aspects, the cancer therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### B. Administration of Therapeutic Compositions

The therapeutic agents of the disclosure may be administered by the same route of administration or by different routes of administration. In some aspects, the cancer therapy is administered intravenously, intramuscularly, intrapleurally, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, intrapleurally, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 5 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 UM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intrapleural, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

Proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to, intravenous administration. Alternatively, administration may be by oral, orthotopic, intradermal, intrapleural, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### 1. Combination Therapy

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

In some aspects, the first cancer therapy and the second cancer therapy are administered substantially simultaneously. In some aspects, the first cancer therapy and the second cancer therapy are administered sequentially. In some aspects, the first cancer therapy, the second cancer therapy, and a third therapy are administered sequentially. In some aspects, the first cancer therapy is administered before administering the second cancer therapy. In some aspects, the first cancer therapy is administered after administering the second cancer therapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

### XV. Formulations and Culture of the Cells

In particular aspects, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically defined lipid concentrate (Gibco), and Glutamax (Gibco).

In certain aspects, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine) . . . . In specific aspects, one or more of these may be explicitly excluded.

In some aspects, the medium further comprises vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some aspects, the medium comprises one or more of the following: a B-27® supplement, xeno-free B-27® supplement, GS21™ supplement, or combinations thereof. In some aspects, the medium comprises or further comprises amino acids, monosaccharides, inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific aspects, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific aspects, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular aspects the cells are in a formulated cell suspension that is stable at room temperature for 1, 2, 3, or 4 hours or more from time of thawing.

In particular aspects, the cells of the disclosure comprise an exogenous TCR, which may be of a defined antigen specificity. In some aspects, the TCR can be selected based on absent or reduced alloreactivity to the intended recipient. In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some aspects, the choice of exogenous TCR may not necessarily be defined based on lack of alloreactivity. In some aspects, the endogenous TCR genes have been modified by genome editing so that they do not express a protein. Methods of gene editing such as methods using the CRISPR/Cas9 system are known in the art and described herein.

In some aspects, the cells of the disclosure further comprise one or more chimeric antigen receptors (CARs). Examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRVIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### A. Cells

Certain aspects relate to cells comprising polypeptides or nucleic acids of the disclosure. In some aspects the cell is an immune cell. In some aspects, the cell is a T cell. “T cell” includes all types of immune cells expressing CD3 including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### XVI. General Pharmaceutical Compositions

In some aspects, pharmaceutical compositions are administered to a subject. Different aspects may involve administering an effective amount of a composition to a subject. In some aspects, an antibody or antigen binding fragment capable of binding to an antigen may be administered to the subject to protect against or treat a condition (e.g., cancer). Alternatively, an expression vector encoding one or more such antibodies or polypeptides or peptides and/or one or more viral proteins may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intrapleural, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to, intravenous administration. Alternatively, administration may be by oral, orthotopic, intradermal, intrapleural, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### XVII. Kits

Any of the compositions described herein may be comprised in a kit. In a non-limiting example, cells, reagents to produce cells, vectors, and reagents to produce vectors and/or components thereof may be comprised in a kit. In certain aspects, NK cells may be comprised in a kit, and they may or may not yet express viral protein(s) and/or antigen-targeting receptors, an optional cytokine, or an optional suicide gene. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode one or more viral proteins antibodies, and/or CARs, suicide gene products, and/or cytokines may be included in the kit. Proteins, such as cytokines or antibodies, including monoclonal antibodies, may be included in the kit. Nucleotides that encode components of viral proteins, engineered antibodies, and/or CARs may be included in the kit, including reagents to generate same.

In particular aspects, the kit comprises an NK cell therapy of the disclosure and also another cancer therapy. In some cases, the kit, in addition to the cell therapy aspects, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also may generally contain a second, third or other additional container into which the additional components may be separately placed. However, various combinations of components may be comprised in a vial. The kits of the present disclosure also will typically include a means for containing the composition and any other reagent containers in close confinement for commercial sale. Such containers may include injection or blow-molded plastic containers into which the desired vials are retained.

### EXAMPLES

The following examples are included to demonstrate certain aspects of the disclosure. It should be appreciated by those of skill in the art that the techniques disclosed in the examples which follow represent techniques discovered by the inventor to function well in the practice of the disclosure, and thus can be considered to constitute certain modes for its practice. However, those of skill in the art should, in light of the present disclosure, appreciate that many changes can be made in the specific aspects which are disclosed and still obtain a like or similar result without departing from the spirit and scope of the disclosure.

### Example 1—Using Viral Genes to Enhance the Metabolic Fitness of CAR-NK Cells

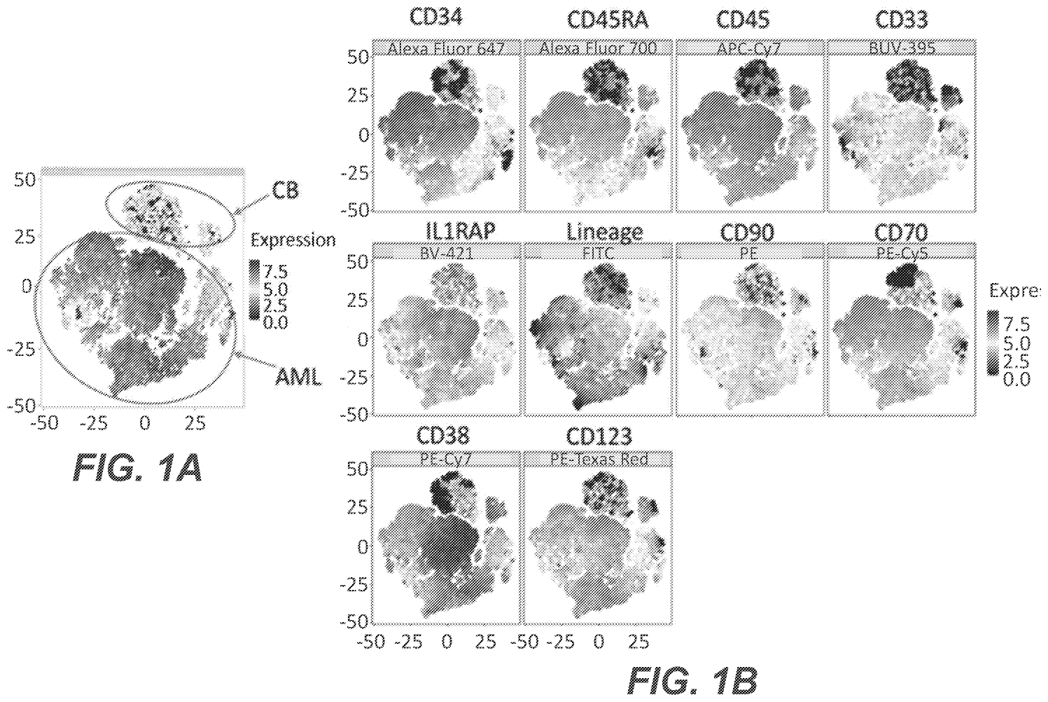

CD70 expression was confirmed on AML patient samples. A Tsne plot from mass cytometry data showed high expression of CD70 on primary AML samples (n=54) but not on healthy CB CD34+ cells (n=10) (FIG. 1A). As shown in FIG. 1B, Tsne plots also showed various myeloid markers on lineage neg cells from CB (upper CB population from FIG. 1A) and AML samples (lower AML population from FIG. 1A). CD70 expression in AML samples was similar to other well-characterized myeloid markers.

To test enhancement of metabolic fitness and anti-leukemic activity of CAR-NK cells into which the viral gene E4ORF-1 has been introduced into the CAR construct, CD70-targeting CAR-NK cells were used and their efficacy tested against CD70 positive AML, with and without E4ORF-1 modification of the CAR construct.

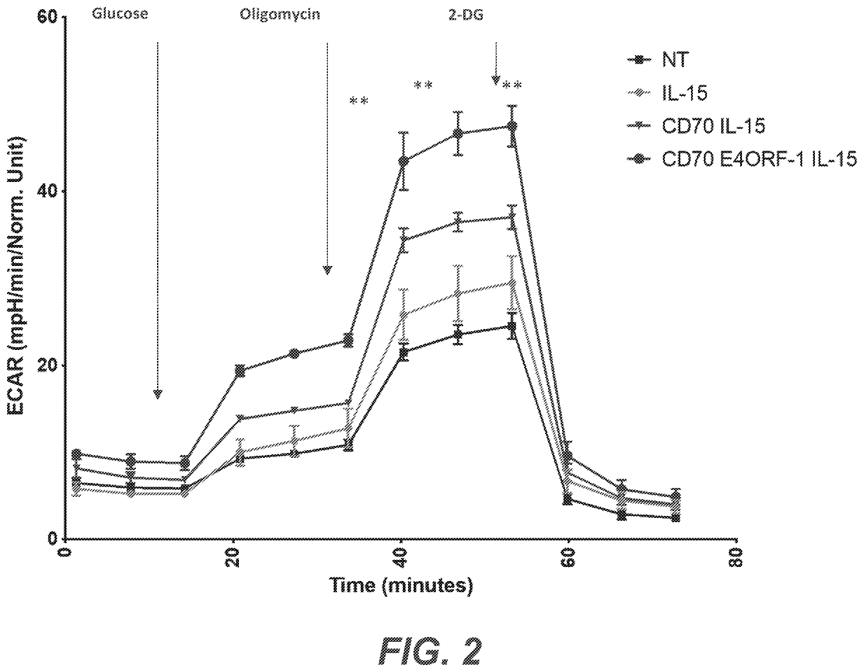

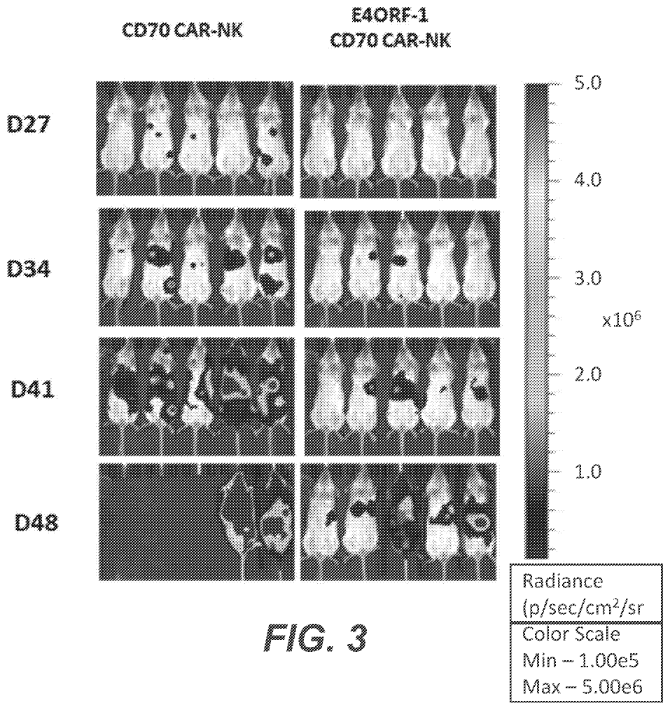

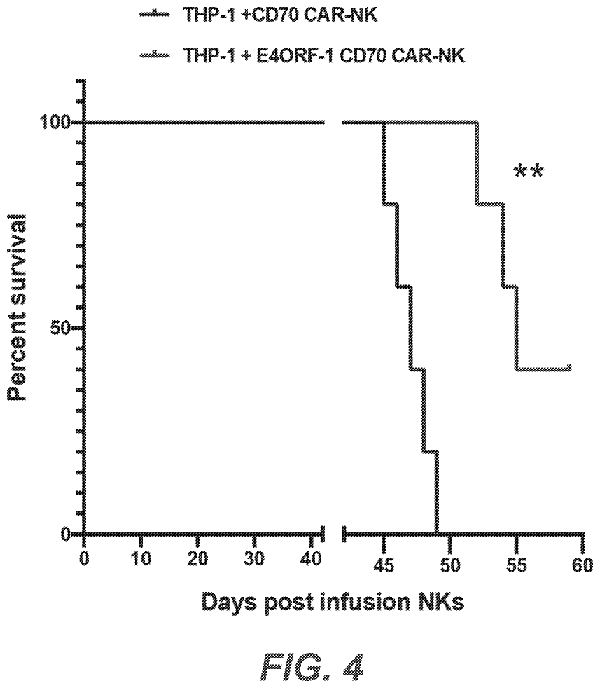

Inserting E4ORF-1 in the CAR construct enhances glycolysis by the CAR-NK cells (FIG. 2). Furthermore, E4ORF-1 enhances CAR-NK cell anti-tumor activity, as shown in an in vivo NSG mouse model of THP-1 AML, where administration of E4ORF-1-modified CAR-NK cells resulted in enhanced tumor control and survival compared to control CAR-NK cells (FIG. 3, FIG. 4).

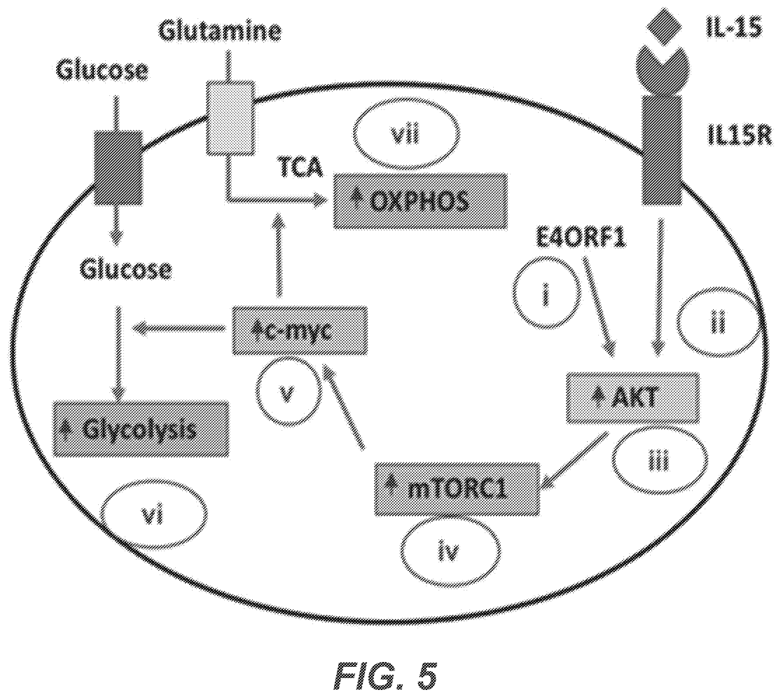

The proposed mechanism by which E4ORF-1 enhances anti-leukemic activity of CAR-NK cells is depicted in the diagram shown in FIG. 5.

In some aspects, the vaccinia virus gene encoding C16, a protein which promotes HIF-1α stabilization through binding to the prolylhydroxylase domain-containing protein (PHD) 2, a cellular oxygen sensor, is also inserted into the CAR construct. In some aspects, inserting E4ORF-1 in the CAR construct enhances glycolysis by the CAR-NK cells. In some aspects, C16 enhances CAR-NK cell anti-tumor activity, as shown, for example, in an in vivo NSG mouse model of THP-1 AML, where in some aspects, administration of C16-modified CAR-NK cells results in enhanced tumor control and survival compared to control CAR-NK cells.

In some aspects, the Dengue virus gene encoding the nonstructural protein 3 (NS3), which recruits fatty acid synthase to enhance fatty acid metabolism, is also inserted into the CAR construct. In some aspects, inserting NS3 in the CAR construct enhances glycolysis by the CAR-NK cells. In some aspects, NS3 enhances CAR-NK cell anti-tumor activity, as shown, for example, in an in vivo NSG mouse model of THP-1 AML, where in some aspects, administration of NS3-modified CAR-NK cells results in enhanced tumor control and survival compared to control CAR-NK cells.

### Example 2—E4ORF-1 Enhances the Glycolytic Potential and Glutamine Metabolism of CAR27/IL-15 NK Cells

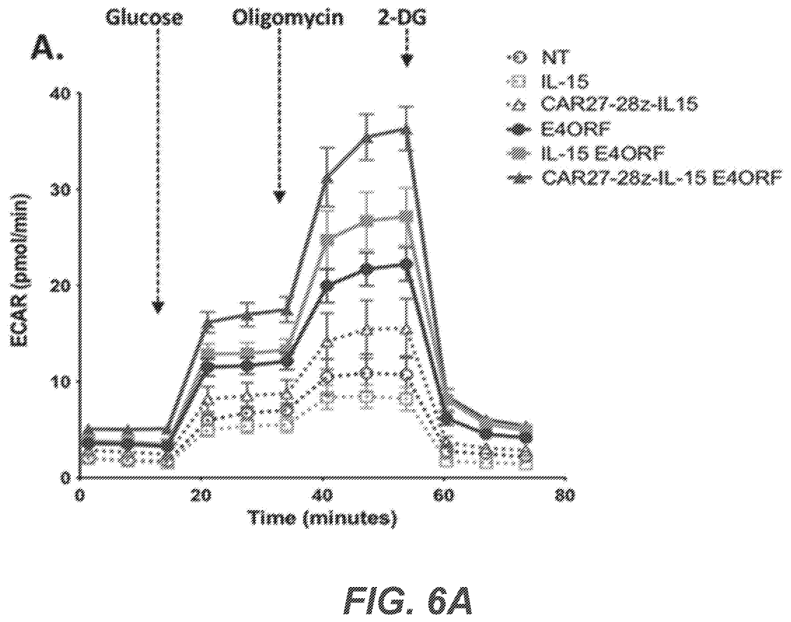

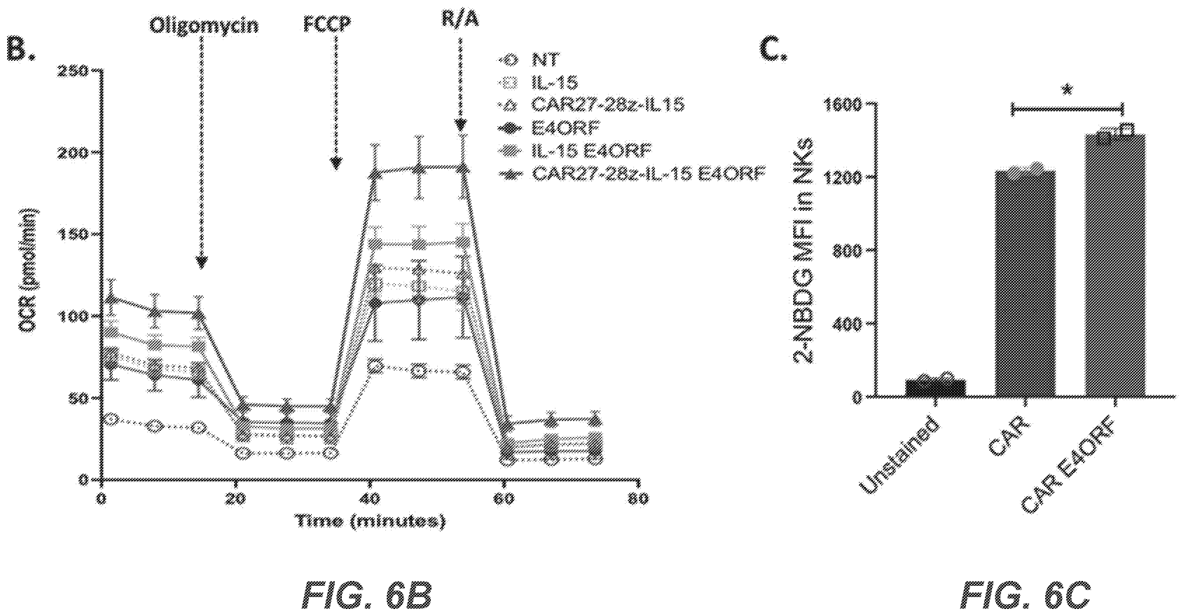

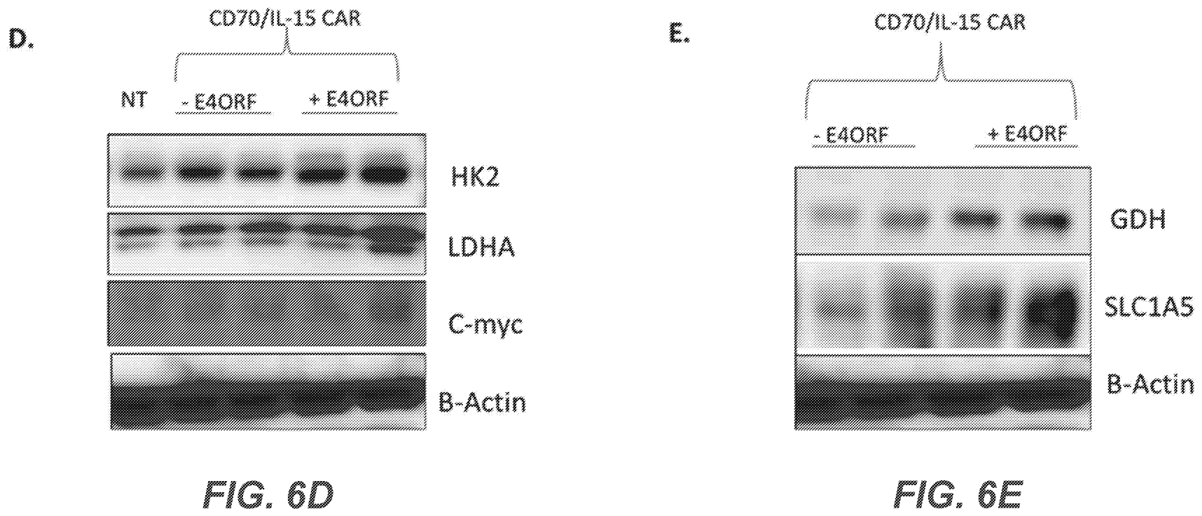

To evaluate the effect of E4ORF-1 on the metabolism of CAR27/IL-15 NK cells, seahorse assays were performed to evaluate their extracellular acidification rate (ECAR), a measure of glycolytic capacity, and their oxygen consumption rate (OCR), a measure of oxidative phosphorylation and mitochondrial function. E4ORF-1-modified CAR27/IL-15 NK cells showed higher glycolytic capacity as evidenced by significantly increased ECAR compared to their respective controls (FIG. 6A). E4ORF-1 modification also enhanced the glycolytic capacity of non-engineered NK cells and NK cells engineered to express IL-15 alone (without the CAR construct) (FIG. 6B). The same trend was observed for OXPHOS as shown by increased OCR in E4ORF-1 expressing NK cells (FIG. 6B). Using a flow cytometry assay after incubation with the fluorescent glucose analog 2-NBDG (Cayman chemicals) for 30 min at 37 degrees, glucose uptake was found to be significantly enhanced in E4ORF-1-expressing CAR27/IL-15 CAR-NK cells compared to control CAR27/IL-15 CAR-NK cells (FIG. 6C).

To further evaluate the effect of E4ORF-1 on the glycolytic capacity of CAR27/IL-15 NK cells, non-transduced (NT) NK cells, E4ORF-1-expressing or control CAR27/IL-15 NK cells were lysed using RIPA buffer supplemented with protease inhibitor and phosphatase inhibitor to prevent protein degradation and dephosphorylation respectively. The protein lysate was used to perform western blot to evaluate the activity of rate limiting enzymes involved in glycolysis (hexokinase (HK-2), lactate dehydrogenase (LDHA)) and c-myc content (cell signaling technology). E4ORF-1-expressing CAR27/IL-15 NK cells showed significantly enhanced HK-2 and LDHA activity and a higher c-myc content compared to control CAR27/IL-15 NK cells (FIG. 6D).

Since adenovirus-infected cells also acquire higher glutamine metabolism through the action of E4ORF-1, glutamine metabolism in E4ORF-1-expressing CAR27/IL-15 NK cells was also evaluated by western blot, and glutamine metabolism was found to be significantly enhanced in E4ORF-1-expressing CAR27/IL-15 NK cells compared to control CAR27/IL-15 NK cells, as evidenced by increased expression of glutamate dehydrogenase (GDH), the enzyme that catalyzes the conversion of glutamate to α-ketoglutarate, and increased expression of SLC1A5, a glutamine transporter (FIG. 6E).

These data confirm that, in some aspects, introducing E4ORF-1 in NK cells (either non-engineered NK cells or NK cells engineered to express IL-15 or CAR/IL-15) can enhance metabolic fitness of the cells by boosting their ability to uptake glucose, perform aerobic glycolysis, and metabolize glutamine, thereby increasing their capacity to metabolically compete with the tumor.

### Example 3—E40RF-1 Enhances the Anti-Tumor Activity of CAR27/IL-15 NK Cells

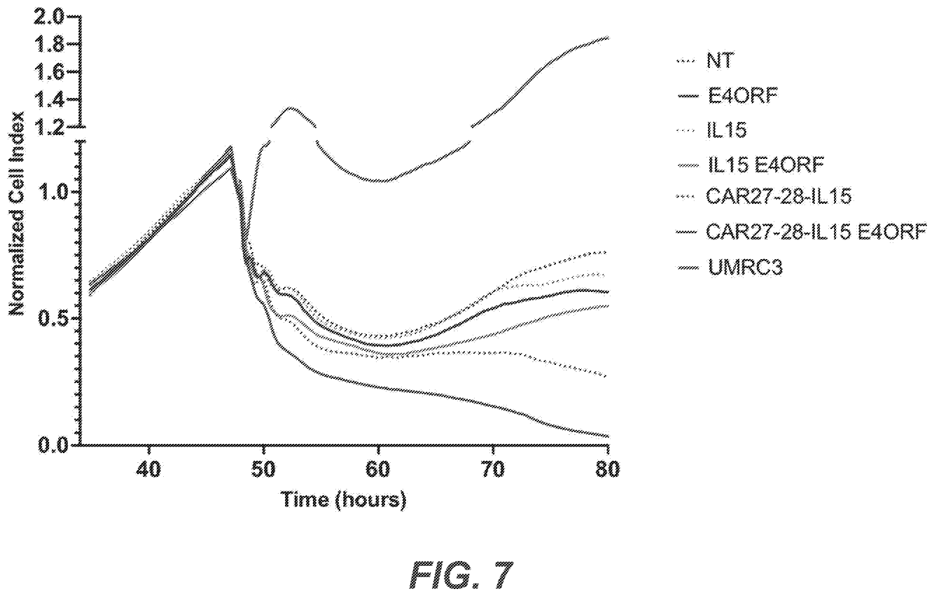

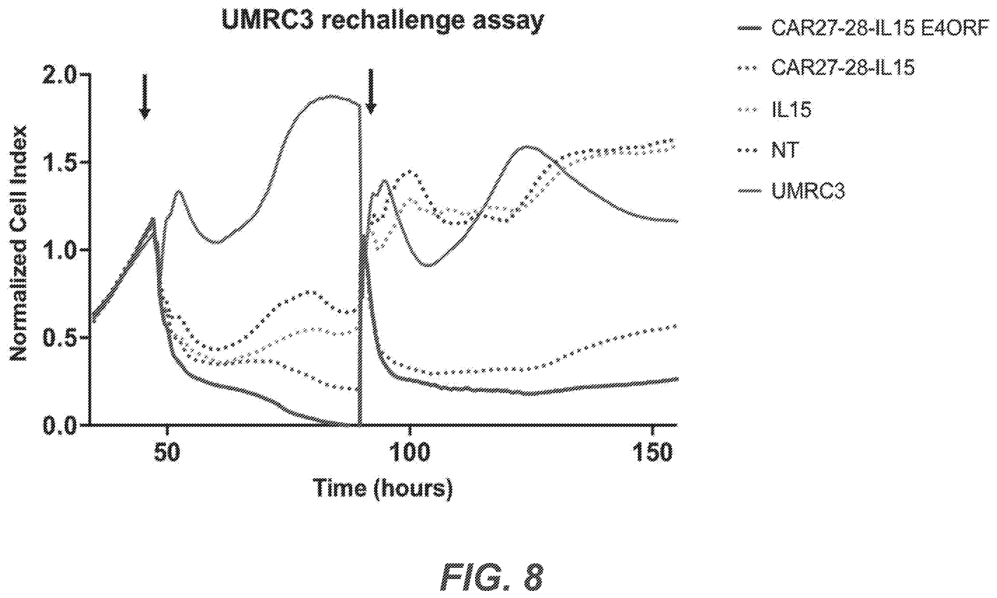

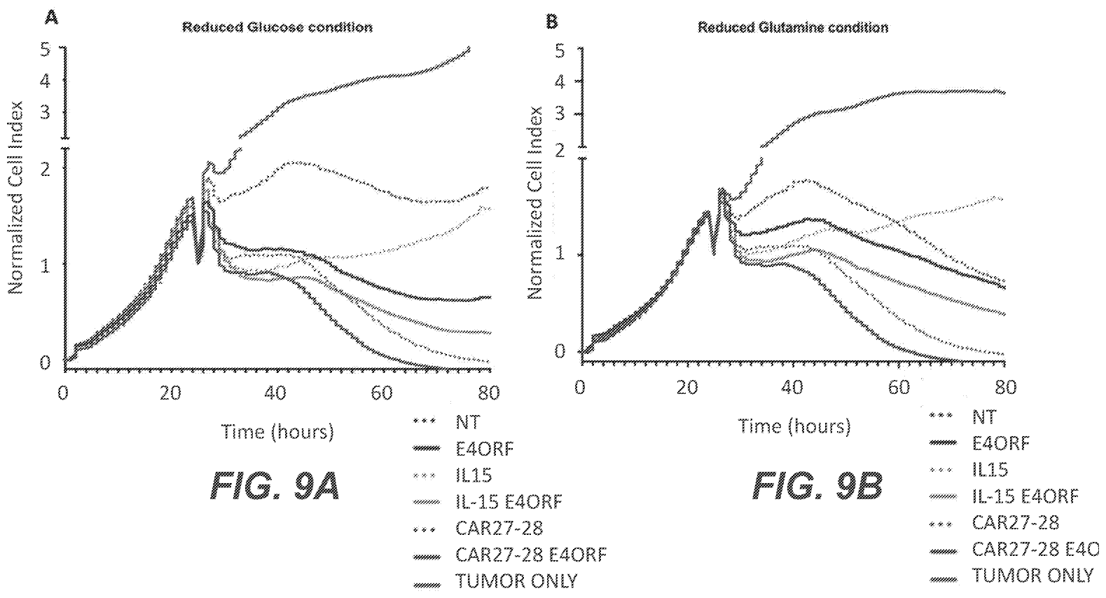

To test the in vitro anti-tumor activity of E4ORF-1-expressing NK cells, including non-engineered, IL-15-engineered and CAR27/IL-15-engineered NK cells, XCELLIGENCE® cytotoxicity assays were performed against different tumor cells. E4ORF-1 enhances the anti-tumor activity of NK cells (non-engineered, IL-15-engineered and CAR27/IL-15-engineered) against a renal cell carcinoma cell line (UMRC3) (FIG. 7). The advantage conferred by E4ORF-1 persists in CAR27/IL-15-engineered NK cells after a second tumor rechallenge (FIG. 8). The advantage provided by E4ORF-1 is demonstrated even more clearly when the NK cells are co-cultured with tumor cells (pancreatic tumor cell line Panc1) under reduced nutrient conditions (low glucose or low glutamine concentration in the culture media) (FIG. 9). This corroborates the fact that, in some aspects, E4ORF-1 enhances the ability of NK cells to metabolically compete with the tumor cells for nutrients by boosting their ability to consume and metabolize glucose and glutamine.

All of the methods disclosed and claimed herein can be made and executed without undue experimentation in light of the present disclosure. While the compositions and methods of this disclosure have been described in terms of preferred aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references and those cited elsewhere herein, to the extent that they provide exemplary procedural or other details supplementary to those set forth herein, are specifically incorporated herein by reference.
1. Donnelly R P, Loftus R M, Keating S E, et al. mTORC1-dependent metabolic reprogramming is a prerequisite for N K cell effector function. J Immunol. 2014; 193 (9): 4477-4484.
2. Finlay D K. Metabolic regulation of natural killer cells. Biochem Soc Trans. 2015; 43 (4): 758-762.
3. Cong J, Wang X, Zheng X, et al. Dysfunction of Natural Killer Cells by FBP1-Induced Inhibition of Glycolysis during Lung Cancer Progression. Cell Metab. 2018; 28 (2): 243-255 e245.
4. Isaacson B, Mandelboim O. Sweet Killers: NK Cells Need Glycolysis to Kill Tumors. Cell Metab. 2018; 28 (2): 183-184.
5. van der Windt G J, Pearce E L. Metabolic switching and fuel choice during T-cell differentiation and memory development. Immunol Rev. 2012; 249 (1): 27-42.
6. Delgoffe G M. Filling the Tank: Keeping Antitumor T Cells Metabolically Fit for the Long Haul. Cancer Immunol Res. 2016; 4 (12): 1001-1006.
7. Daher M, Basar R, Gokdemir E, et al. Targeting a cytokine checkpoint enhances the fitness of armored cord blood CAR-NK cells. Blood. 2020.
8 Thaker S K, Ch′ng J, Christofk H R. Viral hijacking of cellular metabolism. BMC Biol. 2019; 17 (1): 59.
9. Thai M, Graham N A, Braas D, et al. Adenovirus E4ORF1-induced MYC activation promotes host cell anabolic glucose metabolism and virus replication. Cell Metab. 2014; 19 (4): 694-701.
10. Rogers et al. Human adenovirus Ad-36 induces adipogenesis via its E4orf-1 gene. Int J Obes (Lond). 2008 March; 32 (3): 397-406.

What is claimed is:

1. One or more polynucleotides encoding one or more viral, bacterial, and/or fungal genes capable of manipulating cell metabolism and one or more antigen-specific receptors.

2. The one or more polynucleotides of claim 1, wherein the one or more viral, bacterial, and/or fungal genes are capable of increasing glycolysis, oxidative phosphorylation, fatty acid synthesis, glutaminolysis, or a combination thereof in a cell.

3. The one or more polynucleotides of claim 1 or claim 2, wherein the one or more viral, bacterial, and/or fungal genes comprise an adenovirus, vaccinia virus, hepatitis C virus (HCV), hepatitis B virus (HBV), Epstein-Barr virus (EBV), and/or Dengue virus (DENV) gene.

4. The one or more polynucleotides of claim 3, wherein the adenovirus gene comprises E4ORF-1.

5. The one or more polynucleotides of claim 3 or claim 4, wherein the vaccinia virus gene comprises C16.

6. The one or more polynucleotides of any of claims 3-5, wherein the DENV gene comprises NS3.

7. The one or more polynucleotides of any of claims 3-6, wherein the HCV gene comprises NS5A.

8. The one or more polynucleotides of any of claims 3-7, wherein the HBV gene comprises ORFx.

9. The one or more polynucleotides of any of claims 3-8, wherein the EBV gene comprises LMP1.

10. The one or more polynucleotides of any of claims 3-9, wherein the one or more viral genes and one or more antigen-specific receptors are encoded by the same polynucleotide.

11. The one or more polynucleotides of any of claims 3-9, wherein the one or more viral genes and one or more antigen-specific receptors are encoded by different polynucleotides.

12. The one or more polynucleotides of any one of claims 1-9, wherein the one or more antigen-specific receptors each comprise:
(a) one or more antigen binding regions;
(b) a transmembrane domain; and
(c) one or more intracellular domains.

13. The one or more polynucleotides of claim 12, wherein the antigen binding region comprises a linker.

14. The one or more polynucleotides of claim 12 or claim 13, wherein the transmembrane domain is a transmembrane domain from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, DAP10, or DAP12.

15. The one or more polynucleotides of any one of claims 12-14, wherein the transmembrane domain is a CD28 transmembrane domain.

16. The one or more polynucleotides of any one of claims 12-15, wherein the intracellular domain is an intracellular domain from CD3 zeta, CD27, CD28, 4-1BB, DAP12, NKG2D, OX-40 (CD134), DAP10, CD40L, 2B4, DNAM, CS1, CD48, NKp30, NKp44, NKp46, or NKp80.

17. The one or more polynucleotides of any one of claims 12-16, wherein the intracellular domain is a CD28 intracellular domain.

18. The one or more polynucleotides of any one of claims 12-16, wherein the intracellular domain is a CD3 zeta intracellular domain.

19. The one or more polynucleotides of any of claims 12-18, wherein the one or more antigen-specific receptors comprise two or more intracellular domains.

20. The one or more polynucleotides of claim 19, wherein the two or more intracellular domains comprise a CD3 zeta intracellular domain and an additional intracellular domain selected from a CD28, DAP10, DAP12, 4-1BB, NKG2D, ICOS, and 2B4 intracellular domain.

21. The one or more polynucleotides of claim 20, wherein the two or more intracellular domains comprise a CD3 zeta intracellular domain and a CD28 intracellular domain.

22. The one or more polynucleotides of any of claims 1-21, wherein the one or more antigen-specific receptors further comprise a hinge between the antigen binding domain and the transmembrane domain.

23. The one or more polynucleotides of claim 22, wherein the hinge is an IgG hinge, a CD28 hinge, or a CD8a hinge.

24. The one or more polynucleotides of claim 22 or 23, wherein the hinge is an IgG1 hinge, IgG2 hinge, IgG3 hinge, or IgG4 hinge.

25. The one or more polynucleotides of any of claims 22-24, wherein the hinge is an IgG1 hinge.

26. The one or more polynucleotides of claim 22 or 23, wherein the hinge is a CD28 hinge.

27. The one or more polynucleotides of any of claims 1-26, wherein the one or more polynucleotides further encode a signal peptide.

28. The one or more polynucleotides of claim 27, wherein the signal peptide is a signal peptide from CD8, CD27, granulocyte-macrophage colony-stimulating factor receptor (GMSCF-R), Ig heavy chain, a killer cell immunoglobulin-like receptor (KIR), CD3, or CD4.

29. The one or more polynucleotides of claim 27 or claim 28, wherein the signal peptide is a CD8 signal peptide.

30. The one or more polynucleotides of any of claims 1-29, wherein the one or more polynucleotides further encode an additional polypeptide.

31. The one or more polynucleotides of claim 30, wherein the additional polypeptide is a therapeutic protein or a protein that enhances cell activity, expansion, and/or persistence.

32. The one or more polynucleotides of claim 30 or 31, wherein the additional polypeptide is a suicide gene, a cytokine, or a human or viral protein that enhances proliferation, expansion and/or metabolic fitness.

33. The one or more polynucleotides of any of claims 30-32, wherein the additional polypeptide is a cytokine.

34. The one or more polynucleotides of claim 33, wherein the cytokine is IL-15, IL-2, IL-12, IL-18, IL-21, IL-23, or IL-7.

35. The one or more polynucleotides of claim 33 or claim 34, wherein the cytokine is IL-15.

36. The one or more polynucleotides of claim 33 or claim 34, wherein the cytokine is IL-21.

37. The one or more polynucleotides of claim 33 or claim 34, wherein the cytokine is IL-12.

38. The one or more polynucleotides of any of claims 1-37, wherein the one or more antigen-specific engineered receptors comprise a chimeric antigen receptor (CAR).

39. The one or more polynucleotides of any of claims 1-35, wherein the one or more antigen-specific engineered receptors comprise a T-cell receptor (TCR).

40. The one or more polynucleotides of any of claims 1-39, wherein the one or more antigen-specific engineered receptors bind one or more antigens comprising 5T4, 8H9, αvβ6 integrin, BCMA, B7-H3, B7-H6, CAIX, CA9, CD19, CD20, CD22, CD30, CD33, CD3δ, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors.

41. The one or more polynucleotides of any of claims 1-40, wherein the one or more antigen-specific engineered receptors bind one or more antigens comprising CD70, CD5, CD19, CD22, BCMA, CS1, CD123, CD3δ, CLL-1, CD97, and/or HLA-G.

42. The one or more polynucleotides of any of claims 1-41, wherein the one or more antigen-specific engineered receptors bind CD70.

43. A vector comprising the polynucleotide of any one of claims 1-42.

44. The vector of claim 43, wherein the vector is a viral vector.

45. The vector of claim 44, wherein the viral vector is an adenoviral vector, adeno-associated viral vector, lentiviral vector, or retroviral vector.

46. The vector of claim 43, wherein the vector is a non-viral vector.

47. The vector of claim 46, wherein the non-viral vector is a plasmid.

48. An immune cell comprising the polynucleotide of any one of claims 1-39 or the vector of any one of claims 43-47.

49. The immune cell of claim 48, wherein the immune cell is a natural killer (NK) cell, T cell, gamma delta T cell, alpha beta T cell, invariant NKT (INKT) cell, B cell, macrophage, mesenchymal stromal cell, or dendritic cell.

50. The immune cell of claim 49, wherein the immune cell is an NK cell.

51. The immune cell of claim 50, wherein the NK cell is derived from cord blood, peripheral blood, induced pluripotent stem cells, hematopoietic stem cells, bone marrow, or from a cell line.

52. The immune cell of claim 51, wherein the NK cell is derived from a cell line, wherein the NK cell line is NK-92.

53. The immune cell of claim 51, wherein the NK cell is derived from a cord blood mononuclear cell.

54. The immune cell of any of claims 50-53, wherein the NK cell is a CD56⁺ NK cell.

55. The immune cell of any of claims 50-54, wherein the NK cell expresses a recombinant cytokine.

56. The immune cell of claim 55, wherein the cytokine is IL-15, IL-2, IL-12, IL-18, IL-21, IL-7, or IL-23.

57. The immune cell of claim 56, wherein the cytokine is IL-15.

58. The immune cell of claim 56, wherein the cytokine is IL-21.

59. The immune cell of claim 56, wherein the cytokine is IL-12.

60. The immune cell of any of claims 48-59, wherein expression by the immune cell of the one or more viral, bacterial, and/or fungal genes capable of manipulating cell metabolism and/or the one or more antigen-specific receptors encoded by the polynucleotide enhances the metabolic fitness of the immune cell and/or enhances one or more anti-tumor activities of the immune cell.

61. The immune cell of claim 60, wherein the metabolism of the immune cell is increased compared to an immune cell into which the polynucleotide encoding one or more viral, bacterial, and/or fungal genes capable of manipulating cell metabolism has not been introduced.

62. The immune cell of claim 61, wherein glycolysis, oxidative phosphorylation, fatty acid synthesis, glutaminolysis, or a combination thereof is increased by the immune cell.

63. The immune cell of claim 61 or claim 62, wherein glycolysis is increased by the immune cell.

64. A population of immune cells comprising the immune cell of any one of claims 48-63.

65. An immune cell comprising a polynucleotide encoding one or more viral, bacterial, and/or fungal genes capable of manipulating cell metabolism.

66. The immune cell of claim 65, wherein the immune cell is a natural killer (NK) cell, T cell, gamma delta T cell, alpha beta T cell, invariant NKT (INKT) cell, B cell, macrophage, mesenchymal stromal cell, or dendritic cell.

67. The immune cell of claim 66, wherein the immune cell is an NK cell.

68. The immune cell of claim 67, wherein the NK cell is derived from cord blood, peripheral blood, induced pluripotent stem cells, hematopoietic stem cells, bone marrow, or from a cell line.

69. The immune cell of claim 68, wherein the NK cell is derived from a cell line, wherein the NK cell line is NK-92.

70. The immune cell of claim 69, wherein the NK cell is derived from a cord blood mononuclear cell.

71. The immune cell of any of claims 67-70, wherein the NK cell is a CD56⁺ NK cell.

72. The immune cell of any of claims 67-71, wherein the NK cell expresses a recombinant cytokine.

73. The immune cell of claim 72, wherein the cytokine is IL-15, IL-2, IL-12, IL-18, IL-21, IL-7, or IL-23.

74. The immune cell of claim 73, wherein the cytokine is IL-15.

75. The immune cell of claim 73, wherein the cytokine is IL-21.

76. The immune cell of claim 73, wherein the cytokine is IL-12.

77. The immune cell of any of claims 65-76, wherein the one or more viral, bacterial, and/or fungal genes comprise an adenovirus, vaccinia virus, HCV, HBV, and/or DENV gene.

78. The immune cell of claim 77, wherein the adenovirus gene comprises E4ORF-1.

79. The immune cell of claim 77 or 78, wherein the vaccinia virus gene comprises C16.

80. The immune cell of any of claims 77-79, wherein the DENV gene comprises NS3.

81. The immune cell of any of claims 77-80, wherein the HCV gene comprises NS5A.

82. The immune cell of any of claims 77-81, wherein the HBV gene comprises ORFx.

83. The immune cell of any of claims 77-82, wherein the EBV gene comprises LMP1.

84. The immune cell of any of claims 65-83, wherein the polynucleotide encoding one or more viral, bacterial, and/or fungal genes capable of manipulating cell metabolism is comprised in a vector.

85. The immune cell of claim 84, wherein the vector is a viral vector.

86. The immune cell of claim 85, wherein the viral vector is an adenoviral vector, adeno-associated viral vector, lentiviral vector, or retroviral vector.

87. The immune cell of claim 84, wherein the vector is a non-viral vector.

88. The immune cell of claim 87, wherein the non-viral vector is a plasmid.

89. The immune cell of any of claims 65-88, wherein expression by the immune cell of the one or more viral, bacterial, and/or fungal genes capable of manipulating cell metabolism encoded by the polynucleotide enhances the metabolic fitness of the immune cell.

90. The immune cell of claim 89, wherein the metabolism of the immune cell is increased compared to an immune cell into which the polynucleotide encoding one or more viral, bacterial, and/or fungal genes capable of manipulating cell metabolism has not been introduced.

91. The immune cell of claim 90, wherein glycolysis, oxidative phosphorylation, fatty acid synthesis, glutaminolysis, or a combination thereof is increased by the immune cell.

92. The immune cell of claim 90 or claim 91, wherein glycolysis is increased by the immune cell.

93. A population of immune cells comprising the immune cell of any one of claims 65-92.

94. A pharmaceutical composition comprising:
(a) the immune cell of any of claims 48-57, the population of immune cells of claim 64, the immune cell of any of claims 65-88, or the population of immune cells of claim 93; and
(b) a pharmaceutically acceptable excipient.

95. The pharmaceutical composition of claim 94, further comprising an additional therapeutic.

96. The pharmaceutical composition of claim 95, wherein the additional therapeutic is a chemotherapeutic.

97. A method for treating a subject for cancer, the method comprising administering to the subject a therapeutically effective amount of the immune cell of any of claims 48-57, the population of immune cells of claim 64, the immune cell of any of claims 65-88, or the population of immune cells of claim 93, or the pharmaceutical composition of any of claims 94-96.

98. The method of claim 97, wherein administration of a therapeutically effective amount of the immune cell of any of claims 48-57, the population of immune cells of claim 64, the immune cell of any of claims 65-88, or the population of immune cells of claim 93, or the pharmaceutical composition of any of claims 94-96 decreases tumor burden or increases survival of the subject.

99. The method of claim 97 or claim 98, wherein the subject has lymphoma, leukemia, glioblastoma, melanoma, non-small cell lung cancer, renal cell carcinoma, pancreatic cancer, ovarian cancer, or breast cancer.

100. The method of claim 97 or claim 99, further comprising administering to the subject an additional therapy.

101. The method of claim 100, wherein the additional therapy is radiotherapy, chemotherapy, or immunotherapy.

102. A method for enhancing the metabolic fitness of an immune cell, the method comprising introducing into the immune cell a polynucleotide encoding one or more viral, bacterial, and/or fungal genes capable of manipulating cell metabolism, wherein the metabolism of the immune cell is increased compared to an immune cell into which the polynucleotide encoding one or more viral, bacterial, and/or fungal genes capable of manipulating cell metabolism has not been introduced.

103. The method of claim 102, wherein glycolysis, oxidative phosphorylation, fatty acid synthesis, glutaminolysis, or a combination thereof is increased by the immune cell.

104. The method of claim 102 or claim 103, wherein glycolysis is increased by the immune cell.

105. The method of any of claims 102-104, wherein the one or more viral, bacterial, and/or fungal genes comprise an adenovirus, vaccinia virus, HCV, HBV, and/or DENV gene.

106. The method of claim 105, wherein the adenovirus gene comprises E4ORF-1.

107. The method of claim 105 or 106, wherein the vaccinia virus gene comprises C16.

108. The method of any of claims 105-107, wherein the DENV gene comprises NS3.

109. The method of any of claims 105-108, wherein the HCV gene comprises NS5A.

110. The method of any of claims 105-109, wherein the HBV gene comprises ORFx.

111. The method of any of claims 105-110, wherein the EBV gene comprises LMP1.

112. The method of any of claims 102-108, further comprising introducing into the immune cell a polynucleotide encoding one or more antigen-specific engineered receptors.

113. The method of claim 112, wherein the one or more viral, bacterial, and/or fungal genes and one or more antigen-specific receptors are encoded by the same polynucleotide.

114. The method of claim 112, wherein the one or more viral, bacterial, and/or fungal genes and one or more antigen-specific receptors are encoded by different polynucleotides.

115. The method of any one of claims 112-114, wherein the one or more antigen-specific receptors each comprise:
(a) one or more antigen binding regions;
(b) a transmembrane domain; and
(c) one or more intracellular domains.

116. The method of claim 115, wherein the antigen binding region comprises a linker.

117. The method of claim 115 or claim 116, wherein the transmembrane domain is a transmembrane domain from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, DAP10, or DAP12.

118. The method of any one of claims 115-117, wherein the transmembrane domain is a CD28 transmembrane domain.

119. The method of any one of claims 115-118, wherein the intracellular domain is an intracellular domain from CD3 zeta, CD27, CD28, 4-1BB, DAP12, NKG2D, OX-40 (CD134), DAP10, CD40L, 2B4, DNAM, CS1, CD48, NKp30, NKp44, NKp46, or NKp80.

120. The method of any one of claims 115-119, wherein the intracellular domain is a CD28 intracellular domain.

121. The method of any one of claims 115-119, wherein the intracellular domain is a CD3 zeta intracellular domain.

122. The method of any of claims 115-121, wherein the one or more antigen-specific receptors comprise two or more intracellular domains.

123. The method of claim 122, wherein the two or more intracellular domains comprise a CD3 zeta intracellular domain and an additional intracellular domain selected from a CD28, DAP10, DAP12, 4-1BB, NKG2D, ICOS, and 2B4 intracellular domain.

124. The method of claim 123, wherein the two or more intracellular domains comprise a CD3 zeta intracellular domain and a CD28 intracellular domain.

125. The method of any of claims 115-124, wherein the one or more antigen-specific receptors further comprise a hinge between the antigen binding domain and the transmembrane domain.

126. The method of claim 125, wherein the hinge is an IgG hinge, a CD28 hinge, or a CD8a hinge.

127. The method of claim 125 or 126, wherein the hinge is IgG1 hinge, IgG2 hinge, IgG3 hinge, or IgG4 hinge.

128. The method of any of claims 125-127, wherein the hinge is an IgG1 hinge.

129. The one or more polynucleotides of claim 125 or 126, wherein the hinge is a CD28 hinge.

130. The method of any of claims 112-129, wherein the polynucleotide encoding the one or more antigen-specific receptors further encodes a signal peptide.

131. The method of claim 130, wherein the signal peptide is a signal peptide from CD8, CD27, granulocyte-macrophage colony-stimulating factor receptor (GMSCF-R), Ig heavy chain, a killer cell immunoglobulin-like receptor (KIR), CD3, or CD4.

132. The method of claim 130 or claim 131, wherein the signal peptide is a CD8 signal peptide.

133. The method of any of claims 112-132, wherein the polynucleotide encoding the one or more antigen-specific receptors further encodes an additional polypeptide.

134. The method of claim 133, wherein the additional polypeptide is a therapeutic protein or a protein that enhances cell activity, expansion, and/or persistence.

135. The method of claim 133 or 134, wherein the additional polypeptide is a suicide gene, a cytokine, or a human or viral protein that enhances proliferation, expansion and/or metabolic fitness.

136. The method of any of claims 133-135, wherein the additional polypeptide is a cytokine.

137. The method of claim 136, wherein the cytokine is IL-15, IL-2, IL-12, IL-18, IL-21, IL-23, or IL-7.

138. The method of claim 136 or claim 137, wherein the cytokine is IL-15.

139. The method of claim 136 or claim 137, wherein the cytokine is IL-21.

140. The method of claim 136 or claim 137, wherein the cytokine is IL-12.

141. The method of any of claims 112-140, wherein the one or more antigen-specific engineered receptors comprise a chimeric antigen receptor (CAR).

142. The method of any of claims 112-138, wherein the one or more antigen-specific engineered receptors comprise a T-cell receptor (TCR).

143. The method of any of claims 112-142, wherein the one or more antigen-specific engineered receptors bind one or more antigens comprising 5T4, 8H9, αvβ6 integrin, BCMA, B7-H3, B7-H6, CAIX, CA9, CD19, CD20, CD22, CD30, CD33, CD3δ, CD44, CD44v6, CD44v7/8, CD70, CD123, CD138, CD171, CEA, CSPG4, EGFR, EGFR family including ErbB2 (HER2), EGFRVIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors.

144. The polynucleotide of any of claims 112-143, wherein the one or more antigen-specific engineered receptors bind one or more antigens comprising CD70, CD5, CD19, CD22, BCMA, CS1, CD123, CD3δ, CLL-1, CD97, and/or HLA-G.

145. The method of any of claims 112-144, wherein the one or more antigen-specific engineered receptors bind CD70.

146. The method of any one of claims 102-145, wherein the polynucleotide encoding one or more viral, bacterial, and/or fungal genes capable of manipulating cell metabolism is comprised in a vector.

147. The method of claim 146, wherein the vector is a viral vector.

148. The method of claim 147, wherein the viral vector is an adenoviral vector, adeno-associated viral vector, lentiviral vector, or retroviral vector.

149. The method of claim 146, wherein the vector is a non-viral vector.

150. The method of claim 149, wherein the non-viral vector is a plasmid.

151. The method of any of claims 102-150, further comprising administering a therapeutically effective amount of the immune cells having enhanced metabolic fitness or a pharmaceutical composition comprising the immune cells having enhanced metabolic fitness and a pharmaceutically acceptable excipient to a subject having cancer.

152. The method of claim 151, wherein the pharmaceutical composition further comprises an additional therapeutic.

153. The method of claim 152, wherein the additional therapeutic is a chemotherapeutic.

154. The method of any of claims 151-153, wherein administration of a therapeutically effective amount of the immune cells having enhanced metabolic fitness or the pharmaceutical composition comprising the immune cells having enhanced metabolic fitness and a pharmaceutically acceptable excipient decreases tumor burden or increases survival of the subject.

155. The method of any of claims 151-154, wherein the subject has lymphoma, leukemia, glioblastoma, melanoma, non-small cell lung cancer, renal cell carcinoma, pancreatic cancer, ovarian cancer, or breast cancer.

156. The method of any of claims 151-155, further comprising administering to the subject an additional therapy.

157. The method of claim 156, wherein the additional therapy is radiotherapy, chemotherapy, or immunotherapy.


SEQUENCE LISTING (97 sequences)

SEQ ID NO: 1 - DNA, 378 bp, Adenovirus
  source 1..378: mol_type = genomic DNA
atggctgaatctctgtatgctttcatagatagccctggagggatcgctcccgtccaggaaggggctagcaatagatatatcttcttttgccccgaatctttccacattcctccgcatggggtgatattgcttcacctcagagtgagcgtgctggttcctactggatatcagggcagatttatggccttgaatgactaccatgccaggggcatactaacccagtccgatgtgatatttgccgggagaagacatgatctctctgtgctgctctttaaccacacggaccgatttttgtatgtccgcgagggccacccagtgggaaccctgctgctggagagagtgatttttccttcagtgagaatagccaccctggtttag

SEQ ID NO: 2 - DNA, 546 bp, Vaccinia virus
  source 1..546: mol_type = genomic DNA
atgatatcactctcattcctcatccacaacccattaaaaaaatggaaattaaagccctctattagcataaacggctacaggtctacctttacaatggcctttccttgtgcccagttcagaccctgtcattgccacgctactaaggactccctgaataccgtggccgacgtcagacattgtctgactgaatacatcctgtgggtttctcatagatggacccatagagaaagcgcagggtctctctacaggcttctcatctctttcagaactgatgcaacggagctctttggtggtgagttgaaggattcacttccgtgggacaatatcgacaattgcgtggagatcattaaatgtttcatcagaaatgactccatgaaaaccgccgaagaacttcgtgcaatcattggactttgtactcaatcagctatcgtctctggaagagtcttcaacgataagtatatcgacatactacttatgctgcgaaagattctgaacgagaacgactatctcaccctcttggatcatatccgcactgctaaatactaa

SEQ ID NO: 3 - DNA, 558 bp, Dengue virus
  source 1..558: mol_type = genomic DNA
tcaggagtgctatgggacacacccagccctccagaagtggaaagagcagtccttgacaatggcatctatagaattctccaaagaggattgttgggcaggtctcaagtaggagtaggagtttttcaagaaggtgtgttccacacaatgtggcacgtcaccaggggagctgttctcatgtaccaagggaagagattggaaccaagttgggctagtgtcaagaaagacttgatctcatatggaggaggttggagatttcaaggatcctggaacacgggagaagaggtgcaggtgattgctgttgaaccggggaagaaccccaaaaatgtacagacaacgccaggcaccttcaagacccctgaaggtgaaattggagctatagctctagacttcaaacccggcacatctggatctcctatcgtgaacagagagggaaaaatagtgggtctttatggaaatggagtggtgacaacaagtggaacctatgtcagtgccatagctcaagctaaagtatcacaagaagggcccctaccagagattgaggacgaggtgtttaggaaa

SEQ ID NO: 4 - DNA, 816 bp, synthetic construct
  misc_feature 1..816: note = Synthetic Polynucleotide
  source 1..816: mol_type = other DNA
atggccctgcctgtgacagctctgctcctccctctggccctgctgctccatgccgccagaccccaggcagttgtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtggcagcaccagcggctccggcaagcctggctctggcgagggcagcacaaagggagaggtgcagctcgtggagtctgggggaggcttggtgcagcctggggggtctctgagactctcctgtgcagcctctggattcaccttcagtgtctactacatgaactgggtccgccaggctccagggaaggggctcgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctca

SEQ ID NO: 5 - DNA, 816 bp, synthetic construct
  misc_feature 1..816: note = Synthetic Polynucleotide
  source 1..816: mol_type = other DNA
atggccctgcctgtgacagctctgctcctccctctggccctgctgctccatgccgccagacccgaggtgcagctcgtggagtctgggggaggcttggtgcagcctggggggtctctgagactctcctgtgcagcctctggattcaccttcagtgtctactacatgaactgggtccgccaggctccagggaaggggctcgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcaggcagcaccagcggctccggcaagcctggctctggcgagggcagcacaaagggacaggcagtggtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggt

SEQ ID NO: 6 - DNA, 816 bp, synthetic construct
  misc_feature 1..816: note = Synthetic Polynucleotide
  source 1..816: mol_type = other DNA
atggccctgcctgtgacagctctgctcctccctctggccctgctgctccatgccgccagacccgaggtgcagctcgtggagtctgggggaggcttggtgcagcctggggggtctctgagactctcctgtgcagcctctggattcaccttcagtgtctactacatgaactgggtccgccaggctccagggaaggggcttgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcaggcagcaccagcggctccggcaagcctggctctggcgagggcagcacaaagggacaggcagtggtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggt

SEQ ID NO: 7 - DNA, 81 bp, synthetic construct
  misc_feature 1..81: note = Synthetic Polynucleotide
  source 1..81: mol_type = other DNA
ttttgggtgctggtggtggttggtggagtcctggcttgctatagcttgctagtaacagtggcctttattattttctgggtg

SEQ ID NO: 8 - DNA, 340 bp, synthetic construct
  misc_feature 1..340: note = Synthetic Polynucleotide
  source 1..340: mol_type = other DNA
cgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggac

SEQ ID NO: 9 - DNA, 126 bp, synthetic construct
  misc_feature 1..126: note = Synthetic Polynucleotide
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 10 - DNA, 72 bp, synthetic construct
  misc_feature 1..72: note = Synthetic Polynucleotide
  source 1..72: mol_type = other DNA
ctttgcgcacgcccacgccgcagccccgcccaagaagatggcaaagtctacatcaacatgccaggcaggggc

SEQ ID NO: 11 - DNA, 153 bp, synthetic construct
  misc_feature 1..153: note = Synthetic Polynucleotide
  source 1..153: mol_type = other DNA
agcgcgaacgaacgctgcaaaagcaaagtggtgccgtgccgccagaaacagtggcgcaccagctttgatagcaaaaaactggatctgaactataaccattttgaaagcatggaatggagccatcgcagccgccgcggccgcatttggggcatg

SEQ ID NO: 12 - DNA, 156 bp, synthetic construct
  misc_feature 1..156: note = Synthetic Polynucleotide
  source 1..156: mol_type = other DNA
tacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 13 - DNA, 57 bp, synthetic construct
  misc_feature 1..57: note = Synthetic Polynucleotide
  source 1..57: mol_type = other DNA
atggcacggccacatccctggtggctgtgcgttctggggaccctggtggggctctca

SEQ ID NO: 14 - DNA, 66 bp, synthetic construct
  misc_feature 1..66: note = Synthetic Polynucleotide
  source 1..66: mol_type = other DNA
atgcttctcctggtgacaagccttctgctctgtgagttaccacacccagcattcctcctgatccca

SEQ ID NO: 15 - DNA, 516 bp, synthetic construct
  misc_feature 1..516: note = Synthetic Polynucleotide
  source 1..516: mol_type = other DNA
gctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgc

SEQ ID NO: 16 - DNA, 516 bp, synthetic construct
  misc_feature 1..516: note = Synthetic Polynucleotide
  source 1..516: mol_type = other DNA
gctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgc

SEQ ID NO: 17 - DNA, 63 bp, synthetic construct
  misc_feature 1..63: note = Synthetic Polynucleotide
  source 1..63: mol_type = other DNA
atccttgtgatcttctctggaatgttccttgttttcaccctggccggggccctgttcctccat

SEQ ID NO: 18 - DNA, 654 bp, synthetic construct
  misc_feature 1..654: note = Synthetic Polynucleotide
  source 1..654: mol_type = other DNA
atggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcttttgggtgctggtggtggttggtggagtcctggcttgctatagcttgctagtaacagtggcctttattattttctgggtg

SEQ ID NO: 19 - DNA, 2004 bp, synthetic construct
  misc_feature 1..2004: note = Synthetic Polynucleotide
  source 1..2004: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 20 - DNA, 2013 bp, synthetic construct
  misc_feature 1..2013: note = Synthetic Polynucleotide
  source 1..2013: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 21 - DNA, 1950 bp, synthetic construct
  misc_feature 1..1950: note = Synthetic Polynucleotide
  source 1..1950: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcttttgggtgctggtggtggttggtggagtcctggcttgctatagcttgctagtaacagtggcctttattattttctgggtgctttgcgcacgcccacgccgcagccccgcccaagaagatggcaaagtctacatcaacatgccaggcaggggcaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 22 - DNA, 1959 bp, synthetic construct
  misc_feature 1..1959: note = Synthetic Polynucleotide
  source 1..1959: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcttttgggtgctggtggtggttggtggagtcctggcttgctatagcttgctagtaacagtggcctttattattttctgggtgctttgcgcacgcccacgccgcagccccgcccaagaagatggcaaagtctacatcaacatgccaggcaggggcaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 23 - DNA, 2034 bp, synthetic construct
  misc_feature 1..2034: note = Synthetic Polynucleotide
  source 1..2034: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcttttgggtgctggtggtggttggtggagtcctggcttgctatagcttgctagtaacagtggcctttattattttctgggtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaaaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 24 - DNA, 2043 bp, synthetic construct
  misc_feature 1..2043: note = Synthetic Polynucleotide
  source 1..2043: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcttttgggtgctggtggtggttggtggagtcctggcttgctatagcttgctagtaacagtggcctttattattttctgggtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaaaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 25 - DNA, 2031 bp, synthetic construct
  misc_feature 1..2031: note = Synthetic Polynucleotide
  source 1..2031: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcttttgggtgctggtggtggttggtggagtcctggcttgctatagcttgctagtaacagtggcctttattattttctgggtgagcgcgaacgaacgctgcaaaagcaaagtggtgccgtgccgccagaaacagtggcgcaccagctttgatagcaaaaaactggatctgaactataaccattttgaaagcatggaatggagccatcgcagccgccgcggccgcatttggggcatgaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 26 - DNA, 2040 bp, synthetic construct
  misc_feature 1..2040: note = Synthetic Polynucleotide
  source 1..2040: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcttttgggtgctggtggtggttggtggagtcctggcttgctatagcttgctagtaacagtggcctttattattttctgggtgagcgcgaacgaacgctgcaaaagcaaagtggtgccgtgccgccagaaacagtggcgcaccagctttgatagcaaaaaactggatctgaactataaccattttgaaagcatggaatggagccatcgcagccgccgcggccgcatttggggcatgaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 27 - DNA, 1986 bp, synthetic construct
  misc_feature 1..1986: note = Synthetic Polynucleotide
  source 1..1986: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccataaacggggcagaaagaaactcctgtatatattcaaacaaccatttatgagaccagtacaaactactcaagaggaagatggctgtagctgccgatttccagaagaagaagaaggaggatgtgaactgaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 28 - DNA, 1995 bp, synthetic construct
  misc_feature 1..1995: note = Synthetic Polynucleotide
  source 1..1995: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcatactggtcatcttttctggaatgttccttgtgttcaccctggcaggagccctgttccttcacaaacggggcagaaagaaactcctgtatatattcaaacaaccatttatgagaccagtacaaactactcaagaggaagatggctgtagctgccgatttccagaagaagaagaaggaggatgtgaactgaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 29 - DNA, 1860 bp, synthetic construct
  misc_feature 1..1860: note = Synthetic Polynucleotide
  source 1..1860: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccataaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 30 - DNA, 1866 bp, synthetic construct
  misc_feature 1..1866: note = Synthetic Polynucleotide
  source 1..1866: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcatactggtcatcttttctggaatgttccttgtgttcaccctggcaggagccctgttccttcaccgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 31 - DNA, 1980 bp, synthetic construct
  misc_feature 1..1980: note = Synthetic Polynucleotide
  source 1..1980: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccataggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 32 - DNA, 1989 bp, synthetic construct
  misc_feature 1..1989: note = Synthetic Polynucleotide
  source 1..1989: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcatactggtcatcttttctggaatgttccttgtgttcaccctggcaggagccctgttccttcacaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 33 - DNA, 1998 bp, synthetic construct
  misc_feature 1..1998: note = Synthetic Polynucleotide
  source 1..1998: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 34 - DNA, 2007 bp, synthetic construct
  misc_feature 1..2007: note = Synthetic Polynucleotide
  source 1..2007: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 35 - DNA, 1932 bp, synthetic construct
  misc_feature 1..1932: note = Synthetic Polynucleotide
  source 1..1932: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatactggtcatcttttctggaatgttccttgtgttcaccctggcaggagccctgttccttcacctttgcgcacgcccacgccgcagccccgcccaagaagatggcaaagtctacatcaacatgccaggcaggggcaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 36 - DNA, 1941 bp, synthetic construct
  misc_feature 1..1941: note = Synthetic Polynucleotide
  source 1..1941: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcatactggtcatcttttctggaatgttccttgtgttcaccctggcaggagccctgttccttcacctttgcgcacgcccacgccgcagccccgcccaagaagatggcaaagtctacatcaacatgccaggcaggggcaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 37 - DNA, 2016 bp, synthetic construct
  misc_feature 1..2016: note = Synthetic Polynucleotide
  source 1..2016: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccattacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaaaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 38 - DNA, 2025 bp, synthetic construct
  misc_feature 1..2025: note = Synthetic Polynucleotide
  source 1..2025: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcatactggtcatcttttctggaatgttccttgtgttcaccctggcaggagccctgttccttcactacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaaaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 39 - DNA, 2013 bp, synthetic construct
  misc_feature 1..2013: note = Synthetic Polynucleotide
  source 1..2013: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccatagcgcgaacgaacgctgcaaaagcaaagtggtgccgtgccgccagaaacagtggcgcaccagctttgatagcaaaaaactggatctgaactataaccattttgaaagcatggaatggagccatcgcagccgccgcggccgcatttggggcatgaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 40 - DNA, 2022 bp, synthetic construct
  misc_feature 1..2022: note = Synthetic Polynucleotide
  source 1..2022: mol_type = other DNA
atgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgaggatgcttctcctggtgacaagccttctgctctgtgagttaccacacccagcattcctcctgatcccagctacaccggctccgaagtcctgcccggagcggcattattgggcacagggcaagttgtgttgtcaaatgtgtgagccgggaacctttctcgtgaaggattgcgatcagcatcggaaggccgcgcagtgcgacccatgtataccaggggtctcattttccccagatcaccatacgaggccgcactgtgagtcttgcaggcattgtaattccggcttgttggtccgcaactgtactattactgcgaatgcagagtgtgcttgtagaaacggatggcagtgcagggacaaagaatgtacggagtgtgatccactgcctaaccccagtcttacagcaagatcttcacaggccctcagcccgcatcctcaaccaacacatcttccttacgtgtcagaaatgttggaggcgcgaaccgcaggccatatgcagaccctggcggactttcggcagctgccagcacgcacacttagtacacactggccaccacaacgcagcttgtgctcttccgatttcatccgcatactggtcatcttttctggaatgttccttgtgttcaccctggcaggagccctgttccttcacagcgcgaacgaacgctgcaaaagcaaagtggtgccgtgccgccagaaacagtggcgcaccagctttgatagcaaaaaactggatctgaactataaccattttgaaagcatggaatggagccatcgcagccgccgcggccgcatttggggcatgaaacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 41 - protein, 125 aa, Adenovirus
MAESLYAFIDSPGGIAPVQEGASNRYIFFCPESFHIPPHGVILLHLRVSVLVPTGYQGRFMALNDYHARGILTQSDVIFAGRRHDLSVLLFNHTDRFLYVREGHPVGTLLLERVIFPSVRIATLV

SEQ ID NO: 42 - protein, 181 aa, Vaccinia virus
MISLSFLIHNPLKKWKLKPSISINGYRSTFTMAFPCAQFRPCHCHATKDSLNTVADVRHCLTEYILWVSHRWTHRESAGSLYRLLISFRTDATELFGGELKDSLPWDNIDNCVEIIKCFIRNDSMKTAEELRAIIGLCTQSAIVSGRVFNDKYIDILLMLRKILNENDYLTLLDHIRTAKY

SEQ ID NO: 43 - protein, 186 aa, Dengue virus
SGVLWDTPSPPEVERAVLDNGIYRILQRGLLGRSQVGVGVFQEGVFHTMWHVTRGAVLMYQGKRLEPSWASVKKDLISYGGGWRFQGSWNTGEEVQVIAVEPGKNPKNVQTTPGTFKTPEGEIGAIALDFKPGTSGSPIVNREGKIVGLYGNGVVTTSGTYVSAIAQAKVSQEGPLPEIEDEVFRK

SEQ ID NO: 44 - protein, 272 aa, synthetic construct
  REGION 1..272: note = Synthetic Polypeptide
MALPVTALLLPLALLLHAARPQAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVLGGSTSGSGKPGSGEGSTKGEVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSS

SEQ ID NO: 45 - protein, 274 aa, synthetic construct
  REGION 1..274: note = Synthetic Polypeptide
MGMALPVTALLLPLALLLHAARPEVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSSGSTSGSGKPGSGEGSTKGQAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVLG

SEQ ID NO: 46 - protein, 274 aa, synthetic construct; identical to SEQ ID NO: 45
  REGION 1..274: note = Synthetic Polypeptide

SEQ ID NO: 47 - protein, 27 aa, synthetic construct
  REGION 1..27: note = Synthetic Polypeptide
FWVLVVVGGVLACYSLLVTVAFIIFWV

SEQ ID NO: 48 - protein, 66 aa, synthetic construct
  REGION 1..66: note = Synthetic Polypeptide
TTTPAPRPPTPAPTIASQPLSLRPEACRPAAGGAVHTRGLDFACDIYIWAPLAGTCGVLLLSLVIT

SEQ ID NO: 49 - protein, 27 aa, synthetic construct
  REGION 1..27: note = Synthetic Polypeptide
IISFFLALTSTALLFLLFFLTLRFSVV

SEQ ID NO: 50 - protein, 21 aa, synthetic construct
  REGION 1..21: note = Synthetic Polypeptide
LLAGLVAADAVASLLIVGAVF

SEQ ID NO: 51 - protein, 21 aa, synthetic construct
  REGION 1..21: note = Synthetic Polypeptide
GVLAGIVMGDLVLTVLIALAV

SEQ ID NO: 52 - protein, 21 aa, synthetic construct
  REGION 1..21: note = Synthetic Polypeptide
AVMIIFRIGMAVAIFCCFFFP

SEQ ID NO: 53 - protein, 114 aa, synthetic construct
  REGION 1..114: note = Synthetic Polypeptide
TRKKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 54 - protein, 114 aa, synthetic construct
  REGION 1..114: note = Synthetic Polypeptide
KRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 55 - protein, 113 aa, synthetic construct
  REGION 1..113: note = Synthetic Polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 56 - protein, 42 aa, synthetic construct
  REGION 1..42: note = Synthetic Polypeptide
KRGRKKLLYIFKQPFMRPVQTTQEEDGCSCRFPEEEEGGCEL

SEQ ID NO: 57 - protein, 24 aa, synthetic construct
  REGION 1..24: note = Synthetic Polypeptide
LCARPRRSPAQEDGKVYINMPGRG

SEQ ID NO: 58 - protein, 52 aa, synthetic construct
  REGION 1..52: note = Synthetic Polypeptide
YFLGRLVPRGRGAAEAATRKQRITETESPYQELQGQRSDVYSDLNTQRPYYK

SEQ ID NO: 59 - protein, 51 aa, synthetic construct
  REGION 1..51: note = Synthetic Polypeptide
SANERCKSKVVPCRQKQWRTSFDSKKLDLNYNHFESMEWSHRSRRGRIWGM

SEQ ID NO: 60 - protein, 227 aa, synthetic construct
  REGION 1..227: note = Synthetic Polypeptide
TVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEAL

SEQ ID NO: 61 - protein, 39 aa, synthetic construct
  REGION 1..39: note = Synthetic Polypeptide
IEVMYPPPYLDNEKSNGTIIHVKGKHLCPSPLFPGPSKP

SEQ ID NO: 62 - protein, 160 aa, synthetic construct
  REGION 1..160: note = Synthetic Polypeptide
ISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 63 - protein, 20 aa, synthetic construct
  REGION 1..20: note = Synthetic Polypeptide
QCTNYALLKLAGDVESNPGP

SEQ ID NO: 64 - protein, 18 aa, synthetic construct
  REGION 1..18: note = Synthetic Polypeptide
EGRGSLLTCGDVEENPGP

SEQ ID NO: 65 - protein, 19 aa, synthetic construct
  REGION 1..19: note = Synthetic Polypeptide
ATNFSLLKQAGDVEENPGP

SEQ ID NO: 66 - protein, 22 aa, synthetic construct
  REGION 1..22: note = Synthetic Polypeptide
VKQTLNFDLLKLAGDVESNPGP

SEQ ID NO: 67 - protein, 19 aa, synthetic construct
  REGION 1..19: note = Synthetic Polypeptide
MARPHPWWLCVLGTLVGLS

SEQ ID NO: 68 - protein, 22 aa, synthetic construct
  REGION 1..22: note = Synthetic Polypeptide
MLLLVTSLLLCELPHPAFLLIP

SEQ ID NO: 69 - protein, 172 aa, synthetic construct
  REGION 1..172: note = Synthetic Polypeptide
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 70 - protein, 21 aa, synthetic construct
  REGION 1..21: note = Synthetic Polypeptide
ILVIFSGMFLVFTLAGALFLH

SEQ ID NO: 71 - protein, 218 aa, synthetic construct
  REGION 1..218: note = Synthetic Polypeptide
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWV

SEQ ID NO: 72 - protein, 667 aa, synthetic construct
  REGION 1..667: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVKRGRKKLLYIFKQPFMRPVQTTQEEDGCSCRFPEEEEGGCELKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 73 - protein, 670 aa, synthetic construct
  REGION 1..670: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVKRGRKKLLYIFKQPFMRPVQTTQEEDGCSCRFPEEEEGGCELKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 74 - protein, 649 aa, synthetic construct
  REGION 1..649: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVLCARPRRSPAQEDGKVYINMPGRGKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - protein, 652 aa, synthetic construct
  REGION 1..652: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVLCARPRRSPAQEDGKVYINMPGRGKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 76 - protein, 677 aa, synthetic construct
  REGION 1..677: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVYFLGRLVPRGRGAAEAATRKQRITETESPYQELQGQRSDVYSDLNTQRPYYKKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 77 - protein, 680 aa, synthetic construct
  REGION 1..680: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVYFLGRLVPRGRGAAEAATRKQRITETESPYQELQGQRSDVYSDLNTQRPYYKKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 78 - protein, 676 aa, synthetic construct
  REGION 1..676: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVSANERCKSKVVPCRQKQWRTSFDSKKLDLNYNHFESMEWSHRSRRGRIWGMKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 79 - protein, 679 aa, synthetic construct
  REGION 1..679: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVSANERCKSKVVPCRQKQWRTSFDSKKLDLNYNHFESMEWSHRSRRGRIWGMKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 80 - protein, 661 aa, synthetic construct
  REGION 1..661: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHKRGRKKLLYIFKQPFMRPVQTTQEEDGCSCRFPEEEEGGCELKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 81 - protein, 664 aa, synthetic construct
  REGION 1..664: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHKRGRKKLLYIFKQPFMRPVQTTQEEDGCSCRFPEEEEGGCELKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 82 - protein, 619 aa, synthetic construct
  REGION 1..619: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 83 - protein, 622 aa, synthetic construct
  REGION 1..622: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 84 - protein, 659 aa, synthetic construct
  REGION 1..659: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 85 - protein, 662 aa, synthetic construct
  REGION 1..662: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 86 - protein, 665 aa, synthetic construct
  REGION 1..665: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 87 - protein, 668 aa, synthetic construct
  REGION 1..668: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 88 - protein, 643 aa, synthetic construct
  REGION 1..643: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHLCARPRRSPAQEDGKVYINMPGRGKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 89 - protein, 646 aa, synthetic construct
  REGION 1..646: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHLCARPRRSPAQEDGKVYINMPGRGKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 90 - protein, 671 aa, synthetic construct
  REGION 1..671: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHYFLGRLVPRGRGAAEAATRKQRITETESPYQELQGQRSDVYSDLNTQRPYYKKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 91 - protein, 674 aa, synthetic construct
  REGION 1..674: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHYFLGRLVPRGRGAAEAATRKQRITETESPYQELQGQRSDVYSDLNTQRPYYKKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 92 - protein, 670 aa, synthetic construct
  REGION 1..670: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHSANERCKSKVVPCRQKQWRTSFDSKKLDLNYNHFESMEWSHRSRRGRIWGMKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 93 - protein, 673 aa, synthetic construct
  REGION 1..673: note = Synthetic Polypeptide
MTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLPCSRMLLLVTSLLLCELPHPAFLLIPATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHSANERCKSKVVPCRQKQWRTSFDSKKLDLNYNHFESMEWSHRSRRGRIWGMKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 94 - protein, 75 aa, synthetic construct
  REGION 1..75: note = Synthetic Polypeptide
KPTTTPAPRPPTPAPTIASQPLSLRPEACRPAAGGAVHTRGLDFACDIYIWAPLAGTCGVLLLSLVITLYCNHRN

SEQ ID NO: 95 - DNA, 2652 bp, synthetic construct
  misc_feature 1..2652: note = Synthetic Polynucleotide
  source 1..2652: mol_type = other DNA
atggccctgcctgtgacagctctgctcctccctctggccctgctgctccatgccgccagaccccaggcagttgtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtggcagcaccagcggctccggcaagcctggctctggcgagggcagcacaaagggagaggtgcagctcgtggagtctgggggaggcttggtgcagcctggggggtctctgagactctcctgtgcagcctctggattcaccttcagtgtctactacatgaactgggtccgccaggctccagggaaggggctcgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcacgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 96 - DNA, 2652 bp, synthetic construct
  misc_feature 1..2652: note = Synthetic Polynucleotide
  source 1..2652: mol_type = other DNA
atggccctgcctgtgacagctctgctcctccctctggccctgctgctccatgccgccagacccgaggtgcagctcgtggagtctgggggaggcttggtgcagcctggggggtctctgagactctcctgtgcagcctctggattcaccttcagtgtctactacatgaactgggtccgccaggctccagggaaggggctcgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcaggcagcaccagcggctccggcaagcctggctctggcgagggcagcacaaagggacaggcagtggtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtcgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 97 - DNA, 2652 bp, synthetic construct
  misc_feature 1..2652: note = Synthetic Polynucleotide
  source 1..2652: mol_type = other DNA
atggccctgcctgtgacagctctgctcctccctctggccctgctgctccatgccgccagaccccaggcagttgtgacccaggagccttccctgacagtgtctccaggagggacggtcacgctcacctgcggcctcaaatctgggtctgtcacttccgataacttccccacttggtaccagcagacaccaggccaggctccccgattgcttatctacaacacaaacacccgtcactctggcgtccccgaccgcttctccggatccatcctgggcaacaaagccgccctcaccatcacgggggcccaggccgacgacgaggccgaatatttctgtgctctgttcataagtaatcctagtgttgagttcggcggagggacccaactgaccgtcctaggtggcagcaccagcggctccggcaagcctggctctggcgagggcagcacaaagggagaggtgcagctcgtggagtctgggggaggcttggtgcagcctggggggtctctgagactctcctgtgcagcctctggattcaccttcagtgtctactacatgaactgggtccgccaggctccagggaaggggcttgagtgggtctcagatattaataatgaaggtggtactacatactatgcagactccgtgaagggccgattcaccatctccagagacaactctaagaacagcctgtatctgcaaatgaacagcctgcgcgccgaggacacggccgtgtactactgcgcgagagatgccggatatagcaaccatgtacccatctttgattcttggggccaggggaccctggtcactgtctcctcacgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga